SEQUENCE LISTING (2340 sequences)

SEQ ID NO: 1 - DNA, 19 bp, Artificial Sequence
  Synthetic Polynucleotide
tctccaagtatgatcgtct

SEQ ID NO: 2 - DNA, 19 bp, Artificial Sequence
  Synthetic Polynucleotide
tgcagaacaagatcgtcca

SEQ ID NO: 3 - DNA, 19 bp, Artificial Sequence
  Synthetic Polynucleotide
ccacttttctgccaatgcc

SEQ ID NO: 4 - DNA, 19 bp, Artificial Sequence
  Synthetic Polynucleotide
acggctcccagtgcgggtt

SEQ ID NO: 5 - DNA, 19 bp, Artificial Sequence
  Synthetic Polynucleotide
cacggagattggcattgag

SEQ ID NO: 6 - DNA, 19 bp, Artificial Sequence
  Synthetic Polynucleotide
acggagattggcattgaga

SEQ ID NO: 7 - DNA, 19 bp, Artificial Sequence
  Synthetic Polynucleotide
agagaagatgacattgcca

SEQ ID NO: 8 - DNA, 19 bp, Artificial Sequence
  Synthetic Polynucleotide
ccagtggcatgagagtttt

SEQ ID NO: 9 - DNA, 19 bp, Artificial Sequence
  Synthetic Polynucleotide
tgcaggcctctggtgaggc

SEQ ID NO: 10 - DNA, 19 bp, Artificial Sequence
  Synthetic Polynucleotide
tggtgaggccgtgtactgt

SEQ ID NO: 11 - DNA, 19 bp, Artificial Sequence
  Synthetic Polynucleotide
cgggcccacgccaagatca

SEQ ID NO: 12 - DNA, 19 bp, Artificial Sequence
  Synthetic Polynucleotide
ccaagatcaagtccataga

SEQ ID NO: 13 - DNA, 19 bp, Artificial Sequence
  Synthetic Polynucleotide
gttgggcatatcattggtg

SEQ ID NO: 14 - DNA, 19 bp, Artificial Sequence
  Synthetic Polynucleotide
cattggtgctgtggttgct

SEQ ID NO: 15 - DNA, 19 bp, Artificial Sequence
  Synthetic Polynucleotide
aaaccggattgtggttcga

SEQ ID NO: 16 - DNA, 19 bp, Artificial Sequence
  Synthetic Polynucleotide
ccggattgtggttcgagtg

SEQ ID NO: 17 - DNA, 19 bp, Artificial Sequence
  Synthetic Polynucleotide
gattgtggttcgagtgaag

SEQ ID NO: 18 - DNA, 19 bp, Artificial Sequence
  Synthetic Polynucleotide
gctggaccgtgatgaggac

SEQ ID NO: 19 - DNA, 19 bp, Artificial Sequence
  Synthetic Polynucleotide
tgaggacatgctgataact

SEQ ID NO: 20 - DNA, 19 bp, Artificial Sequence
  Synthetic Polynucleotide
cagatacaaggttggcttc

SEQ ID NO: 21 - DNA, 19 bp, Artificial Sequence
  Synthetic Polynucleotide
ctctcagagtattatggaa

SEQ ID NO: 22 - DNA, 19 bp, Artificial Sequence
  Synthetic Polynucleotide
aaccttccctccaacacgg

SEQ ID NO: 23 - DNA, 19 bp, Artificial Sequence
  Synthetic Polynucleotide
ctagcaagctctcagtatc

SEQ ID NO: 24 - DNA, 19 bp, Artificial Sequence
  Synthetic Polynucleotide
gcaagctctcagtatcatg

SEQ ID NO: 25 - DNA, 19 bp, Artificial Sequence
  Synthetic Polynucleotide
tcagtatcatgctcggaag

SEQ ID NO: 26 - DNA, 19 bp, Artificial Sequence
  Synthetic Polynucleotide
ccaccaagtttggaataag

SEQ ID NO: 27 - DNA, 19 bp, Artificial Sequence
  Synthetic Polynucleotide
tccgcacagatattgtcat

SEQ ID NO: 28 - DNA, 19 bp, Artificial Sequence
  Synthetic Polynucleotide
caagatcccggcatttggc

SEQ ID NO: 29 - DNA, 19 bp, Artificial Sequence
  Synthetic Polynucleotide
agcacacaggtaataacgt

SEQ ID NO: 30 - DNA, 19 bp, Artificial Sequence
  Synthetic Polynucleotide
accctggtctgtgagggtc

SEQ ID NO: 31 - DNA, 19 bp, Artificial Sequence
  Synthetic Polynucleotide
ccacagaacatggatctat

SEQ ID NO: 32 - DNA, 19 bp, Artificial Sequence
  Synthetic Polynucleotide
acatggatctattaaagtc

SEQ ID NO: 33 - DNA, 19 bp, Artificial Sequence
  Synthetic Polynucleotide
atggatctattaaagtcac

SEQ ID NO: 34 - DNA, 19 bp, Artificial Sequence
  Synthetic Polynucleotide
ggaagggtttgtgctattc

SEQ ID NO: 35 - DNA, 19 bp, Artificial Sequence
  Synthetic Polynucleotide
gaagggtttgtgctattcc

SEQ ID NO: 36 - DNA, 19 bp, Artificial Sequence
  Synthetic Polynucleotide
ctcaagttctgatggtgtc

SEQ ID NO: 37 - DNA, 19 bp, Artificial Sequence
  Synthetic Polynucleotide
tcaagttctgatggtgtct

SEQ ID NO: 38 - DNA, 19 bp, Artificial Sequence
  Synthetic Polynucleotide
cccacaaacctctagaagc

SEQ ID NO: 39 - DNA, 19 bp, Artificial Sequence
  Synthetic Polynucleotide
cacaaacctctagaagctt

SEQ ID NO: 40 - DNA, 19 bp, Artificial Sequence
  Synthetic Polynucleotide
ccttcaaaccaatgaacag

SEQ ID NO: 41 - DNA, 19 bp, Artificial Sequence
  Synthetic Polynucleotide
aatgaacagcaaagcataa

SEQ ID NO: 42 - DNA, 19 bp, Artificial Sequence
  Synthetic Polynucleotide
aacaaattcaaggttaatt

SEQ ID NO: 43 - DNA, 19 bp, Artificial Sequence
  Synthetic Polynucleotide
aagctgcataaagcaagat

SEQ ID NO: 44 - DNA, 19 bp, Artificial Sequence
  Synthetic Polynucleotide
tgcataaagcaagattact

SEQ ID NO: 45 - DNA, 19 bp, Artificial Sequence
  Synthetic Polynucleotide
aaagattatgtttaatctt

SEQ ID NO: 46 - DNA, 19 bp, Artificial Sequence
  Synthetic Polynucleotide
aaggcttggttttcttact

SEQ ID NO: 47 - DNA, 19 bp, Artificial Sequence
  Synthetic Polynucleotide
ggttttcttactgtcatat

SEQ ID NO: 48 - DNA, 19 bp, Artificial Sequence
  Synthetic Polynucleotide
tcaagtttctgcggttggt

SEQ ID NO: 49 - DNA, 19 bp, Artificial Sequence
  Synthetic Polynucleotide
caagtttctgcggttggta

SEQ ID NO: 50 - DNA, 19 bp, Artificial Sequence
  Synthetic Polynucleotide
ttggactggtgcttgatgt

SEQ ID NO: 51 - DNA, 21 bp, Artificial Sequence
  Synthetic Polynucleotide
gctctccaagtatgatcgtct

SEQ ID NO: 52 - DNA, 21 bp, Artificial Sequence
  Synthetic Polynucleotide
tctgcagaacaagatcgtcca

SEQ ID NO: 53 - DNA, 21 bp, Artificial Sequence
  Synthetic Polynucleotide
gtccacttttctgccaatgcc

SEQ ID NO: 54 - DNA, 21 bp, Artificial Sequence
  Synthetic Polynucleotide
ccacggctcccagtgcgggtt

SEQ ID NO: 55 - DNA, 21 bp, Artificial Sequence
  Synthetic Polynucleotide
aacacggagattggcattgag

SEQ ID NO: 56 - DNA, 21 bp, Artificial Sequence
  Synthetic Polynucleotide
acacggagattggcattgaga

SEQ ID NO: 57 - DNA, 21 bp, Artificial Sequence
  Synthetic Polynucleotide
ggagagaagatgacattgcca

SEQ ID NO: 58 - DNA, 21 bp, Artificial Sequence
  Synthetic Polynucleotide
aaccagtggcatgagagtttt

SEQ ID NO: 59 - DNA, 21 bp, Artificial Sequence
  Synthetic Polynucleotide
catgcaggcctctggtgaggc

SEQ ID NO: 60 - DNA, 21 bp, Artificial Sequence
  Synthetic Polynucleotide
tctggtgaggccgtgtactgt

SEQ ID NO: 61 - DNA, 21 bp, Artificial Sequence
  Synthetic Polynucleotide
cccgggcccacgccaagatca

SEQ ID NO: 62 - DNA, 21 bp, Artificial Sequence
  Synthetic Polynucleotide
cgccaagatcaagtccataga

SEQ ID NO: 63 - DNA, 21 bp, Artificial Sequence
  Synthetic Polynucleotide
gtgttgggcatatcattggtg

SEQ ID NO: 64 - DNA, 21 bp, Artificial Sequence
  Synthetic Polynucleotide
atcattggtgctgtggttgct

SEQ ID NO: 65 - DNA, 21 bp, Artificial Sequence
  Synthetic Polynucleotide
gcaaaccggattgtggttcga

SEQ ID NO: 66 - DNA, 21 bp, Artificial Sequence
  Synthetic Polynucleotide
aaccggattgtggttcgagtg

SEQ ID NO: 67 - DNA, 21 bp, Artificial Sequence
  Synthetic Polynucleotide
cggattgtggttcgagtgaag

SEQ ID NO: 68 - DNA, 21 bp, Artificial Sequence
  Synthetic Polynucleotide
atgctggaccgtgatgaggac

SEQ ID NO: 69 - DNA, 21 bp, Artificial Sequence
  Synthetic Polynucleotide
gatgaggacatgctgataact

SEQ ID NO: 70 - DNA, 21 bp, Artificial Sequence
  Synthetic Polynucleotide
gccagatacaaggttggcttc

SEQ ID NO: 71 - DNA, 21 bp, Artificial Sequence
  Synthetic Polynucleotide
ctctctcagagtattatggaa

SEQ ID NO: 72 - DNA, 21 bp, Artificial Sequence
  Synthetic Polynucleotide
ccaaccttccctccaacacgg

SEQ ID NO: 73 - DNA, 21 bp, Artificial Sequence
  Synthetic Polynucleotide
gcctagcaagctctcagtatc

SEQ ID NO: 74 - DNA, 21 bp, Artificial Sequence
  Synthetic Polynucleotide
tagcaagctctcagtatcatg

SEQ ID NO: 75 - DNA, 21 bp, Artificial Sequence
  Synthetic Polynucleotide
tctcagtatcatgctcggaag

SEQ ID NO: 76 - DNA, 21 bp, Artificial Sequence
  Synthetic Polynucleotide
tcccaccaagtttggaataag

SEQ ID NO: 77 - DNA, 21 bp, Artificial Sequence
  Synthetic Polynucleotide
cctccgcacagatattgtcat

SEQ ID NO: 78 - DNA, 21 bp, Artificial Sequence
  Synthetic Polynucleotide
tacaagatcccggcatttggc

SEQ ID NO: 79 - DNA, 21 bp, Artificial Sequence
  Synthetic Polynucleotide
tcagcacacaggtaataacgt

SEQ ID NO: 80 - DNA, 21 bp, Artificial Sequence
  Synthetic Polynucleotide
aaaccctggtctgtgagggtc

SEQ ID NO: 81 - DNA, 21 bp, Artificial Sequence
  Synthetic Polynucleotide
taccacagaacatggatctat

SEQ ID NO: 82 - DNA, 21 bp, Artificial Sequence
  Synthetic Polynucleotide
gaacatggatctattaaagtc

SEQ ID NO: 83 - DNA, 21 bp, Artificial Sequence
  Synthetic Polynucleotide
acatggatctattaaagtcac

SEQ ID NO: 84 - DNA, 21 bp, Artificial Sequence
  Synthetic Polynucleotide
cgggaagggtttgtgctattc

SEQ ID NO: 85 - DNA, 21 bp, Artificial Sequence
  Synthetic Polynucleotide
gggaagggtttgtgctattcc

SEQ ID NO: 86 - DNA, 21 bp, Artificial Sequence
  Synthetic Polynucleotide
acctcaagttctgatggtgtc

SEQ ID NO: 87 - DNA, 21 bp, Artificial Sequence
  Synthetic Polynucleotide
cctcaagttctgatggtgtct

SEQ ID NO: 88 - DNA, 21 bp, Artificial Sequence
  Synthetic Polynucleotide
ttcccacaaacctctagaagc

SEQ ID NO: 89 - DNA, 21 bp, Artificial Sequence
  Synthetic Polynucleotide
cccacaaacctctagaagctt

SEQ ID NO: 90 - DNA, 21 bp, Artificial Sequence
  Synthetic Polynucleotide
ggccttcaaaccaatgaacag

SEQ ID NO: 91 - DNA, 21 bp, Artificial Sequence
  Synthetic Polynucleotide
ccaatgaacagcaaagcataa

SEQ ID NO: 92 - DNA, 21 bp, Artificial Sequence
  Synthetic Polynucleotide
gaaacaaattcaaggttaatt

SEQ ID NO: 93 - DNA, 21 bp, Artificial Sequence
  Synthetic Polynucleotide
tgaagctgcataaagcaagat

SEQ ID NO: 94 - DNA, 21 bp, Artificial Sequence
  Synthetic Polynucleotide
gctgcataaagcaagattact

SEQ ID NO: 95 - DNA, 21 bp, Artificial Sequence
  Synthetic Polynucleotide
aaaaagattatgtttaatctt

SEQ ID NO: 96 - DNA, 21 bp, Artificial Sequence
  Synthetic Polynucleotide
ctaaggcttggttttcttact

SEQ ID NO: 97 - DNA, 21 bp, Artificial Sequence
  Synthetic Polynucleotide
ttggttttcttactgtcatat

SEQ ID NO: 98 - DNA, 21 bp, Artificial Sequence
  Synthetic Polynucleotide
cctcaagtttctgcggttggt

SEQ ID NO: 99 - DNA, 21 bp, Artificial Sequence
  Synthetic Polynucleotide
ctcaagtttctgcggttggta

SEQ ID NO: 100 - DNA, 21 bp, Artificial Sequence
  Synthetic Polynucleotide
agttggactggtgcttgatgt

SEQ ID NO: 101 - DNA, 19 bp, Artificial Sequence
  Synthetic Polynucleotide
agacgatcatacttggaga

SEQ ID NO: 102 - DNA, 19 bp, Artificial Sequence
  Synthetic Polynucleotide
tggacgatcttgttctgca

SEQ ID NO: 103 - DNA, 19 bp, Artificial Sequence
  Synthetic Polynucleotide
ggcattggcagaaaagtgg

SEQ ID NO: 104 - DNA, 19 bp, Artificial Sequence
  Synthetic Polynucleotide
aacccgcactgggagccgt

SEQ ID NO: 105 - DNA, 19 bp, Artificial Sequence
  Synthetic Polynucleotide
ctcaatgccaatctccgtg

SEQ ID NO: 106 - DNA, 19 bp, Artificial Sequence
  Synthetic Polynucleotide
tctcaatgccaatctccgt

SEQ ID NO: 107 - DNA, 19 bp, Artificial Sequence
  Synthetic Polynucleotide
tggcaatgtcatcttctct

SEQ ID NO: 108 - DNA, 19 bp, Artificial Sequence
  Synthetic Polynucleotide
aaaactctcatgccactgg

SEQ ID NO: 109 - DNA, 19 bp, Artificial Sequence
  Synthetic Polynucleotide
gcctcaccagaggcctgca

SEQ ID NO: 110 - DNA, 19 bp, Artificial Sequence
  Synthetic Polynucleotide
acagtacacggcctcacca

SEQ ID NO: 111 - DNA, 19 bp, Artificial Sequence
  Synthetic Polynucleotide
tgatcttggcgtgggcccg

SEQ ID NO: 112 - DNA, 19 bp, Artificial Sequence
  Synthetic Polynucleotide
tctatggacttgatcttgg

SEQ ID NO: 113 - DNA, 19 bp, Artificial Sequence
  Synthetic Polynucleotide
caccaatgatatgcccaac

SEQ ID NO: 114 - DNA, 19 bp, Artificial Sequence
  Synthetic Polynucleotide
agcaaccacagcaccaatg

SEQ ID NO: 115 - DNA, 19 bp, Artificial Sequence
  Synthetic Polynucleotide
tcgaaccacaatccggttt

SEQ ID NO: 116 - DNA, 19 bp, Artificial Sequence
  Synthetic Polynucleotide
cactcgaaccacaatccgg

SEQ ID NO: 117 - DNA, 19 bp, Artificial Sequence
  Synthetic Polynucleotide
cttcactcgaaccacaatc

SEQ ID NO: 118 - DNA, 19 bp, Artificial Sequence
  Synthetic Polynucleotide
gtcctcatcacggtccagc

SEQ ID NO: 119 - DNA, 19 bp, Artificial Sequence
  Synthetic Polynucleotide
agttatcagcatgtcctca

SEQ ID NO: 120 - DNA, 19 bp, Artificial Sequence
  Synthetic Polynucleotide
gaagccaaccttgtatctg

SEQ ID NO: 121 - DNA, 19 bp, Artificial Sequence
  Synthetic Polynucleotide
ttccataatactctgagag

SEQ ID NO: 122 - DNA, 19 bp, Artificial Sequence
  Synthetic Polynucleotide
ccgtgttggagggaaggtt

SEQ ID NO: 123 - DNA, 19 bp, Artificial Sequence
  Synthetic Polynucleotide
gatactgagagcttgctag

SEQ ID NO: 124 - DNA, 19 bp, Artificial Sequence
  Synthetic Polynucleotide
catgatactgagagcttgc

SEQ ID NO: 125 - DNA, 19 bp, Artificial Sequence
  Synthetic Polynucleotide
cttccgagcatgatactga

SEQ ID NO: 126 - DNA, 19 bp, Artificial Sequence
  Synthetic Polynucleotide
cttattccaaacttggtgg

SEQ ID NO: 127 - DNA, 19 bp, Artificial Sequence
  Synthetic Polynucleotide
atgacaatatctgtgcgga

SEQ ID NO: 128 - DNA, 19 bp, Artificial Sequence
  Synthetic Polynucleotide
gccaaatgccgggatcttg

SEQ ID NO: 129 - DNA, 19 bp, Artificial Sequence
  Synthetic Polynucleotide
acgttattacctgtgtgct

SEQ ID NO: 130 - DNA, 19 bp, Artificial Sequence
  Synthetic Polynucleotide
gaccctcacagaccagggt

SEQ ID NO: 131 - DNA, 19 bp, Artificial Sequence
  Synthetic Polynucleotide
atagatccatgttctgtgg

SEQ ID NO: 132 - DNA, 19 bp, Artificial Sequence
  Synthetic Polynucleotide
gactttaatagatccatgt

SEQ ID NO: 133 - DNA, 19 bp, Artificial Sequence
  Synthetic Polynucleotide
gtgactttaatagatccat

SEQ ID NO: 134 - DNA, 19 bp, Artificial Sequence
  Synthetic Polynucleotide
gaatagcacaaacccttcc

SEQ ID NO: 135 - DNA, 19 bp, Artificial Sequence
  Synthetic Polynucleotide
ggaatagcacaaacccttc

SEQ ID NO: 136 - DNA, 19 bp, Artificial Sequence
  Synthetic Polynucleotide
gacaccatcagaacttgag

SEQ ID NO: 137 - DNA, 19 bp, Artificial Sequence
  Synthetic Polynucleotide
agacaccatcagaacttga

SEQ ID NO: 138 - DNA, 19 bp, Artificial Sequence
  Synthetic Polynucleotide
gcttctagaggtttgtggg

SEQ ID NO: 139 - DNA, 19 bp, Artificial Sequence
  Synthetic Polynucleotide
aagcttctagaggtttgtg

SEQ ID NO: 140 - DNA, 19 bp, Artificial Sequence
  Synthetic Polynucleotide
ctgttcattggtttgaagg

SEQ ID NO: 141 - DNA, 19 bp, Artificial Sequence
  Synthetic Polynucleotide
ttatgctttgctgttcatt

SEQ ID NO: 142 - DNA, 19 bp, Artificial Sequence
  Synthetic Polynucleotide
aattaaccttgaatttgtt

SEQ ID NO: 143 - DNA, 19 bp, Artificial Sequence
  Synthetic Polynucleotide
atcttgctttatgcagctt

SEQ ID NO: 144 - DNA, 19 bp, Artificial Sequence
  Synthetic Polynucleotide
agtaatcttgctttatgca

SEQ ID NO: 145 - DNA, 19 bp, Artificial Sequence
  Synthetic Polynucleotide
aagattaaacataatcttt

SEQ ID NO: 146 - DNA, 19 bp, Artificial Sequence
  Synthetic Polynucleotide
agtaagaaaaccaagcctt

SEQ ID NO: 147 - DNA, 19 bp, Artificial Sequence
  Synthetic Polynucleotide
atatgacagtaagaaaacc

SEQ ID NO: 148 - DNA, 19 bp, Artificial Sequence
  Synthetic Polynucleotide
accaaccgcagaaacttga

SEQ ID NO: 149 - DNA, 19 bp, Artificial Sequence
  Synthetic Polynucleotide
taccaaccgcagaaacttg

SEQ ID NO: 150 - DNA, 19 bp, Artificial Sequence
  Synthetic Polynucleotide
acatcaagcaccagtccaa

SEQ ID NO: 151 - DNA, 23 bp, Artificial Sequence
  Synthetic Polynucleotide
agacgatcatacttggagagcat

SEQ ID NO: 152 - DNA, 23 bp, Artificial Sequence
  Synthetic Polynucleotide
tggacgatcttgttctgcagacg

SEQ ID NO: 153 - DNA, 23 bp, Artificial Sequence
  Synthetic Polynucleotide
ggcattggcagaaaagtggacga

SEQ ID NO: 154 - DNA, 23 bp, Artificial Sequence
  Synthetic Polynucleotide
aacccgcactgggagccgtggct

SEQ ID NO: 155 - DNA, 23 bp, Artificial Sequence
  Synthetic Polynucleotide
ctcaatgccaatctccgtgttcc

SEQ ID NO: 156 - DNA, 23 bp, Artificial Sequence
  Synthetic Polynucleotide
tctcaatgccaatctccgtgttc

SEQ ID NO: 157 - DNA, 23 bp, Artificial Sequence
  Synthetic Polynucleotide
tggcaatgtcatcttctctccgg

SEQ ID NO: 158 - DNA, 23 bp, Artificial Sequence
  Synthetic Polynucleotide
aaaactctcatgccactggttac

SEQ ID NO: 159 - DNA, 23 bp, Artificial Sequence
  Synthetic Polynucleotide
gcctcaccagaggcctgcatgtc

SEQ ID NO: 160 - DNA, 23 bp, Artificial Sequence
  Synthetic Polynucleotide
acagtacacggcctcaccagagg

SEQ ID NO: 161 - DNA, 23 bp, Artificial Sequence
  Synthetic Polynucleotide
tgatcttggcgtgggcccgggtg

SEQ ID NO: 162 - DNA, 23 bp, Artificial Sequence
  Synthetic Polynucleotide
tctatggacttgatcttggcgtg

SEQ ID NO: 163 - DNA, 23 bp, Artificial Sequence
  Synthetic Polynucleotide
caccaatgatatgcccaacacaa

SEQ ID NO: 164 - DNA, 23 bp, Artificial Sequence
  Synthetic Polynucleotide
agcaaccacagcaccaatgatat

SEQ ID NO: 165 - DNA, 23 bp, Artificial Sequence
  Synthetic Polynucleotide
tcgaaccacaatccggtttgctg

SEQ ID NO: 166 - DNA, 23 bp, Artificial Sequence
  Synthetic Polynucleotide
cactcgaaccacaatccggtttg

SEQ ID NO: 167 - DNA, 23 bp, Artificial Sequence
  Synthetic Polynucleotide
cttcactcgaaccacaatccggt

SEQ ID NO: 168 - DNA, 23 bp, Artificial Sequence
  Synthetic Polynucleotide
gtcctcatcacggtccagcatgc

SEQ ID NO: 169 - DNA, 23 bp, Artificial Sequence
  Synthetic Polynucleotide
agttatcagcatgtcctcatcac

SEQ ID NO: 170 - DNA, 23 bp, Artificial Sequence
  Synthetic Polynucleotide
gaagccaaccttgtatctggcca

SEQ ID NO: 171 - DNA, 23 bp, Artificial Sequence
  Synthetic Polynucleotide
ttccataatactctgagagagat

SEQ ID NO: 172 - DNA, 23 bp, Artificial Sequence
  Synthetic Polynucleotide
ccgtgttggagggaaggttggtt

SEQ ID NO: 173 - DNA, 23 bp, Artificial Sequence
  Synthetic Polynucleotide
gatactgagagcttgctaggcat

SEQ ID NO: 174 - DNA, 23 bp, Artificial Sequence
  Synthetic Polynucleotide
catgatactgagagcttgctagg

SEQ ID NO: 175 - DNA, 23 bp, Artificial Sequence
  Synthetic Polynucleotide
cttccgagcatgatactgagagc

SEQ ID NO: 176 - DNA, 23 bp, Artificial Sequence
  Synthetic Polynucleotide
cttattccaaacttggtgggaat

SEQ ID NO: 177 - DNA, 23 bp, Artificial Sequence
  Synthetic Polynucleotide
atgacaatatctgtgcggaggtt

SEQ ID NO: 178 - DNA, 23 bp, Artificial Sequence
  Synthetic Polynucleotide
gccaaatgccgggatcttgtagg

SEQ ID NO: 179 - DNA, 23 bp, Artificial Sequence
  Synthetic Polynucleotide
acgttattacctgtgtgctgagc

SEQ ID NO: 180 - DNA, 23 bp, Artificial Sequence
  Synthetic Polynucleotide
gaccctcacagaccagggtttgc

SEQ ID NO: 181 - DNA, 23 bp, Artificial Sequence
  Synthetic Polynucleotide
atagatccatgttctgtggtatg

SEQ ID NO: 182 - DNA, 23 bp, Artificial Sequence
  Synthetic Polynucleotide
gactttaatagatccatgttctg

SEQ ID NO: 183 - DNA, 23 bp, Artificial Sequence
  Synthetic Polynucleotide
gtgactttaatagatccatgttc

SEQ ID NO: 184 - DNA, 23 bp, Artificial Sequence
  Synthetic Polynucleotide
gaatagcacaaacccttcccgac

SEQ ID NO: 185 - DNA, 23 bp, Artificial Sequence
  Synthetic Polynucleotide
ggaatagcacaaacccttcccga

SEQ ID NO: 186 - DNA, 23 bp, Artificial Sequence
  Synthetic Polynucleotide
gacaccatcagaacttgaggtta

SEQ ID NO: 187 - DNA, 23 bp, Artificial Sequence
  Synthetic Polynucleotide
agacaccatcagaacttgaggtt

SEQ ID NO: 188 - DNA, 23 bp, Artificial Sequence
  Synthetic Polynucleotide
gcttctagaggtttgtgggaatc

SEQ ID NO: 189 - DNA, 23 bp, Artificial Sequence
  Synthetic Polynucleotide
aagcttctagaggtttgtgggaa

SEQ ID NO: 190 - DNA, 23 bp, Artificial Sequence
  Synthetic Polynucleotide
ctgttcattggtttgaaggccag

SEQ ID NO: 191 - DNA, 23 bp, Artificial Sequence
  Synthetic Polynucleotide
ttatgctttgctgttcattggtt

SEQ ID NO: 192 - DNA, 23 bp, Artificial Sequence
  Synthetic Polynucleotide
aattaaccttgaatttgtttcat

SEQ ID NO: 193 - DNA, 23 bp, Artificial Sequence
  Synthetic Polynucleotide
atcttgctttatgcagcttcaca

SEQ ID NO: 194 - DNA, 23 bp, Artificial Sequence
  Synthetic Polynucleotide
agtaatcttgctttatgcagctt

SEQ ID NO: 195 - DNA, 23 bp, Artificial Sequence
  Synthetic Polynucleotide
aagattaaacataatcttttttg

SEQ ID NO: 196 - DNA, 23 bp, Artificial Sequence
  Synthetic Polynucleotide
agtaagaaaaccaagccttagat

SEQ ID NO: 197 - DNA, 23 bp, Artificial Sequence
  Synthetic Polynucleotide
atatgacagtaagaaaaccaagc

SEQ ID NO: 198 - DNA, 23 bp, Artificial Sequence
  Synthetic Polynucleotide
accaaccgcagaaacttgaggtg

SEQ ID NO: 199 - DNA, 23 bp, Artificial Sequence
  Synthetic Polynucleotide
taccaaccgcagaaacttgaggt

SEQ ID NO: 200 - DNA, 23 bp, Artificial Sequence
  Synthetic Polynucleotide
acatcaagcaccagtccaactat

SEQ ID NO: 201 - DNA, 19 bp, Artificial Sequence
  Synthetic Polynucleotide
tttggaagacaagtgaatg

SEQ ID NO: 202 - DNA, 19 bp, Artificial Sequence
  Synthetic Polynucleotide
gtgatgctctccaagtatg

SEQ ID NO: 203 - DNA, 19 bp, Artificial Sequence
  Synthetic Polynucleotide
accggattgtggttcgagt

SEQ ID NO: 204 - DNA, 19 bp, Artificial Sequence
  Synthetic Polynucleotide
tttctgcggttggtaaaga

SEQ ID NO: 205 - DNA, 19 bp, Artificial Sequence
  Synthetic Polynucleotide
cttctatcttctttgccat

SEQ ID NO: 206 - DNA, 19 bp, Artificial Sequence
  Synthetic Polynucleotide
gaacataccacagaacatg

SEQ ID NO: 207 - DNA, 19 bp, Artificial Sequence
  Synthetic Polynucleotide
ttatggacctgagctgaag

SEQ ID NO: 208 - DNA, 19 bp, Artificial Sequence
  Synthetic Polynucleotide
ggcccacgccaagatcaag

SEQ ID NO: 209 - DNA, 19 bp, Artificial Sequence
  Synthetic Polynucleotide
aagggtttgtgctattccc

SEQ ID NO: 210 - DNA, 19 bp, Artificial Sequence
  Synthetic Polynucleotide
tgtgatccattttactgca

SEQ ID NO: 211 - DNA, 19 bp, Artificial Sequence
  Synthetic Polynucleotide
accaactcaattattgagc

SEQ ID NO: 212 - DNA, 19 bp, Artificial Sequence
  Synthetic Polynucleotide
tctgcagctatctaaggct

SEQ ID NO: 213 - DNA, 19 bp, Artificial Sequence
  Synthetic Polynucleotide
ccgaagcagataatgttgt

SEQ ID NO: 214 - DNA, 19 bp, Artificial Sequence
  Synthetic Polynucleotide
tgtacacactgctccggaa

SEQ ID NO: 215 - DNA, 19 bp, Artificial Sequence
  Synthetic Polynucleotide
cagagacaacccttttggc

SEQ ID NO: 216 - DNA, 19 bp, Artificial Sequence
  Synthetic Polynucleotide
ccggcatttggcagcatcc

SEQ ID NO: 217 - DNA, 19 bp, Artificial Sequence
  Synthetic Polynucleotide
gttacttgtgttgggcata

SEQ ID NO: 218 - DNA, 19 bp, Artificial Sequence
  Synthetic Polynucleotide
gggaagaatgcctagcaag

SEQ ID NO: 219 - DNA, 19 bp, Artificial Sequence
  Synthetic Polynucleotide
tggaagacaagtgaatgca

SEQ ID NO: 220 - DNA, 19 bp, Artificial Sequence
  Synthetic Polynucleotide
cataaagcaagattactct

SEQ ID NO: 221 - DNA, 19 bp, Artificial Sequence
  Synthetic Polynucleotide
accaagtttggaataagct

SEQ ID NO: 222 - DNA, 19 bp, Artificial Sequence
  Synthetic Polynucleotide
gttcaacaaggagaattgt

SEQ ID NO: 223 - DNA, 19 bp, Artificial Sequence
  Synthetic Polynucleotide
gagaaaaatgcagatccag

SEQ ID NO: 224 - DNA, 19 bp, Artificial Sequence
  Synthetic Polynucleotide
agcaagctctcagtatcat

SEQ ID NO: 225 - DNA, 19 bp, Artificial Sequence
  Synthetic Polynucleotide
gcatcgtcatgagtatgta

SEQ ID NO: 226 - DNA, 19 bp, Artificial Sequence
  Synthetic Polynucleotide
gctccttgcaccatgttgc

SEQ ID NO: 227 - DNA, 19 bp, Artificial Sequence
  Synthetic Polynucleotide
aaccaactcaattattgag

SEQ ID NO: 228 - DNA, 19 bp, Artificial Sequence
  Synthetic Polynucleotide
cttgtgttgggcatatcat

SEQ ID NO: 229 - DNA, 19 bp, Artificial Sequence
  Synthetic Polynucleotide
gataaggttacttgtgttg

SEQ ID NO: 230 - DNA, 19 bp, Artificial Sequence
  Synthetic Polynucleotide
ggcttgcacagtgatgctc

SEQ ID NO: 231 - DNA, 19 bp, Artificial Sequence
  Synthetic Polynucleotide
gaaacaaattcaaggttaa

SEQ ID NO: 232 - DNA, 19 bp, Artificial Sequence
  Synthetic Polynucleotide
tttgtgatcttggactgtc

SEQ ID NO: 233 - DNA, 19 bp, Artificial Sequence
  Synthetic Polynucleotide
ggagaaaaatgcagatcca

SEQ ID NO: 234 - DNA, 19 bp, Artificial Sequence
  Synthetic Polynucleotide
agataatgttgtgtcaggg

SEQ ID NO: 235 - DNA, 19 bp, Artificial Sequence
  Synthetic Polynucleotide
caccaagtttggaataagc

SEQ ID NO: 236 - DNA, 19 bp, Artificial Sequence
  Synthetic Polynucleotide
aaagcataaccttgaatct

SEQ ID NO: 237 - DNA, 19 bp, Artificial Sequence
  Synthetic Polynucleotide
cacaggtaataacgtgaag

SEQ ID NO: 238 - DNA, 19 bp, Artificial Sequence
  Synthetic Polynucleotide
cagccattatcacaattga

SEQ ID NO: 239 - DNA, 19 bp, Artificial Sequence
  Synthetic Polynucleotide
aaaaatccaaccaactcaa

SEQ ID NO: 240 - DNA, 19 bp, Artificial Sequence
  Synthetic Polynucleotide
ataggaagcaccaagacga

SEQ ID NO: 241 - DNA, 19 bp, Artificial Sequence
  Synthetic Polynucleotide
gcgctgacctcaatggaca

SEQ ID NO: 242 - DNA, 19 bp, Artificial Sequence
  Synthetic Polynucleotide
cccggagagaagatgacat

SEQ ID NO: 243 - DNA, 19 bp, Artificial Sequence
  Synthetic Polynucleotide
cattgatattggacaggtg

SEQ ID NO: 244 - DNA, 19 bp, Artificial Sequence
  Synthetic Polynucleotide
cacacaggtaataacgtga

SEQ ID NO: 245 - DNA, 19 bp, Artificial Sequence
  Synthetic Polynucleotide
cctaaatggtgaatatgca

SEQ ID NO: 246 - DNA, 19 bp, Artificial Sequence
  Synthetic Polynucleotide
gccactgggttttatagaa

SEQ ID NO: 247 - DNA, 19 bp, Artificial Sequence
  Synthetic Polynucleotide
cacactgctccggaatcag

SEQ ID NO: 248 - DNA, 19 bp, Artificial Sequence
  Synthetic Polynucleotide
tacttgtgttgggcatatc

SEQ ID NO: 249 - DNA, 19 bp, Artificial Sequence
  Synthetic Polynucleotide
aggccgtgtactgtgacga

SEQ ID NO: 250 - DNA, 19 bp, Artificial Sequence
  Synthetic Polynucleotide
tagcaagctctcagtatca

SEQ ID NO: 251 - DNA, 19 bp, Artificial Sequence
  Synthetic Polynucleotide
ggcattgagatgaagttca

SEQ ID NO: 252 - DNA, 19 bp, Artificial Sequence
  Synthetic Polynucleotide
aacacccaatctgggctac

SEQ ID NO: 253 - DNA, 19 bp, Artificial Sequence
  Synthetic Polynucleotide
tttgtttcatttccgctga

SEQ ID NO: 254 - DNA, 19 bp, Artificial Sequence
  Synthetic Polynucleotide
ccactacttcagctatggg

SEQ ID NO: 255 - DNA, 19 bp, Artificial Sequence
  Synthetic Polynucleotide
accacagaacatggatcta

SEQ ID NO: 256 - DNA, 19 bp, Artificial Sequence
  Synthetic Polynucleotide
agaacctccgcacagatat

SEQ ID NO: 257 - DNA, 19 bp, Artificial Sequence
  Synthetic Polynucleotide
tacaagatcccggcatttg

SEQ ID NO: 258 - DNA, 19 bp, Artificial Sequence
  Synthetic Polynucleotide
tatgtacacactgctccgg

SEQ ID NO: 259 - DNA, 19 bp, Artificial Sequence
  Synthetic Polynucleotide
tccttgcaccatgttgcag

SEQ ID NO: 260 - DNA, 19 bp, Artificial Sequence
  Synthetic Polynucleotide
aagtaatttccacctagaa

SEQ ID NO: 261 - DNA, 19 bp, Artificial Sequence
  Synthetic Polynucleotide
ccacggctcccagtgcggg

SEQ ID NO: 262 - DNA, 19 bp, Artificial Sequence
  Synthetic Polynucleotide
cttgtctgccactgggttt

SEQ ID NO: 263 - DNA, 19 bp, Artificial Sequence
  Synthetic Polynucleotide
tgcttgatgtcactaataa

SEQ ID NO: 264 - DNA, 19 bp, Artificial Sequence
  Synthetic Polynucleotide
atcaaagatgccatccgtg

SEQ ID NO: 265 - DNA, 19 bp, Artificial Sequence
  Synthetic Polynucleotide
ctccaagtatgatcgtctg

SEQ ID NO: 266 - DNA, 19 bp, Artificial Sequence
  Synthetic Polynucleotide
agcaggaggaacataccac

SEQ ID NO: 267 - DNA, 19 bp, Artificial Sequence
  Synthetic Polynucleotide
ctctctcagagtattatgg

SEQ ID NO: 268 - DNA, 19 bp, Artificial Sequence
  Synthetic Polynucleotide
tcggaagagtgaggttgac

SEQ ID NO: 269 - DNA, 19 bp, Artificial Sequence
  Synthetic Polynucleotide
agcgctgacctcaatggac

SEQ ID NO: 270 - DNA, 19 bp, Artificial Sequence
  Synthetic Polynucleotide
attccacatggacaactgc

SEQ ID NO: 271 - DNA, 19 bp, Artificial Sequence
  Synthetic Polynucleotide
caagaaggccatctatgca

SEQ ID NO: 272 - DNA, 19 bp, Artificial Sequence
  Synthetic Polynucleotide
ccagccattatcacaattg

SEQ ID NO: 273 - DNA, 19 bp, Artificial Sequence
  Synthetic Polynucleotide
gacacagtgagcttgtctg

SEQ ID NO: 274 - DNA, 19 bp, Artificial Sequence
  Synthetic Polynucleotide
aacctccgcacagatattg

SEQ ID NO: 275 - DNA, 19 bp, Artificial Sequence
  Synthetic Polynucleotide
agcttgtctgccactgggt

SEQ ID NO: 276 - DNA, 19 bp, Artificial Sequence
  Synthetic Polynucleotide
tgctaagcttcctgcccaa

SEQ ID NO: 277 - DNA, 19 bp, Artificial Sequence
  Synthetic Polynucleotide
cggctgtgcaaaaccaacc

SEQ ID NO: 278 - DNA, 19 bp, Artificial Sequence
  Synthetic Polynucleotide
catcgtcatgagtatgtac

SEQ ID NO: 279 - DNA, 19 bp, Artificial Sequence
  Synthetic Polynucleotide
gctaagcttcctgcccaaa

SEQ ID NO: 280 - DNA, 19 bp, Artificial Sequence
  Synthetic Polynucleotide
caaggtaaccagtggcatg

SEQ ID NO: 281 - DNA, 19 bp, Artificial Sequence
  Synthetic Polynucleotide
gtgctgtggttgctgacac

SEQ ID NO: 282 - DNA, 19 bp, Artificial Sequence
  Synthetic Polynucleotide
ggcagaaaggtggtggaga

SEQ ID NO: 283 - DNA, 19 bp, Artificial Sequence
  Synthetic Polynucleotide
gaagcaccaagacgaggct

SEQ ID NO: 284 - DNA, 19 bp, Artificial Sequence
  Synthetic Polynucleotide
agactaactcagggaaccc

SEQ ID NO: 285 - DNA, 19 bp, Artificial Sequence
  Synthetic Polynucleotide
aaaccctggtctgtgaggg

SEQ ID NO: 286 - DNA, 19 bp, Artificial Sequence
  Synthetic Polynucleotide
tctacacagaacaccatga

SEQ ID NO: 287 - DNA, 19 bp, Artificial Sequence
  Synthetic Polynucleotide
gggtgaaaatcacctatga

SEQ ID NO: 288 - DNA, 19 bp, Artificial Sequence
  Synthetic Polynucleotide
cctgccattgatattggac

SEQ ID NO: 289 - DNA, 19 bp, Artificial Sequence
  Synthetic Polynucleotide
gttggctccagtctaaacc

SEQ ID NO: 290 - DNA, 19 bp, Artificial Sequence
  Synthetic Polynucleotide
gacatgctgataactggtg

SEQ ID NO: 291 - DNA, 19 bp, Artificial Sequence
  Synthetic Polynucleotide
tttatggacctgagctgaa

SEQ ID NO: 292 - DNA, 19 bp, Artificial Sequence
  Synthetic Polynucleotide
gctctcagtatcatgctcg

SEQ ID NO: 293 - DNA, 19 bp, Artificial Sequence
  Synthetic Polynucleotide
acctgaaaactccagttac

SEQ ID NO: 294 - DNA, 19 bp, Artificial Sequence
  Synthetic Polynucleotide
tagttggactggtgcttga

SEQ ID NO: 295 - DNA, 19 bp, Artificial Sequence
  Synthetic Polynucleotide
cccgggcccacgccaagat

SEQ ID NO: 296 - DNA, 19 bp, Artificial Sequence
  Synthetic Polynucleotide
agtttctgcggttggtaaa

SEQ ID NO: 297 - DNA, 19 bp, Artificial Sequence
  Synthetic Polynucleotide
atttggaagacaagtgaat

SEQ ID NO: 298 - DNA, 19 bp, Artificial Sequence
  Synthetic Polynucleotide
ttatagaacacccaatctg

SEQ ID NO: 299 - DNA, 19 bp, Artificial Sequence
  Synthetic Polynucleotide
agtgatgctctccaagtat

SEQ ID NO: 300 - DNA, 19 bp, Artificial Sequence
  Synthetic Polynucleotide
tgacacacttcaaccagaa

SEQ ID NO: 301 - DNA, 19 bp, Artificial Sequence
  Synthetic Polynucleotide
accaagctcggctgtggag

SEQ ID NO: 302 - DNA, 19 bp, Artificial Sequence
  Synthetic Polynucleotide
gttttccgaagcagataat

SEQ ID NO: 303 - DNA, 19 bp, Artificial Sequence
  Synthetic Polynucleotide
tccaagtatgatcgtctgc

SEQ ID NO: 304 - DNA, 19 bp, Artificial Sequence
  Synthetic Polynucleotide
ggaagaatgcctagcaagc

SEQ ID NO: 305 - DNA, 19 bp, Artificial Sequence
  Synthetic Polynucleotide
acttttctgccaatgcctg

SEQ ID NO: 306 - DNA, 19 bp, Artificial Sequence
  Synthetic Polynucleotide
aatatgctgtttcctatga

SEQ ID NO: 307 - DNA, 19 bp, Artificial Sequence
  Synthetic Polynucleotide
aaccaagctcggctgtgga

SEQ ID NO: 308 - DNA, 19 bp, Artificial Sequence
  Synthetic Polynucleotide
ctaacactgtgcccaacac

SEQ ID NO: 309 - DNA, 19 bp, Artificial Sequence
  Synthetic Polynucleotide
ttgttgcaaaaatgttggg

SEQ ID NO: 310 - DNA, 19 bp, Artificial Sequence
  Synthetic Polynucleotide
ttgtttcatttccgctgat

SEQ ID NO: 311 - DNA, 19 bp, Artificial Sequence
  Synthetic Polynucleotide
tttgccatcaaagatgcca

SEQ ID NO: 312 - DNA, 19 bp, Artificial Sequence
  Synthetic Polynucleotide
gggctacagctttgagact

SEQ ID NO: 313 - DNA, 19 bp, Artificial Sequence
  Synthetic Polynucleotide
acacaggtaataacgtgaa

SEQ ID NO: 314 - DNA, 19 bp, Artificial Sequence
  Synthetic Polynucleotide
ttgtgttgggcatatcatt

SEQ ID NO: 315 - DNA, 19 bp, Artificial Sequence
  Synthetic Polynucleotide
gctgcacaggctacagacc

SEQ ID NO: 316 - DNA, 19 bp, Artificial Sequence
  Synthetic Polynucleotide
ggaataggaagcaccaaga

SEQ ID NO: 317 - DNA, 19 bp, Artificial Sequence
  Synthetic Polynucleotide
aagtttctgcggttggtaa

SEQ ID NO: 318 - DNA, 19 bp, Artificial Sequence
  Synthetic Polynucleotide
aaggaataggaagcaccaa

SEQ ID NO: 319 - DNA, 19 bp, Artificial Sequence
  Synthetic Polynucleotide
acctctagaagcttaaacc

SEQ ID NO: 320 - DNA, 19 bp, Artificial Sequence
  Synthetic Polynucleotide
tgttggctccagtctaaac

SEQ ID NO: 321 - DNA, 19 bp, Artificial Sequence
  Synthetic Polynucleotide
acatggacaactgctataa

SEQ ID NO: 322 - DNA, 19 bp, Artificial Sequence
  Synthetic Polynucleotide
ggacatgctgataactggt

SEQ ID NO: 323 - DNA, 19 bp, Artificial Sequence
  Synthetic Polynucleotide
ctctcagtatcatgctcgg

SEQ ID NO: 324 - DNA, 19 bp, Artificial Sequence
  Synthetic Polynucleotide
ctggacagcctgtataacc

SEQ ID NO: 325 - DNA, 19 bp, Artificial Sequence
  Synthetic Polynucleotide
aaccggattgtggttcgag

SEQ ID NO: 326 - DNA, 19 bp, Artificial Sequence
  Synthetic Polynucleotide
tattccacatggacaactg

SEQ ID NO: 327 - DNA, 19 bp, Artificial Sequence
  Synthetic Polynucleotide
tgcaccattgctgttccaa

SEQ ID NO: 328 - DNA, 19 bp, Artificial Sequence
  Synthetic Polynucleotide
ccagagacaacccttttgg

SEQ ID NO: 329 - DNA, 19 bp, Artificial Sequence
  Synthetic Polynucleotide
agttggactggtgcttgat

SEQ ID NO: 330 - DNA, 19 bp, Artificial Sequence
  Synthetic Polynucleotide
tgagcccggaggagatact

SEQ ID NO: 331 - DNA, 19 bp, Artificial Sequence
  Synthetic Polynucleotide
aattcccaccaagtttgga

SEQ ID NO: 332 - DNA, 19 bp, Artificial Sequence
  Synthetic Polynucleotide
gcttgtcagaccatcttga

SEQ ID NO: 333 - DNA, 19 bp, Artificial Sequence
  Synthetic Polynucleotide
atctgcagctatctaaggc

SEQ ID NO: 334 - DNA, 19 bp, Artificial Sequence
  Synthetic Polynucleotide
tacacactgctccggaatc

SEQ ID NO: 335 - DNA, 19 bp, Artificial Sequence
  Synthetic Polynucleotide
agaattgccaaaagccacg

SEQ ID NO: 336 - DNA, 19 bp, Artificial Sequence
  Synthetic Polynucleotide
gatactgctctccatagag

SEQ ID NO: 337 - DNA, 19 bp, Artificial Sequence
  Synthetic Polynucleotide
ctgccagtgtctcttagga

SEQ ID NO: 338 - DNA, 19 bp, Artificial Sequence
  Synthetic Polynucleotide
ctcttaggagtgaggtacc

SEQ ID NO: 339 - DNA, 19 bp, Artificial Sequence
  Synthetic Polynucleotide
ttcgccagtgcaactttac

SEQ ID NO: 340 - DNA, 19 bp, Artificial Sequence
  Synthetic Polynucleotide
gcacacaggtaataacgtg

SEQ ID NO: 341 - DNA, 19 bp, Artificial Sequence
  Synthetic Polynucleotide
tcccggagagaagatgaca

SEQ ID NO: 342 - DNA, 19 bp, Artificial Sequence
  Synthetic Polynucleotide
ggttttccgaagcagataa

SEQ ID NO: 343 - DNA, 19 bp, Artificial Sequence
  Synthetic Polynucleotide
gcacttctacctggagact

SEQ ID NO: 344 - DNA, 19 bp, Artificial Sequence
  Synthetic Polynucleotide
cttccctccaacacggcct

SEQ ID NO: 345 - DNA, 19 bp, Artificial Sequence
  Synthetic Polynucleotide
ccaagctcggctgtggaga

SEQ ID NO: 346 - DNA, 19 bp, Artificial Sequence
  Synthetic Polynucleotide
cagtatcatgctcggaaga

SEQ ID NO: 347 - DNA, 19 bp, Artificial Sequence
  Synthetic Polynucleotide
agctctcagtatcatgctc

SEQ ID NO: 348 - DNA, 19 bp, Artificial Sequence
  Synthetic Polynucleotide
gtctaaaccctgccattga

SEQ ID NO: 349 - DNA, 19 bp, Artificial Sequence
  Synthetic Polynucleotide
tgagctgtctctccggctg

SEQ ID NO: 350 - DNA, 19 bp, Artificial Sequence
  Synthetic Polynucleotide
gcatgctggaccgtgatga

SEQ ID NO: 351 - DNA, 19 bp, Artificial Sequence
  Synthetic Polynucleotide
aagaacctccgcacagata

SEQ ID NO: 352 - DNA, 19 bp, Artificial Sequence
  Synthetic Polynucleotide
agtaatttccacctagaaa

SEQ ID NO: 353 - DNA, 19 bp, Artificial Sequence
  Synthetic Polynucleotide
gaacctccgcacagatatt

SEQ ID NO: 354 - DNA, 19 bp, Artificial Sequence
  Synthetic Polynucleotide
tataaatccaaccttctgc

SEQ ID NO: 355 - DNA, 19 bp, Artificial Sequence
  Synthetic Polynucleotide
cagggtttgtttgtttcat

SEQ ID NO: 356 - DNA, 19 bp, Artificial Sequence
  Synthetic Polynucleotide
gagaattgccaaaagccac

SEQ ID NO: 357 - DNA, 19 bp, Artificial Sequence
  Synthetic Polynucleotide
gacaagcactaacactgtg

SEQ ID NO: 358 - DNA, 19 bp, Artificial Sequence
  Synthetic Polynucleotide
tcccggcatttggcagcat

SEQ ID NO: 359 - DNA, 19 bp, Artificial Sequence
  Synthetic Polynucleotide
ccggagagaagatgacatt

SEQ ID NO: 360 - DNA, 19 bp, Artificial Sequence
  Synthetic Polynucleotide
ttcccaccaagtttggaat

SEQ ID NO: 361 - DNA, 19 bp, Artificial Sequence
  Synthetic Polynucleotide
ctttacatttgaagccaaa

SEQ ID NO: 362 - DNA, 19 bp, Artificial Sequence
  Synthetic Polynucleotide
catggacaactgctataaa

SEQ ID NO: 363 - DNA, 19 bp, Artificial Sequence
  Synthetic Polynucleotide
aaactgcaaaccctggtct

SEQ ID NO: 364 - DNA, 19 bp, Artificial Sequence
  Synthetic Polynucleotide
tctttacatttgaagccaa

SEQ ID NO: 365 - DNA, 19 bp, Artificial Sequence
  Synthetic Polynucleotide
ttctcagcattcaagcagg

SEQ ID NO: 366 - DNA, 19 bp, Artificial Sequence
  Synthetic Polynucleotide
acaaattcaaggttaattg

SEQ ID NO: 367 - DNA, 19 bp, Artificial Sequence
  Synthetic Polynucleotide
gtcactaataaatgaaact

SEQ ID NO: 368 - DNA, 19 bp, Artificial Sequence
  Synthetic Polynucleotide
ttctgcggttggtaaagag

SEQ ID NO: 369 - DNA, 19 bp, Artificial Sequence
  Synthetic Polynucleotide
atcttctttgccatcaaag

SEQ ID NO: 370 - DNA, 19 bp, Artificial Sequence
  Synthetic Polynucleotide
tataacctcaagttctgat

SEQ ID NO: 371 - DNA, 19 bp, Artificial Sequence
  Synthetic Polynucleotide
taagaacctccgcacagat

SEQ ID NO: 372 - DNA, 19 bp, Artificial Sequence
  Synthetic Polynucleotide
atcgtcatgagtatgtaca

SEQ ID NO: 373 - DNA, 19 bp, Artificial Sequence
  Synthetic Polynucleotide
cagtgacaactgtggaagg

SEQ ID NO: 374 - DNA, 19 bp, Artificial Sequence
  Synthetic Polynucleotide
tatggacctgagctgaaga

SEQ ID NO: 375 - DNA, 19 bp, Artificial Sequence
  Synthetic Polynucleotide
ttcaagaatatgctgtttc

SEQ ID NO: 376 - DNA, 19 bp, Artificial Sequence
  Synthetic Polynucleotide
ctcaaccctcaaggagctg

SEQ ID NO: 377 - DNA, 19 bp, Artificial Sequence
  Synthetic Polynucleotide
aacataccacagaacatgg

SEQ ID NO: 378 - DNA, 19 bp, Artificial Sequence
  Synthetic Polynucleotide
gtcatgagtatgtacacac

SEQ ID NO: 379 - DNA, 19 bp, Artificial Sequence
  Synthetic Polynucleotide
gacacacttcaaccagaag

SEQ ID NO: 380 - DNA, 19 bp, Artificial Sequence
  Synthetic Polynucleotide
tcagcacacaggtaataac

SEQ ID NO: 381 - DNA, 19 bp, Artificial Sequence
  Synthetic Polynucleotide
catggatctattaaagtca

SEQ ID NO: 382 - DNA, 19 bp, Artificial Sequence
  Synthetic Polynucleotide
gaaccaagctcggctgtgg

SEQ ID NO: 383 - DNA, 19 bp, Artificial Sequence
  Synthetic Polynucleotide
gccattgatattggacagg

SEQ ID NO: 384 - DNA, 19 bp, Artificial Sequence
  Synthetic Polynucleotide
caccctcagcttcttcttc

SEQ ID NO: 385 - DNA, 19 bp, Artificial Sequence
  Synthetic Polynucleotide
aggagagaattgccaaaag

SEQ ID NO: 386 - DNA, 19 bp, Artificial Sequence
  Synthetic Polynucleotide
catgctgataactggtggc

SEQ ID NO: 387 - DNA, 19 bp, Artificial Sequence
  Synthetic Polynucleotide
agattatgtttaatcttta

SEQ ID NO: 388 - DNA, 19 bp, Artificial Sequence
  Synthetic Polynucleotide
ggagagaattgccaaaagc

SEQ ID NO: 389 - DNA, 19 bp, Artificial Sequence
  Synthetic Polynucleotide
aagattatgtttaatcttt

SEQ ID NO: 390 - DNA, 19 bp, Artificial Sequence
  Synthetic Polynucleotide
gccttcataccaaaatggt

SEQ ID NO: 391 - DNA, 19 bp, Artificial Sequence
  Synthetic Polynucleotide
cgctggtttgctgggaagc

SEQ ID NO: 392 - DNA, 19 bp, Artificial Sequence
  Synthetic Polynucleotide
tgttctagatttctttccc

SEQ ID NO: 393 - DNA, 19 bp, Artificial Sequence
  Synthetic Polynucleotide
ctcgagctcagcacacagg

SEQ ID NO: 394 - DNA, 19 bp, Artificial Sequence
  Synthetic Polynucleotide
ccattgctgttccaaaagg

SEQ ID NO: 395 - DNA, 19 bp, Artificial Sequence
  Synthetic Polynucleotide
agatcgtccacttttctgc

SEQ ID NO: 396 - DNA, 19 bp, Artificial Sequence
  Synthetic Polynucleotide
ggtaataacgtgaaggaac

SEQ ID NO: 397 - DNA, 19 bp, Artificial Sequence
  Synthetic Polynucleotide
ggcatgagagttttattca

SEQ ID NO: 398 - DNA, 19 bp, Artificial Sequence
  Synthetic Polynucleotide
tcaaaccaatgaacagcaa

SEQ ID NO: 399 - DNA, 19 bp, Artificial Sequence
  Synthetic Polynucleotide
caaaccaatgaacagcaaa

SEQ ID NO: 400 - DNA, 19 bp, Artificial Sequence
  Synthetic Polynucleotide
ccaaccaactcaattattg

SEQ ID NO: 401 - DNA, 19 bp, Artificial Sequence
  Synthetic Polynucleotide
aaaaagattatgtttaatc

SEQ ID NO: 402 - DNA, 19 bp, Artificial Sequence
  Synthetic Polynucleotide
gtgggatgcctgcagagga

SEQ ID NO: 403 - DNA, 19 bp, Artificial Sequence
  Synthetic Polynucleotide
ctcaagaccactcagaggc

SEQ ID NO: 404 - DNA, 19 bp, Artificial Sequence
  Synthetic Polynucleotide
gatgcctgcagaggaggtg

SEQ ID NO: 405 - DNA, 19 bp, Artificial Sequence
  Synthetic Polynucleotide
agtggcatgagagttttat

SEQ ID NO: 406 - DNA, 19 bp, Artificial Sequence
  Synthetic Polynucleotide
atctattaaagtcacagaa

SEQ ID NO: 407 - DNA, 19 bp, Artificial Sequence
  Synthetic Polynucleotide
tcctgtgatccattttact

SEQ ID NO: 408 - DNA, 19 bp, Artificial Sequence
  Synthetic Polynucleotide
ggctgcttctatcttcttt

SEQ ID NO: 409 - DNA, 19 bp, Artificial Sequence
  Synthetic Polynucleotide
tacacagatggctctgtgc

SEQ ID NO: 410 - DNA, 19 bp, Artificial Sequence
  Synthetic Polynucleotide
aagttctacctgacagtcc

SEQ ID NO: 411 - DNA, 21 bp, Artificial Sequence
  Synthetic Polynucleotide
gatttggaagacaagtgaatg

SEQ ID NO: 412 - DNA, 21 bp, Artificial Sequence
  Synthetic Polynucleotide
cagtgatgctctccaagtatg

SEQ ID NO: 413 - DNA, 21 bp, Artificial Sequence
  Synthetic Polynucleotide
aaaccggattgtggttcgagt

SEQ ID NO: 414 - DNA, 21 bp, Artificial Sequence
  Synthetic Polynucleotide
agtttctgcggttggtaaaga

SEQ ID NO: 415 - DNA, 21 bp, Artificial Sequence
  Synthetic Polynucleotide
tgcttctatcttctttgccat

SEQ ID NO: 416 - DNA, 21 bp, Artificial Sequence
  Synthetic Polynucleotide
aggaacataccacagaacatg

SEQ ID NO: 417 - DNA, 21 bp, Artificial Sequence
  Synthetic Polynucleotide
ttttatggacctgagctgaag

SEQ ID NO: 418 - DNA, 21 bp, Artificial Sequence
  Synthetic Polynucleotide
cgggcccacgccaagatcaag

SEQ ID NO: 419 - DNA, 21 bp, Artificial Sequence
  Synthetic Polynucleotide
ggaagggtttgtgctattccc

SEQ ID NO: 420 - DNA, 21 bp, Artificial Sequence
  Synthetic Polynucleotide
cctgtgatccattttactgca

SEQ ID NO: 421 - DNA, 21 bp, Artificial Sequence
  Synthetic Polynucleotide
caaccaactcaattattgagc

SEQ ID NO: 422 - DNA, 21 bp, Artificial Sequence
  Synthetic Polynucleotide
gatctgcagctatctaaggct

SEQ ID NO: 423 - DNA, 21 bp, Artificial Sequence
  Synthetic Polynucleotide
ttccgaagcagataatgttgt

SEQ ID NO: 424 - DNA, 21 bp, Artificial Sequence
  Synthetic Polynucleotide
tatgtacacactgctccggaa

SEQ ID NO: 425 - DNA, 21 bp, Artificial Sequence
  Synthetic Polynucleotide
tccagagacaacccttttggc

SEQ ID NO: 426 - DNA, 21 bp, Artificial Sequence
  Synthetic Polynucleotide
tcccggcatttggcagcatcc

SEQ ID NO: 427 - DNA, 21 bp, Artificial Sequence
  Synthetic Polynucleotide
aggttacttgtgttgggcata

SEQ ID NO: 428 - DNA, 21 bp, Artificial Sequence
  Synthetic Polynucleotide
ctgggaagaatgcctagcaag

SEQ ID NO: 429 - DNA, 21 bp, Artificial Sequence
  Synthetic Polynucleotide
tttggaagacaagtgaatgca

SEQ ID NO: 430 - DNA, 21 bp, Artificial Sequence
  Synthetic Polynucleotide
tgcataaagcaagattactct

SEQ ID NO: 431 - DNA, 21 bp, Artificial Sequence
  Synthetic Polynucleotide
ccaccaagtttggaataagct

SEQ ID NO: 432 - DNA, 21 bp, Artificial Sequence
  Synthetic Polynucleotide
aagttcaacaaggagaattgt

SEQ ID NO: 433 - DNA, 21 bp, Artificial Sequence
  Synthetic Polynucleotide
tggagaaaaatgcagatccag

SEQ ID NO: 434 - DNA, 21 bp, Artificial Sequence
  Synthetic Polynucleotide
ctagcaagctctcagtatcat

SEQ ID NO: 435 - DNA, 21 bp, Artificial Sequence
  Synthetic Polynucleotide
tggcatcgtcatgagtatgta

SEQ ID NO: 436 - DNA, 21 bp, Artificial Sequence
  Synthetic Polynucleotide
ctgctccttgcaccatgttgc

SEQ ID NO: 437 - DNA, 21 bp, Artificial Sequence
  Synthetic Polynucleotide
ccaaccaactcaattattgag

SEQ ID NO: 438 - DNA, 21 bp, Artificial Sequence
  Synthetic Polynucleotide
tacttgtgttgggcatatcat

SEQ ID NO: 439 - DNA, 21 bp, Artificial Sequence
  Synthetic Polynucleotide
aggataaggttacttgtgttg

SEQ ID NO: 440 - DNA, 21 bp, Artificial Sequence
  Synthetic Polynucleotide
ggggcttgcacagtgatgctc

SEQ ID NO: 441 - DNA, 21 bp, Artificial Sequence
  Synthetic Polynucleotide
atgaaacaaattcaaggttaa

SEQ ID NO: 442 - DNA, 21 bp, Artificial Sequence
  Synthetic Polynucleotide
tctttgtgatcttggactgtc

SEQ ID NO: 443 - DNA, 21 bp, Artificial Sequence
  Synthetic Polynucleotide
gtggagaaaaatgcagatcca

SEQ ID NO: 444 - DNA, 21 bp, Artificial Sequence
  Synthetic Polynucleotide
gcagataatgttgtgtcaggg

SEQ ID NO: 445 - DNA, 21 bp, Artificial Sequence
  Synthetic Polynucleotide
cccaccaagtttggaataagc

SEQ ID NO: 446 - DNA, 21 bp, Artificial Sequence
  Synthetic Polynucleotide
gcaaagcataaccttgaatct

SEQ ID NO: 447 - DNA, 21 bp, Artificial Sequence
  Synthetic Polynucleotide
cacacaggtaataacgtgaag

SEQ ID NO: 448 - DNA, 21 bp, Artificial Sequence
  Synthetic Polynucleotide
accagccattatcacaattga

SEQ ID NO: 449 - DNA, 21 bp, Artificial Sequence
  Synthetic Polynucleotide
acaaaaatccaaccaactcaa

SEQ ID NO: 450 - DNA, 21 bp, Artificial Sequence
  Synthetic Polynucleotide
gaataggaagcaccaagacga

SEQ ID NO: 451 - DNA, 21 bp, Artificial Sequence
  Synthetic Polynucleotide
cagcgctgacctcaatggaca

SEQ ID NO: 452 - DNA, 21 bp, Artificial Sequence
  Synthetic Polynucleotide
ctcccggagagaagatgacat

SEQ ID NO: 453 - DNA, 21 bp, Artificial Sequence
  Synthetic Polynucleotide
gccattgatattggacaggtg

SEQ ID NO: 454 - DNA, 21 bp, Artificial Sequence
  Synthetic Polynucleotide
agcacacaggtaataacgtga

SEQ ID NO: 455 - DNA, 21 bp, Artificial Sequence
  Synthetic Polynucleotide
tgcctaaatggtgaatatgca

SEQ ID NO: 456 - DNA, 21 bp, Artificial Sequence
  Synthetic Polynucleotide
ctgccactgggttttatagaa

SEQ ID NO: 457 - DNA, 21 bp, Artificial Sequence
  Synthetic Polynucleotide
tacacactgctccggaatcag

SEQ ID NO: 458 - DNA, 21 bp, Artificial Sequence
  Synthetic Polynucleotide
gttacttgtgttgggcatatc

SEQ ID NO: 459 - DNA, 21 bp, Artificial Sequence
  Synthetic Polynucleotide
tgaggccgtgtactgtgacga

SEQ ID NO: 460 - DNA, 21 bp, Artificial Sequence
  Synthetic Polynucleotide
cctagcaagctctcagtatca

SEQ ID NO: 461 - DNA, 21 bp, Artificial Sequence
  Synthetic Polynucleotide
ttggcattgagatgaagttca

SEQ ID NO: 462 - DNA, 21 bp, Artificial Sequence
  Synthetic Polynucleotide
agaacacccaatctgggctac

SEQ ID NO: 463 - DNA, 21 bp, Artificial Sequence
  Synthetic Polynucleotide
tgtttgtttcatttccgctga

SEQ ID NO: 464 - DNA, 21 bp, Artificial Sequence
  Synthetic Polynucleotide
ttccactacttcagctatggg

SEQ ID NO: 465 - DNA, 21 bp, Artificial Sequence
  Synthetic Polynucleotide
ataccacagaacatggatcta

SEQ ID NO: 466 - DNA, 21 bp, Artificial Sequence
  Synthetic Polynucleotide
taagaacctccgcacagatat

SEQ ID NO: 467 - DNA, 21 bp, Artificial Sequence
  Synthetic Polynucleotide
cctacaagatcccggcatttg

SEQ ID NO: 468 - DNA, 21 bp, Artificial Sequence
  Synthetic Polynucleotide
agtatgtacacactgctccgg

SEQ ID NO: 469 - DNA, 21 bp, Artificial Sequence
  Synthetic Polynucleotide
gctccttgcaccatgttgcag

SEQ ID NO: 470 - DNA, 21 bp, Artificial Sequence
  Synthetic Polynucleotide
caaagtaatttccacctagaa

SEQ ID NO: 471 - DNA, 21 bp, Artificial Sequence
  Synthetic Polynucleotide
agccacggctcccagtgcggg

SEQ ID NO: 472 - DNA, 21 bp, Artificial Sequence
  Synthetic Polynucleotide
agcttgtctgccactgggttt

SEQ ID NO: 473 - DNA, 21 bp, Artificial Sequence
  Synthetic Polynucleotide
ggtgcttgatgtcactaataa

SEQ ID NO: 474 - DNA, 21 bp, Artificial Sequence
  Synthetic Polynucleotide
ccatcaaagatgccatccgtg

SEQ ID NO: 475 - DNA, 21 bp, Artificial Sequence
  Synthetic Polynucleotide
ctctccaagtatgatcgtctg

SEQ ID NO: 476 - DNA, 21 bp, Artificial Sequence
  Synthetic Polynucleotide
ggagcaggaggaacataccac

SEQ ID NO: 477 - DNA, 21 bp, Artificial Sequence
  Synthetic Polynucleotide
atctctctcagagtattatgg

SEQ ID NO: 478 - DNA, 21 bp, Artificial Sequence
  Synthetic Polynucleotide
gctcggaagagtgaggttgac

SEQ ID NO: 479 - DNA, 21 bp, Artificial Sequence
  Synthetic Polynucleotide
tcagcgctgacctcaatggac

SEQ ID NO: 480 - DNA, 21 bp, Artificial Sequence
  Synthetic Polynucleotide
ttattccacatggacaactgc

SEQ ID NO: 481 - DNA, 21 bp, Artificial Sequence
  Synthetic Polynucleotide
aacaagaaggccatctatgca

SEQ ID NO: 482 - DNA, 21 bp, Artificial Sequence
  Synthetic Polynucleotide
taccagccattatcacaattg

SEQ ID NO: 483 - DNA, 21 bp, Artificial Sequence
  Synthetic Polynucleotide
tggacacagtgagcttgtctg

SEQ ID NO: 484 - DNA, 21 bp, Artificial Sequence
  Synthetic Polynucleotide
agaacctccgcacagatattg

SEQ ID NO: 485 - DNA, 21 bp, Artificial Sequence
  Synthetic Polynucleotide
tgagcttgtctgccactgggt

SEQ ID NO: 486 - DNA, 21 bp, Artificial Sequence
  Synthetic Polynucleotide
gttgctaagcttcctgcccaa

SEQ ID NO: 487 - DNA, 21 bp, Artificial Sequence
  Synthetic Polynucleotide
ggcggctgtgcaaaaccaacc

SEQ ID NO: 488 - DNA, 21 bp, Artificial Sequence
  Synthetic Polynucleotide
ggcatcgtcatgagtatgtac

SEQ ID NO: 489 - DNA, 21 bp, Artificial Sequence
  Synthetic Polynucleotide
ttgctaagcttcctgcccaaa

SEQ ID NO: 490 - DNA, 21 bp, Artificial Sequence
  Synthetic Polynucleotide
gccaaggtaaccagtggcatg

SEQ ID NO: 491 - DNA, 21 bp, Artificial Sequence
  Synthetic Polynucleotide
tggtgctgtggttgctgacac

SEQ ID NO: 492 - DNA, 21 bp, Artificial Sequence
  Synthetic Polynucleotide
atggcagaaaggtggtggaga

SEQ ID NO: 493 - DNA, 21 bp, Artificial Sequence
  Synthetic Polynucleotide
aggaagcaccaagacgaggct

SEQ ID NO: 494 - DNA, 21 bp, Artificial Sequence
  Synthetic Polynucleotide
tgagactaactcagggaaccc

SEQ ID NO: 495 - DNA, 21 bp, Artificial Sequence
  Synthetic Polynucleotide
gcaaaccctggtctgtgaggg

SEQ ID NO: 496 - DNA, 21 bp, Artificial Sequence
  Synthetic Polynucleotide
tgtctacacagaacaccatga

SEQ ID NO: 497 - DNA, 21 bp, Artificial Sequence
  Synthetic Polynucleotide
aggggtgaaaatcacctatga

SEQ ID NO: 498 - DNA, 21 bp, Artificial Sequence
  Synthetic Polynucleotide
accctgccattgatattggac

SEQ ID NO: 499 - DNA, 21 bp, Artificial Sequence
  Synthetic Polynucleotide
atgttggctccagtctaaacc

SEQ ID NO: 500 - DNA, 21 bp, Artificial Sequence
  Synthetic Polynucleotide
aggacatgctgataactggtg

SEQ ID NO: 501 - DNA, 21 bp, Artificial Sequence
  Synthetic Polynucleotide
cttttatggacctgagctgaa

SEQ ID NO: 502 - DNA, 21 bp, Artificial Sequence
  Synthetic Polynucleotide
aagctctcagtatcatgctcg

SEQ ID NO: 503 - DNA, 21 bp, Artificial Sequence
  Synthetic Polynucleotide
tgacctgaaaactccagttac

SEQ ID NO: 504 - DNA, 21 bp, Artificial Sequence
  Synthetic Polynucleotide
tatagttggactggtgcttga

SEQ ID NO: 505 - DNA, 21 bp, Artificial Sequence
  Synthetic Polynucleotide
cacccgggcccacgccaagat

SEQ ID NO: 506 - DNA, 21 bp, Artificial Sequence
  Synthetic Polynucleotide
caagtttctgcggttggtaaa

SEQ ID NO: 507 - DNA, 21 bp, Artificial Sequence
  Synthetic Polynucleotide
ggatttggaagacaagtgaat

SEQ ID NO: 508 - DNA, 21 bp, Artificial Sequence
  Synthetic Polynucleotide
ttttatagaacacccaatctg

SEQ ID NO: 509 - DNA, 21 bp, Artificial Sequence
  Synthetic Polynucleotide
acagtgatgctctccaagtat

SEQ ID NO: 510 - DNA, 21 bp, Artificial Sequence
  Synthetic Polynucleotide
cctgacacacttcaaccagaa

SEQ ID NO: 511 - DNA, 21 bp, Artificial Sequence
  Synthetic Polynucleotide
gaaccaagctcggctgtggag

SEQ ID NO: 512 - DNA, 21 bp, Artificial Sequence
  Synthetic Polynucleotide
gggttttccgaagcagataat

SEQ ID NO: 513 - DNA, 21 bp, Artificial Sequence
  Synthetic Polynucleotide
tctccaagtatgatcgtctgc

SEQ ID NO: 514 - DNA, 21 bp, Artificial Sequence
  Synthetic Polynucleotide
tgggaagaatgcctagcaagc

SEQ ID NO: 515 - DNA, 21 bp, Artificial Sequence
  Synthetic Polynucleotide
ccacttttctgccaatgcctg

SEQ ID NO: 516 - DNA, 21 bp, Artificial Sequence
  Synthetic Polynucleotide
agaatatgctgtttcctatga

SEQ ID NO: 517 - DNA, 21 bp, Artificial Sequence
  Synthetic Polynucleotide
ggaaccaagctcggctgtgga

SEQ ID NO: 518 - DNA, 21 bp, Artificial Sequence
  Synthetic Polynucleotide
cactaacactgtgcccaacac

SEQ ID NO: 519 - DNA, 21 bp, Artificial Sequence
  Synthetic Polynucleotide
ctttgttgcaaaaatgttggg

SEQ ID NO: 520 - DNA, 21 bp, Artificial Sequence
  Synthetic Polynucleotide
gtttgtttcatttccgctgat

SEQ ID NO: 521 - DNA, 21 bp, Artificial Sequence
  Synthetic Polynucleotide
tctttgccatcaaagatgcca

SEQ ID NO: 522 - DNA, 21 bp, Artificial Sequence
  Synthetic Polynucleotide
ctgggctacagctttgagact

SEQ ID NO: 523 - DNA, 21 bp, Artificial Sequence
  Synthetic Polynucleotide
gcacacaggtaataacgtgaa

SEQ ID NO: 524 - DNA, 21 bp, Artificial Sequence
  Synthetic Polynucleotide
acttgtgttgggcatatcatt

SEQ ID NO: 525 - DNA, 21 bp, Artificial Sequence
  Synthetic Polynucleotide
ccgctgcacaggctacagacc

SEQ ID NO: 526 - DNA, 21 bp, Artificial Sequence
  Synthetic Polynucleotide
aaggaataggaagcaccaaga

SEQ ID NO: 527 - DNA, 21 bp, Artificial Sequence
  Synthetic Polynucleotide
tcaagtttctgcggttggtaa

SEQ ID NO: 528 - DNA, 21 bp, Artificial Sequence
  Synthetic Polynucleotide
ggaaggaataggaagcaccaa

SEQ ID NO: 529 - DNA, 21 bp, Artificial Sequence
  Synthetic Polynucleotide
aaacctctagaagcttaaacc

SEQ ID NO: 530 - DNA, 21 bp, Artificial Sequence
  Synthetic Polynucleotide
gatgttggctccagtctaaac

SEQ ID NO: 531 - DNA, 21 bp, Artificial Sequence
  Synthetic Polynucleotide
ccacatggacaactgctataa

SEQ ID NO: 532 - DNA, 21 bp, Artificial Sequence
  Synthetic Polynucleotide
gaggacatgctgataactggt

SEQ ID NO: 533 - DNA, 21 bp, Artificial Sequence
  Synthetic Polynucleotide
agctctcagtatcatgctcgg

SEQ ID NO: 534 - DNA, 21 bp, Artificial Sequence
  Synthetic Polynucleotide
tactggacagcctgtataacc

SEQ ID NO: 535 - DNA, 21 bp, Artificial Sequence
  Synthetic Polynucleotide
caaaccggattgtggttcgag

SEQ ID NO: 536 - DNA, 21 bp, Artificial Sequence
  Synthetic Polynucleotide
tttattccacatggacaactg

SEQ ID NO: 537 - DNA, 21 bp, Artificial Sequence
  Synthetic Polynucleotide
actgcaccattgctgttccaa

SEQ ID NO: 538 - DNA, 21 bp, Artificial Sequence
  Synthetic Polynucleotide
atccagagacaacccttttgg

SEQ ID NO: 539 - DNA, 21 bp, Artificial Sequence
  Synthetic Polynucleotide
atagttggactggtgcttgat

SEQ ID NO: 540 - DNA, 21 bp, Artificial Sequence
  Synthetic Polynucleotide
gctgagcccggaggagatact

SEQ ID NO: 541 - DNA, 21 bp, Artificial Sequence
  Synthetic Polynucleotide
ataattcccaccaagtttgga

SEQ ID NO: 542 - DNA, 21 bp, Artificial Sequence
  Synthetic Polynucleotide
cggcttgtcagaccatcttga

SEQ ID NO: 543 - DNA, 21 bp, Artificial Sequence
  Synthetic Polynucleotide
ggatctgcagctatctaaggc

SEQ ID NO: 544 - DNA, 21 bp, Artificial Sequence
  Synthetic Polynucleotide
tgtacacactgctccggaatc

SEQ ID NO: 545 - DNA, 21 bp, Artificial Sequence
  Synthetic Polynucleotide
agagaattgccaaaagccacg

SEQ ID NO: 546 - DNA, 21 bp, Artificial Sequence
  Synthetic Polynucleotide
gagatactgctctccatagag

SEQ ID NO: 547 - DNA, 21 bp, Artificial Sequence
  Synthetic Polynucleotide
acctgccagtgtctcttagga

SEQ ID NO: 548 - DNA, 21 bp, Artificial Sequence
  Synthetic Polynucleotide
gtctcttaggagtgaggtacc

SEQ ID NO: 549 - DNA, 21 bp, Artificial Sequence
  Synthetic Polynucleotide
ctttcgccagtgcaactttac

SEQ ID NO: 550 - DNA, 21 bp, Artificial Sequence
  Synthetic Polynucleotide
cagcacacaggtaataacgtg

SEQ ID NO: 551 - DNA, 21 bp, Artificial Sequence
  Synthetic Polynucleotide
cctcccggagagaagatgaca

SEQ ID NO: 552 - DNA, 21 bp, Artificial Sequence
  Synthetic Polynucleotide
ggggttttccgaagcagataa

SEQ ID NO: 553 - DNA, 21 bp, Artificial Sequence
  Synthetic Polynucleotide
gagcacttctacctggagact

SEQ ID NO: 554 - DNA, 21 bp, Artificial Sequence
  Synthetic Polynucleotide
accttccctccaacacggcct

SEQ ID NO: 555 - DNA, 21 bp, Artificial Sequence
  Synthetic Polynucleotide
aaccaagctcggctgtggaga

SEQ ID NO: 556 - DNA, 21 bp, Artificial Sequence
  Synthetic Polynucleotide
ctcagtatcatgctcggaaga

SEQ ID NO: 557 - DNA, 21 bp, Artificial Sequence
  Synthetic Polynucleotide
caagctctcagtatcatgctc

SEQ ID NO: 558 - DNA, 21 bp, Artificial Sequence
  Synthetic Polynucleotide
cagtctaaaccctgccattga

SEQ ID NO: 559 - DNA, 21 bp, Artificial Sequence
  Synthetic Polynucleotide
aatgagctgtctctccggctg

SEQ ID NO: 560 - DNA, 21 bp, Artificial Sequence
  Synthetic Polynucleotide
atgcatgctggaccgtgatga

SEQ ID NO: 561 - DNA, 21 bp, Artificial Sequence
  Synthetic Polynucleotide
ataagaacctccgcacagata

SEQ ID NO: 562 - DNA, 21 bp, Artificial Sequence
  Synthetic Polynucleotide
aaagtaatttccacctagaaa

SEQ ID NO: 563 - DNA, 21 bp, Artificial Sequence
  Synthetic Polynucleotide
aagaacctccgcacagatatt

SEQ ID NO: 564 - DNA, 21 bp, Artificial Sequence
  Synthetic Polynucleotide
tgtataaatccaaccttctgc

SEQ ID NO: 565 - DNA, 21 bp, Artificial Sequence
  Synthetic Polynucleotide
tccagggtttgtttgtttcat

SEQ ID NO: 566 - DNA, 21 bp, Artificial Sequence
  Synthetic Polynucleotide
gagagaattgccaaaagccac

SEQ ID NO: 567 - DNA, 21 bp, Artificial Sequence
  Synthetic Polynucleotide
gagacaagcactaacactgtg

SEQ ID NO: 568 - DNA, 21 bp, Artificial Sequence
  Synthetic Polynucleotide
gatcccggcatttggcagcat

SEQ ID NO: 569 - DNA, 21 bp, Artificial Sequence
  Synthetic Polynucleotide
tcccggagagaagatgacatt

SEQ ID NO: 570 - DNA, 21 bp, Artificial Sequence
  Synthetic Polynucleotide
aattcccaccaagtttggaat

SEQ ID NO: 571 - DNA, 21 bp, Artificial Sequence
  Synthetic Polynucleotide
atctttacatttgaagccaaa

SEQ ID NO: 572 - DNA, 21 bp, Artificial Sequence
  Synthetic Polynucleotide
cacatggacaactgctataaa

SEQ ID NO: 573 - DNA, 21 bp, Artificial Sequence
  Synthetic Polynucleotide
gaaaactgcaaaccctggtct

SEQ ID NO: 574 - DNA, 21 bp, Artificial Sequence
  Synthetic Polynucleotide
aatctttacatttgaagccaa

SEQ ID NO: 575 - DNA, 21 bp, Artificial Sequence
  Synthetic Polynucleotide
atttctcagcattcaagcagg

SEQ ID NO: 576 - DNA, 21 bp, Artificial Sequence
  Synthetic Polynucleotide
aaacaaattcaaggttaattg

SEQ ID NO: 577 - DNA, 21 bp, Artificial Sequence
  Synthetic Polynucleotide
atgtcactaataaatgaaact

SEQ ID NO: 578 - DNA, 21 bp, Artificial Sequence
  Synthetic Polynucleotide
gtttctgcggttggtaaagag

SEQ ID NO: 579 - DNA, 21 bp, Artificial Sequence
  Synthetic Polynucleotide
ctatcttctttgccatcaaag

SEQ ID NO: 580 - DNA, 21 bp, Artificial Sequence
  Synthetic Polynucleotide
tgtataacctcaagttctgat

SEQ ID NO: 581 - DNA, 21 bp, Artificial Sequence
  Synthetic Polynucleotide
cataagaacctccgcacagat

SEQ ID NO: 582 - DNA, 21 bp, Artificial Sequence
  Synthetic Polynucleotide
gcatcgtcatgagtatgtaca

SEQ ID NO: 583 - DNA, 21 bp, Artificial Sequence
  Synthetic Polynucleotide
tgcagtgacaactgtggaagg

SEQ ID NO: 584 - DNA, 21 bp, Artificial Sequence
  Synthetic Polynucleotide
tttatggacctgagctgaaga

SEQ ID NO: 585 - DNA, 21 bp, Artificial Sequence
  Synthetic Polynucleotide
agttcaagaatatgctgtttc

SEQ ID NO: 586 - DNA, 21 bp, Artificial Sequence
  Synthetic Polynucleotide
gcctcaaccctcaaggagctg

SEQ ID NO: 587 - DNA, 21 bp, Artificial Sequence
  Synthetic Polynucleotide
ggaacataccacagaacatgg

SEQ ID NO: 588 - DNA, 21 bp, Artificial Sequence
  Synthetic Polynucleotide
tcgtcatgagtatgtacacac

SEQ ID NO: 589 - DNA, 21 bp, Artificial Sequence
  Synthetic Polynucleotide
ctgacacacttcaaccagaag

SEQ ID NO: 590 - DNA, 21 bp, Artificial Sequence
  Synthetic Polynucleotide
gctcagcacacaggtaataac

SEQ ID NO: 591 - DNA, 21 bp, Artificial Sequence
  Synthetic Polynucleotide
aacatggatctattaaagtca

SEQ ID NO: 592 - DNA, 21 bp, Artificial Sequence
  Synthetic Polynucleotide
tggaaccaagctcggctgtgg

SEQ ID NO: 593 - DNA, 21 bp, Artificial Sequence
  Synthetic Polynucleotide
ctgccattgatattggacagg

SEQ ID NO: 594 - DNA, 21 bp, Artificial Sequence
  Synthetic Polynucleotide
ctcaccctcagcttcttcttc

SEQ ID NO: 595 - DNA, 21 bp, Artificial Sequence
  Synthetic Polynucleotide
gcaggagagaattgccaaaag

SEQ ID NO: 596 - DNA, 21 bp, Artificial Sequence
  Synthetic Polynucleotide
gacatgctgataactggtggc

SEQ ID NO: 597 - DNA, 21 bp, Artificial Sequence
  Synthetic Polynucleotide
aaagattatgtttaatcttta

SEQ ID NO: 598 - DNA, 21 bp, Artificial Sequence
  Synthetic Polynucleotide
caggagagaattgccaaaagc

SEQ ID NO: 599 - DNA, 21 bp, Artificial Sequence
  Synthetic Polynucleotide
aaaagattatgtttaatcttt

SEQ ID NO: 600 - DNA, 21 bp, Artificial Sequence
  Synthetic Polynucleotide
aggccttcataccaaaatggt

SEQ ID NO: 601 - DNA, 21 bp, Artificial Sequence
  Synthetic Polynucleotide
tgcgctggtttgctgggaagc

SEQ ID NO: 602 - DNA, 21 bp, Artificial Sequence
  Synthetic Polynucleotide
aatgttctagatttctttccc

SEQ ID NO: 603 - DNA, 21 bp, Artificial Sequence
  Synthetic Polynucleotide
agctcgagctcagcacacagg

SEQ ID NO: 604 - DNA, 21 bp, Artificial Sequence
  Synthetic Polynucleotide
caccattgctgttccaaaagg

SEQ ID NO: 605 - DNA, 21 bp, Artificial Sequence
  Synthetic Polynucleotide
caagatcgtccacttttctgc

SEQ ID NO: 606 - DNA, 21 bp, Artificial Sequence
  Synthetic Polynucleotide
caggtaataacgtgaaggaac

SEQ ID NO: 607 - DNA, 21 bp, Artificial Sequence
  Synthetic Polynucleotide
gtggcatgagagttttattca

SEQ ID NO: 608 - DNA, 21 bp, Artificial Sequence
  Synthetic Polynucleotide
cttcaaaccaatgaacagcaa

SEQ ID NO: 609 - DNA, 21 bp, Artificial Sequence
  Synthetic Polynucleotide
ttcaaaccaatgaacagcaaa

SEQ ID NO: 610 - DNA, 21 bp, Artificial Sequence
  Synthetic Polynucleotide
atccaaccaactcaattattg

SEQ ID NO: 611 - DNA, 21 bp, Artificial Sequence
  Synthetic Polynucleotide
caaaaaagattatgtttaatc

SEQ ID NO: 612 - DNA, 21 bp, Artificial Sequence
  Synthetic Polynucleotide
ctgtgggatgcctgcagagga

SEQ ID NO: 613 - DNA, 21 bp, Artificial Sequence
  Synthetic Polynucleotide
ccctcaagaccactcagaggc

SEQ ID NO: 614 - DNA, 21 bp, Artificial Sequence
  Synthetic Polynucleotide
gggatgcctgcagaggaggtg

SEQ ID NO: 615 - DNA, 21 bp, Artificial Sequence
  Synthetic Polynucleotide
ccagtggcatgagagttttat

SEQ ID NO: 616 - DNA, 21 bp, Artificial Sequence
  Synthetic Polynucleotide
ggatctattaaagtcacagaa

SEQ ID NO: 617 - DNA, 21 bp, Artificial Sequence
  Synthetic Polynucleotide
cttcctgtgatccattttact

SEQ ID NO: 618 - DNA, 21 bp, Artificial Sequence
  Synthetic Polynucleotide
ctggctgcttctatcttcttt

SEQ ID NO: 619 - DNA, 21 bp, Artificial Sequence
  Synthetic Polynucleotide
tgtacacagatggctctgtgc

SEQ ID NO: 620 - DNA, 21 bp, Artificial Sequence
  Synthetic Polynucleotide
tcaagttctacctgacagtcc

SEQ ID NO: 621 - DNA, 19 bp, Artificial Sequence
  Synthetic Polynucleotide
cattcacttgtcttccaaa

SEQ ID NO: 622 - DNA, 19 bp, Artificial Sequence
  Synthetic Polynucleotide
catacttggagagcatcac

SEQ ID NO: 623 - DNA, 19 bp, Artificial Sequence
  Synthetic Polynucleotide
actcgaaccacaatccggt

SEQ ID NO: 624 - DNA, 19 bp, Artificial Sequence
  Synthetic Polynucleotide
tctttaccaaccgcagaaa

SEQ ID NO: 625 - DNA, 19 bp, Artificial Sequence
  Synthetic Polynucleotide
atggcaaagaagatagaag

SEQ ID NO: 626 - DNA, 19 bp, Artificial Sequence
  Synthetic Polynucleotide
catgttctgtggtatgttc

SEQ ID NO: 627 - DNA, 19 bp, Artificial Sequence
  Synthetic Polynucleotide
cttcagctcaggtccataa

SEQ ID NO: 628 - DNA, 19 bp, Artificial Sequence
  Synthetic Polynucleotide
cttgatcttggcgtgggcc

SEQ ID NO: 629 - DNA, 19 bp, Artificial Sequence
  Synthetic Polynucleotide
gggaatagcacaaaccctt

SEQ ID NO: 630 - DNA, 19 bp, Artificial Sequence
  Synthetic Polynucleotide
tgcagtaaaatggatcaca

SEQ ID NO: 631 - DNA, 19 bp, Artificial Sequence
  Synthetic Polynucleotide
gctcaataattgagttggt

SEQ ID NO: 632 - DNA, 19 bp, Artificial Sequence
  Synthetic Polynucleotide
agccttagatagctgcaga

SEQ ID NO: 633 - DNA, 19 bp, Artificial Sequence
  Synthetic Polynucleotide
acaacattatctgcttcgg

SEQ ID NO: 634 - DNA, 19 bp, Artificial Sequence
  Synthetic Polynucleotide
ttccggagcagtgtgtaca

SEQ ID NO: 635 - DNA, 19 bp, Artificial Sequence
  Synthetic Polynucleotide
gccaaaagggttgtctctg

SEQ ID NO: 636 - DNA, 19 bp, Artificial Sequence
  Synthetic Polynucleotide
ggatgctgccaaatgccgg

SEQ ID NO: 637 - DNA, 19 bp, Artificial Sequence
  Synthetic Polynucleotide
tatgcccaacacaagtaac

SEQ ID NO: 638 - DNA, 19 bp, Artificial Sequence
  Synthetic Polynucleotide
cttgctaggcattcttccc

SEQ ID NO: 639 - DNA, 19 bp, Artificial Sequence
  Synthetic Polynucleotide
tgcattcacttgtcttcca

SEQ ID NO: 640 - DNA, 19 bp, Artificial Sequence
  Synthetic Polynucleotide
agagtaatcttgctttatg

SEQ ID NO: 641 - DNA, 19 bp, Artificial Sequence
  Synthetic Polynucleotide
agcttattccaaacttggt

SEQ ID NO: 642 - DNA, 19 bp, Artificial Sequence
  Synthetic Polynucleotide
acaattctccttgttgaac

SEQ ID NO: 643 - DNA, 19 bp, Artificial Sequence
  Synthetic Polynucleotide
ctggatctgcatttttctc

SEQ ID NO: 644 - DNA, 19 bp, Artificial Sequence
  Synthetic Polynucleotide
atgatactgagagcttgct

SEQ ID NO: 645 - DNA, 19 bp, Artificial Sequence
  Synthetic Polynucleotide
tacatactcatgacgatgc

SEQ ID NO: 646 - DNA, 19 bp, Artificial Sequence
  Synthetic Polynucleotide
gcaacatggtgcaaggagc

SEQ ID NO: 647 - DNA, 19 bp, Artificial Sequence
  Synthetic Polynucleotide
ctcaataattgagttggtt

SEQ ID NO: 648 - DNA, 19 bp, Artificial Sequence
  Synthetic Polynucleotide
atgatatgcccaacacaag

SEQ ID NO: 649 - DNA, 19 bp, Artificial Sequence
  Synthetic Polynucleotide
caacacaagtaaccttatc

SEQ ID NO: 650 - DNA, 19 bp, Artificial Sequence
  Synthetic Polynucleotide
gagcatcactgtgcaagcc

SEQ ID NO: 651 - DNA, 19 bp, Artificial Sequence
  Synthetic Polynucleotide
ttaaccttgaatttgtttc

SEQ ID NO: 652 - DNA, 19 bp, Artificial Sequence
  Synthetic Polynucleotide
gacagtccaagatcacaaa

SEQ ID NO: 653 - DNA, 19 bp, Artificial Sequence
  Synthetic Polynucleotide
tggatctgcatttttctcc

SEQ ID NO: 654 - DNA, 19 bp, Artificial Sequence
  Synthetic Polynucleotide
ccctgacacaacattatct

SEQ ID NO: 655 - DNA, 19 bp, Artificial Sequence
  Synthetic Polynucleotide
gcttattccaaacttggtg

SEQ ID NO: 656 - DNA, 19 bp, Artificial Sequence
  Synthetic Polynucleotide
agattcaaggttatgcttt

SEQ ID NO: 657 - DNA, 19 bp, Artificial Sequence
  Synthetic Polynucleotide
cttcacgttattacctgtg

SEQ ID NO: 658 - DNA, 19 bp, Artificial Sequence
  Synthetic Polynucleotide
tcaattgtgataatggctg

SEQ ID NO: 659 - DNA, 19 bp, Artificial Sequence
  Synthetic Polynucleotide
ttgagttggttggattttt

SEQ ID NO: 660 - DNA, 19 bp, Artificial Sequence
  Synthetic Polynucleotide
tcgtcttggtgcttcctat

SEQ ID NO: 661 - DNA, 19 bp, Artificial Sequence
  Synthetic Polynucleotide
tgtccattgaggtcagcgc

SEQ ID NO: 662 - DNA, 19 bp, Artificial Sequence
  Synthetic Polynucleotide
atgtcatcttctctccggg

SEQ ID NO: 663 - DNA, 19 bp, Artificial Sequence
  Synthetic Polynucleotide
cacctgtccaatatcaatg

SEQ ID NO: 664 - DNA, 19 bp, Artificial Sequence
  Synthetic Polynucleotide
tcacgttattacctgtgtg

SEQ ID NO: 665 - DNA, 19 bp, Artificial Sequence
  Synthetic Polynucleotide
tgcatattcaccatttagg

SEQ ID NO: 666 - DNA, 19 bp, Artificial Sequence
  Synthetic Polynucleotide
ttctataaaacccagtggc

SEQ ID NO: 667 - DNA, 19 bp, Artificial Sequence
  Synthetic Polynucleotide
ctgattccggagcagtgtg

SEQ ID NO: 668 - DNA, 19 bp, Artificial Sequence
  Synthetic Polynucleotide
gatatgcccaacacaagta

SEQ ID NO: 669 - DNA, 19 bp, Artificial Sequence
  Synthetic Polynucleotide
tcgtcacagtacacggcct

SEQ ID NO: 670 - DNA, 19 bp, Artificial Sequence
  Synthetic Polynucleotide
tgatactgagagcttgcta

SEQ ID NO: 671 - DNA, 19 bp, Artificial Sequence
  Synthetic Polynucleotide
tgaacttcatctcaatgcc

SEQ ID NO: 672 - DNA, 19 bp, Artificial Sequence
  Synthetic Polynucleotide
gtagcccagattgggtgtt

SEQ ID NO: 673 - DNA, 19 bp, Artificial Sequence
  Synthetic Polynucleotide
tcagcggaaatgaaacaaa

SEQ ID NO: 674 - DNA, 19 bp, Artificial Sequence
  Synthetic Polynucleotide
cccatagctgaagtagtgg

SEQ ID NO: 675 - DNA, 19 bp, Artificial Sequence
  Synthetic Polynucleotide
tagatccatgttctgtggt

SEQ ID NO: 676 - DNA, 19 bp, Artificial Sequence
  Synthetic Polynucleotide
atatctgtgcggaggttct

SEQ ID NO: 677 - DNA, 19 bp, Artificial Sequence
  Synthetic Polynucleotide
caaatgccgggatcttgta

SEQ ID NO: 678 - DNA, 19 bp, Artificial Sequence
  Synthetic Polynucleotide
ccggagcagtgtgtacata

SEQ ID NO: 679 - DNA, 19 bp, Artificial Sequence
  Synthetic Polynucleotide
ctgcaacatggtgcaagga

SEQ ID NO: 680 - DNA, 19 bp, Artificial Sequence
  Synthetic Polynucleotide
ttctaggtggaaattactt

SEQ ID NO: 681 - DNA, 19 bp, Artificial Sequence
  Synthetic Polynucleotide
cccgcactgggagccgtgg

SEQ ID NO: 682 - DNA, 19 bp, Artificial Sequence
  Synthetic Polynucleotide
aaacccagtggcagacaag

SEQ ID NO: 683 - DNA, 19 bp, Artificial Sequence
  Synthetic Polynucleotide
ttattagtgacatcaagca

SEQ ID NO: 684 - DNA, 19 bp, Artificial Sequence
  Synthetic Polynucleotide
cacggatggcatctttgat

SEQ ID NO: 685 - DNA, 19 bp, Artificial Sequence
  Synthetic Polynucleotide
cagacgatcatacttggag

SEQ ID NO: 686 - DNA, 19 bp, Artificial Sequence
  Synthetic Polynucleotide
gtggtatgttcctcctgct

SEQ ID NO: 687 - DNA, 19 bp, Artificial Sequence
  Synthetic Polynucleotide
ccataatactctgagagag

SEQ ID NO: 688 - DNA, 19 bp, Artificial Sequence
  Synthetic Polynucleotide
gtcaacctcactcttccga

SEQ ID NO: 689 - DNA, 19 bp, Artificial Sequence
  Synthetic Polynucleotide
gtccattgaggtcagcgct

SEQ ID NO: 690 - DNA, 19 bp, Artificial Sequence
  Synthetic Polynucleotide
gcagttgtccatgtggaat

SEQ ID NO: 691 - DNA, 19 bp, Artificial Sequence
  Synthetic Polynucleotide
tgcatagatggccttcttg

SEQ ID NO: 692 - DNA, 19 bp, Artificial Sequence
  Synthetic Polynucleotide
caattgtgataatggctgg

SEQ ID NO: 693 - DNA, 19 bp, Artificial Sequence
  Synthetic Polynucleotide
cagacaagctcactgtgtc

SEQ ID NO: 694 - DNA, 19 bp, Artificial Sequence
  Synthetic Polynucleotide
caatatctgtgcggaggtt

SEQ ID NO: 695 - DNA, 19 bp, Artificial Sequence
  Synthetic Polynucleotide
acccagtggcagacaagct

SEQ ID NO: 696 - DNA, 19 bp, Artificial Sequence
  Synthetic Polynucleotide
ttgggcaggaagcttagca

SEQ ID NO: 697 - DNA, 19 bp, Artificial Sequence
  Synthetic Polynucleotide
ggttggttttgcacagccg

SEQ ID NO: 698 - DNA, 19 bp, Artificial Sequence
  Synthetic Polynucleotide
gtacatactcatgacgatg

SEQ ID NO: 699 - DNA, 19 bp, Artificial Sequence
  Synthetic Polynucleotide
tttgggcaggaagcttagc

SEQ ID NO: 700 - DNA, 19 bp, Artificial Sequence
  Synthetic Polynucleotide
catgccactggttaccttg

SEQ ID NO: 701 - DNA, 19 bp, Artificial Sequence
  Synthetic Polynucleotide
gtgtcagcaaccacagcac

SEQ ID NO: 702 - DNA, 19 bp, Artificial Sequence
  Synthetic Polynucleotide
tctccaccacctttctgcc

SEQ ID NO: 703 - DNA, 19 bp, Artificial Sequence
  Synthetic Polynucleotide
agcctcgtcttggtgcttc

SEQ ID NO: 704 - DNA, 19 bp, Artificial Sequence
  Synthetic Polynucleotide
gggttccctgagttagtct

SEQ ID NO: 705 - DNA, 19 bp, Artificial Sequence
  Synthetic Polynucleotide
ccctcacagaccagggttt

SEQ ID NO: 706 - DNA, 19 bp, Artificial Sequence
  Synthetic Polynucleotide
tcatggtgttctgtgtaga

SEQ ID NO: 707 - DNA, 19 bp, Artificial Sequence
  Synthetic Polynucleotide
tcataggtgattttcaccc

SEQ ID NO: 708 - DNA, 19 bp, Artificial Sequence
  Synthetic Polynucleotide
gtccaatatcaatggcagg

SEQ ID NO: 709 - DNA, 19 bp, Artificial Sequence
  Synthetic Polynucleotide
ggtttagactggagccaac

SEQ ID NO: 710 - DNA, 19 bp, Artificial Sequence
  Synthetic Polynucleotide
caccagttatcagcatgtc

SEQ ID NO: 711 - DNA, 19 bp, Artificial Sequence
  Synthetic Polynucleotide
ttcagctcaggtccataaa

SEQ ID NO: 712 - DNA, 19 bp, Artificial Sequence
  Synthetic Polynucleotide
cgagcatgatactgagagc

SEQ ID NO: 713 - DNA, 19 bp, Artificial Sequence
  Synthetic Polynucleotide
gtaactggagttttcaggt

SEQ ID NO: 714 - DNA, 19 bp, Artificial Sequence
  Synthetic Polynucleotide
tcaagcaccagtccaacta

SEQ ID NO: 715 - DNA, 19 bp, Artificial Sequence
  Synthetic Polynucleotide
atcttggcgtgggcccggg

SEQ ID NO: 716 - DNA, 19 bp, Artificial Sequence
  Synthetic Polynucleotide
tttaccaaccgcagaaact

SEQ ID NO: 717 - DNA, 19 bp, Artificial Sequence
  Synthetic Polynucleotide
attcacttgtcttccaaat

SEQ ID NO: 718 - DNA, 19 bp, Artificial Sequence
  Synthetic Polynucleotide
cagattgggtgttctataa

SEQ ID NO: 719 - DNA, 19 bp, Artificial Sequence
  Synthetic Polynucleotide
atacttggagagcatcact

SEQ ID NO: 720 - DNA, 19 bp, Artificial Sequence
  Synthetic Polynucleotide
ttctggttgaagtgtgtca

SEQ ID NO: 721 - DNA, 19 bp, Artificial Sequence
  Synthetic Polynucleotide
ctccacagccgagcttggt

SEQ ID NO: 722 - DNA, 19 bp, Artificial Sequence
  Synthetic Polynucleotide
attatctgcttcggaaaac

SEQ ID NO: 723 - DNA, 19 bp, Artificial Sequence
  Synthetic Polynucleotide
gcagacgatcatacttgga

SEQ ID NO: 724 - DNA, 19 bp, Artificial Sequence
  Synthetic Polynucleotide
gcttgctaggcattcttcc

SEQ ID NO: 725 - DNA, 19 bp, Artificial Sequence
  Synthetic Polynucleotide
caggcattggcagaaaagt

SEQ ID NO: 726 - DNA, 19 bp, Artificial Sequence
  Synthetic Polynucleotide
tcataggaaacagcatatt

SEQ ID NO: 727 - DNA, 19 bp, Artificial Sequence
  Synthetic Polynucleotide
tccacagccgagcttggtt

SEQ ID NO: 728 - DNA, 19 bp, Artificial Sequence
  Synthetic Polynucleotide
gtgttgggcacagtgttag

SEQ ID NO: 729 - DNA, 19 bp, Artificial Sequence
  Synthetic Polynucleotide
cccaacatttttgcaacaa

SEQ ID NO: 730 - DNA, 19 bp, Artificial Sequence
  Synthetic Polynucleotide
atcagcggaaatgaaacaa

SEQ ID NO: 731 - DNA, 19 bp, Artificial Sequence
  Synthetic Polynucleotide
tggcatctttgatggcaaa

SEQ ID NO: 732 - DNA, 19 bp, Artificial Sequence
  Synthetic Polynucleotide
agtctcaaagctgtagccc

SEQ ID NO: 733 - DNA, 19 bp, Artificial Sequence
  Synthetic Polynucleotide
ttcacgttattacctgtgt

SEQ ID NO: 734 - DNA, 19 bp, Artificial Sequence
  Synthetic Polynucleotide
aatgatatgcccaacacaa

SEQ ID NO: 735 - DNA, 19 bp, Artificial Sequence
  Synthetic Polynucleotide
ggtctgtagcctgtgcagc

SEQ ID NO: 736 - DNA, 19 bp, Artificial Sequence
  Synthetic Polynucleotide
tcttggtgcttcctattcc

SEQ ID NO: 737 - DNA, 19 bp, Artificial Sequence
  Synthetic Polynucleotide
ttaccaaccgcagaaactt

SEQ ID NO: 738 - DNA, 19 bp, Artificial Sequence
  Synthetic Polynucleotide
ttggtgcttcctattcctt

SEQ ID NO: 739 - DNA, 19 bp, Artificial Sequence
  Synthetic Polynucleotide
ggtttaagcttctagaggt

SEQ ID NO: 740 - DNA, 19 bp, Artificial Sequence
  Synthetic Polynucleotide
gtttagactggagccaaca

SEQ ID NO: 741 - DNA, 19 bp, Artificial Sequence
  Synthetic Polynucleotide
ttatagcagttgtccatgt

SEQ ID NO: 742 - DNA, 19 bp, Artificial Sequence
  Synthetic Polynucleotide
accagttatcagcatgtcc

SEQ ID NO: 743 - DNA, 19 bp, Artificial Sequence
  Synthetic Polynucleotide
ccgagcatgatactgagag

SEQ ID NO: 744 - DNA, 19 bp, Artificial Sequence
  Synthetic Polynucleotide
ggttatacaggctgtccag

SEQ ID NO: 745 - DNA, 19 bp, Artificial Sequence
  Synthetic Polynucleotide
ctcgaaccacaatccggtt

SEQ ID NO: 746 - DNA, 19 bp, Artificial Sequence
  Synthetic Polynucleotide
cagttgtccatgtggaata

SEQ ID NO: 747 - DNA, 19 bp, Artificial Sequence
  Synthetic Polynucleotide
ttggaacagcaatggtgca

SEQ ID NO: 748 - DNA, 19 bp, Artificial Sequence
  Synthetic Polynucleotide
ccaaaagggttgtctctgg

SEQ ID NO: 749 - DNA, 19 bp, Artificial Sequence
  Synthetic Polynucleotide
atcaagcaccagtccaact

SEQ ID NO: 750 - DNA, 19 bp, Artificial Sequence
  Synthetic Polynucleotide
agtatctcctccgggctca

SEQ ID NO: 751 - DNA, 19 bp, Artificial Sequence
  Synthetic Polynucleotide
tccaaacttggtgggaatt

SEQ ID NO: 752 - DNA, 19 bp, Artificial Sequence
  Synthetic Polynucleotide
tcaagatggtctgacaagc

SEQ ID NO: 753 - DNA, 19 bp, Artificial Sequence
  Synthetic Polynucleotide
gccttagatagctgcagat

SEQ ID NO: 754 - DNA, 19 bp, Artificial Sequence
  Synthetic Polynucleotide
gattccggagcagtgtgta

SEQ ID NO: 755 - DNA, 19 bp, Artificial Sequence
  Synthetic Polynucleotide
cgtggcttttggcaattct

SEQ ID NO: 756 - DNA, 19 bp, Artificial Sequence
  Synthetic Polynucleotide
ctctatggagagcagtatc

SEQ ID NO: 757 - DNA, 19 bp, Artificial Sequence
  Synthetic Polynucleotide
tcctaagagacactggcag

SEQ ID NO: 758 - DNA, 19 bp, Artificial Sequence
  Synthetic Polynucleotide
ggtacctcactcctaagag

SEQ ID NO: 759 - DNA, 19 bp, Artificial Sequence
  Synthetic Polynucleotide
gtaaagttgcactggcgaa

SEQ ID NO: 760 - DNA, 19 bp, Artificial Sequence
  Synthetic Polynucleotide
cacgttattacctgtgtgc

SEQ ID NO: 761 - DNA, 19 bp, Artificial Sequence
  Synthetic Polynucleotide
tgtcatcttctctccggga

SEQ ID NO: 762 - DNA, 19 bp, Artificial Sequence
  Synthetic Polynucleotide
ttatctgcttcggaaaacc

SEQ ID NO: 763 - DNA, 19 bp, Artificial Sequence
  Synthetic Polynucleotide
agtctccaggtagaagtgc

SEQ ID NO: 764 - DNA, 19 bp, Artificial Sequence
  Synthetic Polynucleotide
aggccgtgttggagggaag

SEQ ID NO: 765 - DNA, 19 bp, Artificial Sequence
  Synthetic Polynucleotide
tctccacagccgagcttgg

SEQ ID NO: 766 - DNA, 19 bp, Artificial Sequence
  Synthetic Polynucleotide
tcttccgagcatgatactg

SEQ ID NO: 767 - DNA, 19 bp, Artificial Sequence
  Synthetic Polynucleotide
gagcatgatactgagagct

SEQ ID NO: 768 - DNA, 19 bp, Artificial Sequence
  Synthetic Polynucleotide
tcaatggcagggtttagac

SEQ ID NO: 769 - DNA, 19 bp, Artificial Sequence
  Synthetic Polynucleotide
cagccggagagacagctca

SEQ ID NO: 770 - DNA, 19 bp, Artificial Sequence
  Synthetic Polynucleotide
tcatcacggtccagcatgc

SEQ ID NO: 771 - DNA, 19 bp, Artificial Sequence
  Synthetic Polynucleotide
tatctgtgcggaggttctt

SEQ ID NO: 772 - DNA, 19 bp, Artificial Sequence
  Synthetic Polynucleotide
tttctaggtggaaattact

SEQ ID NO: 773 - DNA, 19 bp, Artificial Sequence
  Synthetic Polynucleotide
aatatctgtgcggaggttc

SEQ ID NO: 774 - DNA, 19 bp, Artificial Sequence
  Synthetic Polynucleotide
gcagaaggttggatttata

SEQ ID NO: 775 - DNA, 19 bp, Artificial Sequence
  Synthetic Polynucleotide
atgaaacaaacaaaccctg

SEQ ID NO: 776 - DNA, 19 bp, Artificial Sequence
  Synthetic Polynucleotide
gtggcttttggcaattctc

SEQ ID NO: 777 - DNA, 19 bp, Artificial Sequence
  Synthetic Polynucleotide
cacagtgttagtgcttgtc

SEQ ID NO: 778 - DNA, 19 bp, Artificial Sequence
  Synthetic Polynucleotide
atgctgccaaatgccggga

SEQ ID NO: 779 - DNA, 19 bp, Artificial Sequence
  Synthetic Polynucleotide
aatgtcatcttctctccgg

SEQ ID NO: 780 - DNA, 19 bp, Artificial Sequence
  Synthetic Polynucleotide
attccaaacttggtgggaa

SEQ ID NO: 781 - DNA, 19 bp, Artificial Sequence
  Synthetic Polynucleotide
tttggcttcaaatgtaaag

SEQ ID NO: 782 - DNA, 19 bp, Artificial Sequence
  Synthetic Polynucleotide
tttatagcagttgtccatg

SEQ ID NO: 783 - DNA, 19 bp, Artificial Sequence
  Synthetic Polynucleotide
agaccagggtttgcagttt

SEQ ID NO: 784 - DNA, 19 bp, Artificial Sequence
  Synthetic Polynucleotide
ttggcttcaaatgtaaaga

SEQ ID NO: 785 - DNA, 19 bp, Artificial Sequence
  Synthetic Polynucleotide
cctgcttgaatgctgagaa

SEQ ID NO: 786 - DNA, 19 bp, Artificial Sequence
  Synthetic Polynucleotide
caattaaccttgaatttgt

SEQ ID NO: 787 - DNA, 19 bp, Artificial Sequence
  Synthetic Polynucleotide
agtttcatttattagtgac

SEQ ID NO: 788 - DNA, 19 bp, Artificial Sequence
  Synthetic Polynucleotide
ctctttaccaaccgcagaa

SEQ ID NO: 789 - DNA, 19 bp, Artificial Sequence
  Synthetic Polynucleotide
ctttgatggcaaagaagat

SEQ ID NO: 790 - DNA, 19 bp, Artificial Sequence
  Synthetic Polynucleotide
atcagaacttgaggttata

SEQ ID NO: 791 - DNA, 19 bp, Artificial Sequence
  Synthetic Polynucleotide
atctgtgcggaggttctta

SEQ ID NO: 792 - DNA, 19 bp, Artificial Sequence
  Synthetic Polynucleotide
tgtacatactcatgacgat

SEQ ID NO: 793 - DNA, 19 bp, Artificial Sequence
  Synthetic Polynucleotide
ccttccacagttgtcactg

SEQ ID NO: 794 - DNA, 19 bp, Artificial Sequence
  Synthetic Polynucleotide
tcttcagctcaggtccata

SEQ ID NO: 795 - DNA, 19 bp, Artificial Sequence
  Synthetic Polynucleotide
gaaacagcatattcttgaa

SEQ ID NO: 796 - DNA, 19 bp, Artificial Sequence
  Synthetic Polynucleotide
cagctccttgagggttgag

SEQ ID NO: 797 - DNA, 19 bp, Artificial Sequence
  Synthetic Polynucleotide
ccatgttctgtggtatgtt

SEQ ID NO: 798 - DNA, 19 bp, Artificial Sequence
  Synthetic Polynucleotide
gtgtgtacatactcatgac

SEQ ID NO: 799 - DNA, 19 bp, Artificial Sequence
  Synthetic Polynucleotide
cttctggttgaagtgtgtc

SEQ ID NO: 800 - DNA, 19 bp, Artificial Sequence
  Synthetic Polynucleotide
gttattacctgtgtgctga

SEQ ID NO: 801 - DNA, 19 bp, Artificial Sequence
  Synthetic Polynucleotide
tgactttaatagatccatg

SEQ ID NO: 802 - DNA, 19 bp, Artificial Sequence
  Synthetic Polynucleotide
ccacagccgagcttggttc

SEQ ID NO: 803 - DNA, 19 bp, Artificial Sequence
  Synthetic Polynucleotide
cctgtccaatatcaatggc

SEQ ID NO: 804 - DNA, 19 bp, Artificial Sequence
  Synthetic Polynucleotide
gaagaagaagctgagggtg

SEQ ID NO: 805 - DNA, 19 bp, Artificial Sequence
  Synthetic Polynucleotide
cttttggcaattctctcct

SEQ ID NO: 806 - DNA, 19 bp, Artificial Sequence
  Synthetic Polynucleotide
gccaccagttatcagcatg

SEQ ID NO: 807 - DNA, 19 bp, Artificial Sequence
  Synthetic Polynucleotide
taaagattaaacataatct

SEQ ID NO: 808 - DNA, 19 bp, Artificial Sequence
  Synthetic Polynucleotide
gcttttggcaattctctcc

SEQ ID NO: 809 - DNA, 19 bp, Artificial Sequence
  Synthetic Polynucleotide
aaagattaaacataatctt

SEQ ID NO: 810 - DNA, 19 bp, Artificial Sequence
  Synthetic Polynucleotide
accattttggtatgaaggc

SEQ ID NO: 811 - DNA, 19 bp, Artificial Sequence
  Synthetic Polynucleotide
gcttcccagcaaaccagcg

SEQ ID NO: 812 - DNA, 19 bp, Artificial Sequence
  Synthetic Polynucleotide
gggaaagaaatctagaaca

SEQ ID NO: 813 - DNA, 19 bp, Artificial Sequence
  Synthetic Polynucleotide
cctgtgtgctgagctcgag

SEQ ID NO: 814 - DNA, 19 bp, Artificial Sequence
  Synthetic Polynucleotide
ccttttggaacagcaatgg

SEQ ID NO: 815 - DNA, 19 bp, Artificial Sequence
  Synthetic Polynucleotide
gcagaaaagtggacgatct

SEQ ID NO: 816 - DNA, 19 bp, Artificial Sequence
  Synthetic Polynucleotide
gttccttcacgttattacc

SEQ ID NO: 817 - DNA, 19 bp, Artificial Sequence
  Synthetic Polynucleotide
tgaataaaactctcatgcc

SEQ ID NO: 818 - DNA, 19 bp, Artificial Sequence
  Synthetic Polynucleotide
ttgctgttcattggtttga

SEQ ID NO: 819 - DNA, 19 bp, Artificial Sequence
  Synthetic Polynucleotide
tttgctgttcattggtttg

SEQ ID NO: 820 - DNA, 19 bp, Artificial Sequence
  Synthetic Polynucleotide
caataattgagttggttgg

SEQ ID NO: 821 - DNA, 19 bp, Artificial Sequence
  Synthetic Polynucleotide
gattaaacataatcttttt

SEQ ID NO: 822 - DNA, 19 bp, Artificial Sequence
  Synthetic Polynucleotide
tcctctgcaggcatcccac

SEQ ID NO: 823 - DNA, 19 bp, Artificial Sequence
  Synthetic Polynucleotide
gcctctgagtggtcttgag

SEQ ID NO: 824 - DNA, 19 bp, Artificial Sequence
  Synthetic Polynucleotide
cacctcctctgcaggcatc

SEQ ID NO: 825 - DNA, 19 bp, Artificial Sequence
  Synthetic Polynucleotide
ataaaactctcatgccact

SEQ ID NO: 826 - DNA, 19 bp, Artificial Sequence
  Synthetic Polynucleotide
ttctgtgactttaatagat

SEQ ID NO: 827 - DNA, 19 bp, Artificial Sequence
  Synthetic Polynucleotide
agtaaaatggatcacagga

SEQ ID NO: 828 - DNA, 19 bp, Artificial Sequence
  Synthetic Polynucleotide
aaagaagatagaagcagcc

SEQ ID NO: 829 - DNA, 19 bp, Artificial Sequence
  Synthetic Polynucleotide
gcacagagccatctgtgta

SEQ ID NO: 830 - DNA, 19 bp, Artificial Sequence
  Synthetic Polynucleotide
ggactgtcaggtagaactt

SEQ ID NO: 831 - DNA, 23 bp, Artificial Sequence
  Synthetic Polynucleotide
cattcacttgtcttccaaatccc

SEQ ID NO: 832 - DNA, 23 bp, Artificial Sequence
  Synthetic Polynucleotide
catacttggagagcatcactgtg

SEQ ID NO: 833 - DNA, 23 bp, Artificial Sequence
  Synthetic Polynucleotide
actcgaaccacaatccggtttgc

SEQ ID NO: 834 - DNA, 23 bp, Artificial Sequence
  Synthetic Polynucleotide
tctttaccaaccgcagaaacttg

SEQ ID NO: 835 - DNA, 23 bp, Artificial Sequence
  Synthetic Polynucleotide
atggcaaagaagatagaagcagc

SEQ ID NO: 836 - DNA, 23 bp, Artificial Sequence
  Synthetic Polynucleotide
catgttctgtggtatgttcctcc

SEQ ID NO: 837 - DNA, 23 bp, Artificial Sequence
  Synthetic Polynucleotide
cttcagctcaggtccataaaagg

SEQ ID NO: 838 - DNA, 23 bp, Artificial Sequence
  Synthetic Polynucleotide
cttgatcttggcgtgggcccggg

SEQ ID NO: 839 - DNA, 23 bp, Artificial Sequence
  Synthetic Polynucleotide
gggaatagcacaaacccttcccg

SEQ ID NO: 840 - DNA, 23 bp, Artificial Sequence
  Synthetic Polynucleotide
tgcagtaaaatggatcacaggaa

SEQ ID NO: 841 - DNA, 23 bp, Artificial Sequence
  Synthetic Polynucleotide
gctcaataattgagttggttgga

SEQ ID NO: 842 - DNA, 23 bp, Artificial Sequence
  Synthetic Polynucleotide
agccttagatagctgcagatcct

SEQ ID NO: 843 - DNA, 23 bp, Artificial Sequence
  Synthetic Polynucleotide
acaacattatctgcttcggaaaa

SEQ ID NO: 844 - DNA, 23 bp, Artificial Sequence
  Synthetic Polynucleotide
ttccggagcagtgtgtacatact

SEQ ID NO: 845 - DNA, 23 bp, Artificial Sequence
  Synthetic Polynucleotide
gccaaaagggttgtctctggatc

SEQ ID NO: 846 - DNA, 23 bp, Artificial Sequence
  Synthetic Polynucleotide
ggatgctgccaaatgccgggatc

SEQ ID NO: 847 - DNA, 23 bp, Artificial Sequence
  Synthetic Polynucleotide
tatgcccaacacaagtaacctta

SEQ ID NO: 848 - DNA, 23 bp, Artificial Sequence
  Synthetic Polynucleotide
cttgctaggcattcttcccagca

SEQ ID NO: 849 - DNA, 23 bp, Artificial Sequence
  Synthetic Polynucleotide
tgcattcacttgtcttccaaatc

SEQ ID NO: 850 - DNA, 23 bp, Artificial Sequence
  Synthetic Polynucleotide
agagtaatcttgctttatgcagc

SEQ ID NO: 851 - DNA, 23 bp, Artificial Sequence
  Synthetic Polynucleotide
agcttattccaaacttggtggga

SEQ ID NO: 852 - DNA, 23 bp, Artificial Sequence
  Synthetic Polynucleotide
acaattctccttgttgaacttgt

SEQ ID NO: 853 - DNA, 23 bp, Artificial Sequence
  Synthetic Polynucleotide
ctggatctgcatttttctccacc

SEQ ID NO: 854 - DNA, 23 bp, Artificial Sequence
  Synthetic Polynucleotide
atgatactgagagcttgctaggc

SEQ ID NO: 855 - DNA, 23 bp, Artificial Sequence
  Synthetic Polynucleotide
tacatactcatgacgatgccagg

SEQ ID NO: 856 - DNA, 23 bp, Artificial Sequence
  Synthetic Polynucleotide
gcaacatggtgcaaggagcagat

SEQ ID NO: 857 - DNA, 23 bp, Artificial Sequence
  Synthetic Polynucleotide
ctcaataattgagttggttggat

SEQ ID NO: 858 - DNA, 23 bp, Artificial Sequence
  Synthetic Polynucleotide
atgatatgcccaacacaagtaac

SEQ ID NO: 859 - DNA, 23 bp, Artificial Sequence
  Synthetic Polynucleotide
caacacaagtaaccttatccttc

SEQ ID NO: 860 - DNA, 23 bp, Artificial Sequence
  Synthetic Polynucleotide
gagcatcactgtgcaagccccgc

SEQ ID NO: 861 - DNA, 23 bp, Artificial Sequence
  Synthetic Polynucleotide
ttaaccttgaatttgtttcattc

SEQ ID NO: 862 - DNA, 23 bp, Artificial Sequence
  Synthetic Polynucleotide
gacagtccaagatcacaaagata

SEQ ID NO: 863 - DNA, 23 bp, Artificial Sequence
  Synthetic Polynucleotide
tggatctgcatttttctccacca

SEQ ID NO: 864 - DNA, 23 bp, Artificial Sequence
  Synthetic Polynucleotide
ccctgacacaacattatctgctt

SEQ ID NO: 865 - DNA, 23 bp, Artificial Sequence
  Synthetic Polynucleotide
gcttattccaaacttggtgggaa

SEQ ID NO: 866 - DNA, 23 bp, Artificial Sequence
  Synthetic Polynucleotide
agattcaaggttatgctttgctg

SEQ ID NO: 867 - DNA, 23 bp, Artificial Sequence
  Synthetic Polynucleotide
cttcacgttattacctgtgtgct

SEQ ID NO: 868 - DNA, 23 bp, Artificial Sequence
  Synthetic Polynucleotide
tcaattgtgataatggctggtag

SEQ ID NO: 869 - DNA, 23 bp, Artificial Sequence
  Synthetic Polynucleotide
ttgagttggttggatttttgtat

SEQ ID NO: 870 - DNA, 23 bp, Artificial Sequence
  Synthetic Polynucleotide
tcgtcttggtgcttcctattcct

SEQ ID NO: 871 - DNA, 23 bp, Artificial Sequence
  Synthetic Polynucleotide
tgtccattgaggtcagcgctgac

SEQ ID NO: 872 - DNA, 23 bp, Artificial Sequence
  Synthetic Polynucleotide
atgtcatcttctctccgggaggc

SEQ ID NO: 873 - DNA, 23 bp, Artificial Sequence
  Synthetic Polynucleotide
cacctgtccaatatcaatggcag

SEQ ID NO: 874 - DNA, 23 bp, Artificial Sequence
  Synthetic Polynucleotide
tcacgttattacctgtgtgctga

SEQ ID NO: 875 - DNA, 23 bp, Artificial Sequence
  Synthetic Polynucleotide
tgcatattcaccatttaggcata

SEQ ID NO: 876 - DNA, 23 bp, Artificial Sequence
  Synthetic Polynucleotide
ttctataaaacccagtggcagac

SEQ ID NO: 877 - DNA, 23 bp, Artificial Sequence
  Synthetic Polynucleotide
ctgattccggagcagtgtgtaca

SEQ ID NO: 878 - DNA, 23 bp, Artificial Sequence
  Synthetic Polynucleotide
gatatgcccaacacaagtaacct

SEQ ID NO: 879 - DNA, 23 bp, Artificial Sequence
  Synthetic Polynucleotide
tcgtcacagtacacggcctcacc

SEQ ID NO: 880 - DNA, 23 bp, Artificial Sequence
  Synthetic Polynucleotide
tgatactgagagcttgctaggca

SEQ ID NO: 881 - DNA, 23 bp, Artificial Sequence
  Synthetic Polynucleotide
tgaacttcatctcaatgccaatc

SEQ ID NO: 882 - DNA, 23 bp, Artificial Sequence
  Synthetic Polynucleotide
gtagcccagattgggtgttctat

SEQ ID NO: 883 - DNA, 23 bp, Artificial Sequence
  Synthetic Polynucleotide
tcagcggaaatgaaacaaacaaa

SEQ ID NO: 884 - DNA, 23 bp, Artificial Sequence
  Synthetic Polynucleotide
cccatagctgaagtagtggaagg

SEQ ID NO: 885 - DNA, 23 bp, Artificial Sequence
  Synthetic Polynucleotide
tagatccatgttctgtggtatgt

SEQ ID NO: 886 - DNA, 23 bp, Artificial Sequence
  Synthetic Polynucleotide
atatctgtgcggaggttcttatg

SEQ ID NO: 887 - DNA, 23 bp, Artificial Sequence
  Synthetic Polynucleotide
caaatgccgggatcttgtaggtg

SEQ ID NO: 888 - DNA, 23 bp, Artificial Sequence
  Synthetic Polynucleotide
ccggagcagtgtgtacatactca

SEQ ID NO: 889 - DNA, 23 bp, Artificial Sequence
  Synthetic Polynucleotide
ctgcaacatggtgcaaggagcag

SEQ ID NO: 890 - DNA, 23 bp, Artificial Sequence
  Synthetic Polynucleotide
ttctaggtggaaattactttggc

SEQ ID NO: 891 - DNA, 23 bp, Artificial Sequence
  Synthetic Polynucleotide
cccgcactgggagccgtggcttt

SEQ ID NO: 892 - DNA, 23 bp, Artificial Sequence
  Synthetic Polynucleotide
aaacccagtggcagacaagctca

SEQ ID NO: 893 - DNA, 23 bp, Artificial Sequence
  Synthetic Polynucleotide
ttattagtgacatcaagcaccag

SEQ ID NO: 894 - DNA, 23 bp, Artificial Sequence
  Synthetic Polynucleotide
cacggatggcatctttgatggca

SEQ ID NO: 895 - DNA, 23 bp, Artificial Sequence
  Synthetic Polynucleotide
cagacgatcatacttggagagca

SEQ ID NO: 896 - DNA, 23 bp, Artificial Sequence
  Synthetic Polynucleotide
gtggtatgttcctcctgctccat

SEQ ID NO: 897 - DNA, 23 bp, Artificial Sequence
  Synthetic Polynucleotide
ccataatactctgagagagatcc

SEQ ID NO: 898 - DNA, 23 bp, Artificial Sequence
  Synthetic Polynucleotide
gtcaacctcactcttccgagcat

SEQ ID NO: 899 - DNA, 23 bp, Artificial Sequence
  Synthetic Polynucleotide
gtccattgaggtcagcgctgaca

SEQ ID NO: 900 - DNA, 23 bp, Artificial Sequence
  Synthetic Polynucleotide
gcagttgtccatgtggaataaag

SEQ ID NO: 901 - DNA, 23 bp, Artificial Sequence
  Synthetic Polynucleotide
tgcatagatggccttcttgttgg

SEQ ID NO: 902 - DNA, 23 bp, Artificial Sequence
  Synthetic Polynucleotide
caattgtgataatggctggtagt

SEQ ID NO: 903 - DNA, 23 bp, Artificial Sequence
  Synthetic Polynucleotide
cagacaagctcactgtgtccatg

SEQ ID NO: 904 - DNA, 23 bp, Artificial Sequence
  Synthetic Polynucleotide
caatatctgtgcggaggttctta

SEQ ID NO: 905 - DNA, 23 bp, Artificial Sequence
  Synthetic Polynucleotide
acccagtggcagacaagctcact

SEQ ID NO: 906 - DNA, 23 bp, Artificial Sequence
  Synthetic Polynucleotide
ttgggcaggaagcttagcaacag

SEQ ID NO: 907 - DNA, 23 bp, Artificial Sequence
  Synthetic Polynucleotide
ggttggttttgcacagccgccca

SEQ ID NO: 908 - DNA, 23 bp, Artificial Sequence
  Synthetic Polynucleotide
gtacatactcatgacgatgccag

SEQ ID NO: 909 - DNA, 23 bp, Artificial Sequence
  Synthetic Polynucleotide
tttgggcaggaagcttagcaaca

SEQ ID NO: 910 - DNA, 23 bp, Artificial Sequence
  Synthetic Polynucleotide
catgccactggttaccttggcaa

SEQ ID NO: 911 - DNA, 23 bp, Artificial Sequence
  Synthetic Polynucleotide
gtgtcagcaaccacagcaccaat

SEQ ID NO: 912 - DNA, 23 bp, Artificial Sequence
  Synthetic Polynucleotide
tctccaccacctttctgccattc

SEQ ID NO: 913 - DNA, 23 bp, Artificial Sequence
  Synthetic Polynucleotide
agcctcgtcttggtgcttcctat

SEQ ID NO: 914 - DNA, 23 bp, Artificial Sequence
  Synthetic Polynucleotide
gggttccctgagttagtctcaaa

SEQ ID NO: 915 - DNA, 23 bp, Artificial Sequence
  Synthetic Polynucleotide
ccctcacagaccagggtttgcag

SEQ ID NO: 916 - DNA, 23 bp, Artificial Sequence
  Synthetic Polynucleotide
tcatggtgttctgtgtagacaca

SEQ ID NO: 917 - DNA, 23 bp, Artificial Sequence
  Synthetic Polynucleotide
tcataggtgattttcaccccttg

SEQ ID NO: 918 - DNA, 23 bp, Artificial Sequence
  Synthetic Polynucleotide
gtccaatatcaatggcagggttt

SEQ ID NO: 919 - DNA, 23 bp, Artificial Sequence
  Synthetic Polynucleotide
ggtttagactggagccaacatcc

SEQ ID NO: 920 - DNA, 23 bp, Artificial Sequence
  Synthetic Polynucleotide
caccagttatcagcatgtcctca

SEQ ID NO: 921 - DNA, 23 bp, Artificial Sequence
  Synthetic Polynucleotide
ttcagctcaggtccataaaagga

SEQ ID NO: 922 - DNA, 23 bp, Artificial Sequence
  Synthetic Polynucleotide
cgagcatgatactgagagcttgc

SEQ ID NO: 923 - DNA, 23 bp, Artificial Sequence
  Synthetic Polynucleotide
gtaactggagttttcaggtcata

SEQ ID NO: 924 - DNA, 23 bp, Artificial Sequence
  Synthetic Polynucleotide
tcaagcaccagtccaactatatc

SEQ ID NO: 925 - DNA, 23 bp, Artificial Sequence
  Synthetic Polynucleotide
atcttggcgtgggcccgggtgct

SEQ ID NO: 926 - DNA, 23 bp, Artificial Sequence
  Synthetic Polynucleotide
tttaccaaccgcagaaacttgag

SEQ ID NO: 927 - DNA, 23 bp, Artificial Sequence
  Synthetic Polynucleotide
attcacttgtcttccaaatccca

SEQ ID NO: 928 - DNA, 23 bp, Artificial Sequence
  Synthetic Polynucleotide
cagattgggtgttctataaaacc

SEQ ID NO: 929 - DNA, 23 bp, Artificial Sequence
  Synthetic Polynucleotide
atacttggagagcatcactgtgc

SEQ ID NO: 930 - DNA, 23 bp, Artificial Sequence
  Synthetic Polynucleotide
ttctggttgaagtgtgtcaggtc

SEQ ID NO: 931 - DNA, 23 bp, Artificial Sequence
  Synthetic Polynucleotide
ctccacagccgagcttggttcca

SEQ ID NO: 932 - DNA, 23 bp, Artificial Sequence
  Synthetic Polynucleotide
attatctgcttcggaaaacccct

SEQ ID NO: 933 - DNA, 23 bp, Artificial Sequence
  Synthetic Polynucleotide
gcagacgatcatacttggagagc

SEQ ID NO: 934 - DNA, 23 bp, Artificial Sequence
  Synthetic Polynucleotide
gcttgctaggcattcttcccagc

SEQ ID NO: 935 - DNA, 23 bp, Artificial Sequence
  Synthetic Polynucleotide
caggcattggcagaaaagtggac

SEQ ID NO: 936 - DNA, 23 bp, Artificial Sequence
  Synthetic Polynucleotide
tcataggaaacagcatattcttg

SEQ ID NO: 937 - DNA, 23 bp, Artificial Sequence
  Synthetic Polynucleotide
tccacagccgagcttggttccac

SEQ ID NO: 938 - DNA, 23 bp, Artificial Sequence
  Synthetic Polynucleotide
gtgttgggcacagtgttagtgct

SEQ ID NO: 939 - DNA, 23 bp, Artificial Sequence
  Synthetic Polynucleotide
cccaacatttttgcaacaaagct

SEQ ID NO: 940 - DNA, 23 bp, Artificial Sequence
  Synthetic Polynucleotide
atcagcggaaatgaaacaaacaa

SEQ ID NO: 941 - DNA, 23 bp, Artificial Sequence
  Synthetic Polynucleotide
tggcatctttgatggcaaagaag

SEQ ID NO: 942 - DNA, 23 bp, Artificial Sequence
  Synthetic Polynucleotide
agtctcaaagctgtagcccagat

SEQ ID NO: 943 - DNA, 23 bp, Artificial Sequence
  Synthetic Polynucleotide
ttcacgttattacctgtgtgctg

SEQ ID NO: 944 - DNA, 23 bp, Artificial Sequence
  Synthetic Polynucleotide
aatgatatgcccaacacaagtaa

SEQ ID NO: 945 - DNA, 23 bp, Artificial Sequence
  Synthetic Polynucleotide
ggtctgtagcctgtgcagcggca

SEQ ID NO: 946 - DNA, 23 bp, Artificial Sequence
  Synthetic Polynucleotide
tcttggtgcttcctattccttcc

SEQ ID NO: 947 - DNA, 23 bp, Artificial Sequence
  Synthetic Polynucleotide
ttaccaaccgcagaaacttgagg

SEQ ID NO: 948 - DNA, 23 bp, Artificial Sequence
  Synthetic Polynucleotide
ttggtgcttcctattccttccac

SEQ ID NO: 949 - DNA, 23 bp, Artificial Sequence
  Synthetic Polynucleotide
ggtttaagcttctagaggtttgt

SEQ ID NO: 950 - DNA, 23 bp, Artificial Sequence
  Synthetic Polynucleotide
gtttagactggagccaacatcca

SEQ ID NO: 951 - DNA, 23 bp, Artificial Sequence
  Synthetic Polynucleotide
ttatagcagttgtccatgtggaa

SEQ ID NO: 952 - DNA, 23 bp, Artificial Sequence
  Synthetic Polynucleotide
accagttatcagcatgtcctcat

SEQ ID NO: 953 - DNA, 23 bp, Artificial Sequence
  Synthetic Polynucleotide
ccgagcatgatactgagagcttg

SEQ ID NO: 954 - DNA, 23 bp, Artificial Sequence
  Synthetic Polynucleotide
ggttatacaggctgtccagtaag

SEQ ID NO: 955 - DNA, 23 bp, Artificial Sequence
  Synthetic Polynucleotide
ctcgaaccacaatccggtttgct

SEQ ID NO: 956 - DNA, 23 bp, Artificial Sequence
  Synthetic Polynucleotide
cagttgtccatgtggaataaagc

SEQ ID NO: 957 - DNA, 23 bp, Artificial Sequence
  Synthetic Polynucleotide
ttggaacagcaatggtgcagtga

SEQ ID NO: 958 - DNA, 23 bp, Artificial Sequence
  Synthetic Polynucleotide
ccaaaagggttgtctctggatct

SEQ ID NO: 959 - DNA, 23 bp, Artificial Sequence
  Synthetic Polynucleotide
atcaagcaccagtccaactatat

SEQ ID NO: 960 - DNA, 23 bp, Artificial Sequence
  Synthetic Polynucleotide
agtatctcctccgggctcagcag

SEQ ID NO: 961 - DNA, 23 bp, Artificial Sequence
  Synthetic Polynucleotide
tccaaacttggtgggaattatgc

SEQ ID NO: 962 - DNA, 23 bp, Artificial Sequence
  Synthetic Polynucleotide
tcaagatggtctgacaagccgca

SEQ ID NO: 963 - DNA, 23 bp, Artificial Sequence
  Synthetic Polynucleotide
gccttagatagctgcagatcctt

SEQ ID NO: 964 - DNA, 23 bp, Artificial Sequence
  Synthetic Polynucleotide
gattccggagcagtgtgtacata

SEQ ID NO: 965 - DNA, 23 bp, Artificial Sequence
  Synthetic Polynucleotide
cgtggcttttggcaattctctcc

SEQ ID NO: 966 - DNA, 23 bp, Artificial Sequence
  Synthetic Polynucleotide
ctctatggagagcagtatctcct

SEQ ID NO: 967 - DNA, 23 bp, Artificial Sequence
  Synthetic Polynucleotide
tcctaagagacactggcaggtag

SEQ ID NO: 968 - DNA, 23 bp, Artificial Sequence
  Synthetic Polynucleotide
ggtacctcactcctaagagacac

SEQ ID NO: 969 - DNA, 23 bp, Artificial Sequence
  Synthetic Polynucleotide
gtaaagttgcactggcgaaagtg

SEQ ID NO: 970 - DNA, 23 bp, Artificial Sequence
  Synthetic Polynucleotide
cacgttattacctgtgtgctgag

SEQ ID NO: 971 - DNA, 23 bp, Artificial Sequence
  Synthetic Polynucleotide
tgtcatcttctctccgggaggcc

SEQ ID NO: 972 - DNA, 23 bp, Artificial Sequence
  Synthetic Polynucleotide
ttatctgcttcggaaaacccctt

SEQ ID NO: 973 - DNA, 23 bp, Artificial Sequence
  Synthetic Polynucleotide
agtctccaggtagaagtgctctt

SEQ ID NO: 974 - DNA, 23 bp, Artificial Sequence
  Synthetic Polynucleotide
aggccgtgttggagggaaggttg

SEQ ID NO: 975 - DNA, 23 bp, Artificial Sequence
  Synthetic Polynucleotide
tctccacagccgagcttggttcc

SEQ ID NO: 976 - DNA, 23 bp, Artificial Sequence
  Synthetic Polynucleotide
tcttccgagcatgatactgagag

SEQ ID NO: 977 - DNA, 23 bp, Artificial Sequence
  Synthetic Polynucleotide
gagcatgatactgagagcttgct

SEQ ID NO: 978 - DNA, 23 bp, Artificial Sequence
  Synthetic Polynucleotide
tcaatggcagggtttagactgga

SEQ ID NO: 979 - DNA, 23 bp, Artificial Sequence
  Synthetic Polynucleotide
cagccggagagacagctcattct

SEQ ID NO: 980 - DNA, 23 bp, Artificial Sequence
  Synthetic Polynucleotide
tcatcacggtccagcatgcatcg

SEQ ID NO: 981 - DNA, 23 bp, Artificial Sequence
  Synthetic Polynucleotide
tatctgtgcggaggttcttatga

SEQ ID NO: 982 - DNA, 23 bp, Artificial Sequence
  Synthetic Polynucleotide
tttctaggtggaaattactttgg

SEQ ID NO: 983 - DNA, 23 bp, Artificial Sequence
  Synthetic Polynucleotide
aatatctgtgcggaggttcttat

SEQ ID NO: 984 - DNA, 23 bp, Artificial Sequence
  Synthetic Polynucleotide
gcagaaggttggatttatacagt

SEQ ID NO: 985 - DNA, 23 bp, Artificial Sequence
  Synthetic Polynucleotide
atgaaacaaacaaaccctggaac

SEQ ID NO: 986 - DNA, 23 bp, Artificial Sequence
  Synthetic Polynucleotide
gtggcttttggcaattctctcct

SEQ ID NO: 987 - DNA, 23 bp, Artificial Sequence
  Synthetic Polynucleotide
cacagtgttagtgcttgtctcgc

SEQ ID NO: 988 - DNA, 23 bp, Artificial Sequence
  Synthetic Polynucleotide
atgctgccaaatgccgggatctt

SEQ ID NO: 989 - DNA, 23 bp, Artificial Sequence
  Synthetic Polynucleotide
aatgtcatcttctctccgggagg

SEQ ID NO: 990 - DNA, 23 bp, Artificial Sequence
  Synthetic Polynucleotide
attccaaacttggtgggaattat

SEQ ID NO: 991 - DNA, 23 bp, Artificial Sequence
  Synthetic Polynucleotide
tttggcttcaaatgtaaagatta

SEQ ID NO: 992 - DNA, 23 bp, Artificial Sequence
  Synthetic Polynucleotide
tttatagcagttgtccatgtgga

SEQ ID NO: 993 - DNA, 23 bp, Artificial Sequence
  Synthetic Polynucleotide
agaccagggtttgcagttttctg

SEQ ID NO: 994 - DNA, 23 bp, Artificial Sequence
  Synthetic Polynucleotide
ttggcttcaaatgtaaagattaa

SEQ ID NO: 995 - DNA, 23 bp, Artificial Sequence
  Synthetic Polynucleotide
cctgcttgaatgctgagaaatac

SEQ ID NO: 996 - DNA, 23 bp, Artificial Sequence
  Synthetic Polynucleotide
caattaaccttgaatttgtttca

SEQ ID NO: 997 - DNA, 23 bp, Artificial Sequence
  Synthetic Polynucleotide
agtttcatttattagtgacatca

SEQ ID NO: 998 - DNA, 23 bp, Artificial Sequence
  Synthetic Polynucleotide
ctctttaccaaccgcagaaactt

SEQ ID NO: 999 - DNA, 23 bp, Artificial Sequence
  Synthetic Polynucleotide
ctttgatggcaaagaagatagaa

SEQ ID NO: 1000 - DNA, 23 bp, Artificial Sequence
  Synthetic Polynucleotide
atcagaacttgaggttatacagg

SEQ ID NO: 1001 - DNA, 23 bp, Artificial Sequence
  Synthetic Polynucleotide
atctgtgcggaggttcttatgat

SEQ ID NO: 1002 - DNA, 23 bp, Artificial Sequence
  Synthetic Polynucleotide
tgtacatactcatgacgatgcca

SEQ ID NO: 1003 - DNA, 23 bp, Artificial Sequence
  Synthetic Polynucleotide
ccttccacagttgtcactgcaac

SEQ ID NO: 1004 - DNA, 23 bp, Artificial Sequence
  Synthetic Polynucleotide
tcttcagctcaggtccataaaag

SEQ ID NO: 1005 - DNA, 23 bp, Artificial Sequence
  Synthetic Polynucleotide
gaaacagcatattcttgaacttc

SEQ ID NO: 1006 - DNA, 23 bp, Artificial Sequence
  Synthetic Polynucleotide
cagctccttgagggttgaggcct

SEQ ID NO: 1007 - DNA, 23 bp, Artificial Sequence
  Synthetic Polynucleotide
ccatgttctgtggtatgttcctc

SEQ ID NO: 1008 - DNA, 23 bp, Artificial Sequence
  Synthetic Polynucleotide
gtgtgtacatactcatgacgatg

SEQ ID NO: 1009 - DNA, 23 bp, Artificial Sequence
  Synthetic Polynucleotide
cttctggttgaagtgtgtcaggt

SEQ ID NO: 1010 - DNA, 23 bp, Artificial Sequence
  Synthetic Polynucleotide
gttattacctgtgtgctgagctc

SEQ ID NO: 1011 - DNA, 23 bp, Artificial Sequence
  Synthetic Polynucleotide
tgactttaatagatccatgttct

SEQ ID NO: 1012 - DNA, 23 bp, Artificial Sequence
  Synthetic Polynucleotide
ccacagccgagcttggttccact

SEQ ID NO: 1013 - DNA, 23 bp, Artificial Sequence
  Synthetic Polynucleotide
cctgtccaatatcaatggcaggg

SEQ ID NO: 1014 - DNA, 23 bp, Artificial Sequence
  Synthetic Polynucleotide
gaagaagaagctgagggtgaggg

SEQ ID NO: 1015 - DNA, 23 bp, Artificial Sequence
  Synthetic Polynucleotide
cttttggcaattctctcctgcac

SEQ ID NO: 1016 - DNA, 23 bp, Artificial Sequence
  Synthetic Polynucleotide
gccaccagttatcagcatgtcct

SEQ ID NO: 1017 - DNA, 23 bp, Artificial Sequence
  Synthetic Polynucleotide
taaagattaaacataatcttttt

SEQ ID NO: 1018 - DNA, 23 bp, Artificial Sequence
  Synthetic Polynucleotide
gcttttggcaattctctcctgca

SEQ ID NO: 1019 - DNA, 23 bp, Artificial Sequence
  Synthetic Polynucleotide
aaagattaaacataatctttttt

SEQ ID NO: 1020 - DNA, 23 bp, Artificial Sequence
  Synthetic Polynucleotide
accattttggtatgaaggccttg

SEQ ID NO: 1021 - DNA, 23 bp, Artificial Sequence
  Synthetic Polynucleotide
gcttcccagcaaaccagcgcagc

SEQ ID NO: 1022 - DNA, 23 bp, Artificial Sequence
  Synthetic Polynucleotide
gggaaagaaatctagaacattgt

SEQ ID NO: 1023 - DNA, 23 bp, Artificial Sequence
  Synthetic Polynucleotide
cctgtgtgctgagctcgagctgc

SEQ ID NO: 1024 - DNA, 23 bp, Artificial Sequence
  Synthetic Polynucleotide
ccttttggaacagcaatggtgca

SEQ ID NO: 1025 - DNA, 23 bp, Artificial Sequence
  Synthetic Polynucleotide
gcagaaaagtggacgatcttgtt

SEQ ID NO: 1026 - DNA, 23 bp, Artificial Sequence
  Synthetic Polynucleotide
gttccttcacgttattacctgtg

SEQ ID NO: 1027 - DNA, 23 bp, Artificial Sequence
  Synthetic Polynucleotide
tgaataaaactctcatgccactg

SEQ ID NO: 1028 - DNA, 23 bp, Artificial Sequence
  Synthetic Polynucleotide
ttgctgttcattggtttgaaggc

SEQ ID NO: 1029 - DNA, 23 bp, Artificial Sequence
  Synthetic Polynucleotide
tttgctgttcattggtttgaagg

SEQ ID NO: 1030 - DNA, 23 bp, Artificial Sequence
  Synthetic Polynucleotide
caataattgagttggttggattt

SEQ ID NO: 1031 - DNA, 23 bp, Artificial Sequence
  Synthetic Polynucleotide
gattaaacataatcttttttgta

SEQ ID NO: 1032 - DNA, 23 bp, Artificial Sequence
  Synthetic Polynucleotide
tcctctgcaggcatcccacaggt

SEQ ID NO: 1033 - DNA, 23 bp, Artificial Sequence
  Synthetic Polynucleotide
gcctctgagtggtcttgagggct

SEQ ID NO: 1034 - DNA, 23 bp, Artificial Sequence
  Synthetic Polynucleotide
cacctcctctgcaggcatcccac

SEQ ID NO: 1035 - DNA, 23 bp, Artificial Sequence
  Synthetic Polynucleotide
ataaaactctcatgccactggtt

SEQ ID NO: 1036 - DNA, 23 bp, Artificial Sequence
  Synthetic Polynucleotide
ttctgtgactttaatagatccat

SEQ ID NO: 1037 - DNA, 23 bp, Artificial Sequence
  Synthetic Polynucleotide
agtaaaatggatcacaggaaggg

SEQ ID NO: 1038 - DNA, 23 bp, Artificial Sequence
  Synthetic Polynucleotide
aaagaagatagaagcagccagga

SEQ ID NO: 1039 - DNA, 23 bp, Artificial Sequence
  Synthetic Polynucleotide
gcacagagccatctgtgtacaca

SEQ ID NO: 1040 - DNA, 23 bp, Artificial Sequence
  Synthetic Polynucleotide
ggactgtcaggtagaacttgaag

SEQ ID NO: 1041 - DNA, 23 bp, Artificial Sequence
  Synthetic Polynucleotide
atgctctccaagtatgatcgtct

SEQ ID NO: 1042 - DNA, 23 bp, Artificial Sequence
  Synthetic Polynucleotide
cgtctgcagaacaagatcgtcca

SEQ ID NO: 1043 - DNA, 23 bp, Artificial Sequence
  Synthetic Polynucleotide
tcgtccacttttctgccaatgcc

SEQ ID NO: 1044 - DNA, 23 bp, Artificial Sequence
  Synthetic Polynucleotide
agccacggctcccagtgcgggtt

SEQ ID NO: 1045 - DNA, 23 bp, Artificial Sequence
  Synthetic Polynucleotide
ggaacacggagattggcattgag

SEQ ID NO: 1046 - DNA, 23 bp, Artificial Sequence
  Synthetic Polynucleotide
gaacacggagattggcattgaga

SEQ ID NO: 1047 - DNA, 23 bp, Artificial Sequence
  Synthetic Polynucleotide
ccggagagaagatgacattgcca

SEQ ID NO: 1048 - DNA, 23 bp, Artificial Sequence
  Synthetic Polynucleotide
gtaaccagtggcatgagagtttt

SEQ ID NO: 1049 - DNA, 23 bp, Artificial Sequence
  Synthetic Polynucleotide
gacatgcaggcctctggtgaggc

SEQ ID NO: 1050 - DNA, 23 bp, Artificial Sequence
  Synthetic Polynucleotide
cctctggtgaggccgtgtactgt

SEQ ID NO: 1051 - DNA, 23 bp, Artificial Sequence
  Synthetic Polynucleotide
cacccgggcccacgccaagatca

SEQ ID NO: 1052 - DNA, 23 bp, Artificial Sequence
  Synthetic Polynucleotide
cacgccaagatcaagtccataga

SEQ ID NO: 1053 - DNA, 23 bp, Artificial Sequence
  Synthetic Polynucleotide
ttgtgttgggcatatcattggtg

SEQ ID NO: 1054 - DNA, 23 bp, Artificial Sequence
  Synthetic Polynucleotide
atatcattggtgctgtggttgct

SEQ ID NO: 1055 - DNA, 23 bp, Artificial Sequence
  Synthetic Polynucleotide
cagcaaaccggattgtggttcga

SEQ ID NO: 1056 - DNA, 23 bp, Artificial Sequence
  Synthetic Polynucleotide
caaaccggattgtggttcgagtg

SEQ ID NO: 1057 - DNA, 23 bp, Artificial Sequence
  Synthetic Polynucleotide
accggattgtggttcgagtgaag

SEQ ID NO: 1058 - DNA, 23 bp, Artificial Sequence
  Synthetic Polynucleotide
gcatgctggaccgtgatgaggac

SEQ ID NO: 1059 - DNA, 23 bp, Artificial Sequence
  Synthetic Polynucleotide
gtgatgaggacatgctgataact

SEQ ID NO: 1060 - DNA, 23 bp, Artificial Sequence
  Synthetic Polynucleotide
tggccagatacaaggttggcttc

SEQ ID NO: 1061 - DNA, 23 bp, Artificial Sequence
  Synthetic Polynucleotide
atctctctcagagtattatggaa

SEQ ID NO: 1062 - DNA, 23 bp, Artificial Sequence
  Synthetic Polynucleotide
aaccaaccttccctccaacacgg

SEQ ID NO: 1063 - DNA, 23 bp, Artificial Sequence
  Synthetic Polynucleotide
atgcctagcaagctctcagtatc

SEQ ID NO: 1064 - DNA, 23 bp, Artificial Sequence
  Synthetic Polynucleotide
cctagcaagctctcagtatcatg

SEQ ID NO: 1065 - DNA, 23 bp, Artificial Sequence
  Synthetic Polynucleotide
gctctcagtatcatgctcggaag

SEQ ID NO: 1066 - DNA, 23 bp, Artificial Sequence
  Synthetic Polynucleotide
attcccaccaagtttggaataag

SEQ ID NO: 1067 - DNA, 23 bp, Artificial Sequence
  Synthetic Polynucleotide
aacctccgcacagatattgtcat

SEQ ID NO: 1068 - DNA, 23 bp, Artificial Sequence
  Synthetic Polynucleotide
cctacaagatcccggcatttggc

SEQ ID NO: 1069 - DNA, 23 bp, Artificial Sequence
  Synthetic Polynucleotide
gctcagcacacaggtaataacgt

SEQ ID NO: 1070 - DNA, 23 bp, Artificial Sequence
  Synthetic Polynucleotide
gcaaaccctggtctgtgagggtc

SEQ ID NO: 1071 - DNA, 23 bp, Artificial Sequence
  Synthetic Polynucleotide
cataccacagaacatggatctat

SEQ ID NO: 1072 - DNA, 23 bp, Artificial Sequence
  Synthetic Polynucleotide
cagaacatggatctattaaagtc

SEQ ID NO: 1073 - DNA, 23 bp, Artificial Sequence
  Synthetic Polynucleotide
gaacatggatctattaaagtcac

SEQ ID NO: 1074 - DNA, 23 bp, Artificial Sequence
  Synthetic Polynucleotide
gtcgggaagggtttgtgctattc

SEQ ID NO: 1075 - DNA, 23 bp, Artificial Sequence
  Synthetic Polynucleotide
tcgggaagggtttgtgctattcc

SEQ ID NO: 1076 - DNA, 23 bp, Artificial Sequence
  Synthetic Polynucleotide
taacctcaagttctgatggtgtc

SEQ ID NO: 1077 - DNA, 23 bp, Artificial Sequence
  Synthetic Polynucleotide
aacctcaagttctgatggtgtct

SEQ ID NO: 1078 - DNA, 23 bp, Artificial Sequence
  Synthetic Polynucleotide
gattcccacaaacctctagaagc

SEQ ID NO: 1079 - DNA, 23 bp, Artificial Sequence
  Synthetic Polynucleotide
ttcccacaaacctctagaagctt

SEQ ID NO: 1080 - DNA, 23 bp, Artificial Sequence
  Synthetic Polynucleotide
ctggccttcaaaccaatgaacag

SEQ ID NO: 1081 - DNA, 23 bp, Artificial Sequence
  Synthetic Polynucleotide
aaccaatgaacagcaaagcataa

SEQ ID NO: 1082 - DNA, 23 bp, Artificial Sequence
  Synthetic Polynucleotide
atgaaacaaattcaaggttaatt

SEQ ID NO: 1083 - DNA, 23 bp, Artificial Sequence
  Synthetic Polynucleotide
tgtgaagctgcataaagcaagat

SEQ ID NO: 1084 - DNA, 23 bp, Artificial Sequence
  Synthetic Polynucleotide
aagctgcataaagcaagattact

SEQ ID NO: 1085 - DNA, 23 bp, Artificial Sequence
  Synthetic Polynucleotide
caaaaaagattatgtttaatctt

SEQ ID NO: 1086 - DNA, 23 bp, Artificial Sequence
  Synthetic Polynucleotide
atctaaggcttggttttcttact

SEQ ID NO: 1087 - DNA, 23 bp, Artificial Sequence
  Synthetic Polynucleotide
gcttggttttcttactgtcatat

SEQ ID NO: 1088 - DNA, 23 bp, Artificial Sequence
  Synthetic Polynucleotide
cacctcaagtttctgcggttggt

SEQ ID NO: 1089 - DNA, 23 bp, Artificial Sequence
  Synthetic Polynucleotide
acctcaagtttctgcggttggta

SEQ ID NO: 1090 - DNA, 23 bp, Artificial Sequence
  Synthetic Polynucleotide
atagttggactggtgcttgatgt

SEQ ID NO: 1091 - DNA, 23 bp, Artificial Sequence
  Synthetic Polynucleotide
gggatttggaagacaagtgaatg

SEQ ID NO: 1092 - DNA, 23 bp, Artificial Sequence
  Synthetic Polynucleotide
cacagtgatgctctccaagtatg

SEQ ID NO: 1093 - DNA, 23 bp, Artificial Sequence
  Synthetic Polynucleotide
gcaaaccggattgtggttcgagt

SEQ ID NO: 1094 - DNA, 23 bp, Artificial Sequence
  Synthetic Polynucleotide
caagtttctgcggttggtaaaga

SEQ ID NO: 1095 - DNA, 23 bp, Artificial Sequence
  Synthetic Polynucleotide
gctgcttctatcttctttgccat

SEQ ID NO: 1096 - DNA, 23 bp, Artificial Sequence
  Synthetic Polynucleotide
ggaggaacataccacagaacatg

SEQ ID NO: 1097 - DNA, 23 bp, Artificial Sequence
  Synthetic Polynucleotide
ccttttatggacctgagctgaag

SEQ ID NO: 1098 - DNA, 23 bp, Artificial Sequence
  Synthetic Polynucleotide
cccgggcccacgccaagatcaag

SEQ ID NO: 1099 - DNA, 23 bp, Artificial Sequence
  Synthetic Polynucleotide
cgggaagggtttgtgctattccc

SEQ ID NO: 1100 - DNA, 23 bp, Artificial Sequence
  Synthetic Polynucleotide
ttcctgtgatccattttactgca

SEQ ID NO: 1101 - DNA, 23 bp, Artificial Sequence
  Synthetic Polynucleotide
tccaaccaactcaattattgagc

SEQ ID NO: 1102 - DNA, 23 bp, Artificial Sequence
  Synthetic Polynucleotide
aggatctgcagctatctaaggct

SEQ ID NO: 1103 - DNA, 23 bp, Artificial Sequence
  Synthetic Polynucleotide
ttttccgaagcagataatgttgt

SEQ ID NO: 1104 - DNA, 23 bp, Artificial Sequence
  Synthetic Polynucleotide
agtatgtacacactgctccggaa

SEQ ID NO: 1105 - DNA, 23 bp, Artificial Sequence
  Synthetic Polynucleotide
gatccagagacaacccttttggc

SEQ ID NO: 1106 - DNA, 23 bp, Artificial Sequence
  Synthetic Polynucleotide
gatcccggcatttggcagcatcc

SEQ ID NO: 1107 - DNA, 23 bp, Artificial Sequence
  Synthetic Polynucleotide
taaggttacttgtgttgggcata

SEQ ID NO: 1108 - DNA, 23 bp, Artificial Sequence
  Synthetic Polynucleotide
tgctgggaagaatgcctagcaag

SEQ ID NO: 1109 - DNA, 23 bp, Artificial Sequence
  Synthetic Polynucleotide
gatttggaagacaagtgaatgca

SEQ ID NO: 1110 - DNA, 23 bp, Artificial Sequence
  Synthetic Polynucleotide
gctgcataaagcaagattactct

SEQ ID NO: 1111 - DNA, 23 bp, Artificial Sequence
  Synthetic Polynucleotide
tcccaccaagtttggaataagct

SEQ ID NO: 1112 - DNA, 23 bp, Artificial Sequence
  Synthetic Polynucleotide
acaagttcaacaaggagaattgt

SEQ ID NO: 1113 - DNA, 23 bp, Artificial Sequence
  Synthetic Polynucleotide
ggtggagaaaaatgcagatccag

SEQ ID NO: 1114 - DNA, 23 bp, Artificial Sequence
  Synthetic Polynucleotide
gcctagcaagctctcagtatcat

SEQ ID NO: 1115 - DNA, 23 bp, Artificial Sequence
  Synthetic Polynucleotide
cctggcatcgtcatgagtatgta

SEQ ID NO: 1116 - DNA, 23 bp, Artificial Sequence
  Synthetic Polynucleotide
atctgctccttgcaccatgttgc

SEQ ID NO: 1117 - DNA, 23 bp, Artificial Sequence
  Synthetic Polynucleotide
atccaaccaactcaattattgag

SEQ ID NO: 1118 - DNA, 23 bp, Artificial Sequence
  Synthetic Polynucleotide
gttacttgtgttgggcatatcat

SEQ ID NO: 1119 - DNA, 23 bp, Artificial Sequence
  Synthetic Polynucleotide
gaaggataaggttacttgtgttg

SEQ ID NO: 1120 - DNA, 23 bp, Artificial Sequence
  Synthetic Polynucleotide
gcggggcttgcacagtgatgctc

SEQ ID NO: 1121 - DNA, 23 bp, Artificial Sequence
  Synthetic Polynucleotide
gaatgaaacaaattcaaggttaa

SEQ ID NO: 1122 - DNA, 23 bp, Artificial Sequence
  Synthetic Polynucleotide
tatctttgtgatcttggactgtc

SEQ ID NO: 1123 - DNA, 23 bp, Artificial Sequence
  Synthetic Polynucleotide
tggtggagaaaaatgcagatcca

SEQ ID NO: 1124 - DNA, 23 bp, Artificial Sequence
  Synthetic Polynucleotide
aagcagataatgttgtgtcaggg

SEQ ID NO: 1125 - DNA, 23 bp, Artificial Sequence
  Synthetic Polynucleotide
ttcccaccaagtttggaataagc

SEQ ID NO: 1126 - DNA, 23 bp, Artificial Sequence
  Synthetic Polynucleotide
cagcaaagcataaccttgaatct

SEQ ID NO: 1127 - DNA, 23 bp, Artificial Sequence
  Synthetic Polynucleotide
agcacacaggtaataacgtgaag

SEQ ID NO: 1128 - DNA, 23 bp, Artificial Sequence
  Synthetic Polynucleotide
ctaccagccattatcacaattga

SEQ ID NO: 1129 - DNA, 23 bp, Artificial Sequence
  Synthetic Polynucleotide
atacaaaaatccaaccaactcaa

SEQ ID NO: 1130 - DNA, 23 bp, Artificial Sequence
  Synthetic Polynucleotide
aggaataggaagcaccaagacga

SEQ ID NO: 1131 - DNA, 23 bp, Artificial Sequence
  Synthetic Polynucleotide
gtcagcgctgacctcaatggaca

SEQ ID NO: 1132 - DNA, 23 bp, Artificial Sequence
  Synthetic Polynucleotide
gcctcccggagagaagatgacat

SEQ ID NO: 1133 - DNA, 23 bp, Artificial Sequence
  Synthetic Polynucleotide
ctgccattgatattggacaggtg

SEQ ID NO: 1134 - DNA, 23 bp, Artificial Sequence
  Synthetic Polynucleotide
tcagcacacaggtaataacgtga

SEQ ID NO: 1135 - DNA, 23 bp, Artificial Sequence
  Synthetic Polynucleotide
tatgcctaaatggtgaatatgca

SEQ ID NO: 1136 - DNA, 23 bp, Artificial Sequence
  Synthetic Polynucleotide
gtctgccactgggttttatagaa

SEQ ID NO: 1137 - DNA, 23 bp, Artificial Sequence
  Synthetic Polynucleotide
tgtacacactgctccggaatcag

SEQ ID NO: 1138 - DNA, 23 bp, Artificial Sequence
  Synthetic Polynucleotide
aggttacttgtgttgggcatatc

SEQ ID NO: 1139 - DNA, 23 bp, Artificial Sequence
  Synthetic Polynucleotide
ggtgaggccgtgtactgtgacga

SEQ ID NO: 1140 - DNA, 23 bp, Artificial Sequence
  Synthetic Polynucleotide
tgcctagcaagctctcagtatca

SEQ ID NO: 1141 - DNA, 23 bp, Artificial Sequence
  Synthetic Polynucleotide
gattggcattgagatgaagttca

SEQ ID NO: 1142 - DNA, 23 bp, Artificial Sequence
  Synthetic Polynucleotide
atagaacacccaatctgggctac

SEQ ID NO: 1143 - DNA, 23 bp, Artificial Sequence
  Synthetic Polynucleotide
tttgtttgtttcatttccgctga

SEQ ID NO: 1144 - DNA, 23 bp, Artificial Sequence
  Synthetic Polynucleotide
ccttccactacttcagctatggg

SEQ ID NO: 1145 - DNA, 23 bp, Artificial Sequence
  Synthetic Polynucleotide
acataccacagaacatggatcta

SEQ ID NO: 1146 - DNA, 23 bp, Artificial Sequence
  Synthetic Polynucleotide
cataagaacctccgcacagatat

SEQ ID NO: 1147 - DNA, 23 bp, Artificial Sequence
  Synthetic Polynucleotide
cacctacaagatcccggcatttg

SEQ ID NO: 1148 - DNA, 23 bp, Artificial Sequence
  Synthetic Polynucleotide
tgagtatgtacacactgctccgg

SEQ ID NO: 1149 - DNA, 23 bp, Artificial Sequence
  Synthetic Polynucleotide
ctgctccttgcaccatgttgcag

SEQ ID NO: 1150 - DNA, 23 bp, Artificial Sequence
  Synthetic Polynucleotide
gccaaagtaatttccacctagaa

SEQ ID NO: 1151 - DNA, 23 bp, Artificial Sequence
  Synthetic Polynucleotide
aaagccacggctcccagtgcggg

SEQ ID NO: 1152 - DNA, 23 bp, Artificial Sequence
  Synthetic Polynucleotide
tgagcttgtctgccactgggttt

SEQ ID NO: 1153 - DNA, 23 bp, Artificial Sequence
  Synthetic Polynucleotide
ctggtgcttgatgtcactaataa

SEQ ID NO: 1154 - DNA, 23 bp, Artificial Sequence
  Synthetic Polynucleotide
tgccatcaaagatgccatccgtg

SEQ ID NO: 1155 - DNA, 23 bp, Artificial Sequence
  Synthetic Polynucleotide
tgctctccaagtatgatcgtctg

SEQ ID NO: 1156 - DNA, 23 bp, Artificial Sequence
  Synthetic Polynucleotide
atggagcaggaggaacataccac

SEQ ID NO: 1157 - DNA, 23 bp, Artificial Sequence
  Synthetic Polynucleotide
ggatctctctcagagtattatgg

SEQ ID NO: 1158 - DNA, 23 bp, Artificial Sequence
  Synthetic Polynucleotide
atgctcggaagagtgaggttgac

SEQ ID NO: 1159 - DNA, 23 bp, Artificial Sequence
  Synthetic Polynucleotide
tgtcagcgctgacctcaatggac

SEQ ID NO: 1160 - DNA, 23 bp, Artificial Sequence
  Synthetic Polynucleotide
ctttattccacatggacaactgc

SEQ ID NO: 1161 - DNA, 23 bp, Artificial Sequence
  Synthetic Polynucleotide
ccaacaagaaggccatctatgca

SEQ ID NO: 1162 - DNA, 23 bp, Artificial Sequence
  Synthetic Polynucleotide
actaccagccattatcacaattg

SEQ ID NO: 1163 - DNA, 23 bp, Artificial Sequence
  Synthetic Polynucleotide
catggacacagtgagcttgtctg

SEQ ID NO: 1164 - DNA, 23 bp, Artificial Sequence
  Synthetic Polynucleotide
taagaacctccgcacagatattg

SEQ ID NO: 1165 - DNA, 23 bp, Artificial Sequence
  Synthetic Polynucleotide
agtgagcttgtctgccactgggt

SEQ ID NO: 1166 - DNA, 23 bp, Artificial Sequence
  Synthetic Polynucleotide
ctgttgctaagcttcctgcccaa

SEQ ID NO: 1167 - DNA, 23 bp, Artificial Sequence
  Synthetic Polynucleotide
tgggcggctgtgcaaaaccaacc

SEQ ID NO: 1168 - DNA, 23 bp, Artificial Sequence
  Synthetic Polynucleotide
ctggcatcgtcatgagtatgtac

SEQ ID NO: 1169 - DNA, 23 bp, Artificial Sequence
  Synthetic Polynucleotide
tgttgctaagcttcctgcccaaa

SEQ ID NO: 1170 - DNA, 23 bp, Artificial Sequence
  Synthetic Polynucleotide
ttgccaaggtaaccagtggcatg

SEQ ID NO: 1171 - DNA, 23 bp, Artificial Sequence
  Synthetic Polynucleotide
attggtgctgtggttgctgacac

SEQ ID NO: 1172 - DNA, 23 bp, Artificial Sequence
  Synthetic Polynucleotide
gaatggcagaaaggtggtggaga

SEQ ID NO: 1173 - DNA, 23 bp, Artificial Sequence
  Synthetic Polynucleotide
ataggaagcaccaagacgaggct

SEQ ID NO: 1174 - DNA, 23 bp, Artificial Sequence
  Synthetic Polynucleotide
tttgagactaactcagggaaccc

SEQ ID NO: 1175 - DNA, 23 bp, Artificial Sequence
  Synthetic Polynucleotide
ctgcaaaccctggtctgtgaggg

SEQ ID NO: 1176 - DNA, 23 bp, Artificial Sequence
  Synthetic Polynucleotide
tgtgtctacacagaacaccatga

SEQ ID NO: 1177 - DNA, 23 bp, Artificial Sequence
  Synthetic Polynucleotide
caaggggtgaaaatcacctatga

SEQ ID NO: 1178 - DNA, 23 bp, Artificial Sequence
  Synthetic Polynucleotide
aaaccctgccattgatattggac

SEQ ID NO: 1179 - DNA, 23 bp, Artificial Sequence
  Synthetic Polynucleotide
ggatgttggctccagtctaaacc

SEQ ID NO: 1180 - DNA, 23 bp, Artificial Sequence
  Synthetic Polynucleotide
tgaggacatgctgataactggtg

SEQ ID NO: 1181 - DNA, 23 bp, Artificial Sequence
  Synthetic Polynucleotide
tccttttatggacctgagctgaa

SEQ ID NO: 1182 - DNA, 23 bp, Artificial Sequence
  Synthetic Polynucleotide
gcaagctctcagtatcatgctcg

SEQ ID NO: 1183 - DNA, 23 bp, Artificial Sequence
  Synthetic Polynucleotide
tatgacctgaaaactccagttac

SEQ ID NO: 1184 - DNA, 23 bp, Artificial Sequence
  Synthetic Polynucleotide
gatatagttggactggtgcttga

SEQ ID NO: 1185 - DNA, 23 bp, Artificial Sequence
  Synthetic Polynucleotide
agcacccgggcccacgccaagat

SEQ ID NO: 1186 - DNA, 23 bp, Artificial Sequence
  Synthetic Polynucleotide
ctcaagtttctgcggttggtaaa

SEQ ID NO: 1187 - DNA, 23 bp, Artificial Sequence
  Synthetic Polynucleotide
tgggatttggaagacaagtgaat

SEQ ID NO: 1188 - DNA, 23 bp, Artificial Sequence
  Synthetic Polynucleotide
ggttttatagaacacccaatctg

SEQ ID NO: 1189 - DNA, 23 bp, Artificial Sequence
  Synthetic Polynucleotide
gcacagtgatgctctccaagtat

SEQ ID NO: 1190 - DNA, 23 bp, Artificial Sequence
  Synthetic Polynucleotide
gacctgacacacttcaaccagaa

SEQ ID NO: 1191 - DNA, 23 bp, Artificial Sequence
  Synthetic Polynucleotide
tggaaccaagctcggctgtggag

SEQ ID NO: 1192 - DNA, 23 bp, Artificial Sequence
  Synthetic Polynucleotide
aggggttttccgaagcagataat

SEQ ID NO: 1193 - DNA, 23 bp, Artificial Sequence
  Synthetic Polynucleotide
gctctccaagtatgatcgtctgc

SEQ ID NO: 1194 - DNA, 23 bp, Artificial Sequence
  Synthetic Polynucleotide
gctgggaagaatgcctagcaagc

SEQ ID NO: 1195 - DNA, 23 bp, Artificial Sequence
  Synthetic Polynucleotide
gtccacttttctgccaatgcctg

SEQ ID NO: 1196 - DNA, 23 bp, Artificial Sequence
  Synthetic Polynucleotide
caagaatatgctgtttcctatga

SEQ ID NO: 1197 - DNA, 23 bp, Artificial Sequence
  Synthetic Polynucleotide
gtggaaccaagctcggctgtgga

SEQ ID NO: 1198 - DNA, 23 bp, Artificial Sequence
  Synthetic Polynucleotide
agcactaacactgtgcccaacac

SEQ ID NO: 1199 - DNA, 23 bp, Artificial Sequence
  Synthetic Polynucleotide
agctttgttgcaaaaatgttggg

SEQ ID NO: 1200 - DNA, 23 bp, Artificial Sequence
  Synthetic Polynucleotide
ttgtttgtttcatttccgctgat

SEQ ID NO: 1201 - DNA, 23 bp, Artificial Sequence
  Synthetic Polynucleotide
cttctttgccatcaaagatgcca

SEQ ID NO: 1202 - DNA, 23 bp, Artificial Sequence
  Synthetic Polynucleotide
atctgggctacagctttgagact

SEQ ID NO: 1203 - DNA, 23 bp, Artificial Sequence
  Synthetic Polynucleotide
cagcacacaggtaataacgtgaa

SEQ ID NO: 1204 - DNA, 23 bp, Artificial Sequence
  Synthetic Polynucleotide
ttacttgtgttgggcatatcatt

SEQ ID NO: 1205 - DNA, 23 bp, Artificial Sequence
  Synthetic Polynucleotide
tgccgctgcacaggctacagacc

SEQ ID NO: 1206 - DNA, 23 bp, Artificial Sequence
  Synthetic Polynucleotide
ggaaggaataggaagcaccaaga

SEQ ID NO: 1207 - DNA, 23 bp, Artificial Sequence
  Synthetic Polynucleotide
cctcaagtttctgcggttggtaa

SEQ ID NO: 1208 - DNA, 23 bp, Artificial Sequence
  Synthetic Polynucleotide
gtggaaggaataggaagcaccaa

SEQ ID NO: 1209 - DNA, 23 bp, Artificial Sequence
  Synthetic Polynucleotide
acaaacctctagaagcttaaacc

SEQ ID NO: 1210 - DNA, 23 bp, Artificial Sequence
  Synthetic Polynucleotide
tggatgttggctccagtctaaac

SEQ ID NO: 1211 - DNA, 23 bp, Artificial Sequence
  Synthetic Polynucleotide
ttccacatggacaactgctataa

SEQ ID NO: 1212 - DNA, 23 bp, Artificial Sequence
  Synthetic Polynucleotide
atgaggacatgctgataactggt

SEQ ID NO: 1213 - DNA, 23 bp, Artificial Sequence
  Synthetic Polynucleotide
caagctctcagtatcatgctcgg

SEQ ID NO: 1214 - DNA, 23 bp, Artificial Sequence
  Synthetic Polynucleotide
cttactggacagcctgtataacc

SEQ ID NO: 1215 - DNA, 23 bp, Artificial Sequence
  Synthetic Polynucleotide
agcaaaccggattgtggttcgag

SEQ ID NO: 1216 - DNA, 23 bp, Artificial Sequence
  Synthetic Polynucleotide
gctttattccacatggacaactg

SEQ ID NO: 1217 - DNA, 23 bp, Artificial Sequence
  Synthetic Polynucleotide
tcactgcaccattgctgttccaa

SEQ ID NO: 1218 - DNA, 23 bp, Artificial Sequence
  Synthetic Polynucleotide
agatccagagacaacccttttgg

SEQ ID NO: 1219 - DNA, 23 bp, Artificial Sequence
  Synthetic Polynucleotide
atatagttggactggtgcttgat

SEQ ID NO: 1220 - DNA, 23 bp, Artificial Sequence
  Synthetic Polynucleotide
ctgctgagcccggaggagatact

SEQ ID NO: 1221 - DNA, 23 bp, Artificial Sequence
  Synthetic Polynucleotide
gcataattcccaccaagtttgga

SEQ ID NO: 1222 - DNA, 23 bp, Artificial Sequence
  Synthetic Polynucleotide
tgcggcttgtcagaccatcttga

SEQ ID NO: 1223 - DNA, 23 bp, Artificial Sequence
  Synthetic Polynucleotide
aaggatctgcagctatctaaggc

SEQ ID NO: 1224 - DNA, 23 bp, Artificial Sequence
  Synthetic Polynucleotide
tatgtacacactgctccggaatc

SEQ ID NO: 1225 - DNA, 23 bp, Artificial Sequence
  Synthetic Polynucleotide
ggagagaattgccaaaagccacg

SEQ ID NO: 1226 - DNA, 23 bp, Artificial Sequence
  Synthetic Polynucleotide
aggagatactgctctccatagag

SEQ ID NO: 1227 - DNA, 23 bp, Artificial Sequence
  Synthetic Polynucleotide
ctacctgccagtgtctcttagga

SEQ ID NO: 1228 - DNA, 23 bp, Artificial Sequence
  Synthetic Polynucleotide
gtgtctcttaggagtgaggtacc

SEQ ID NO: 1229 - DNA, 23 bp, Artificial Sequence
  Synthetic Polynucleotide
cactttcgccagtgcaactttac

SEQ ID NO: 1230 - DNA, 23 bp, Artificial Sequence
  Synthetic Polynucleotide
ctcagcacacaggtaataacgtg

SEQ ID NO: 1231 - DNA, 23 bp, Artificial Sequence
  Synthetic Polynucleotide
ggcctcccggagagaagatgaca

SEQ ID NO: 1232 - DNA, 23 bp, Artificial Sequence
  Synthetic Polynucleotide
aaggggttttccgaagcagataa

SEQ ID NO: 1233 - DNA, 23 bp, Artificial Sequence
  Synthetic Polynucleotide
aagagcacttctacctggagact

SEQ ID NO: 1234 - DNA, 23 bp, Artificial Sequence
  Synthetic Polynucleotide
caaccttccctccaacacggcct

SEQ ID NO: 1235 - DNA, 23 bp, Artificial Sequence
  Synthetic Polynucleotide
ggaaccaagctcggctgtggaga

SEQ ID NO: 1236 - DNA, 23 bp, Artificial Sequence
  Synthetic Polynucleotide
ctctcagtatcatgctcggaaga

SEQ ID NO: 1237 - DNA, 23 bp, Artificial Sequence
  Synthetic Polynucleotide
agcaagctctcagtatcatgctc

SEQ ID NO: 1238 - DNA, 23 bp, Artificial Sequence
  Synthetic Polynucleotide
tccagtctaaaccctgccattga

SEQ ID NO: 1239 - DNA, 23 bp, Artificial Sequence
  Synthetic Polynucleotide
agaatgagctgtctctccggctg

SEQ ID NO: 1240 - DNA, 23 bp, Artificial Sequence
  Synthetic Polynucleotide
cgatgcatgctggaccgtgatga

SEQ ID NO: 1241 - DNA, 23 bp, Artificial Sequence
  Synthetic Polynucleotide
tcataagaacctccgcacagata

SEQ ID NO: 1242 - DNA, 23 bp, Artificial Sequence
  Synthetic Polynucleotide
ccaaagtaatttccacctagaaa

SEQ ID NO: 1243 - DNA, 23 bp, Artificial Sequence
  Synthetic Polynucleotide
ataagaacctccgcacagatatt

SEQ ID NO: 1244 - DNA, 23 bp, Artificial Sequence
  Synthetic Polynucleotide
actgtataaatccaaccttctgc

SEQ ID NO: 1245 - DNA, 23 bp, Artificial Sequence
  Synthetic Polynucleotide
gttccagggtttgtttgtttcat

SEQ ID NO: 1246 - DNA, 23 bp, Artificial Sequence
  Synthetic Polynucleotide
aggagagaattgccaaaagccac

SEQ ID NO: 1247 - DNA, 23 bp, Artificial Sequence
  Synthetic Polynucleotide
gcgagacaagcactaacactgtg

SEQ ID NO: 1248 - DNA, 23 bp, Artificial Sequence
  Synthetic Polynucleotide
aagatcccggcatttggcagcat

SEQ ID NO: 1249 - DNA, 23 bp, Artificial Sequence
  Synthetic Polynucleotide
cctcccggagagaagatgacatt

SEQ ID NO: 1250 - DNA, 23 bp, Artificial Sequence
  Synthetic Polynucleotide
ataattcccaccaagtttggaat

SEQ ID NO: 1251 - DNA, 23 bp, Artificial Sequence
  Synthetic Polynucleotide
taatctttacatttgaagccaaa

SEQ ID NO: 1252 - DNA, 23 bp, Artificial Sequence
  Synthetic Polynucleotide
tccacatggacaactgctataaa

SEQ ID NO: 1253 - DNA, 23 bp, Artificial Sequence
  Synthetic Polynucleotide
cagaaaactgcaaaccctggtct

SEQ ID NO: 1254 - DNA, 23 bp, Artificial Sequence
  Synthetic Polynucleotide
ttaatctttacatttgaagccaa

SEQ ID NO: 1255 - DNA, 23 bp, Artificial Sequence
  Synthetic Polynucleotide
gtatttctcagcattcaagcagg

SEQ ID NO: 1256 - DNA, 23 bp, Artificial Sequence
  Synthetic Polynucleotide
tgaaacaaattcaaggttaattg

SEQ ID NO: 1257 - DNA, 23 bp, Artificial Sequence
  Synthetic Polynucleotide
tgatgtcactaataaatgaaact

SEQ ID NO: 1258 - DNA, 23 bp, Artificial Sequence
  Synthetic Polynucleotide
aagtttctgcggttggtaaagag

SEQ ID NO: 1259 - DNA, 23 bp, Artificial Sequence
  Synthetic Polynucleotide
ttctatcttctttgccatcaaag

SEQ ID NO: 1260 - DNA, 23 bp, Artificial Sequence
  Synthetic Polynucleotide
cctgtataacctcaagttctgat

SEQ ID NO: 1261 - DNA, 23 bp, Artificial Sequence
  Synthetic Polynucleotide
atcataagaacctccgcacagat

SEQ ID NO: 1262 - DNA, 23 bp, Artificial Sequence
  Synthetic Polynucleotide
tggcatcgtcatgagtatgtaca

SEQ ID NO: 1263 - DNA, 23 bp, Artificial Sequence
  Synthetic Polynucleotide
gttgcagtgacaactgtggaagg

SEQ ID NO: 1264 - DNA, 23 bp, Artificial Sequence
  Synthetic Polynucleotide
cttttatggacctgagctgaaga

SEQ ID NO: 1265 - DNA, 23 bp, Artificial Sequence
  Synthetic Polynucleotide
gaagttcaagaatatgctgtttc

SEQ ID NO: 1266 - DNA, 23 bp, Artificial Sequence
  Synthetic Polynucleotide
aggcctcaaccctcaaggagctg

SEQ ID NO: 1267 - DNA, 23 bp, Artificial Sequence
  Synthetic Polynucleotide
gaggaacataccacagaacatgg

SEQ ID NO: 1268 - DNA, 23 bp, Artificial Sequence
  Synthetic Polynucleotide
catcgtcatgagtatgtacacac

SEQ ID NO: 1269 - DNA, 23 bp, Artificial Sequence
  Synthetic Polynucleotide
acctgacacacttcaaccagaag

SEQ ID NO: 1270 - DNA, 23 bp, Artificial Sequence
  Synthetic Polynucleotide
gagctcagcacacaggtaataac

SEQ ID NO: 1271 - DNA, 23 bp, Artificial Sequence
  Synthetic Polynucleotide
agaacatggatctattaaagtca

SEQ ID NO: 1272 - DNA, 23 bp, Artificial Sequence
  Synthetic Polynucleotide
agtggaaccaagctcggctgtgg

SEQ ID NO: 1273 - DNA, 23 bp, Artificial Sequence
  Synthetic Polynucleotide
ccctgccattgatattggacagg

SEQ ID NO: 1274 - DNA, 23 bp, Artificial Sequence
  Synthetic Polynucleotide
ccctcaccctcagcttcttcttc

SEQ ID NO: 1275 - DNA, 23 bp, Artificial Sequence
  Synthetic Polynucleotide
gtgcaggagagaattgccaaaag

SEQ ID NO: 1276 - DNA, 23 bp, Artificial Sequence
  Synthetic Polynucleotide
aggacatgctgataactggtggc

SEQ ID NO: 1277 - DNA, 23 bp, Artificial Sequence
  Synthetic Polynucleotide
aaaaagattatgtttaatcttta

SEQ ID NO: 1278 - DNA, 23 bp, Artificial Sequence
  Synthetic Polynucleotide
tgcaggagagaattgccaaaagc

SEQ ID NO: 1279 - DNA, 23 bp, Artificial Sequence
  Synthetic Polynucleotide
aaaaaagattatgtttaatcttt

SEQ ID NO: 1280 - DNA, 23 bp, Artificial Sequence
  Synthetic Polynucleotide
caaggccttcataccaaaatggt

SEQ ID NO: 1281 - DNA, 23 bp, Artificial Sequence
  Synthetic Polynucleotide
gctgcgctggtttgctgggaagc

SEQ ID NO: 1282 - DNA, 23 bp, Artificial Sequence
  Synthetic Polynucleotide
acaatgttctagatttctttccc

SEQ ID NO: 1283 - DNA, 23 bp, Artificial Sequence
  Synthetic Polynucleotide
gcagctcgagctcagcacacagg

SEQ ID NO: 1284 - DNA, 23 bp, Artificial Sequence
  Synthetic Polynucleotide
tgcaccattgctgttccaaaagg

SEQ ID NO: 1285 - DNA, 23 bp, Artificial Sequence
  Synthetic Polynucleotide
aacaagatcgtccacttttctgc

SEQ ID NO: 1286 - DNA, 23 bp, Artificial Sequence
  Synthetic Polynucleotide
cacaggtaataacgtgaaggaac

SEQ ID NO: 1287 - DNA, 23 bp, Artificial Sequence
  Synthetic Polynucleotide
cagtggcatgagagttttattca

SEQ ID NO: 1288 - DNA, 23 bp, Artificial Sequence
  Synthetic Polynucleotide
gccttcaaaccaatgaacagcaa

SEQ ID NO: 1289 - DNA, 23 bp, Artificial Sequence
  Synthetic Polynucleotide
ccttcaaaccaatgaacagcaaa

SEQ ID NO: 1290 - DNA, 23 bp, Artificial Sequence
  Synthetic Polynucleotide
aaatccaaccaactcaattattg

SEQ ID NO: 1291 - DNA, 23 bp, Artificial Sequence
  Synthetic Polynucleotide
tacaaaaaagattatgtttaatc

SEQ ID NO: 1292 - DNA, 23 bp, Artificial Sequence
  Synthetic Polynucleotide
acctgtgggatgcctgcagagga

SEQ ID NO: 1293 - DNA, 23 bp, Artificial Sequence
  Synthetic Polynucleotide
agccctcaagaccactcagaggc

SEQ ID NO: 1294 - DNA, 23 bp, Artificial Sequence
  Synthetic Polynucleotide
gtgggatgcctgcagaggaggtg

SEQ ID NO: 1295 - DNA, 23 bp, Artificial Sequence
  Synthetic Polynucleotide
aaccagtggcatgagagttttat

SEQ ID NO: 1296 - DNA, 23 bp, Artificial Sequence
  Synthetic Polynucleotide
atggatctattaaagtcacagaa

SEQ ID NO: 1297 - DNA, 23 bp, Artificial Sequence
  Synthetic Polynucleotide
cccttcctgtgatccattttact

SEQ ID NO: 1298 - DNA, 23 bp, Artificial Sequence
  Synthetic Polynucleotide
tcctggctgcttctatcttcttt

SEQ ID NO: 1299 - DNA, 23 bp, Artificial Sequence
  Synthetic Polynucleotide
tgtgtacacagatggctctgtgc

SEQ ID NO: 1300 - DNA, 23 bp, Artificial Sequence
  Synthetic Polynucleotide
cttcaagttctacctgacagtcc

SEQ ID NO: 1301 - DNA, 21 bp, Artificial Sequence
  Synthetic Polynucleotide
gctctccaagtatgatcgtct

SEQ ID NO: 1302 - DNA, 21 bp, Artificial Sequence
  Synthetic Polynucleotide
tctgcagaacaagatcgtcca

SEQ ID NO: 1303 - DNA, 21 bp, Artificial Sequence
  Synthetic Polynucleotide
gtccacttttctgccaatgcc

SEQ ID NO: 1304 - DNA, 21 bp, Artificial Sequence
  Synthetic Polynucleotide
ccacggctcccagtgcgggtt

SEQ ID NO: 1305 - DNA, 21 bp, Artificial Sequence
  Synthetic Polynucleotide
aacacggagattggcattgag

SEQ ID NO: 1306 - DNA, 21 bp, Artificial Sequence
  Synthetic Polynucleotide
acacggagattggcattgaga

SEQ ID NO: 1307 - DNA, 21 bp, Artificial Sequence
  Synthetic Polynucleotide
ggagagaagatgacattgcca

SEQ ID NO: 1308 - DNA, 21 bp, Artificial Sequence
  Synthetic Polynucleotide
aaccagtggcatgagagtttt

SEQ ID NO: 1309 - DNA, 21 bp, Artificial Sequence
  Synthetic Polynucleotide
catgcaggcctctggtgaggc

SEQ ID NO: 1310 - DNA, 21 bp, Artificial Sequence
  Synthetic Polynucleotide
tctggtgaggccgtgtactgt

SEQ ID NO: 1311 - DNA, 21 bp, Artificial Sequence
  Synthetic Polynucleotide
cccgggcccacgccaagatca

SEQ ID NO: 1312 - DNA, 21 bp, Artificial Sequence
  Synthetic Polynucleotide
cgccaagatcaagtccataga

SEQ ID NO: 1313 - DNA, 21 bp, Artificial Sequence
  Synthetic Polynucleotide
gtgttgggcatatcattggtg

SEQ ID NO: 1314 - DNA, 21 bp, Artificial Sequence
  Synthetic Polynucleotide
atcattggtgctgtggttgct

SEQ ID NO: 1315 - DNA, 21 bp, Artificial Sequence
  Synthetic Polynucleotide
gcaaaccggattgtggttcga

SEQ ID NO: 1316 - DNA, 21 bp, Artificial Sequence
  Synthetic Polynucleotide
aaccggattgtggttcgagtg

SEQ ID NO: 1317 - DNA, 21 bp, Artificial Sequence
  Synthetic Polynucleotide
cggattgtggttcgagtgaag

SEQ ID NO: 1318 - DNA, 21 bp, Artificial Sequence
  Synthetic Polynucleotide
atgctggaccgtgatgaggac

SEQ ID NO: 1319 - DNA, 21 bp, Artificial Sequence
  Synthetic Polynucleotide
gatgaggacatgctgataact

SEQ ID NO: 1320 - DNA, 21 bp, Artificial Sequence
  Synthetic Polynucleotide
gccagatacaaggttggcttc

SEQ ID NO: 1321 - DNA, 21 bp, Artificial Sequence
  Synthetic Polynucleotide
ctctctcagagtattatggaa

SEQ ID NO: 1322 - DNA, 21 bp, Artificial Sequence
  Synthetic Polynucleotide
ccaaccttccctccaacacgg

SEQ ID NO: 1323 - DNA, 21 bp, Artificial Sequence
  Synthetic Polynucleotide
gcctagcaagctctcagtatc

SEQ ID NO: 1324 - DNA, 21 bp, Artificial Sequence
  Synthetic Polynucleotide
tagcaagctctcagtatcatg

SEQ ID NO: 1325 - DNA, 21 bp, Artificial Sequence
  Synthetic Polynucleotide
tctcagtatcatgctcggaag

SEQ ID NO: 1326 - DNA, 21 bp, Artificial Sequence
  Synthetic Polynucleotide
tcccaccaagtttggaataag

SEQ ID NO: 1327 - DNA, 21 bp, Artificial Sequence
  Synthetic Polynucleotide
cctccgcacagatattgtcat

SEQ ID NO: 1328 - DNA, 21 bp, Artificial Sequence
  Synthetic Polynucleotide
tacaagatcccggcatttggc

SEQ ID NO: 1329 - DNA, 21 bp, Artificial Sequence
  Synthetic Polynucleotide
tcagcacacaggtaataacgt

SEQ ID NO: 1330 - DNA, 21 bp, Artificial Sequence
  Synthetic Polynucleotide
aaaccctggtctgtgagggtc

SEQ ID NO: 1331 - DNA, 21 bp, Artificial Sequence
  Synthetic Polynucleotide
taccacagaacatggatctat

SEQ ID NO: 1332 - DNA, 21 bp, Artificial Sequence
  Synthetic Polynucleotide
gaacatggatctattaaagtc

SEQ ID NO: 1333 - DNA, 21 bp, Artificial Sequence
  Synthetic Polynucleotide
acatggatctattaaagtcac

SEQ ID NO: 1334 - DNA, 21 bp, Artificial Sequence
  Synthetic Polynucleotide
cgggaagggtttgtgctattc

SEQ ID NO: 1335 - DNA, 21 bp, Artificial Sequence
  Synthetic Polynucleotide
gggaagggtttgtgctattcc

SEQ ID NO: 1336 - DNA, 21 bp, Artificial Sequence
  Synthetic Polynucleotide
acctcaagttctgatggtgtc

SEQ ID NO: 1337 - DNA, 21 bp, Artificial Sequence
  Synthetic Polynucleotide
cctcaagttctgatggtgtct

SEQ ID NO: 1338 - DNA, 21 bp, Artificial Sequence
  Synthetic Polynucleotide
ttcccacaaacctctagaagc

SEQ ID NO: 1339 - DNA, 21 bp, Artificial Sequence
  Synthetic Polynucleotide
cccacaaacctctagaagctt

SEQ ID NO: 1340 - DNA, 21 bp, Artificial Sequence
  Synthetic Polynucleotide
ggccttcaaaccaatgaacag

SEQ ID NO: 1341 - DNA, 21 bp, Artificial Sequence
  Synthetic Polynucleotide
ccaatgaacagcaaagcataa

SEQ ID NO: 1342 - DNA, 21 bp, Artificial Sequence
  Synthetic Polynucleotide
gaaacaaattcaaggttaatt

SEQ ID NO: 1343 - DNA, 21 bp, Artificial Sequence
  Synthetic Polynucleotide
tgaagctgcataaagcaagat

SEQ ID NO: 1344 - DNA, 21 bp, Artificial Sequence
  Synthetic Polynucleotide
gctgcataaagcaagattact

SEQ ID NO: 1345 - DNA, 21 bp, Artificial Sequence
  Synthetic Polynucleotide
aaaaagattatgtttaatctt

SEQ ID NO: 1346 - DNA, 21 bp, Artificial Sequence
  Synthetic Polynucleotide
ctaaggcttggttttcttact

SEQ ID NO: 1347 - DNA, 21 bp, Artificial Sequence
  Synthetic Polynucleotide
ttggttttcttactgtcatat

SEQ ID NO: 1348 - DNA, 21 bp, Artificial Sequence
  Synthetic Polynucleotide
cctcaagtttctgcggttggt

SEQ ID NO: 1349 - DNA, 21 bp, Artificial Sequence
  Synthetic Polynucleotide
ctcaagtttctgcggttggta

SEQ ID NO: 1350 - DNA, 21 bp, Artificial Sequence
  Synthetic Polynucleotide
agttggactggtgcttgatgt

SEQ ID NO: 1351 - DNA, 21 bp, Artificial Sequence
  Synthetic Polynucleotide
gatttggaagacaagtgaatg

SEQ ID NO: 1352 - DNA, 21 bp, Artificial Sequence
  Synthetic Polynucleotide
cagtgatgctctccaagtatg

SEQ ID NO: 1353 - DNA, 21 bp, Artificial Sequence
  Synthetic Polynucleotide
aaaccggattgtggttcgagt

SEQ ID NO: 1354 - DNA, 21 bp, Artificial Sequence
  Synthetic Polynucleotide
agtttctgcggttggtaaaga

SEQ ID NO: 1355 - DNA, 21 bp, Artificial Sequence
  Synthetic Polynucleotide
tgcttctatcttctttgccat

SEQ ID NO: 1356 - DNA, 21 bp, Artificial Sequence
  Synthetic Polynucleotide
aggaacataccacagaacatg

SEQ ID NO: 1357 - DNA, 21 bp, Artificial Sequence
  Synthetic Polynucleotide
ttttatggacctgagctgaag

SEQ ID NO: 1358 - DNA, 21 bp, Artificial Sequence
  Synthetic Polynucleotide
cgggcccacgccaagatcaag

SEQ ID NO: 1359 - DNA, 21 bp, Artificial Sequence
  Synthetic Polynucleotide
ggaagggtttgtgctattccc

SEQ ID NO: 1360 - DNA, 21 bp, Artificial Sequence
  Synthetic Polynucleotide
cctgtgatccattttactgca

SEQ ID NO: 1361 - DNA, 21 bp, Artificial Sequence
  Synthetic Polynucleotide
caaccaactcaattattgagc

SEQ ID NO: 1362 - DNA, 21 bp, Artificial Sequence
  Synthetic Polynucleotide
gatctgcagctatctaaggct

SEQ ID NO: 1363 - DNA, 21 bp, Artificial Sequence
  Synthetic Polynucleotide
ttccgaagcagataatgttgt

SEQ ID NO: 1364 - DNA, 21 bp, Artificial Sequence
  Synthetic Polynucleotide
tatgtacacactgctccggaa

SEQ ID NO: 1365 - DNA, 21 bp, Artificial Sequence
  Synthetic Polynucleotide
tccagagacaacccttttggc

SEQ ID NO: 1366 - DNA, 21 bp, Artificial Sequence
  Synthetic Polynucleotide
tcccggcatttggcagcatcc

SEQ ID NO: 1367 - DNA, 21 bp, Artificial Sequence
  Synthetic Polynucleotide
aggttacttgtgttgggcata

SEQ ID NO: 1368 - DNA, 21 bp, Artificial Sequence
  Synthetic Polynucleotide
ctgggaagaatgcctagcaag

SEQ ID NO: 1369 - DNA, 21 bp, Artificial Sequence
  Synthetic Polynucleotide
tttggaagacaagtgaatgca

SEQ ID NO: 1370 - DNA, 21 bp, Artificial Sequence
  Synthetic Polynucleotide
tgcataaagcaagattactct

SEQ ID NO: 1371 - DNA, 21 bp, Artificial Sequence
  Synthetic Polynucleotide
ccaccaagtttggaataagct

SEQ ID NO: 1372 - DNA, 21 bp, Artificial Sequence
  Synthetic Polynucleotide
aagttcaacaaggagaattgt

SEQ ID NO: 1373 - DNA, 21 bp, Artificial Sequence
  Synthetic Polynucleotide
tggagaaaaatgcagatccag

SEQ ID NO: 1374 - DNA, 21 bp, Artificial Sequence
  Synthetic Polynucleotide
ctagcaagctctcagtatcat

SEQ ID NO: 1375 - DNA, 21 bp, Artificial Sequence
  Synthetic Polynucleotide
tggcatcgtcatgagtatgta

SEQ ID NO: 1376 - DNA, 21 bp, Artificial Sequence
  Synthetic Polynucleotide
ctgctccttgcaccatgttgc

SEQ ID NO: 1377 - DNA, 21 bp, Artificial Sequence
  Synthetic Polynucleotide
ccaaccaactcaattattgag

SEQ ID NO: 1378 - DNA, 21 bp, Artificial Sequence
  Synthetic Polynucleotide
tacttgtgttgggcatatcat

SEQ ID NO: 1379 - DNA, 21 bp, Artificial Sequence
  Synthetic Polynucleotide
aggataaggttacttgtgttg

SEQ ID NO: 1380 - DNA, 21 bp, Artificial Sequence
  Synthetic Polynucleotide
ggggcttgcacagtgatgctc

SEQ ID NO: 1381 - DNA, 21 bp, Artificial Sequence
  Synthetic Polynucleotide
atgaaacaaattcaaggttaa

SEQ ID NO: 1382 - DNA, 21 bp, Artificial Sequence
  Synthetic Polynucleotide
tctttgtgatcttggactgtc

SEQ ID NO: 1383 - DNA, 21 bp, Artificial Sequence
  Synthetic Polynucleotide
gtggagaaaaatgcagatcca

SEQ ID NO: 1384 - DNA, 21 bp, Artificial Sequence
  Synthetic Polynucleotide
gcagataatgttgtgtcaggg

SEQ ID NO: 1385 - DNA, 21 bp, Artificial Sequence
  Synthetic Polynucleotide
cccaccaagtttggaataagc

SEQ ID NO: 1386 - DNA, 21 bp, Artificial Sequence
  Synthetic Polynucleotide
gcaaagcataaccttgaatct

SEQ ID NO: 1387 - DNA, 21 bp, Artificial Sequence
  Synthetic Polynucleotide
cacacaggtaataacgtgaag

SEQ ID NO: 1388 - DNA, 21 bp, Artificial Sequence
  Synthetic Polynucleotide
accagccattatcacaattga

SEQ ID NO: 1389 - DNA, 21 bp, Artificial Sequence
  Synthetic Polynucleotide
acaaaaatccaaccaactcaa

SEQ ID NO: 1390 - DNA, 21 bp, Artificial Sequence
  Synthetic Polynucleotide
gaataggaagcaccaagacga

SEQ ID NO: 1391 - DNA, 21 bp, Artificial Sequence
  Synthetic Polynucleotide
cagcgctgacctcaatggaca

SEQ ID NO: 1392 - DNA, 21 bp, Artificial Sequence
  Synthetic Polynucleotide
ctcccggagagaagatgacat

SEQ ID NO: 1393 - DNA, 21 bp, Artificial Sequence
  Synthetic Polynucleotide
gccattgatattggacaggtg

SEQ ID NO: 1394 - DNA, 21 bp, Artificial Sequence
  Synthetic Polynucleotide
agcacacaggtaataacgtga

SEQ ID NO: 1395 - DNA, 21 bp, Artificial Sequence
  Synthetic Polynucleotide
tgcctaaatggtgaatatgca

SEQ ID NO: 1396 - DNA, 21 bp, Artificial Sequence
  Synthetic Polynucleotide
ctgccactgggttttatagaa

SEQ ID NO: 1397 - DNA, 21 bp, Artificial Sequence
  Synthetic Polynucleotide
tacacactgctccggaatcag

SEQ ID NO: 1398 - DNA, 21 bp, Artificial Sequence
  Synthetic Polynucleotide
gttacttgtgttgggcatatc

SEQ ID NO: 1399 - DNA, 21 bp, Artificial Sequence
  Synthetic Polynucleotide
tgaggccgtgtactgtgacga

SEQ ID NO: 1400 - DNA, 21 bp, Artificial Sequence
  Synthetic Polynucleotide
cctagcaagctctcagtatca

SEQ ID NO: 1401 - DNA, 21 bp, Artificial Sequence
  Synthetic Polynucleotide
ttggcattgagatgaagttca

SEQ ID NO: 1402 - DNA, 21 bp, Artificial Sequence
  Synthetic Polynucleotide
agaacacccaatctgggctac

SEQ ID NO: 1403 - DNA, 21 bp, Artificial Sequence
  Synthetic Polynucleotide
tgtttgtttcatttccgctga

SEQ ID NO: 1404 - DNA, 21 bp, Artificial Sequence
  Synthetic Polynucleotide
ttccactacttcagctatggg

SEQ ID NO: 1405 - DNA, 21 bp, Artificial Sequence
  Synthetic Polynucleotide
ataccacagaacatggatcta

SEQ ID NO: 1406 - DNA, 21 bp, Artificial Sequence
  Synthetic Polynucleotide
taagaacctccgcacagatat

SEQ ID NO: 1407 - DNA, 21 bp, Artificial Sequence
  Synthetic Polynucleotide
cctacaagatcccggcatttg

SEQ ID NO: 1408 - DNA, 21 bp, Artificial Sequence
  Synthetic Polynucleotide
agtatgtacacactgctccgg

SEQ ID NO: 1409 - DNA, 21 bp, Artificial Sequence
  Synthetic Polynucleotide
gctccttgcaccatgttgcag

SEQ ID NO: 1410 - DNA, 21 bp, Artificial Sequence
  Synthetic Polynucleotide
caaagtaatttccacctagaa

SEQ ID NO: 1411 - DNA, 21 bp, Artificial Sequence
  Synthetic Polynucleotide
agccacggctcccagtgcggg

SEQ ID NO: 1412 - DNA, 21 bp, Artificial Sequence
  Synthetic Polynucleotide
agcttgtctgccactgggttt

SEQ ID NO: 1413 - DNA, 21 bp, Artificial Sequence
  Synthetic Polynucleotide
ggtgcttgatgtcactaataa

SEQ ID NO: 1414 - DNA, 21 bp, Artificial Sequence
  Synthetic Polynucleotide
ccatcaaagatgccatccgtg

SEQ ID NO: 1415 - DNA, 21 bp, Artificial Sequence
  Synthetic Polynucleotide
ctctccaagtatgatcgtctg

SEQ ID NO: 1416 - DNA, 21 bp, Artificial Sequence
  Synthetic Polynucleotide
ggagcaggaggaacataccac

SEQ ID NO: 1417 - DNA, 21 bp, Artificial Sequence
  Synthetic Polynucleotide
atctctctcagagtattatgg

SEQ ID NO: 1418 - DNA, 21 bp, Artificial Sequence
  Synthetic Polynucleotide
gctcggaagagtgaggttgac

SEQ ID NO: 1419 - DNA, 21 bp, Artificial Sequence
  Synthetic Polynucleotide
tcagcgctgacctcaatggac

SEQ ID NO: 1420 - DNA, 21 bp, Artificial Sequence
  Synthetic Polynucleotide
ttattccacatggacaactgc

SEQ ID NO: 1421 - DNA, 21 bp, Artificial Sequence
  Synthetic Polynucleotide
aacaagaaggccatctatgca

SEQ ID NO: 1422 - DNA, 21 bp, Artificial Sequence
  Synthetic Polynucleotide
taccagccattatcacaattg

SEQ ID NO: 1423 - DNA, 21 bp, Artificial Sequence
  Synthetic Polynucleotide
tggacacagtgagcttgtctg

SEQ ID NO: 1424 - DNA, 21 bp, Artificial Sequence
  Synthetic Polynucleotide
agaacctccgcacagatattg

SEQ ID NO: 1425 - DNA, 21 bp, Artificial Sequence
  Synthetic Polynucleotide
tgagcttgtctgccactgggt

SEQ ID NO: 1426 - DNA, 21 bp, Artificial Sequence
  Synthetic Polynucleotide
gttgctaagcttcctgcccaa

SEQ ID NO: 1427 - DNA, 21 bp, Artificial Sequence
  Synthetic Polynucleotide
ggcggctgtgcaaaaccaacc

SEQ ID NO: 1428 - DNA, 21 bp, Artificial Sequence
  Synthetic Polynucleotide
ggcatcgtcatgagtatgtac

SEQ ID NO: 1429 - DNA, 21 bp, Artificial Sequence
  Synthetic Polynucleotide
ttgctaagcttcctgcccaaa

SEQ ID NO: 1430 - DNA, 21 bp, Artificial Sequence
  Synthetic Polynucleotide
gccaaggtaaccagtggcatg

SEQ ID NO: 1431 - DNA, 21 bp, Artificial Sequence
  Synthetic Polynucleotide
tggtgctgtggttgctgacac

SEQ ID NO: 1432 - DNA, 21 bp, Artificial Sequence
  Synthetic Polynucleotide
atggcagaaaggtggtggaga

SEQ ID NO: 1433 - DNA, 21 bp, Artificial Sequence
  Synthetic Polynucleotide
aggaagcaccaagacgaggct

SEQ ID NO: 1434 - DNA, 21 bp, Artificial Sequence
  Synthetic Polynucleotide
tgagactaactcagggaaccc

SEQ ID NO: 1435 - DNA, 21 bp, Artificial Sequence
  Synthetic Polynucleotide
gcaaaccctggtctgtgaggg

SEQ ID NO: 1436 - DNA, 21 bp, Artificial Sequence
  Synthetic Polynucleotide
tgtctacacagaacaccatga

SEQ ID NO: 1437 - DNA, 21 bp, Artificial Sequence
  Synthetic Polynucleotide
aggggtgaaaatcacctatga

SEQ ID NO: 1438 - DNA, 21 bp, Artificial Sequence
  Synthetic Polynucleotide
accctgccattgatattggac

SEQ ID NO: 1439 - DNA, 21 bp, Artificial Sequence
  Synthetic Polynucleotide
atgttggctccagtctaaacc

SEQ ID NO: 1440 - DNA, 21 bp, Artificial Sequence
  Synthetic Polynucleotide
aggacatgctgataactggtg

SEQ ID NO: 1441 - DNA, 21 bp, Artificial Sequence
  Synthetic Polynucleotide
cttttatggacctgagctgaa

SEQ ID NO: 1442 - DNA, 21 bp, Artificial Sequence
  Synthetic Polynucleotide
aagctctcagtatcatgctcg

SEQ ID NO: 1443 - DNA, 21 bp, Artificial Sequence
  Synthetic Polynucleotide
tgacctgaaaactccagttac

SEQ ID NO: 1444 - DNA, 21 bp, Artificial Sequence
  Synthetic Polynucleotide
tatagttggactggtgcttga

SEQ ID NO: 1445 - DNA, 21 bp, Artificial Sequence
  Synthetic Polynucleotide
cacccgggcccacgccaagat

SEQ ID NO: 1446 - DNA, 21 bp, Artificial Sequence
  Synthetic Polynucleotide
caagtttctgcggttggtaaa

SEQ ID NO: 1447 - DNA, 21 bp, Artificial Sequence
  Synthetic Polynucleotide
ggatttggaagacaagtgaat

SEQ ID NO: 1448 - DNA, 21 bp, Artificial Sequence
  Synthetic Polynucleotide
ttttatagaacacccaatctg

SEQ ID NO: 1449 - DNA, 21 bp, Artificial Sequence
  Synthetic Polynucleotide
acagtgatgctctccaagtat

SEQ ID NO: 1450 - DNA, 21 bp, Artificial Sequence
  Synthetic Polynucleotide
cctgacacacttcaaccagaa

SEQ ID NO: 1451 - DNA, 21 bp, Artificial Sequence
  Synthetic Polynucleotide
gaaccaagctcggctgtggag

SEQ ID NO: 1452 - DNA, 21 bp, Artificial Sequence
  Synthetic Polynucleotide
gggttttccgaagcagataat

SEQ ID NO: 1453 - DNA, 21 bp, Artificial Sequence
  Synthetic Polynucleotide
tctccaagtatgatcgtctgc

SEQ ID NO: 1454 - DNA, 21 bp, Artificial Sequence
  Synthetic Polynucleotide
tgggaagaatgcctagcaagc

SEQ ID NO: 1455 - DNA, 21 bp, Artificial Sequence
  Synthetic Polynucleotide
ccacttttctgccaatgcctg

SEQ ID NO: 1456 - DNA, 21 bp, Artificial Sequence
  Synthetic Polynucleotide
agaatatgctgtttcctatga

SEQ ID NO: 1457 - DNA, 21 bp, Artificial Sequence
  Synthetic Polynucleotide
ggaaccaagctcggctgtgga

SEQ ID NO: 1458 - DNA, 21 bp, Artificial Sequence
  Synthetic Polynucleotide
cactaacactgtgcccaacac

SEQ ID NO: 1459 - DNA, 21 bp, Artificial Sequence
  Synthetic Polynucleotide
ctttgttgcaaaaatgttggg

SEQ ID NO: 1460 - DNA, 21 bp, Artificial Sequence
  Synthetic Polynucleotide
gtttgtttcatttccgctgat

SEQ ID NO: 1461 - DNA, 21 bp, Artificial Sequence
  Synthetic Polynucleotide
tctttgccatcaaagatgcca

SEQ ID NO: 1462 - DNA, 21 bp, Artificial Sequence
  Synthetic Polynucleotide
ctgggctacagctttgagact

SEQ ID NO: 1463 - DNA, 21 bp, Artificial Sequence
  Synthetic Polynucleotide
gcacacaggtaataacgtgaa

SEQ ID NO: 1464 - DNA, 21 bp, Artificial Sequence
  Synthetic Polynucleotide
acttgtgttgggcatatcatt

SEQ ID NO: 1465 - DNA, 21 bp, Artificial Sequence
  Synthetic Polynucleotide
ccgctgcacaggctacagacc

SEQ ID NO: 1466 - DNA, 21 bp, Artificial Sequence
  Synthetic Polynucleotide
aaggaataggaagcaccaaga

SEQ ID NO: 1467 - DNA, 21 bp, Artificial Sequence
  Synthetic Polynucleotide
tcaagtttctgcggttggtaa

SEQ ID NO: 1468 - DNA, 21 bp, Artificial Sequence
  Synthetic Polynucleotide
ggaaggaataggaagcaccaa

SEQ ID NO: 1469 - DNA, 21 bp, Artificial Sequence
  Synthetic Polynucleotide
aaacctctagaagcttaaacc

SEQ ID NO: 1470 - DNA, 21 bp, Artificial Sequence
  Synthetic Polynucleotide
gatgttggctccagtctaaac

SEQ ID NO: 1471 - DNA, 21 bp, Artificial Sequence
  Synthetic Polynucleotide
ccacatggacaactgctataa

SEQ ID NO: 1472 - DNA, 21 bp, Artificial Sequence
  Synthetic Polynucleotide
gaggacatgctgataactggt

SEQ ID NO: 1473 - DNA, 21 bp, Artificial Sequence
  Synthetic Polynucleotide
agctctcagtatcatgctcgg

SEQ ID NO: 1474 - DNA, 21 bp, Artificial Sequence
  Synthetic Polynucleotide
tactggacagcctgtataacc

SEQ ID NO: 1475 - DNA, 21 bp, Artificial Sequence
  Synthetic Polynucleotide
caaaccggattgtggttcgag

SEQ ID NO: 1476 - DNA, 21 bp, Artificial Sequence
  Synthetic Polynucleotide
tttattccacatggacaactg

SEQ ID NO: 1477 - DNA, 21 bp, Artificial Sequence
  Synthetic Polynucleotide
actgcaccattgctgttccaa

SEQ ID NO: 1478 - DNA, 21 bp, Artificial Sequence
  Synthetic Polynucleotide
atccagagacaacccttttgg

SEQ ID NO: 1479 - DNA, 21 bp, Artificial Sequence
  Synthetic Polynucleotide
atagttggactggtgcttgat

SEQ ID NO: 1480 - DNA, 21 bp, Artificial Sequence
  Synthetic Polynucleotide
gctgagcccggaggagatact

SEQ ID NO: 1481 - DNA, 21 bp, Artificial Sequence
  Synthetic Polynucleotide
ataattcccaccaagtttgga

SEQ ID NO: 1482 - DNA, 21 bp, Artificial Sequence
  Synthetic Polynucleotide
cggcttgtcagaccatcttga

SEQ ID NO: 1483 - DNA, 21 bp, Artificial Sequence
  Synthetic Polynucleotide
ggatctgcagctatctaaggc

SEQ ID NO: 1484 - DNA, 21 bp, Artificial Sequence
  Synthetic Polynucleotide
tgtacacactgctccggaatc

SEQ ID NO: 1485 - DNA, 21 bp, Artificial Sequence
  Synthetic Polynucleotide
agagaattgccaaaagccacg

SEQ ID NO: 1486 - DNA, 21 bp, Artificial Sequence
  Synthetic Polynucleotide
gagatactgctctccatagag

SEQ ID NO: 1487 - DNA, 21 bp, Artificial Sequence
  Synthetic Polynucleotide
acctgccagtgtctcttagga

SEQ ID NO: 1488 - DNA, 21 bp, Artificial Sequence
  Synthetic Polynucleotide
gtctcttaggagtgaggtacc

SEQ ID NO: 1489 - DNA, 21 bp, Artificial Sequence
  Synthetic Polynucleotide
ctttcgccagtgcaactttac

SEQ ID NO: 1490 - DNA, 21 bp, Artificial Sequence
  Synthetic Polynucleotide
cagcacacaggtaataacgtg

SEQ ID NO: 1491 - DNA, 21 bp, Artificial Sequence
  Synthetic Polynucleotide
cctcccggagagaagatgaca

SEQ ID NO: 1492 - DNA, 21 bp, Artificial Sequence
  Synthetic Polynucleotide
ggggttttccgaagcagataa

SEQ ID NO: 1493 - DNA, 21 bp, Artificial Sequence
  Synthetic Polynucleotide
gagcacttctacctggagact

SEQ ID NO: 1494 - DNA, 21 bp, Artificial Sequence
  Synthetic Polynucleotide
accttccctccaacacggcct

SEQ ID NO: 1495 - DNA, 21 bp, Artificial Sequence
  Synthetic Polynucleotide
aaccaagctcggctgtggaga

SEQ ID NO: 1496 - DNA, 21 bp, Artificial Sequence
  Synthetic Polynucleotide
ctcagtatcatgctcggaaga

SEQ ID NO: 1497 - DNA, 21 bp, Artificial Sequence
  Synthetic Polynucleotide
caagctctcagtatcatgctc

SEQ ID NO: 1498 - DNA, 21 bp, Artificial Sequence
  Synthetic Polynucleotide
cagtctaaaccctgccattga

SEQ ID NO: 1499 - DNA, 21 bp, Artificial Sequence
  Synthetic Polynucleotide
aatgagctgtctctccggctg

SEQ ID NO: 1500 - DNA, 21 bp, Artificial Sequence
  Synthetic Polynucleotide
atgcatgctggaccgtgatga

SEQ ID NO: 1501 - DNA, 21 bp, Artificial Sequence
  Synthetic Polynucleotide
ataagaacctccgcacagata

SEQ ID NO: 1502 - DNA, 21 bp, Artificial Sequence
  Synthetic Polynucleotide
aaagtaatttccacctagaaa

SEQ ID NO: 1503 - DNA, 21 bp, Artificial Sequence
  Synthetic Polynucleotide
aagaacctccgcacagatatt

SEQ ID NO: 1504 - DNA, 21 bp, Artificial Sequence
  Synthetic Polynucleotide
tgtataaatccaaccttctgc

SEQ ID NO: 1505 - DNA, 21 bp, Artificial Sequence
  Synthetic Polynucleotide
tccagggtttgtttgtttcat

SEQ ID NO: 1506 - DNA, 21 bp, Artificial Sequence
  Synthetic Polynucleotide
gagagaattgccaaaagccac

SEQ ID NO: 1507 - DNA, 21 bp, Artificial Sequence
  Synthetic Polynucleotide
gagacaagcactaacactgtg

SEQ ID NO: 1508 - DNA, 21 bp, Artificial Sequence
  Synthetic Polynucleotide
gatcccggcatttggcagcat

SEQ ID NO: 1509 - DNA, 21 bp, Artificial Sequence
  Synthetic Polynucleotide
tcccggagagaagatgacatt

SEQ ID NO: 1510 - DNA, 21 bp, Artificial Sequence
  Synthetic Polynucleotide
aattcccaccaagtttggaat

SEQ ID NO: 1511 - DNA, 21 bp, Artificial Sequence
  Synthetic Polynucleotide
atctttacatttgaagccaaa

SEQ ID NO: 1512 - DNA, 21 bp, Artificial Sequence
  Synthetic Polynucleotide
cacatggacaactgctataaa

SEQ ID NO: 1513 - DNA, 21 bp, Artificial Sequence
  Synthetic Polynucleotide
gaaaactgcaaaccctggtct

SEQ ID NO: 1514 - DNA, 21 bp, Artificial Sequence
  Synthetic Polynucleotide
aatctttacatttgaagccaa

SEQ ID NO: 1515 - DNA, 21 bp, Artificial Sequence
  Synthetic Polynucleotide
atttctcagcattcaagcagg

SEQ ID NO: 1516 - DNA, 21 bp, Artificial Sequence
  Synthetic Polynucleotide
aaacaaattcaaggttaattg

SEQ ID NO: 1517 - DNA, 21 bp, Artificial Sequence
  Synthetic Polynucleotide
atgtcactaataaatgaaact

SEQ ID NO: 1518 - DNA, 21 bp, Artificial Sequence
  Synthetic Polynucleotide
gtttctgcggttggtaaagag

SEQ ID NO: 1519 - DNA, 21 bp, Artificial Sequence
  Synthetic Polynucleotide
ctatcttctttgccatcaaag

SEQ ID NO: 1520 - DNA, 21 bp, Artificial Sequence
  Synthetic Polynucleotide
tgtataacctcaagttctgat

SEQ ID NO: 1521 - DNA, 21 bp, Artificial Sequence
  Synthetic Polynucleotide
cataagaacctccgcacagat

SEQ ID NO: 1522 - DNA, 21 bp, Artificial Sequence
  Synthetic Polynucleotide
gcatcgtcatgagtatgtaca

SEQ ID NO: 1523 - DNA, 21 bp, Artificial Sequence
  Synthetic Polynucleotide
tgcagtgacaactgtggaagg

SEQ ID NO: 1524 - DNA, 21 bp, Artificial Sequence
  Synthetic Polynucleotide
tttatggacctgagctgaaga

SEQ ID NO: 1525 - DNA, 21 bp, Artificial Sequence
  Synthetic Polynucleotide
agttcaagaatatgctgtttc

SEQ ID NO: 1526 - DNA, 21 bp, Artificial Sequence
  Synthetic Polynucleotide
gcctcaaccctcaaggagctg

SEQ ID NO: 1527 - DNA, 21 bp, Artificial Sequence
  Synthetic Polynucleotide
ggaacataccacagaacatgg

SEQ ID NO: 1528 - DNA, 21 bp, Artificial Sequence
  Synthetic Polynucleotide
tcgtcatgagtatgtacacac

SEQ ID NO: 1529 - DNA, 21 bp, Artificial Sequence
  Synthetic Polynucleotide
ctgacacacttcaaccagaag

SEQ ID NO: 1530 - DNA, 21 bp, Artificial Sequence
  Synthetic Polynucleotide
gctcagcacacaggtaataac

SEQ ID NO: 1531 - DNA, 21 bp, Artificial Sequence
  Synthetic Polynucleotide
aacatggatctattaaagtca

SEQ ID NO: 1532 - DNA, 21 bp, Artificial Sequence
  Synthetic Polynucleotide
tggaaccaagctcggctgtgg

SEQ ID NO: 1533 - DNA, 21 bp, Artificial Sequence
  Synthetic Polynucleotide
ctgccattgatattggacagg

SEQ ID NO: 1534 - DNA, 21 bp, Artificial Sequence
  Synthetic Polynucleotide
ctcaccctcagcttcttcttc

SEQ ID NO: 1535 - DNA, 21 bp, Artificial Sequence
  Synthetic Polynucleotide
gcaggagagaattgccaaaag

SEQ ID NO: 1536 - DNA, 21 bp, Artificial Sequence
  Synthetic Polynucleotide
gacatgctgataactggtggc

SEQ ID NO: 1537 - DNA, 21 bp, Artificial Sequence
  Synthetic Polynucleotide
aaagattatgtttaatcttta

SEQ ID NO: 1538 - DNA, 21 bp, Artificial Sequence
  Synthetic Polynucleotide
caggagagaattgccaaaagc

SEQ ID NO: 1539 - DNA, 21 bp, Artificial Sequence
  Synthetic Polynucleotide
aaaagattatgtttaatcttt

SEQ ID NO: 1540 - DNA, 21 bp, Artificial Sequence
  Synthetic Polynucleotide
aggccttcataccaaaatggt

SEQ ID NO: 1541 - DNA, 21 bp, Artificial Sequence
  Synthetic Polynucleotide
tgcgctggtttgctgggaagc

SEQ ID NO: 1542 - DNA, 21 bp, Artificial Sequence
  Synthetic Polynucleotide
aatgttctagatttctttccc

SEQ ID NO: 1543 - DNA, 21 bp, Artificial Sequence
  Synthetic Polynucleotide
agctcgagctcagcacacagg

SEQ ID NO: 1544 - DNA, 21 bp, Artificial Sequence
  Synthetic Polynucleotide
caccattgctgttccaaaagg

SEQ ID NO: 1545 - DNA, 21 bp, Artificial Sequence
  Synthetic Polynucleotide
caagatcgtccacttttctgc

SEQ ID NO: 1546 - DNA, 21 bp, Artificial Sequence
  Synthetic Polynucleotide
caggtaataacgtgaaggaac

SEQ ID NO: 1547 - DNA, 21 bp, Artificial Sequence
  Synthetic Polynucleotide
gtggcatgagagttttattca

SEQ ID NO: 1548 - DNA, 21 bp, Artificial Sequence
  Synthetic Polynucleotide
cttcaaaccaatgaacagcaa

SEQ ID NO: 1549 - DNA, 21 bp, Artificial Sequence
  Synthetic Polynucleotide
ttcaaaccaatgaacagcaaa

SEQ ID NO: 1550 - DNA, 21 bp, Artificial Sequence
  Synthetic Polynucleotide
atccaaccaactcaattattg

SEQ ID NO: 1551 - DNA, 21 bp, Artificial Sequence
  Synthetic Polynucleotide
caaaaaagattatgtttaatc

SEQ ID NO: 1552 - DNA, 21 bp, Artificial Sequence
  Synthetic Polynucleotide
ctgtgggatgcctgcagagga

SEQ ID NO: 1553 - DNA, 21 bp, Artificial Sequence
  Synthetic Polynucleotide
ccctcaagaccactcagaggc

SEQ ID NO: 1554 - DNA, 21 bp, Artificial Sequence
  Synthetic Polynucleotide
gggatgcctgcagaggaggtg

SEQ ID NO: 1555 - DNA, 21 bp, Artificial Sequence
  Synthetic Polynucleotide
ccagtggcatgagagttttat

SEQ ID NO: 1556 - DNA, 21 bp, Artificial Sequence
  Synthetic Polynucleotide
ggatctattaaagtcacagaa

SEQ ID NO: 1557 - DNA, 21 bp, Artificial Sequence
  Synthetic Polynucleotide
cttcctgtgatccattttact

SEQ ID NO: 1558 - DNA, 21 bp, Artificial Sequence
  Synthetic Polynucleotide
ctggctgcttctatcttcttt

SEQ ID NO: 1559 - DNA, 21 bp, Artificial Sequence
  Synthetic Polynucleotide
tgtacacagatggctctgtgc

SEQ ID NO: 1560 - DNA, 21 bp, Artificial Sequence
  Synthetic Polynucleotide
tcaagttctacctgacagtcc

SEQ ID NO: 1561 - DNA, 23 bp, Artificial Sequence
  Synthetic Polynucleotide
agacgatcatacttggagagcat

SEQ ID NO: 1562 - DNA, 23 bp, Artificial Sequence
  Synthetic Polynucleotide
tggacgatcttgttctgcagacg

SEQ ID NO: 1563 - DNA, 23 bp, Artificial Sequence
  Synthetic Polynucleotide
ggcattggcagaaaagtggacga

SEQ ID NO: 1564 - DNA, 23 bp, Artificial Sequence
  Synthetic Polynucleotide
aacccgcactgggagccgtggct

SEQ ID NO: 1565 - DNA, 23 bp, Artificial Sequence
  Synthetic Polynucleotide
ctcaatgccaatctccgtgttcc

SEQ ID NO: 1566 - DNA, 23 bp, Artificial Sequence
  Synthetic Polynucleotide
tctcaatgccaatctccgtgttc

SEQ ID NO: 1567 - DNA, 23 bp, Artificial Sequence
  Synthetic Polynucleotide
tggcaatgtcatcttctctccgg

SEQ ID NO: 1568 - DNA, 23 bp, Artificial Sequence
  Synthetic Polynucleotide
aaaactctcatgccactggttac

SEQ ID NO: 1569 - DNA, 23 bp, Artificial Sequence
  Synthetic Polynucleotide
gcctcaccagaggcctgcatgtc

SEQ ID NO: 1570 - DNA, 23 bp, Artificial Sequence
  Synthetic Polynucleotide
acagtacacggcctcaccagagg

SEQ ID NO: 1571 - DNA, 23 bp, Artificial Sequence
  Synthetic Polynucleotide
tgatcttggcgtgggcccgggtg

SEQ ID NO: 1572 - DNA, 23 bp, Artificial Sequence
  Synthetic Polynucleotide
tctatggacttgatcttggcgtg

SEQ ID NO: 1573 - DNA, 23 bp, Artificial Sequence
  Synthetic Polynucleotide
caccaatgatatgcccaacacaa

SEQ ID NO: 1574 - DNA, 23 bp, Artificial Sequence
  Synthetic Polynucleotide
agcaaccacagcaccaatgatat

SEQ ID NO: 1575 - DNA, 23 bp, Artificial Sequence
  Synthetic Polynucleotide
tcgaaccacaatccggtttgctg

SEQ ID NO: 1576 - DNA, 23 bp, Artificial Sequence
  Synthetic Polynucleotide
cactcgaaccacaatccggtttg

SEQ ID NO: 1577 - DNA, 23 bp, Artificial Sequence
  Synthetic Polynucleotide
cttcactcgaaccacaatccggt

SEQ ID NO: 1578 - DNA, 23 bp, Artificial Sequence
  Synthetic Polynucleotide
gtcctcatcacggtccagcatgc

SEQ ID NO: 1579 - DNA, 23 bp, Artificial Sequence
  Synthetic Polynucleotide
agttatcagcatgtcctcatcac

SEQ ID NO: 1580 - DNA, 23 bp, Artificial Sequence
  Synthetic Polynucleotide
gaagccaaccttgtatctggcca

SEQ ID NO: 1581 - DNA, 23 bp, Artificial Sequence
  Synthetic Polynucleotide
ttccataatactctgagagagat

SEQ ID NO: 1582 - DNA, 23 bp, Artificial Sequence
  Synthetic Polynucleotide
ccgtgttggagggaaggttggtt

SEQ ID NO: 1583 - DNA, 23 bp, Artificial Sequence
  Synthetic Polynucleotide
gatactgagagcttgctaggcat

SEQ ID NO: 1584 - DNA, 23 bp, Artificial Sequence
  Synthetic Polynucleotide
catgatactgagagcttgctagg

SEQ ID NO: 1585 - DNA, 23 bp, Artificial Sequence
  Synthetic Polynucleotide
cttccgagcatgatactgagagc

SEQ ID NO: 1586 - DNA, 23 bp, Artificial Sequence
  Synthetic Polynucleotide
cttattccaaacttggtgggaat

SEQ ID NO: 1587 - DNA, 23 bp, Artificial Sequence
  Synthetic Polynucleotide
atgacaatatctgtgcggaggtt

SEQ ID NO: 1588 - DNA, 23 bp, Artificial Sequence
  Synthetic Polynucleotide
gccaaatgccgggatcttgtagg

SEQ ID NO: 1589 - DNA, 23 bp, Artificial Sequence
  Synthetic Polynucleotide
acgttattacctgtgtgctgagc

SEQ ID NO: 1590 - DNA, 23 bp, Artificial Sequence
  Synthetic Polynucleotide
gaccctcacagaccagggtttgc

SEQ ID NO: 1591 - DNA, 23 bp, Artificial Sequence
  Synthetic Polynucleotide
atagatccatgttctgtggtatg

SEQ ID NO: 1592 - DNA, 23 bp, Artificial Sequence
  Synthetic Polynucleotide
gactttaatagatccatgttctg

SEQ ID NO: 1593 - DNA, 23 bp, Artificial Sequence
  Synthetic Polynucleotide
gtgactttaatagatccatgttc

SEQ ID NO: 1594 - DNA, 23 bp, Artificial Sequence
  Synthetic Polynucleotide
gaatagcacaaacccttcccgac

SEQ ID NO: 1595 - DNA, 23 bp, Artificial Sequence
  Synthetic Polynucleotide
ggaatagcacaaacccttcccga

SEQ ID NO: 1596 - DNA, 23 bp, Artificial Sequence
  Synthetic Polynucleotide
gacaccatcagaacttgaggtta

SEQ ID NO: 1597 - DNA, 23 bp, Artificial Sequence
  Synthetic Polynucleotide
agacaccatcagaacttgaggtt

SEQ ID NO: 1598 - DNA, 23 bp, Artificial Sequence
  Synthetic Polynucleotide
gcttctagaggtttgtgggaatc

SEQ ID NO: 1599 - DNA, 23 bp, Artificial Sequence
  Synthetic Polynucleotide
aagcttctagaggtttgtgggaa

SEQ ID NO: 1600 - DNA, 23 bp, Artificial Sequence
  Synthetic Polynucleotide
ctgttcattggtttgaaggccag

SEQ ID NO: 1601 - DNA, 23 bp, Artificial Sequence
  Synthetic Polynucleotide
ttatgctttgctgttcattggtt

SEQ ID NO: 1602 - DNA, 23 bp, Artificial Sequence
  Synthetic Polynucleotide
aattaaccttgaatttgtttcat

SEQ ID NO: 1603 - DNA, 23 bp, Artificial Sequence
  Synthetic Polynucleotide
atcttgctttatgcagcttcaca

SEQ ID NO: 1604 - DNA, 23 bp, Artificial Sequence
  Synthetic Polynucleotide
agtaatcttgctttatgcagctt

SEQ ID NO: 1605 - DNA, 23 bp, Artificial Sequence
  Synthetic Polynucleotide
aagattaaacataatcttttttg

SEQ ID NO: 1606 - DNA, 23 bp, Artificial Sequence
  Synthetic Polynucleotide
agtaagaaaaccaagccttagat

SEQ ID NO: 1607 - DNA, 23 bp, Artificial Sequence
  Synthetic Polynucleotide
atatgacagtaagaaaaccaagc

SEQ ID NO: 1608 - DNA, 23 bp, Artificial Sequence
  Synthetic Polynucleotide
accaaccgcagaaacttgaggtg

SEQ ID NO: 1609 - DNA, 23 bp, Artificial Sequence
  Synthetic Polynucleotide
taccaaccgcagaaacttgaggt

SEQ ID NO: 1610 - DNA, 23 bp, Artificial Sequence
  Synthetic Polynucleotide
acatcaagcaccagtccaactat

SEQ ID NO: 1611 - DNA, 23 bp, Artificial Sequence
  Synthetic Polynucleotide
cattcacttgtcttccaaatccc

SEQ ID NO: 1612 - DNA, 23 bp, Artificial Sequence
  Synthetic Polynucleotide
catacttggagagcatcactgtg

SEQ ID NO: 1613 - DNA, 23 bp, Artificial Sequence
  Synthetic Polynucleotide
actcgaaccacaatccggtttgc

SEQ ID NO: 1614 - DNA, 23 bp, Artificial Sequence
  Synthetic Polynucleotide
tctttaccaaccgcagaaacttg

SEQ ID NO: 1615 - DNA, 23 bp, Artificial Sequence
  Synthetic Polynucleotide
atggcaaagaagatagaagcagc

SEQ ID NO: 1616 - DNA, 23 bp, Artificial Sequence
  Synthetic Polynucleotide
catgttctgtggtatgttcctcc

SEQ ID NO: 1617 - DNA, 23 bp, Artificial Sequence
  Synthetic Polynucleotide
cttcagctcaggtccataaaagg

SEQ ID NO: 1618 - DNA, 23 bp, Artificial Sequence
  Synthetic Polynucleotide
cttgatcttggcgtgggcccggg

SEQ ID NO: 1619 - DNA, 23 bp, Artificial Sequence
  Synthetic Polynucleotide
gggaatagcacaaacccttcccg

SEQ ID NO: 1620 - DNA, 23 bp, Artificial Sequence
  Synthetic Polynucleotide
tgcagtaaaatggatcacaggaa

SEQ ID NO: 1621 - DNA, 23 bp, Artificial Sequence
  Synthetic Polynucleotide
gctcaataattgagttggttgga

SEQ ID NO: 1622 - DNA, 23 bp, Artificial Sequence
  Synthetic Polynucleotide
agccttagatagctgcagatcct

SEQ ID NO: 1623 - DNA, 23 bp, Artificial Sequence
  Synthetic Polynucleotide
acaacattatctgcttcggaaaa

SEQ ID NO: 1624 - DNA, 23 bp, Artificial Sequence
  Synthetic Polynucleotide
ttccggagcagtgtgtacatact

SEQ ID NO: 1625 - DNA, 23 bp, Artificial Sequence
  Synthetic Polynucleotide
gccaaaagggttgtctctggatc

SEQ ID NO: 1626 - DNA, 23 bp, Artificial Sequence
  Synthetic Polynucleotide
ggatgctgccaaatgccgggatc

SEQ ID NO: 1627 - DNA, 23 bp, Artificial Sequence
  Synthetic Polynucleotide
tatgcccaacacaagtaacctta

SEQ ID NO: 1628 - DNA, 23 bp, Artificial Sequence
  Synthetic Polynucleotide
cttgctaggcattcttcccagca

SEQ ID NO: 1629 - DNA, 23 bp, Artificial Sequence
  Synthetic Polynucleotide
tgcattcacttgtcttccaaatc

SEQ ID NO: 1630 - DNA, 23 bp, Artificial Sequence
  Synthetic Polynucleotide
agagtaatcttgctttatgcagc

SEQ ID NO: 1631 - DNA, 23 bp, Artificial Sequence
  Synthetic Polynucleotide
agcttattccaaacttggtggga

SEQ ID NO: 1632 - DNA, 23 bp, Artificial Sequence
  Synthetic Polynucleotide
acaattctccttgttgaacttgt

SEQ ID NO: 1633 - DNA, 23 bp, Artificial Sequence
  Synthetic Polynucleotide
ctggatctgcatttttctccacc

SEQ ID NO: 1634 - DNA, 23 bp, Artificial Sequence
  Synthetic Polynucleotide
atgatactgagagcttgctaggc

SEQ ID NO: 1635 - DNA, 23 bp, Artificial Sequence
  Synthetic Polynucleotide
tacatactcatgacgatgccagg

SEQ ID NO: 1636 - DNA, 23 bp, Artificial Sequence
  Synthetic Polynucleotide
gcaacatggtgcaaggagcagat

SEQ ID NO: 1637 - DNA, 23 bp, Artificial Sequence
  Synthetic Polynucleotide
ctcaataattgagttggttggat

SEQ ID NO: 1638 - DNA, 23 bp, Artificial Sequence
  Synthetic Polynucleotide
atgatatgcccaacacaagtaac

SEQ ID NO: 1639 - DNA, 23 bp, Artificial Sequence
  Synthetic Polynucleotide
caacacaagtaaccttatccttc

SEQ ID NO: 1640 - DNA, 23 bp, Artificial Sequence
  Synthetic Polynucleotide
gagcatcactgtgcaagccccgc

SEQ ID NO: 1641 - DNA, 23 bp, Artificial Sequence
  Synthetic Polynucleotide
ttaaccttgaatttgtttcattc

SEQ ID NO: 1642 - DNA, 23 bp, Artificial Sequence
  Synthetic Polynucleotide
gacagtccaagatcacaaagata

SEQ ID NO: 1643 - DNA, 23 bp, Artificial Sequence
  Synthetic Polynucleotide
tggatctgcatttttctccacca

SEQ ID NO: 1644 - DNA, 23 bp, Artificial Sequence
  Synthetic Polynucleotide
ccctgacacaacattatctgctt

SEQ ID NO: 1645 - DNA, 23 bp, Artificial Sequence
  Synthetic Polynucleotide
gcttattccaaacttggtgggaa

SEQ ID NO: 1646 - DNA, 23 bp, Artificial Sequence
  Synthetic Polynucleotide
agattcaaggttatgctttgctg

SEQ ID NO: 1647 - DNA, 23 bp, Artificial Sequence
  Synthetic Polynucleotide
cttcacgttattacctgtgtgct

SEQ ID NO: 1648 - DNA, 23 bp, Artificial Sequence
  Synthetic Polynucleotide
tcaattgtgataatggctggtag

SEQ ID NO: 1649 - DNA, 23 bp, Artificial Sequence
  Synthetic Polynucleotide
ttgagttggttggatttttgtat

SEQ ID NO: 1650 - DNA, 23 bp, Artificial Sequence
  Synthetic Polynucleotide
tcgtcttggtgcttcctattcct

SEQ ID NO: 1651 - DNA, 23 bp, Artificial Sequence
  Synthetic Polynucleotide
tgtccattgaggtcagcgctgac

SEQ ID NO: 1652 - DNA, 23 bp, Artificial Sequence
  Synthetic Polynucleotide
atgtcatcttctctccgggaggc

SEQ ID NO: 1653 - DNA, 23 bp, Artificial Sequence
  Synthetic Polynucleotide
cacctgtccaatatcaatggcag

SEQ ID NO: 1654 - DNA, 23 bp, Artificial Sequence
  Synthetic Polynucleotide
tcacgttattacctgtgtgctga

SEQ ID NO: 1655 - DNA, 23 bp, Artificial Sequence
  Synthetic Polynucleotide
tgcatattcaccatttaggcata

SEQ ID NO: 1656 - DNA, 23 bp, Artificial Sequence
  Synthetic Polynucleotide
ttctataaaacccagtggcagac

SEQ ID NO: 1657 - DNA, 23 bp, Artificial Sequence
  Synthetic Polynucleotide
ctgattccggagcagtgtgtaca

SEQ ID NO: 1658 - DNA, 23 bp, Artificial Sequence
  Synthetic Polynucleotide
gatatgcccaacacaagtaacct

SEQ ID NO: 1659 - DNA, 23 bp, Artificial Sequence
  Synthetic Polynucleotide
tcgtcacagtacacggcctcacc

SEQ ID NO: 1660 - DNA, 23 bp, Artificial Sequence
  Synthetic Polynucleotide
tgatactgagagcttgctaggca

SEQ ID NO: 1661 - DNA, 23 bp, Artificial Sequence
  Synthetic Polynucleotide
tgaacttcatctcaatgccaatc

SEQ ID NO: 1662 - DNA, 23 bp, Artificial Sequence
  Synthetic Polynucleotide
gtagcccagattgggtgttctat

SEQ ID NO: 1663 - DNA, 23 bp, Artificial Sequence
  Synthetic Polynucleotide
tcagcggaaatgaaacaaacaaa

SEQ ID NO: 1664 - DNA, 23 bp, Artificial Sequence
  Synthetic Polynucleotide
cccatagctgaagtagtggaagg

SEQ ID NO: 1665 - DNA, 23 bp, Artificial Sequence
  Synthetic Polynucleotide
tagatccatgttctgtggtatgt

SEQ ID NO: 1666 - DNA, 23 bp, Artificial Sequence
  Synthetic Polynucleotide
atatctgtgcggaggttcttatg

SEQ ID NO: 1667 - DNA, 23 bp, Artificial Sequence
  Synthetic Polynucleotide
caaatgccgggatcttgtaggtg

SEQ ID NO: 1668 - DNA, 23 bp, Artificial Sequence
  Synthetic Polynucleotide
ccggagcagtgtgtacatactca

SEQ ID NO: 1669 - DNA, 23 bp, Artificial Sequence
  Synthetic Polynucleotide
ctgcaacatggtgcaaggagcag

SEQ ID NO: 1670 - DNA, 23 bp, Artificial Sequence
  Synthetic Polynucleotide
ttctaggtggaaattactttggc

SEQ ID NO: 1671 - DNA, 23 bp, Artificial Sequence
  Synthetic Polynucleotide
cccgcactgggagccgtggcttt

SEQ ID NO: 1672 - DNA, 23 bp, Artificial Sequence
  Synthetic Polynucleotide
aaacccagtggcagacaagctca

SEQ ID NO: 1673 - DNA, 23 bp, Artificial Sequence
  Synthetic Polynucleotide
ttattagtgacatcaagcaccag

SEQ ID NO: 1674 - DNA, 23 bp, Artificial Sequence
  Synthetic Polynucleotide
cacggatggcatctttgatggca

SEQ ID NO: 1675 - DNA, 23 bp, Artificial Sequence
  Synthetic Polynucleotide
cagacgatcatacttggagagca

SEQ ID NO: 1676 - DNA, 23 bp, Artificial Sequence
  Synthetic Polynucleotide
gtggtatgttcctcctgctccat

SEQ ID NO: 1677 - DNA, 23 bp, Artificial Sequence
  Synthetic Polynucleotide
ccataatactctgagagagatcc

SEQ ID NO: 1678 - DNA, 23 bp, Artificial Sequence
  Synthetic Polynucleotide
gtcaacctcactcttccgagcat

SEQ ID NO: 1679 - DNA, 23 bp, Artificial Sequence
  Synthetic Polynucleotide
gtccattgaggtcagcgctgaca

SEQ ID NO: 1680 - DNA, 23 bp, Artificial Sequence
  Synthetic Polynucleotide
gcagttgtccatgtggaataaag

SEQ ID NO: 1681 - DNA, 23 bp, Artificial Sequence
  Synthetic Polynucleotide
tgcatagatggccttcttgttgg

SEQ ID NO: 1682 - DNA, 23 bp, Artificial Sequence
  Synthetic Polynucleotide
caattgtgataatggctggtagt

SEQ ID NO: 1683 - DNA, 23 bp, Artificial Sequence
  Synthetic Polynucleotide
cagacaagctcactgtgtccatg

SEQ ID NO: 1684 - DNA, 23 bp, Artificial Sequence
  Synthetic Polynucleotide
caatatctgtgcggaggttctta

SEQ ID NO: 1685 - DNA, 23 bp, Artificial Sequence
  Synthetic Polynucleotide
acccagtggcagacaagctcact

SEQ ID NO: 1686 - DNA, 23 bp, Artificial Sequence
  Synthetic Polynucleotide
ttgggcaggaagcttagcaacag

SEQ ID NO: 1687 - DNA, 23 bp, Artificial Sequence
  Synthetic Polynucleotide
ggttggttttgcacagccgccca

SEQ ID NO: 1688 - DNA, 23 bp, Artificial Sequence
  Synthetic Polynucleotide
gtacatactcatgacgatgccag

SEQ ID NO: 1689 - DNA, 23 bp, Artificial Sequence
  Synthetic Polynucleotide
tttgggcaggaagcttagcaaca

SEQ ID NO: 1690 - DNA, 23 bp, Artificial Sequence
  Synthetic Polynucleotide
catgccactggttaccttggcaa

SEQ ID NO: 1691 - DNA, 23 bp, Artificial Sequence
  Synthetic Polynucleotide
gtgtcagcaaccacagcaccaat

SEQ ID NO: 1692 - DNA, 23 bp, Artificial Sequence
  Synthetic Polynucleotide
tctccaccacctttctgccattc

SEQ ID NO: 1693 - DNA, 23 bp, Artificial Sequence
  Synthetic Polynucleotide
agcctcgtcttggtgcttcctat

SEQ ID NO: 1694 - DNA, 23 bp, Artificial Sequence
  Synthetic Polynucleotide
gggttccctgagttagtctcaaa

SEQ ID NO: 1695 - DNA, 23 bp, Artificial Sequence
  Synthetic Polynucleotide
ccctcacagaccagggtttgcag

SEQ ID NO: 1696 - DNA, 23 bp, Artificial Sequence
  Synthetic Polynucleotide
tcatggtgttctgtgtagacaca

SEQ ID NO: 1697 - DNA, 23 bp, Artificial Sequence
  Synthetic Polynucleotide
tcataggtgattttcaccccttg

SEQ ID NO: 1698 - DNA, 23 bp, Artificial Sequence
  Synthetic Polynucleotide
gtccaatatcaatggcagggttt

SEQ ID NO: 1699 - DNA, 23 bp, Artificial Sequence
  Synthetic Polynucleotide
ggtttagactggagccaacatcc

SEQ ID NO: 1700 - DNA, 23 bp, Artificial Sequence
  Synthetic Polynucleotide
caccagttatcagcatgtcctca

SEQ ID NO: 1701 - DNA, 23 bp, Artificial Sequence
  Synthetic Polynucleotide
ttcagctcaggtccataaaagga

SEQ ID NO: 1702 - DNA, 23 bp, Artificial Sequence
  Synthetic Polynucleotide
cgagcatgatactgagagcttgc

SEQ ID NO: 1703 - DNA, 23 bp, Artificial Sequence
  Synthetic Polynucleotide
gtaactggagttttcaggtcata

SEQ ID NO: 1704 - DNA, 23 bp, Artificial Sequence
  Synthetic Polynucleotide
tcaagcaccagtccaactatatc

SEQ ID NO: 1705 - DNA, 23 bp, Artificial Sequence
  Synthetic Polynucleotide
atcttggcgtgggcccgggtgct

SEQ ID NO: 1706 - DNA, 23 bp, Artificial Sequence
  Synthetic Polynucleotide
tttaccaaccgcagaaacttgag

SEQ ID NO: 1707 - DNA, 23 bp, Artificial Sequence
  Synthetic Polynucleotide
attcacttgtcttccaaatccca

SEQ ID NO: 1708 - DNA, 23 bp, Artificial Sequence
  Synthetic Polynucleotide
cagattgggtgttctataaaacc

SEQ ID NO: 1709 - DNA, 23 bp, Artificial Sequence
  Synthetic Polynucleotide
atacttggagagcatcactgtgc

SEQ ID NO: 1710 - DNA, 23 bp, Artificial Sequence
  Synthetic Polynucleotide
ttctggttgaagtgtgtcaggtc

SEQ ID NO: 1711 - DNA, 23 bp, Artificial Sequence
  Synthetic Polynucleotide
ctccacagccgagcttggttcca

SEQ ID NO: 1712 - DNA, 23 bp, Artificial Sequence
  Synthetic Polynucleotide
attatctgcttcggaaaacccct

SEQ ID NO: 1713 - DNA, 23 bp, Artificial Sequence
  Synthetic Polynucleotide
gcagacgatcatacttggagagc

SEQ ID NO: 1714 - DNA, 23 bp, Artificial Sequence
  Synthetic Polynucleotide
gcttgctaggcattcttcccagc

SEQ ID NO: 1715 - DNA, 23 bp, Artificial Sequence
  Synthetic Polynucleotide
caggcattggcagaaaagtggac

SEQ ID NO: 1716 - DNA, 23 bp, Artificial Sequence
  Synthetic Polynucleotide
tcataggaaacagcatattcttg

SEQ ID NO: 1717 - DNA, 23 bp, Artificial Sequence
  Synthetic Polynucleotide
tccacagccgagcttggttccac

SEQ ID NO: 1718 - DNA, 23 bp, Artificial Sequence
  Synthetic Polynucleotide
gtgttgggcacagtgttagtgct

SEQ ID NO: 1719 - DNA, 23 bp, Artificial Sequence
  Synthetic Polynucleotide
cccaacatttttgcaacaaagct

SEQ ID NO: 1720 - DNA, 23 bp, Artificial Sequence
  Synthetic Polynucleotide
atcagcggaaatgaaacaaacaa

SEQ ID NO: 1721 - DNA, 23 bp, Artificial Sequence
  Synthetic Polynucleotide
tggcatctttgatggcaaagaag

SEQ ID NO: 1722 - DNA, 23 bp, Artificial Sequence
  Synthetic Polynucleotide
agtctcaaagctgtagcccagat

SEQ ID NO: 1723 - DNA, 23 bp, Artificial Sequence
  Synthetic Polynucleotide
ttcacgttattacctgtgtgctg

SEQ ID NO: 1724 - DNA, 23 bp, Artificial Sequence
  Synthetic Polynucleotide
aatgatatgcccaacacaagtaa

SEQ ID NO: 1725 - DNA, 23 bp, Artificial Sequence
  Synthetic Polynucleotide
ggtctgtagcctgtgcagcggca

SEQ ID NO: 1726 - DNA, 23 bp, Artificial Sequence
  Synthetic Polynucleotide
tcttggtgcttcctattccttcc

SEQ ID NO: 1727 - DNA, 23 bp, Artificial Sequence
  Synthetic Polynucleotide
ttaccaaccgcagaaacttgagg

SEQ ID NO: 1728 - DNA, 23 bp, Artificial Sequence
  Synthetic Polynucleotide
ttggtgcttcctattccttccac

SEQ ID NO: 1729 - DNA, 23 bp, Artificial Sequence
  Synthetic Polynucleotide
ggtttaagcttctagaggtttgt

SEQ ID NO: 1730 - DNA, 23 bp, Artificial Sequence
  Synthetic Polynucleotide
gtttagactggagccaacatcca

SEQ ID NO: 1731 - DNA, 23 bp, Artificial Sequence
  Synthetic Polynucleotide
ttatagcagttgtccatgtggaa

SEQ ID NO: 1732 - DNA, 23 bp, Artificial Sequence
  Synthetic Polynucleotide
accagttatcagcatgtcctcat

SEQ ID NO: 1733 - DNA, 23 bp, Artificial Sequence
  Synthetic Polynucleotide
ccgagcatgatactgagagcttg

SEQ ID NO: 1734 - DNA, 23 bp, Artificial Sequence
  Synthetic Polynucleotide
ggttatacaggctgtccagtaag

SEQ ID NO: 1735 - DNA, 23 bp, Artificial Sequence
  Synthetic Polynucleotide
ctcgaaccacaatccggtttgct

SEQ ID NO: 1736 - DNA, 23 bp, Artificial Sequence
  Synthetic Polynucleotide
cagttgtccatgtggaataaagc

SEQ ID NO: 1737 - DNA, 23 bp, Artificial Sequence
  Synthetic Polynucleotide
ttggaacagcaatggtgcagtga

SEQ ID NO: 1738 - DNA, 23 bp, Artificial Sequence
  Synthetic Polynucleotide
ccaaaagggttgtctctggatct

SEQ ID NO: 1739 - DNA, 23 bp, Artificial Sequence
  Synthetic Polynucleotide
atcaagcaccagtccaactatat

SEQ ID NO: 1740 - DNA, 23 bp, Artificial Sequence
  Synthetic Polynucleotide
agtatctcctccgggctcagcag

SEQ ID NO: 1741 - DNA, 23 bp, Artificial Sequence
  Synthetic Polynucleotide
tccaaacttggtgggaattatgc

SEQ ID NO: 1742 - DNA, 23 bp, Artificial Sequence
  Synthetic Polynucleotide
tcaagatggtctgacaagccgca

SEQ ID NO: 1743 - DNA, 23 bp, Artificial Sequence
  Synthetic Polynucleotide
gccttagatagctgcagatcctt

SEQ ID NO: 1744 - DNA, 23 bp, Artificial Sequence
  Synthetic Polynucleotide
gattccggagcagtgtgtacata

SEQ ID NO: 1745 - DNA, 23 bp, Artificial Sequence
  Synthetic Polynucleotide
cgtggcttttggcaattctctcc

SEQ ID NO: 1746 - DNA, 23 bp, Artificial Sequence
  Synthetic Polynucleotide
ctctatggagagcagtatctcct

SEQ ID NO: 1747 - DNA, 23 bp, Artificial Sequence
  Synthetic Polynucleotide
tcctaagagacactggcaggtag

SEQ ID NO: 1748 - DNA, 23 bp, Artificial Sequence
  Synthetic Polynucleotide
ggtacctcactcctaagagacac

SEQ ID NO: 1749 - DNA, 23 bp, Artificial Sequence
  Synthetic Polynucleotide
gtaaagttgcactggcgaaagtg

SEQ ID NO: 1750 - DNA, 23 bp, Artificial Sequence
  Synthetic Polynucleotide
cacgttattacctgtgtgctgag

SEQ ID NO: 1751 - DNA, 23 bp, Artificial Sequence
  Synthetic Polynucleotide
tgtcatcttctctccgggaggcc

SEQ ID NO: 1752 - DNA, 23 bp, Artificial Sequence
  Synthetic Polynucleotide
ttatctgcttcggaaaacccctt

SEQ ID NO: 1753 - DNA, 23 bp, Artificial Sequence
  Synthetic Polynucleotide
agtctccaggtagaagtgctctt

SEQ ID NO: 1754 - DNA, 23 bp, Artificial Sequence
  Synthetic Polynucleotide
aggccgtgttggagggaaggttg

SEQ ID NO: 1755 - DNA, 23 bp, Artificial Sequence
  Synthetic Polynucleotide
tctccacagccgagcttggttcc

SEQ ID NO: 1756 - DNA, 23 bp, Artificial Sequence
  Synthetic Polynucleotide
tcttccgagcatgatactgagag

SEQ ID NO: 1757 - DNA, 23 bp, Artificial Sequence
  Synthetic Polynucleotide
gagcatgatactgagagcttgct

SEQ ID NO: 1758 - DNA, 23 bp, Artificial Sequence
  Synthetic Polynucleotide
tcaatggcagggtttagactgga

SEQ ID NO: 1759 - DNA, 23 bp, Artificial Sequence
  Synthetic Polynucleotide
cagccggagagacagctcattct

SEQ ID NO: 1760 - DNA, 23 bp, Artificial Sequence
  Synthetic Polynucleotide
tcatcacggtccagcatgcatcg

SEQ ID NO: 1761 - DNA, 23 bp, Artificial Sequence
  Synthetic Polynucleotide
tatctgtgcggaggttcttatga

SEQ ID NO: 1762 - DNA, 23 bp, Artificial Sequence
  Synthetic Polynucleotide
tttctaggtggaaattactttgg

SEQ ID NO: 1763 - DNA, 23 bp, Artificial Sequence
  Synthetic Polynucleotide
aatatctgtgcggaggttcttat

SEQ ID NO: 1764 - DNA, 23 bp, Artificial Sequence
  Synthetic Polynucleotide
gcagaaggttggatttatacagt

SEQ ID NO: 1765 - DNA, 23 bp, Artificial Sequence
  Synthetic Polynucleotide
atgaaacaaacaaaccctggaac

SEQ ID NO: 1766 - DNA, 23 bp, Artificial Sequence
  Synthetic Polynucleotide
gtggcttttggcaattctctcct

SEQ ID NO: 1767 - DNA, 23 bp, Artificial Sequence
  Synthetic Polynucleotide
cacagtgttagtgcttgtctcgc

SEQ ID NO: 1768 - DNA, 23 bp, Artificial Sequence
  Synthetic Polynucleotide
atgctgccaaatgccgggatctt

SEQ ID NO: 1769 - DNA, 23 bp, Artificial Sequence
  Synthetic Polynucleotide
aatgtcatcttctctccgggagg

SEQ ID NO: 1770 - DNA, 23 bp, Artificial Sequence
  Synthetic Polynucleotide
attccaaacttggtgggaattat

SEQ ID NO: 1771 - DNA, 23 bp, Artificial Sequence
  Synthetic Polynucleotide
tttggcttcaaatgtaaagatta

SEQ ID NO: 1772 - DNA, 23 bp, Artificial Sequence
  Synthetic Polynucleotide
tttatagcagttgtccatgtgga

SEQ ID NO: 1773 - DNA, 23 bp, Artificial Sequence
  Synthetic Polynucleotide
agaccagggtttgcagttttctg

SEQ ID NO: 1774 - DNA, 23 bp, Artificial Sequence
  Synthetic Polynucleotide
ttggcttcaaatgtaaagattaa

SEQ ID NO: 1775 - DNA, 23 bp, Artificial Sequence
  Synthetic Polynucleotide
cctgcttgaatgctgagaaatac

SEQ ID NO: 1776 - DNA, 23 bp, Artificial Sequence
  Synthetic Polynucleotide
caattaaccttgaatttgtttca

SEQ ID NO: 1777 - DNA, 23 bp, Artificial Sequence
  Synthetic Polynucleotide
agtttcatttattagtgacatca

SEQ ID NO: 1778 - DNA, 23 bp, Artificial Sequence
  Synthetic Polynucleotide
ctctttaccaaccgcagaaactt

SEQ ID NO: 1779 - DNA, 23 bp, Artificial Sequence
  Synthetic Polynucleotide
ctttgatggcaaagaagatagaa

SEQ ID NO: 1780 - DNA, 23 bp, Artificial Sequence
  Synthetic Polynucleotide
atcagaacttgaggttatacagg

SEQ ID NO: 1781 - DNA, 23 bp, Artificial Sequence
  Synthetic Polynucleotide
atctgtgcggaggttcttatgat

SEQ ID NO: 1782 - DNA, 23 bp, Artificial Sequence
  Synthetic Polynucleotide
tgtacatactcatgacgatgcca

SEQ ID NO: 1783 - DNA, 23 bp, Artificial Sequence
  Synthetic Polynucleotide
ccttccacagttgtcactgcaac

SEQ ID NO: 1784 - DNA, 23 bp, Artificial Sequence
  Synthetic Polynucleotide
tcttcagctcaggtccataaaag

SEQ ID NO: 1785 - DNA, 23 bp, Artificial Sequence
  Synthetic Polynucleotide
gaaacagcatattcttgaacttc

SEQ ID NO: 1786 - DNA, 23 bp, Artificial Sequence
  Synthetic Polynucleotide
cagctccttgagggttgaggcct

SEQ ID NO: 1787 - DNA, 23 bp, Artificial Sequence
  Synthetic Polynucleotide
ccatgttctgtggtatgttcctc

SEQ ID NO: 1788 - DNA, 23 bp, Artificial Sequence
  Synthetic Polynucleotide
gtgtgtacatactcatgacgatg

SEQ ID NO: 1789 - DNA, 23 bp, Artificial Sequence
  Synthetic Polynucleotide
cttctggttgaagtgtgtcaggt

SEQ ID NO: 1790 - DNA, 23 bp, Artificial Sequence
  Synthetic Polynucleotide
gttattacctgtgtgctgagctc

SEQ ID NO: 1791 - DNA, 23 bp, Artificial Sequence
  Synthetic Polynucleotide
tgactttaatagatccatgttct

SEQ ID NO: 1792 - DNA, 23 bp, Artificial Sequence
  Synthetic Polynucleotide
ccacagccgagcttggttccact

SEQ ID NO: 1793 - DNA, 23 bp, Artificial Sequence
  Synthetic Polynucleotide
cctgtccaatatcaatggcaggg

SEQ ID NO: 1794 - DNA, 23 bp, Artificial Sequence
  Synthetic Polynucleotide
gaagaagaagctgagggtgaggg

SEQ ID NO: 1795 - DNA, 23 bp, Artificial Sequence
  Synthetic Polynucleotide
cttttggcaattctctcctgcac

SEQ ID NO: 1796 - DNA, 23 bp, Artificial Sequence
  Synthetic Polynucleotide
gccaccagttatcagcatgtcct

SEQ ID NO: 1797 - DNA, 23 bp, Artificial Sequence
  Synthetic Polynucleotide
taaagattaaacataatcttttt

SEQ ID NO: 1798 - DNA, 23 bp, Artificial Sequence
  Synthetic Polynucleotide
gcttttggcaattctctcctgca

SEQ ID NO: 1799 - DNA, 23 bp, Artificial Sequence
  Synthetic Polynucleotide
aaagattaaacataatctttttt

SEQ ID NO: 1800 - DNA, 23 bp, Artificial Sequence
  Synthetic Polynucleotide
accattttggtatgaaggccttg

SEQ ID NO: 1801 - DNA, 23 bp, Artificial Sequence
  Synthetic Polynucleotide
gcttcccagcaaaccagcgcagc

SEQ ID NO: 1802 - DNA, 23 bp, Artificial Sequence
  Synthetic Polynucleotide
gggaaagaaatctagaacattgt

SEQ ID NO: 1803 - DNA, 23 bp, Artificial Sequence
  Synthetic Polynucleotide
cctgtgtgctgagctcgagctgc

SEQ ID NO: 1804 - DNA, 23 bp, Artificial Sequence
  Synthetic Polynucleotide
ccttttggaacagcaatggtgca

SEQ ID NO: 1805 - DNA, 23 bp, Artificial Sequence
  Synthetic Polynucleotide
gcagaaaagtggacgatcttgtt

SEQ ID NO: 1806 - DNA, 23 bp, Artificial Sequence
  Synthetic Polynucleotide
gttccttcacgttattacctgtg

SEQ ID NO: 1807 - DNA, 23 bp, Artificial Sequence
  Synthetic Polynucleotide
tgaataaaactctcatgccactg

SEQ ID NO: 1808 - DNA, 23 bp, Artificial Sequence
  Synthetic Polynucleotide
ttgctgttcattggtttgaaggc

SEQ ID NO: 1809 - DNA, 23 bp, Artificial Sequence
  Synthetic Polynucleotide
tttgctgttcattggtttgaagg

SEQ ID NO: 1810 - DNA, 23 bp, Artificial Sequence
  Synthetic Polynucleotide
caataattgagttggttggattt

SEQ ID NO: 1811 - DNA, 23 bp, Artificial Sequence
  Synthetic Polynucleotide
gattaaacataatcttttttgta

SEQ ID NO: 1812 - DNA, 23 bp, Artificial Sequence
  Synthetic Polynucleotide
tcctctgcaggcatcccacaggt

SEQ ID NO: 1813 - DNA, 23 bp, Artificial Sequence
  Synthetic Polynucleotide
gcctctgagtggtcttgagggct

SEQ ID NO: 1814 - DNA, 23 bp, Artificial Sequence
  Synthetic Polynucleotide
cacctcctctgcaggcatcccac

SEQ ID NO: 1815 - DNA, 23 bp, Artificial Sequence
  Synthetic Polynucleotide
ataaaactctcatgccactggtt

SEQ ID NO: 1816 - DNA, 23 bp, Artificial Sequence
  Synthetic Polynucleotide
ttctgtgactttaatagatccat

SEQ ID NO: 1817 - DNA, 23 bp, Artificial Sequence
  Synthetic Polynucleotide
agtaaaatggatcacaggaaggg

SEQ ID NO: 1818 - DNA, 23 bp, Artificial Sequence
  Synthetic Polynucleotide
aaagaagatagaagcagccagga

SEQ ID NO: 1819 - DNA, 23 bp, Artificial Sequence
  Synthetic Polynucleotide
gcacagagccatctgtgtacaca

SEQ ID NO: 1820 - DNA, 23 bp, Artificial Sequence
  Synthetic Polynucleotide
ggactgtcaggtagaacttgaag

SEQ ID NO: 1821 - DNA, 21 bp, Artificial Sequence
  Synthetic Polynucleotide
agacgatcatacttggagagc

SEQ ID NO: 1822 - DNA, 21 bp, Artificial Sequence
  Synthetic Polynucleotide
tggacgatcttgttctgcaga

SEQ ID NO: 1823 - DNA, 21 bp, Artificial Sequence
  Synthetic Polynucleotide
ggcattggcagaaaagtggac

SEQ ID NO: 1824 - DNA, 21 bp, Artificial Sequence
  Synthetic Polynucleotide
aacccgcactgggagccgtgg

SEQ ID NO: 1825 - DNA, 21 bp, Artificial Sequence
  Synthetic Polynucleotide
ctcaatgccaatctccgtgtt

SEQ ID NO: 1826 - DNA, 21 bp, Artificial Sequence
  Synthetic Polynucleotide
tctcaatgccaatctccgtgt

SEQ ID NO: 1827 - DNA, 21 bp, Artificial Sequence
  Synthetic Polynucleotide
tggcaatgtcatcttctctcc

SEQ ID NO: 1828 - DNA, 21 bp, Artificial Sequence
  Synthetic Polynucleotide
aaaactctcatgccactggtt

SEQ ID NO: 1829 - DNA, 21 bp, Artificial Sequence
  Synthetic Polynucleotide
gcctcaccagaggcctgcatg

SEQ ID NO: 1830 - DNA, 21 bp, Artificial Sequence
  Synthetic Polynucleotide
acagtacacggcctcaccaga

SEQ ID NO: 1831 - DNA, 21 bp, Artificial Sequence
  Synthetic Polynucleotide
tgatcttggcgtgggcccggg

SEQ ID NO: 1832 - DNA, 21 bp, Artificial Sequence
  Synthetic Polynucleotide
tctatggacttgatcttggcg

SEQ ID NO: 1833 - DNA, 21 bp, Artificial Sequence
  Synthetic Polynucleotide
caccaatgatatgcccaacac

SEQ ID NO: 1834 - DNA, 21 bp, Artificial Sequence
  Synthetic Polynucleotide
agcaaccacagcaccaatgat

SEQ ID NO: 1835 - DNA, 21 bp, Artificial Sequence
  Synthetic Polynucleotide
tcgaaccacaatccggtttgc

SEQ ID NO: 1836 - DNA, 21 bp, Artificial Sequence
  Synthetic Polynucleotide
cactcgaaccacaatccggtt

SEQ ID NO: 1837 - DNA, 21 bp, Artificial Sequence
  Synthetic Polynucleotide
cttcactcgaaccacaatccg

SEQ ID NO: 1838 - DNA, 21 bp, Artificial Sequence
  Synthetic Polynucleotide
gtcctcatcacggtccagcat

SEQ ID NO: 1839 - DNA, 21 bp, Artificial Sequence
  Synthetic Polynucleotide
agttatcagcatgtcctcatc

SEQ ID NO: 1840 - DNA, 21 bp, Artificial Sequence
  Synthetic Polynucleotide
gaagccaaccttgtatctggc

SEQ ID NO: 1841 - DNA, 21 bp, Artificial Sequence
  Synthetic Polynucleotide
ttccataatactctgagagag

SEQ ID NO: 1842 - DNA, 21 bp, Artificial Sequence
  Synthetic Polynucleotide
ccgtgttggagggaaggttgg

SEQ ID NO: 1843 - DNA, 21 bp, Artificial Sequence
  Synthetic Polynucleotide
gatactgagagcttgctaggc

SEQ ID NO: 1844 - DNA, 21 bp, Artificial Sequence
  Synthetic Polynucleotide
catgatactgagagcttgcta

SEQ ID NO: 1845 - DNA, 21 bp, Artificial Sequence
  Synthetic Polynucleotide
cttccgagcatgatactgaga

SEQ ID NO: 1846 - DNA, 21 bp, Artificial Sequence
  Synthetic Polynucleotide
cttattccaaacttggtggga

SEQ ID NO: 1847 - DNA, 21 bp, Artificial Sequence
  Synthetic Polynucleotide
atgacaatatctgtgcggagg

SEQ ID NO: 1848 - DNA, 21 bp, Artificial Sequence
  Synthetic Polynucleotide
gccaaatgccgggatcttgta

SEQ ID NO: 1849 - DNA, 21 bp, Artificial Sequence
  Synthetic Polynucleotide
acgttattacctgtgtgctga

SEQ ID NO: 1850 - DNA, 21 bp, Artificial Sequence
  Synthetic Polynucleotide
gaccctcacagaccagggttt

SEQ ID NO: 1851 - DNA, 21 bp, Artificial Sequence
  Synthetic Polynucleotide
atagatccatgttctgtggta

SEQ ID NO: 1852 - DNA, 21 bp, Artificial Sequence
  Synthetic Polynucleotide
gactttaatagatccatgttc

SEQ ID NO: 1853 - DNA, 21 bp, Artificial Sequence
  Synthetic Polynucleotide
gtgactttaatagatccatgt

SEQ ID NO: 1854 - DNA, 21 bp, Artificial Sequence
  Synthetic Polynucleotide
gaatagcacaaacccttcccg

SEQ ID NO: 1855 - DNA, 21 bp, Artificial Sequence
  Synthetic Polynucleotide
ggaatagcacaaacccttccc

SEQ ID NO: 1856 - DNA, 21 bp, Artificial Sequence
  Synthetic Polynucleotide
gacaccatcagaacttgaggt

SEQ ID NO: 1857 - DNA, 21 bp, Artificial Sequence
  Synthetic Polynucleotide
agacaccatcagaacttgagg

SEQ ID NO: 1858 - DNA, 21 bp, Artificial Sequence
  Synthetic Polynucleotide
gcttctagaggtttgtgggaa

SEQ ID NO: 1859 - DNA, 21 bp, Artificial Sequence
  Synthetic Polynucleotide
aagcttctagaggtttgtggg

SEQ ID NO: 1860 - DNA, 21 bp, Artificial Sequence
  Synthetic Polynucleotide
ctgttcattggtttgaaggcc

SEQ ID NO: 1861 - DNA, 21 bp, Artificial Sequence
  Synthetic Polynucleotide
ttatgctttgctgttcattgg

SEQ ID NO: 1862 - DNA, 21 bp, Artificial Sequence
  Synthetic Polynucleotide
aattaaccttgaatttgtttc

SEQ ID NO: 1863 - DNA, 21 bp, Artificial Sequence
  Synthetic Polynucleotide
atcttgctttatgcagcttca

SEQ ID NO: 1864 - DNA, 21 bp, Artificial Sequence
  Synthetic Polynucleotide
agtaatcttgctttatgcagc

SEQ ID NO: 1865 - DNA, 21 bp, Artificial Sequence
  Synthetic Polynucleotide
aagattaaacataatcttttt

SEQ ID NO: 1866 - DNA, 21 bp, Artificial Sequence
  Synthetic Polynucleotide
agtaagaaaaccaagccttag

SEQ ID NO: 1867 - DNA, 21 bp, Artificial Sequence
  Synthetic Polynucleotide
atatgacagtaagaaaaccaa

SEQ ID NO: 1868 - DNA, 21 bp, Artificial Sequence
  Synthetic Polynucleotide
accaaccgcagaaacttgagg

SEQ ID NO: 1869 - DNA, 21 bp, Artificial Sequence
  Synthetic Polynucleotide
taccaaccgcagaaacttgag

SEQ ID NO: 1870 - DNA, 21 bp, Artificial Sequence
  Synthetic Polynucleotide
acatcaagcaccagtccaact

SEQ ID NO: 1871 - DNA, 21 bp, Artificial Sequence
  Synthetic Polynucleotide
cattcacttgtcttccaaatc

SEQ ID NO: 1872 - DNA, 21 bp, Artificial Sequence
  Synthetic Polynucleotide
catacttggagagcatcactg

SEQ ID NO: 1873 - DNA, 21 bp, Artificial Sequence
  Synthetic Polynucleotide
actcgaaccacaatccggttt

SEQ ID NO: 1874 - DNA, 21 bp, Artificial Sequence
  Synthetic Polynucleotide
tctttaccaaccgcagaaact

SEQ ID NO: 1875 - DNA, 21 bp, Artificial Sequence
  Synthetic Polynucleotide
atggcaaagaagatagaagca

SEQ ID NO: 1876 - DNA, 21 bp, Artificial Sequence
  Synthetic Polynucleotide
catgttctgtggtatgttcct

SEQ ID NO: 1877 - DNA, 21 bp, Artificial Sequence
  Synthetic Polynucleotide
cttcagctcaggtccataaaa

SEQ ID NO: 1878 - DNA, 21 bp, Artificial Sequence
  Synthetic Polynucleotide
cttgatcttggcgtgggcccg

SEQ ID NO: 1879 - DNA, 21 bp, Artificial Sequence
  Synthetic Polynucleotide
gggaatagcacaaacccttcc

SEQ ID NO: 1880 - DNA, 21 bp, Artificial Sequence
  Synthetic Polynucleotide
tgcagtaaaatggatcacagg

SEQ ID NO: 1881 - DNA, 21 bp, Artificial Sequence
  Synthetic Polynucleotide
gctcaataattgagttggttg

SEQ ID NO: 1882 - DNA, 21 bp, Artificial Sequence
  Synthetic Polynucleotide
agccttagatagctgcagatc

SEQ ID NO: 1883 - DNA, 21 bp, Artificial Sequence
  Synthetic Polynucleotide
acaacattatctgcttcggaa

SEQ ID NO: 1884 - DNA, 21 bp, Artificial Sequence
  Synthetic Polynucleotide
ttccggagcagtgtgtacata

SEQ ID NO: 1885 - DNA, 21 bp, Artificial Sequence
  Synthetic Polynucleotide
gccaaaagggttgtctctgga

SEQ ID NO: 1886 - DNA, 21 bp, Artificial Sequence
  Synthetic Polynucleotide
ggatgctgccaaatgccggga

SEQ ID NO: 1887 - DNA, 21 bp, Artificial Sequence
  Synthetic Polynucleotide
tatgcccaacacaagtaacct

SEQ ID NO: 1888 - DNA, 21 bp, Artificial Sequence
  Synthetic Polynucleotide
cttgctaggcattcttcccag

SEQ ID NO: 1889 - DNA, 21 bp, Artificial Sequence
  Synthetic Polynucleotide
tgcattcacttgtcttccaaa

SEQ ID NO: 1890 - DNA, 21 bp, Artificial Sequence
  Synthetic Polynucleotide
agagtaatcttgctttatgca

SEQ ID NO: 1891 - DNA, 21 bp, Artificial Sequence
  Synthetic Polynucleotide
agcttattccaaacttggtgg

SEQ ID NO: 1892 - DNA, 21 bp, Artificial Sequence
  Synthetic Polynucleotide
acaattctccttgttgaactt

SEQ ID NO: 1893 - DNA, 21 bp, Artificial Sequence
  Synthetic Polynucleotide
ctggatctgcatttttctcca

SEQ ID NO: 1894 - DNA, 21 bp, Artificial Sequence
  Synthetic Polynucleotide
atgatactgagagcttgctag

SEQ ID NO: 1895 - DNA, 21 bp, Artificial Sequence
  Synthetic Polynucleotide
tacatactcatgacgatgcca

SEQ ID NO: 1896 - DNA, 21 bp, Artificial Sequence
  Synthetic Polynucleotide
gcaacatggtgcaaggagcag

SEQ ID NO: 1897 - DNA, 21 bp, Artificial Sequence
  Synthetic Polynucleotide
ctcaataattgagttggttgg

SEQ ID NO: 1898 - DNA, 21 bp, Artificial Sequence
  Synthetic Polynucleotide
atgatatgcccaacacaagta

SEQ ID NO: 1899 - DNA, 21 bp, Artificial Sequence
  Synthetic Polynucleotide
caacacaagtaaccttatcct

SEQ ID NO: 1900 - DNA, 21 bp, Artificial Sequence
  Synthetic Polynucleotide
gagcatcactgtgcaagcccc

SEQ ID NO: 1901 - DNA, 21 bp, Artificial Sequence
  Synthetic Polynucleotide
ttaaccttgaatttgtttcat

SEQ ID NO: 1902 - DNA, 21 bp, Artificial Sequence
  Synthetic Polynucleotide
gacagtccaagatcacaaaga

SEQ ID NO: 1903 - DNA, 21 bp, Artificial Sequence
  Synthetic Polynucleotide
tggatctgcatttttctccac

SEQ ID NO: 1904 - DNA, 21 bp, Artificial Sequence
  Synthetic Polynucleotide
ccctgacacaacattatctgc

SEQ ID NO: 1905 - DNA, 21 bp, Artificial Sequence
  Synthetic Polynucleotide
gcttattccaaacttggtggg

SEQ ID NO: 1906 - DNA, 21 bp, Artificial Sequence
  Synthetic Polynucleotide
agattcaaggttatgctttgc

SEQ ID NO: 1907 - DNA, 21 bp, Artificial Sequence
  Synthetic Polynucleotide
cttcacgttattacctgtgtg

SEQ ID NO: 1908 - DNA, 21 bp, Artificial Sequence
  Synthetic Polynucleotide
tcaattgtgataatggctggt

SEQ ID NO: 1909 - DNA, 21 bp, Artificial Sequence
  Synthetic Polynucleotide
ttgagttggttggatttttgt

SEQ ID NO: 1910 - DNA, 21 bp, Artificial Sequence
  Synthetic Polynucleotide
tcgtcttggtgcttcctattc

SEQ ID NO: 1911 - DNA, 21 bp, Artificial Sequence
  Synthetic Polynucleotide
tgtccattgaggtcagcgctg

SEQ ID NO: 1912 - DNA, 21 bp, Artificial Sequence
  Synthetic Polynucleotide
atgtcatcttctctccgggag

SEQ ID NO: 1913 - DNA, 21 bp, Artificial Sequence
  Synthetic Polynucleotide
cacctgtccaatatcaatggc

SEQ ID NO: 1914 - DNA, 21 bp, Artificial Sequence
  Synthetic Polynucleotide
tcacgttattacctgtgtgct

SEQ ID NO: 1915 - DNA, 21 bp, Artificial Sequence
  Synthetic Polynucleotide
tgcatattcaccatttaggca

SEQ ID NO: 1916 - DNA, 21 bp, Artificial Sequence
  Synthetic Polynucleotide
ttctataaaacccagtggcag

SEQ ID NO: 1917 - DNA, 21 bp, Artificial Sequence
  Synthetic Polynucleotide
ctgattccggagcagtgtgta

SEQ ID NO: 1918 - DNA, 21 bp, Artificial Sequence
  Synthetic Polynucleotide
gatatgcccaacacaagtaac

SEQ ID NO: 1919 - DNA, 21 bp, Artificial Sequence
  Synthetic Polynucleotide
tcgtcacagtacacggcctca

SEQ ID NO: 1920 - DNA, 21 bp, Artificial Sequence
  Synthetic Polynucleotide
tgatactgagagcttgctagg

SEQ ID NO: 1921 - DNA, 21 bp, Artificial Sequence
  Synthetic Polynucleotide
tgaacttcatctcaatgccaa

SEQ ID NO: 1922 - DNA, 21 bp, Artificial Sequence
  Synthetic Polynucleotide
gtagcccagattgggtgttct

SEQ ID NO: 1923 - DNA, 21 bp, Artificial Sequence
  Synthetic Polynucleotide
tcagcggaaatgaaacaaaca

SEQ ID NO: 1924 - DNA, 21 bp, Artificial Sequence
  Synthetic Polynucleotide
cccatagctgaagtagtggaa

SEQ ID NO: 1925 - DNA, 21 bp, Artificial Sequence
  Synthetic Polynucleotide
tagatccatgttctgtggtat

SEQ ID NO: 1926 - DNA, 21 bp, Artificial Sequence
  Synthetic Polynucleotide
atatctgtgcggaggttctta

SEQ ID NO: 1927 - DNA, 21 bp, Artificial Sequence
  Synthetic Polynucleotide
caaatgccgggatcttgtagg

SEQ ID NO: 1928 - DNA, 21 bp, Artificial Sequence
  Synthetic Polynucleotide
ccggagcagtgtgtacatact

SEQ ID NO: 1929 - DNA, 21 bp, Artificial Sequence
  Synthetic Polynucleotide
ctgcaacatggtgcaaggagc

SEQ ID NO: 1930 - DNA, 21 bp, Artificial Sequence
  Synthetic Polynucleotide
ttctaggtggaaattactttg

SEQ ID NO: 1931 - DNA, 21 bp, Artificial Sequence
  Synthetic Polynucleotide
cccgcactgggagccgtggct

SEQ ID NO: 1932 - DNA, 21 bp, Artificial Sequence
  Synthetic Polynucleotide
aaacccagtggcagacaagct

SEQ ID NO: 1933 - DNA, 21 bp, Artificial Sequence
  Synthetic Polynucleotide
ttattagtgacatcaagcacc

SEQ ID NO: 1934 - DNA, 21 bp, Artificial Sequence
  Synthetic Polynucleotide
cacggatggcatctttgatgg

SEQ ID NO: 1935 - DNA, 21 bp, Artificial Sequence
  Synthetic Polynucleotide
cagacgatcatacttggagag

SEQ ID NO: 1936 - DNA, 21 bp, Artificial Sequence
  Synthetic Polynucleotide
gtggtatgttcctcctgctcc

SEQ ID NO: 1937 - DNA, 21 bp, Artificial Sequence
  Synthetic Polynucleotide
ccataatactctgagagagat

SEQ ID NO: 1938 - DNA, 21 bp, Artificial Sequence
  Synthetic Polynucleotide
gtcaacctcactcttccgagc

SEQ ID NO: 1939 - DNA, 21 bp, Artificial Sequence
  Synthetic Polynucleotide
gtccattgaggtcagcgctga

SEQ ID NO: 1940 - DNA, 21 bp, Artificial Sequence
  Synthetic Polynucleotide
gcagttgtccatgtggaataa

SEQ ID NO: 1941 - DNA, 21 bp, Artificial Sequence
  Synthetic Polynucleotide
tgcatagatggccttcttgtt

SEQ ID NO: 1942 - DNA, 21 bp, Artificial Sequence
  Synthetic Polynucleotide
caattgtgataatggctggta

SEQ ID NO: 1943 - DNA, 21 bp, Artificial Sequence
  Synthetic Polynucleotide
cagacaagctcactgtgtcca

SEQ ID NO: 1944 - DNA, 21 bp, Artificial Sequence
  Synthetic Polynucleotide
caatatctgtgcggaggttct

SEQ ID NO: 1945 - DNA, 21 bp, Artificial Sequence
  Synthetic Polynucleotide
acccagtggcagacaagctca

SEQ ID NO: 1946 - DNA, 21 bp, Artificial Sequence
  Synthetic Polynucleotide
ttgggcaggaagcttagcaac

SEQ ID NO: 1947 - DNA, 21 bp, Artificial Sequence
  Synthetic Polynucleotide
ggttggttttgcacagccgcc

SEQ ID NO: 1948 - DNA, 21 bp, Artificial Sequence
  Synthetic Polynucleotide
gtacatactcatgacgatgcc

SEQ ID NO: 1949 - DNA, 21 bp, Artificial Sequence
  Synthetic Polynucleotide
tttgggcaggaagcttagcaa

SEQ ID NO: 1950 - DNA, 21 bp, Artificial Sequence
  Synthetic Polynucleotide
catgccactggttaccttggc

SEQ ID NO: 1951 - DNA, 21 bp, Artificial Sequence
  Synthetic Polynucleotide
gtgtcagcaaccacagcacca

SEQ ID NO: 1952 - DNA, 21 bp, Artificial Sequence
  Synthetic Polynucleotide
tctccaccacctttctgccat

SEQ ID NO: 1953 - DNA, 21 bp, Artificial Sequence
  Synthetic Polynucleotide
agcctcgtcttggtgcttcct

SEQ ID NO: 1954 - DNA, 21 bp, Artificial Sequence
  Synthetic Polynucleotide
gggttccctgagttagtctca

SEQ ID NO: 1955 - DNA, 21 bp, Artificial Sequence
  Synthetic Polynucleotide
ccctcacagaccagggtttgc

SEQ ID NO: 1956 - DNA, 21 bp, Artificial Sequence
  Synthetic Polynucleotide
tcatggtgttctgtgtagaca

SEQ ID NO: 1957 - DNA, 21 bp, Artificial Sequence
  Synthetic Polynucleotide
tcataggtgattttcacccct

SEQ ID NO: 1958 - DNA, 21 bp, Artificial Sequence
  Synthetic Polynucleotide
gtccaatatcaatggcagggt

SEQ ID NO: 1959 - DNA, 21 bp, Artificial Sequence
  Synthetic Polynucleotide
ggtttagactggagccaacat

SEQ ID NO: 1960 - DNA, 21 bp, Artificial Sequence
  Synthetic Polynucleotide
caccagttatcagcatgtcct

SEQ ID NO: 1961 - DNA, 21 bp, Artificial Sequence
  Synthetic Polynucleotide
ttcagctcaggtccataaaag

SEQ ID NO: 1962 - DNA, 21 bp, Artificial Sequence
  Synthetic Polynucleotide
cgagcatgatactgagagctt

SEQ ID NO: 1963 - DNA, 21 bp, Artificial Sequence
  Synthetic Polynucleotide
gtaactggagttttcaggtca

SEQ ID NO: 1964 - DNA, 21 bp, Artificial Sequence
  Synthetic Polynucleotide
tcaagcaccagtccaactata

SEQ ID NO: 1965 - DNA, 21 bp, Artificial Sequence
  Synthetic Polynucleotide
atcttggcgtgggcccgggtg

SEQ ID NO: 1966 - DNA, 21 bp, Artificial Sequence
  Synthetic Polynucleotide
tttaccaaccgcagaaacttg

SEQ ID NO: 1967 - DNA, 21 bp, Artificial Sequence
  Synthetic Polynucleotide
attcacttgtcttccaaatcc

SEQ ID NO: 1968 - DNA, 21 bp, Artificial Sequence
  Synthetic Polynucleotide
cagattgggtgttctataaaa

SEQ ID NO: 1969 - DNA, 21 bp, Artificial Sequence
  Synthetic Polynucleotide
atacttggagagcatcactgt

SEQ ID NO: 1970 - DNA, 21 bp, Artificial Sequence
  Synthetic Polynucleotide
ttctggttgaagtgtgtcagg

SEQ ID NO: 1971 - DNA, 21 bp, Artificial Sequence
  Synthetic Polynucleotide
ctccacagccgagcttggttc

SEQ ID NO: 1972 - DNA, 21 bp, Artificial Sequence
  Synthetic Polynucleotide
attatctgcttcggaaaaccc

SEQ ID NO: 1973 - DNA, 21 bp, Artificial Sequence
  Synthetic Polynucleotide
gcagacgatcatacttggaga

SEQ ID NO: 1974 - DNA, 21 bp, Artificial Sequence
  Synthetic Polynucleotide
gcttgctaggcattcttccca

SEQ ID NO: 1975 - DNA, 21 bp, Artificial Sequence
  Synthetic Polynucleotide
caggcattggcagaaaagtgg

SEQ ID NO: 1976 - DNA, 21 bp, Artificial Sequence
  Synthetic Polynucleotide
tcataggaaacagcatattct

SEQ ID NO: 1977 - DNA, 21 bp, Artificial Sequence
  Synthetic Polynucleotide
tccacagccgagcttggttcc

SEQ ID NO: 1978 - DNA, 21 bp, Artificial Sequence
  Synthetic Polynucleotide
gtgttgggcacagtgttagtg

SEQ ID NO: 1979 - DNA, 21 bp, Artificial Sequence
  Synthetic Polynucleotide
cccaacatttttgcaacaaag

SEQ ID NO: 1980 - DNA, 21 bp, Artificial Sequence
  Synthetic Polynucleotide
atcagcggaaatgaaacaaac

SEQ ID NO: 1981 - DNA, 21 bp, Artificial Sequence
  Synthetic Polynucleotide
tggcatctttgatggcaaaga

SEQ ID NO: 1982 - DNA, 21 bp, Artificial Sequence
  Synthetic Polynucleotide
agtctcaaagctgtagcccag

SEQ ID NO: 1983 - DNA, 21 bp, Artificial Sequence
  Synthetic Polynucleotide
ttcacgttattacctgtgtgc

SEQ ID NO: 1984 - DNA, 21 bp, Artificial Sequence
  Synthetic Polynucleotide
aatgatatgcccaacacaagt

SEQ ID NO: 1985 - DNA, 21 bp, Artificial Sequence
  Synthetic Polynucleotide
ggtctgtagcctgtgcagcgg

SEQ ID NO: 1986 - DNA, 21 bp, Artificial Sequence
  Synthetic Polynucleotide
tcttggtgcttcctattcctt

SEQ ID NO: 1987 - DNA, 21 bp, Artificial Sequence
  Synthetic Polynucleotide
ttaccaaccgcagaaacttga

SEQ ID NO: 1988 - DNA, 21 bp, Artificial Sequence
  Synthetic Polynucleotide
ttggtgcttcctattccttcc

SEQ ID NO: 1989 - DNA, 21 bp, Artificial Sequence
  Synthetic Polynucleotide
ggtttaagcttctagaggttt

SEQ ID NO: 1990 - DNA, 21 bp, Artificial Sequence
  Synthetic Polynucleotide
gtttagactggagccaacatc

SEQ ID NO: 1991 - DNA, 21 bp, Artificial Sequence
  Synthetic Polynucleotide
ttatagcagttgtccatgtgg

SEQ ID NO: 1992 - DNA, 21 bp, Artificial Sequence
  Synthetic Polynucleotide
accagttatcagcatgtcctc

SEQ ID NO: 1993 - DNA, 21 bp, Artificial Sequence
  Synthetic Polynucleotide
ccgagcatgatactgagagct

SEQ ID NO: 1994 - DNA, 21 bp, Artificial Sequence
  Synthetic Polynucleotide
ggttatacaggctgtccagta

SEQ ID NO: 1995 - DNA, 21 bp, Artificial Sequence
  Synthetic Polynucleotide
ctcgaaccacaatccggtttg

SEQ ID NO: 1996 - DNA, 21 bp, Artificial Sequence
  Synthetic Polynucleotide
cagttgtccatgtggaataaa

SEQ ID NO: 1997 - DNA, 21 bp, Artificial Sequence
  Synthetic Polynucleotide
ttggaacagcaatggtgcagt

SEQ ID NO: 1998 - DNA, 21 bp, Artificial Sequence
  Synthetic Polynucleotide
ccaaaagggttgtctctggat

SEQ ID NO: 1999 - DNA, 21 bp, Artificial Sequence
  Synthetic Polynucleotide
atcaagcaccagtccaactat

SEQ ID NO: 2000 - DNA, 21 bp, Artificial Sequence
  Synthetic Polynucleotide
agtatctcctccgggctcagc

SEQ ID NO: 2001 - DNA, 21 bp, Artificial Sequence
  Synthetic Polynucleotide
tccaaacttggtgggaattat

SEQ ID NO: 2002 - DNA, 21 bp, Artificial Sequence
  Synthetic Polynucleotide
tcaagatggtctgacaagccg

SEQ ID NO: 2003 - DNA, 21 bp, Artificial Sequence
  Synthetic Polynucleotide
gccttagatagctgcagatcc

SEQ ID NO: 2004 - DNA, 21 bp, Artificial Sequence
  Synthetic Polynucleotide
gattccggagcagtgtgtaca

SEQ ID NO: 2005 - DNA, 21 bp, Artificial Sequence
  Synthetic Polynucleotide
cgtggcttttggcaattctct

SEQ ID NO: 2006 - DNA, 21 bp, Artificial Sequence
  Synthetic Polynucleotide
ctctatggagagcagtatctc

SEQ ID NO: 2007 - DNA, 21 bp, Artificial Sequence
  Synthetic Polynucleotide
tcctaagagacactggcaggt

SEQ ID NO: 2008 - DNA, 21 bp, Artificial Sequence
  Synthetic Polynucleotide
ggtacctcactcctaagagac

SEQ ID NO: 2009 - DNA, 21 bp, Artificial Sequence
  Synthetic Polynucleotide
gtaaagttgcactggcgaaag

SEQ ID NO: 2010 - DNA, 21 bp, Artificial Sequence
  Synthetic Polynucleotide
cacgttattacctgtgtgctg

SEQ ID NO: 2011 - DNA, 21 bp, Artificial Sequence
  Synthetic Polynucleotide
tgtcatcttctctccgggagg

SEQ ID NO: 2012 - DNA, 21 bp, Artificial Sequence
  Synthetic Polynucleotide
ttatctgcttcggaaaacccc

SEQ ID NO: 2013 - DNA, 21 bp, Artificial Sequence
  Synthetic Polynucleotide
agtctccaggtagaagtgctc

SEQ ID NO: 2014 - DNA, 21 bp, Artificial Sequence
  Synthetic Polynucleotide
aggccgtgttggagggaaggt

SEQ ID NO: 2015 - DNA, 21 bp, Artificial Sequence
  Synthetic Polynucleotide
tctccacagccgagcttggtt

SEQ ID NO: 2016 - DNA, 21 bp, Artificial Sequence
  Synthetic Polynucleotide
tcttccgagcatgatactgag

SEQ ID NO: 2017 - DNA, 21 bp, Artificial Sequence
  Synthetic Polynucleotide
gagcatgatactgagagcttg

SEQ ID NO: 2018 - DNA, 21 bp, Artificial Sequence
  Synthetic Polynucleotide
tcaatggcagggtttagactg

SEQ ID NO: 2019 - DNA, 21 bp, Artificial Sequence
  Synthetic Polynucleotide
cagccggagagacagctcatt

SEQ ID NO: 2020 - DNA, 21 bp, Artificial Sequence
  Synthetic Polynucleotide
tcatcacggtccagcatgcat

SEQ ID NO: 2021 - DNA, 21 bp, Artificial Sequence
  Synthetic Polynucleotide
tatctgtgcggaggttcttat

SEQ ID NO: 2022 - DNA, 21 bp, Artificial Sequence
  Synthetic Polynucleotide
tttctaggtggaaattacttt

SEQ ID NO: 2023 - DNA, 21 bp, Artificial Sequence
  Synthetic Polynucleotide
aatatctgtgcggaggttctt

SEQ ID NO: 2024 - DNA, 21 bp, Artificial Sequence
  Synthetic Polynucleotide
gcagaaggttggatttataca

SEQ ID NO: 2025 - DNA, 21 bp, Artificial Sequence
  Synthetic Polynucleotide
atgaaacaaacaaaccctgga

SEQ ID NO: 2026 - DNA, 21 bp, Artificial Sequence
  Synthetic Polynucleotide
gtggcttttggcaattctctc

SEQ ID NO: 2027 - DNA, 21 bp, Artificial Sequence
  Synthetic Polynucleotide
cacagtgttagtgcttgtctc

SEQ ID NO: 2028 - DNA, 21 bp, Artificial Sequence
  Synthetic Polynucleotide
atgctgccaaatgccgggatc

SEQ ID NO: 2029 - DNA, 21 bp, Artificial Sequence
  Synthetic Polynucleotide
aatgtcatcttctctccggga

SEQ ID NO: 2030 - DNA, 21 bp, Artificial Sequence
  Synthetic Polynucleotide
attccaaacttggtgggaatt

SEQ ID NO: 2031 - DNA, 21 bp, Artificial Sequence
  Synthetic Polynucleotide
tttggcttcaaatgtaaagat

SEQ ID NO: 2032 - DNA, 21 bp, Artificial Sequence
  Synthetic Polynucleotide
tttatagcagttgtccatgtg

SEQ ID NO: 2033 - DNA, 21 bp, Artificial Sequence
  Synthetic Polynucleotide
agaccagggtttgcagttttc

SEQ ID NO: 2034 - DNA, 21 bp, Artificial Sequence
  Synthetic Polynucleotide
ttggcttcaaatgtaaagatt

SEQ ID NO: 2035 - DNA, 21 bp, Artificial Sequence
  Synthetic Polynucleotide
cctgcttgaatgctgagaaat

SEQ ID NO: 2036 - DNA, 21 bp, Artificial Sequence
  Synthetic Polynucleotide
caattaaccttgaatttgttt

SEQ ID NO: 2037 - DNA, 21 bp, Artificial Sequence
  Synthetic Polynucleotide
agtttcatttattagtgacat

SEQ ID NO: 2038 - DNA, 21 bp, Artificial Sequence
  Synthetic Polynucleotide
ctctttaccaaccgcagaaac

SEQ ID NO: 2039 - DNA, 21 bp, Artificial Sequence
  Synthetic Polynucleotide
ctttgatggcaaagaagatag

SEQ ID NO: 2040 - DNA, 21 bp, Artificial Sequence
  Synthetic Polynucleotide
atcagaacttgaggttataca

SEQ ID NO: 2041 - DNA, 21 bp, Artificial Sequence
  Synthetic Polynucleotide
atctgtgcggaggttcttatg

SEQ ID NO: 2042 - DNA, 21 bp, Artificial Sequence
  Synthetic Polynucleotide
tgtacatactcatgacgatgc

SEQ ID NO: 2043 - DNA, 21 bp, Artificial Sequence
  Synthetic Polynucleotide
ccttccacagttgtcactgca

SEQ ID NO: 2044 - DNA, 21 bp, Artificial Sequence
  Synthetic Polynucleotide
tcttcagctcaggtccataaa

SEQ ID NO: 2045 - DNA, 21 bp, Artificial Sequence
  Synthetic Polynucleotide
gaaacagcatattcttgaact

SEQ ID NO: 2046 - DNA, 21 bp, Artificial Sequence
  Synthetic Polynucleotide
cagctccttgagggttgaggc

SEQ ID NO: 2047 - DNA, 21 bp, Artificial Sequence
  Synthetic Polynucleotide
ccatgttctgtggtatgttcc

SEQ ID NO: 2048 - DNA, 21 bp, Artificial Sequence
  Synthetic Polynucleotide
gtgtgtacatactcatgacga

SEQ ID NO: 2049 - DNA, 21 bp, Artificial Sequence
  Synthetic Polynucleotide
cttctggttgaagtgtgtcag

SEQ ID NO: 2050 - DNA, 21 bp, Artificial Sequence
  Synthetic Polynucleotide
gttattacctgtgtgctgagc

SEQ ID NO: 2051 - DNA, 21 bp, Artificial Sequence
  Synthetic Polynucleotide
tgactttaatagatccatgtt

SEQ ID NO: 2052 - DNA, 21 bp, Artificial Sequence
  Synthetic Polynucleotide
ccacagccgagcttggttcca

SEQ ID NO: 2053 - DNA, 21 bp, Artificial Sequence
  Synthetic Polynucleotide
cctgtccaatatcaatggcag

SEQ ID NO: 2054 - DNA, 21 bp, Artificial Sequence
  Synthetic Polynucleotide
gaagaagaagctgagggtgag

SEQ ID NO: 2055 - DNA, 21 bp, Artificial Sequence
  Synthetic Polynucleotide
cttttggcaattctctcctgc

SEQ ID NO: 2056 - DNA, 21 bp, Artificial Sequence
  Synthetic Polynucleotide
gccaccagttatcagcatgtc

SEQ ID NO: 2057 - DNA, 21 bp, Artificial Sequence
  Synthetic Polynucleotide
taaagattaaacataatcttt

SEQ ID NO: 2058 - DNA, 21 bp, Artificial Sequence
  Synthetic Polynucleotide
gcttttggcaattctctcctg

SEQ ID NO: 2059 - DNA, 21 bp, Artificial Sequence
  Synthetic Polynucleotide
aaagattaaacataatctttt

SEQ ID NO: 2060 - DNA, 21 bp, Artificial Sequence
  Synthetic Polynucleotide
accattttggtatgaaggcct

SEQ ID NO: 2061 - DNA, 21 bp, Artificial Sequence
  Synthetic Polynucleotide
gcttcccagcaaaccagcgca

SEQ ID NO: 2062 - DNA, 21 bp, Artificial Sequence
  Synthetic Polynucleotide
gggaaagaaatctagaacatt

SEQ ID NO: 2063 - DNA, 21 bp, Artificial Sequence
  Synthetic Polynucleotide
cctgtgtgctgagctcgagct

SEQ ID NO: 2064 - DNA, 21 bp, Artificial Sequence
  Synthetic Polynucleotide
ccttttggaacagcaatggtg

SEQ ID NO: 2065 - DNA, 21 bp, Artificial Sequence
  Synthetic Polynucleotide
gcagaaaagtggacgatcttg

SEQ ID NO: 2066 - DNA, 21 bp, Artificial Sequence
  Synthetic Polynucleotide
gttccttcacgttattacctg

SEQ ID NO: 2067 - DNA, 21 bp, Artificial Sequence
  Synthetic Polynucleotide
tgaataaaactctcatgccac

SEQ ID NO: 2068 - DNA, 21 bp, Artificial Sequence
  Synthetic Polynucleotide
ttgctgttcattggtttgaag

SEQ ID NO: 2069 - DNA, 21 bp, Artificial Sequence
  Synthetic Polynucleotide
tttgctgttcattggtttgaa

SEQ ID NO: 2070 - DNA, 21 bp, Artificial Sequence
  Synthetic Polynucleotide
caataattgagttggttggat

SEQ ID NO: 2071 - DNA, 21 bp, Artificial Sequence
  Synthetic Polynucleotide
gattaaacataatcttttttg

SEQ ID NO: 2072 - DNA, 21 bp, Artificial Sequence
  Synthetic Polynucleotide
tcctctgcaggcatcccacag

SEQ ID NO: 2073 - DNA, 21 bp, Artificial Sequence
  Synthetic Polynucleotide
gcctctgagtggtcttgaggg

SEQ ID NO: 2074 - DNA, 21 bp, Artificial Sequence
  Synthetic Polynucleotide
cacctcctctgcaggcatccc

SEQ ID NO: 2075 - DNA, 21 bp, Artificial Sequence
  Synthetic Polynucleotide
ataaaactctcatgccactgg

SEQ ID NO: 2076 - DNA, 21 bp, Artificial Sequence
  Synthetic Polynucleotide
ttctgtgactttaatagatcc

SEQ ID NO: 2077 - DNA, 21 bp, Artificial Sequence
  Synthetic Polynucleotide
agtaaaatggatcacaggaag

SEQ ID NO: 2078 - DNA, 21 bp, Artificial Sequence
  Synthetic Polynucleotide
aaagaagatagaagcagccag

SEQ ID NO: 2079 - DNA, 21 bp, Artificial Sequence
  Synthetic Polynucleotide
gcacagagccatctgtgtaca

SEQ ID NO: 2080 - DNA, 21 bp, Artificial Sequence
  Synthetic Polynucleotide
ggactgtcaggtagaacttga

SEQ ID NO: 2081 - DNA, 21 bp, Artificial Sequence
  Synthetic Polynucleotide
tctccaagtatgatcgtctgc

SEQ ID NO: 2082 - DNA, 21 bp, Artificial Sequence
  Synthetic Polynucleotide
tgcagaacaagatcgtccact

SEQ ID NO: 2083 - DNA, 21 bp, Artificial Sequence
  Synthetic Polynucleotide
ccacttttctgccaatgcctg

SEQ ID NO: 2084 - DNA, 21 bp, Artificial Sequence
  Synthetic Polynucleotide
acggctcccagtgcgggttct

SEQ ID NO: 2085 - DNA, 21 bp, Artificial Sequence
  Synthetic Polynucleotide
cacggagattggcattgagat

SEQ ID NO: 2086 - DNA, 21 bp, Artificial Sequence
  Synthetic Polynucleotide
acggagattggcattgagatg

SEQ ID NO: 2087 - DNA, 21 bp, Artificial Sequence
  Synthetic Polynucleotide
agagaagatgacattgccaag

SEQ ID NO: 2088 - DNA, 21 bp, Artificial Sequence
  Synthetic Polynucleotide
ccagtggcatgagagttttat

SEQ ID NO: 2089 - DNA, 21 bp, Artificial Sequence
  Synthetic Polynucleotide
tgcaggcctctggtgaggccg

SEQ ID NO: 2090 - DNA, 21 bp, Artificial Sequence
  Synthetic Polynucleotide
tggtgaggccgtgtactgtga

SEQ ID NO: 2091 - DNA, 21 bp, Artificial Sequence
  Synthetic Polynucleotide
cgggcccacgccaagatcaag

SEQ ID NO: 2092 - DNA, 21 bp, Artificial Sequence
  Synthetic Polynucleotide
ccaagatcaagtccatagata

SEQ ID NO: 2093 - DNA, 21 bp, Artificial Sequence
  Synthetic Polynucleotide
gttgggcatatcattggtgct

SEQ ID NO: 2094 - DNA, 21 bp, Artificial Sequence
  Synthetic Polynucleotide
cattggtgctgtggttgctga

SEQ ID NO: 2095 - DNA, 21 bp, Artificial Sequence
  Synthetic Polynucleotide
aaaccggattgtggttcgagt

SEQ ID NO: 2096 - DNA, 21 bp, Artificial Sequence
  Synthetic Polynucleotide
ccggattgtggttcgagtgaa

SEQ ID NO: 2097 - DNA, 21 bp, Artificial Sequence
  Synthetic Polynucleotide
gattgtggttcgagtgaagag

SEQ ID NO: 2098 - DNA, 21 bp, Artificial Sequence
  Synthetic Polynucleotide
gctggaccgtgatgaggacat

SEQ ID NO: 2099 - DNA, 21 bp, Artificial Sequence
  Synthetic Polynucleotide
tgaggacatgctgataactgg

SEQ ID NO: 2100 - DNA, 21 bp, Artificial Sequence
  Synthetic Polynucleotide
cagatacaaggttggcttcat

SEQ ID NO: 2101 - DNA, 21 bp, Artificial Sequence
  Synthetic Polynucleotide
ctctcagagtattatggaacg

SEQ ID NO: 2102 - DNA, 21 bp, Artificial Sequence
  Synthetic Polynucleotide
aaccttccctccaacacggcc

SEQ ID NO: 2103 - DNA, 21 bp, Artificial Sequence
  Synthetic Polynucleotide
ctagcaagctctcagtatcat

SEQ ID NO: 2104 - DNA, 21 bp, Artificial Sequence
  Synthetic Polynucleotide
gcaagctctcagtatcatgct

SEQ ID NO: 2105 - DNA, 21 bp, Artificial Sequence
  Synthetic Polynucleotide
tcagtatcatgctcggaagag

SEQ ID NO: 2106 - DNA, 21 bp, Artificial Sequence
  Synthetic Polynucleotide
ccaccaagtttggaataagct

SEQ ID NO: 2107 - DNA, 21 bp, Artificial Sequence
  Synthetic Polynucleotide
tccgcacagatattgtcatgg

SEQ ID NO: 2108 - DNA, 21 bp, Artificial Sequence
  Synthetic Polynucleotide
caagatcccggcatttggcag

SEQ ID NO: 2109 - DNA, 21 bp, Artificial Sequence
  Synthetic Polynucleotide
agcacacaggtaataacgtga

SEQ ID NO: 2110 - DNA, 21 bp, Artificial Sequence
  Synthetic Polynucleotide
accctggtctgtgagggtcta

SEQ ID NO: 2111 - DNA, 21 bp, Artificial Sequence
  Synthetic Polynucleotide
ccacagaacatggatctatta

SEQ ID NO: 2112 - DNA, 21 bp, Artificial Sequence
  Synthetic Polynucleotide
acatggatctattaaagtcac

SEQ ID NO: 2113 - DNA, 21 bp, Artificial Sequence
  Synthetic Polynucleotide
atggatctattaaagtcacag

SEQ ID NO: 2114 - DNA, 21 bp, Artificial Sequence
  Synthetic Polynucleotide
ggaagggtttgtgctattccc

SEQ ID NO: 2115 - DNA, 21 bp, Artificial Sequence
  Synthetic Polynucleotide
gaagggtttgtgctattcccc

SEQ ID NO: 2116 - DNA, 21 bp, Artificial Sequence
  Synthetic Polynucleotide
ctcaagttctgatggtgtctg

SEQ ID NO: 2117 - DNA, 21 bp, Artificial Sequence
  Synthetic Polynucleotide
tcaagttctgatggtgtctgt

SEQ ID NO: 2118 - DNA, 21 bp, Artificial Sequence
  Synthetic Polynucleotide
cccacaaacctctagaagctt

SEQ ID NO: 2119 - DNA, 21 bp, Artificial Sequence
  Synthetic Polynucleotide
cacaaacctctagaagcttaa

SEQ ID NO: 2120 - DNA, 21 bp, Artificial Sequence
  Synthetic Polynucleotide
ccttcaaaccaatgaacagca

SEQ ID NO: 2121 - DNA, 21 bp, Artificial Sequence
  Synthetic Polynucleotide
aatgaacagcaaagcataacc

SEQ ID NO: 2122 - DNA, 21 bp, Artificial Sequence
  Synthetic Polynucleotide
aacaaattcaaggttaattgg

SEQ ID NO: 2123 - DNA, 21 bp, Artificial Sequence
  Synthetic Polynucleotide
aagctgcataaagcaagatta

SEQ ID NO: 2124 - DNA, 21 bp, Artificial Sequence
  Synthetic Polynucleotide
tgcataaagcaagattactct

SEQ ID NO: 2125 - DNA, 21 bp, Artificial Sequence
  Synthetic Polynucleotide
aaagattatgtttaatcttta

SEQ ID NO: 2126 - DNA, 21 bp, Artificial Sequence
  Synthetic Polynucleotide
aaggcttggttttcttactgt

SEQ ID NO: 2127 - DNA, 21 bp, Artificial Sequence
  Synthetic Polynucleotide
ggttttcttactgtcatatga

SEQ ID NO: 2128 - DNA, 21 bp, Artificial Sequence
  Synthetic Polynucleotide
tcaagtttctgcggttggtaa

SEQ ID NO: 2129 - DNA, 21 bp, Artificial Sequence
  Synthetic Polynucleotide
caagtttctgcggttggtaaa

SEQ ID NO: 2130 - DNA, 21 bp, Artificial Sequence
  Synthetic Polynucleotide
ttggactggtgcttgatgtca

SEQ ID NO: 2131 - DNA, 21 bp, Artificial Sequence
  Synthetic Polynucleotide
tttggaagacaagtgaatgca

SEQ ID NO: 2132 - DNA, 21 bp, Artificial Sequence
  Synthetic Polynucleotide
gtgatgctctccaagtatgat

SEQ ID NO: 2133 - DNA, 21 bp, Artificial Sequence
  Synthetic Polynucleotide
accggattgtggttcgagtga

SEQ ID NO: 2134 - DNA, 21 bp, Artificial Sequence
  Synthetic Polynucleotide
tttctgcggttggtaaagaga

SEQ ID NO: 2135 - DNA, 21 bp, Artificial Sequence
  Synthetic Polynucleotide
cttctatcttctttgccatca

SEQ ID NO: 2136 - DNA, 21 bp, Artificial Sequence
  Synthetic Polynucleotide
gaacataccacagaacatgga

SEQ ID NO: 2137 - DNA, 21 bp, Artificial Sequence
  Synthetic Polynucleotide
ttatggacctgagctgaagat

SEQ ID NO: 2138 - DNA, 21 bp, Artificial Sequence
  Synthetic Polynucleotide
ggcccacgccaagatcaagtc

SEQ ID NO: 2139 - DNA, 21 bp, Artificial Sequence
  Synthetic Polynucleotide
aagggtttgtgctattcccca

SEQ ID NO: 2140 - DNA, 21 bp, Artificial Sequence
  Synthetic Polynucleotide
tgtgatccattttactgcaaa

SEQ ID NO: 2141 - DNA, 21 bp, Artificial Sequence
  Synthetic Polynucleotide
accaactcaattattgagcac

SEQ ID NO: 2142 - DNA, 21 bp, Artificial Sequence
  Synthetic Polynucleotide
tctgcagctatctaaggcttg

SEQ ID NO: 2143 - DNA, 21 bp, Artificial Sequence
  Synthetic Polynucleotide
ccgaagcagataatgttgtgt

SEQ ID NO: 2144 - DNA, 21 bp, Artificial Sequence
  Synthetic Polynucleotide
tgtacacactgctccggaatc

SEQ ID NO: 2145 - DNA, 21 bp, Artificial Sequence
  Synthetic Polynucleotide
cagagacaacccttttggcct

SEQ ID NO: 2146 - DNA, 21 bp, Artificial Sequence
  Synthetic Polynucleotide
ccggcatttggcagcatcccc

SEQ ID NO: 2147 - DNA, 21 bp, Artificial Sequence
  Synthetic Polynucleotide
gttacttgtgttgggcatatc

SEQ ID NO: 2148 - DNA, 21 bp, Artificial Sequence
  Synthetic Polynucleotide
gggaagaatgcctagcaagct

SEQ ID NO: 2149 - DNA, 21 bp, Artificial Sequence
  Synthetic Polynucleotide
tggaagacaagtgaatgcaat

SEQ ID NO: 2150 - DNA, 21 bp, Artificial Sequence
  Synthetic Polynucleotide
cataaagcaagattactctat

SEQ ID NO: 2151 - DNA, 21 bp, Artificial Sequence
  Synthetic Polynucleotide
accaagtttggaataagcttt

SEQ ID NO: 2152 - DNA, 21 bp, Artificial Sequence
  Synthetic Polynucleotide
gttcaacaaggagaattgttg

SEQ ID NO: 2153 - DNA, 21 bp, Artificial Sequence
  Synthetic Polynucleotide
gagaaaaatgcagatccagag

SEQ ID NO: 2154 - DNA, 21 bp, Artificial Sequence
  Synthetic Polynucleotide
agcaagctctcagtatcatgc

SEQ ID NO: 2155 - DNA, 21 bp, Artificial Sequence
  Synthetic Polynucleotide
gcatcgtcatgagtatgtaca

SEQ ID NO: 2156 - DNA, 21 bp, Artificial Sequence
  Synthetic Polynucleotide
gctccttgcaccatgttgcag

SEQ ID NO: 2157 - DNA, 21 bp, Artificial Sequence
  Synthetic Polynucleotide
aaccaactcaattattgagca

SEQ ID NO: 2158 - DNA, 21 bp, Artificial Sequence
  Synthetic Polynucleotide
cttgtgttgggcatatcattg

SEQ ID NO: 2159 - DNA, 21 bp, Artificial Sequence
  Synthetic Polynucleotide
gataaggttacttgtgttggg

SEQ ID NO: 2160 - DNA, 21 bp, Artificial Sequence
  Synthetic Polynucleotide
ggcttgcacagtgatgctctc

SEQ ID NO: 2161 - DNA, 21 bp, Artificial Sequence
  Synthetic Polynucleotide
gaaacaaattcaaggttaatt

SEQ ID NO: 2162 - DNA, 21 bp, Artificial Sequence
  Synthetic Polynucleotide
tttgtgatcttggactgtcaa

SEQ ID NO: 2163 - DNA, 21 bp, Artificial Sequence
  Synthetic Polynucleotide
ggagaaaaatgcagatccaga

SEQ ID NO: 2164 - DNA, 21 bp, Artificial Sequence
  Synthetic Polynucleotide
agataatgttgtgtcagggga

SEQ ID NO: 2165 - DNA, 21 bp, Artificial Sequence
  Synthetic Polynucleotide
caccaagtttggaataagctt

SEQ ID NO: 2166 - DNA, 21 bp, Artificial Sequence
  Synthetic Polynucleotide
aaagcataaccttgaatctat

SEQ ID NO: 2167 - DNA, 21 bp, Artificial Sequence
  Synthetic Polynucleotide
cacaggtaataacgtgaagga

SEQ ID NO: 2168 - DNA, 21 bp, Artificial Sequence
  Synthetic Polynucleotide
cagccattatcacaattgagg

SEQ ID NO: 2169 - DNA, 21 bp, Artificial Sequence
  Synthetic Polynucleotide
aaaaatccaaccaactcaatt

SEQ ID NO: 2170 - DNA, 21 bp, Artificial Sequence
  Synthetic Polynucleotide
ataggaagcaccaagacgagg

SEQ ID NO: 2171 - DNA, 21 bp, Artificial Sequence
  Synthetic Polynucleotide
gcgctgacctcaatggacagg

SEQ ID NO: 2172 - DNA, 21 bp, Artificial Sequence
  Synthetic Polynucleotide
cccggagagaagatgacattg

SEQ ID NO: 2173 - DNA, 21 bp, Artificial Sequence
  Synthetic Polynucleotide
cattgatattggacaggtgga

SEQ ID NO: 2174 - DNA, 21 bp, Artificial Sequence
  Synthetic Polynucleotide
cacacaggtaataacgtgaag

SEQ ID NO: 2175 - DNA, 21 bp, Artificial Sequence
  Synthetic Polynucleotide
cctaaatggtgaatatgcaat

SEQ ID NO: 2176 - DNA, 21 bp, Artificial Sequence
  Synthetic Polynucleotide
gccactgggttttatagaaca

SEQ ID NO: 2177 - DNA, 21 bp, Artificial Sequence
  Synthetic Polynucleotide
cacactgctccggaatcagcc

SEQ ID NO: 2178 - DNA, 21 bp, Artificial Sequence
  Synthetic Polynucleotide
tacttgtgttgggcatatcat

SEQ ID NO: 2179 - DNA, 21 bp, Artificial Sequence
  Synthetic Polynucleotide
aggccgtgtactgtgacgaca

SEQ ID NO: 2180 - DNA, 21 bp, Artificial Sequence
  Synthetic Polynucleotide
tagcaagctctcagtatcatg

SEQ ID NO: 2181 - DNA, 21 bp, Artificial Sequence
  Synthetic Polynucleotide
ggcattgagatgaagttcaag

SEQ ID NO: 2182 - DNA, 21 bp, Artificial Sequence
  Synthetic Polynucleotide
aacacccaatctgggctacag

SEQ ID NO: 2183 - DNA, 21 bp, Artificial Sequence
  Synthetic Polynucleotide
tttgtttcatttccgctgatg

SEQ ID NO: 2184 - DNA, 21 bp, Artificial Sequence
  Synthetic Polynucleotide
ccactacttcagctatggggt

SEQ ID NO: 2185 - DNA, 21 bp, Artificial Sequence
  Synthetic Polynucleotide
accacagaacatggatctatt

SEQ ID NO: 2186 - DNA, 21 bp, Artificial Sequence
  Synthetic Polynucleotide
agaacctccgcacagatattg

SEQ ID NO: 2187 - DNA, 21 bp, Artificial Sequence
  Synthetic Polynucleotide
tacaagatcccggcatttggc

SEQ ID NO: 2188 - DNA, 21 bp, Artificial Sequence
  Synthetic Polynucleotide
tatgtacacactgctccggaa

SEQ ID NO: 2189 - DNA, 21 bp, Artificial Sequence
  Synthetic Polynucleotide
tccttgcaccatgttgcagtg

SEQ ID NO: 2190 - DNA, 21 bp, Artificial Sequence
  Synthetic Polynucleotide
aagtaatttccacctagaaat

SEQ ID NO: 2191 - DNA, 21 bp, Artificial Sequence
  Synthetic Polynucleotide
ccacggctcccagtgcgggtt

SEQ ID NO: 2192 - DNA, 21 bp, Artificial Sequence
  Synthetic Polynucleotide
cttgtctgccactgggtttta

SEQ ID NO: 2193 - DNA, 21 bp, Artificial Sequence
  Synthetic Polynucleotide
tgcttgatgtcactaataaat

SEQ ID NO: 2194 - DNA, 21 bp, Artificial Sequence
  Synthetic Polynucleotide
atcaaagatgccatccgtgca

SEQ ID NO: 2195 - DNA, 21 bp, Artificial Sequence
  Synthetic Polynucleotide
ctccaagtatgatcgtctgca

SEQ ID NO: 2196 - DNA, 21 bp, Artificial Sequence
  Synthetic Polynucleotide
agcaggaggaacataccacag

SEQ ID NO: 2197 - DNA, 21 bp, Artificial Sequence
  Synthetic Polynucleotide
ctctctcagagtattatggaa

SEQ ID NO: 2198 - DNA, 21 bp, Artificial Sequence
  Synthetic Polynucleotide
tcggaagagtgaggttgacaa

SEQ ID NO: 2199 - DNA, 21 bp, Artificial Sequence
  Synthetic Polynucleotide
agcgctgacctcaatggacag

SEQ ID NO: 2200 - DNA, 21 bp, Artificial Sequence
  Synthetic Polynucleotide
attccacatggacaactgcta

SEQ ID NO: 2201 - DNA, 21 bp, Artificial Sequence
  Synthetic Polynucleotide
caagaaggccatctatgcatc

SEQ ID NO: 2202 - DNA, 21 bp, Artificial Sequence
  Synthetic Polynucleotide
ccagccattatcacaattgag

SEQ ID NO: 2203 - DNA, 21 bp, Artificial Sequence
  Synthetic Polynucleotide
gacacagtgagcttgtctgcc

SEQ ID NO: 2204 - DNA, 21 bp, Artificial Sequence
  Synthetic Polynucleotide
aacctccgcacagatattgtc

SEQ ID NO: 2205 - DNA, 21 bp, Artificial Sequence
  Synthetic Polynucleotide
agcttgtctgccactgggttt

SEQ ID NO: 2206 - DNA, 21 bp, Artificial Sequence
  Synthetic Polynucleotide
tgctaagcttcctgcccaaaa

SEQ ID NO: 2207 - DNA, 21 bp, Artificial Sequence
  Synthetic Polynucleotide
cggctgtgcaaaaccaacctt

SEQ ID NO: 2208 - DNA, 21 bp, Artificial Sequence
  Synthetic Polynucleotide
catcgtcatgagtatgtacac

SEQ ID NO: 2209 - DNA, 21 bp, Artificial Sequence
  Synthetic Polynucleotide
gctaagcttcctgcccaaaag

SEQ ID NO: 2210 - DNA, 21 bp, Artificial Sequence
  Synthetic Polynucleotide
caaggtaaccagtggcatgag

SEQ ID NO: 2211 - DNA, 21 bp, Artificial Sequence
  Synthetic Polynucleotide
gtgctgtggttgctgacaccc

SEQ ID NO: 2212 - DNA, 21 bp, Artificial Sequence
  Synthetic Polynucleotide
ggcagaaaggtggtggagaaa

SEQ ID NO: 2213 - DNA, 21 bp, Artificial Sequence
  Synthetic Polynucleotide
gaagcaccaagacgaggctgc

SEQ ID NO: 2214 - DNA, 21 bp, Artificial Sequence
  Synthetic Polynucleotide
agactaactcagggaacccct

SEQ ID NO: 2215 - DNA, 21 bp, Artificial Sequence
  Synthetic Polynucleotide
aaaccctggtctgtgagggtc

SEQ ID NO: 2216 - DNA, 21 bp, Artificial Sequence
  Synthetic Polynucleotide
tctacacagaacaccatgaag

SEQ ID NO: 2217 - DNA, 21 bp, Artificial Sequence
  Synthetic Polynucleotide
gggtgaaaatcacctatgaag

SEQ ID NO: 2218 - DNA, 21 bp, Artificial Sequence
  Synthetic Polynucleotide
cctgccattgatattggacag

SEQ ID NO: 2219 - DNA, 21 bp, Artificial Sequence
  Synthetic Polynucleotide
gttggctccagtctaaaccct

SEQ ID NO: 2220 - DNA, 21 bp, Artificial Sequence
  Synthetic Polynucleotide
gacatgctgataactggtggc

SEQ ID NO: 2221 - DNA, 21 bp, Artificial Sequence
  Synthetic Polynucleotide
tttatggacctgagctgaaga

SEQ ID NO: 2222 - DNA, 21 bp, Artificial Sequence
  Synthetic Polynucleotide
gctctcagtatcatgctcgga

SEQ ID NO: 2223 - DNA, 21 bp, Artificial Sequence
  Synthetic Polynucleotide
acctgaaaactccagttacat

SEQ ID NO: 2224 - DNA, 21 bp, Artificial Sequence
  Synthetic Polynucleotide
tagttggactggtgcttgatg

SEQ ID NO: 2225 - DNA, 21 bp, Artificial Sequence
  Synthetic Polynucleotide
cccgggcccacgccaagatca

SEQ ID NO: 2226 - DNA, 21 bp, Artificial Sequence
  Synthetic Polynucleotide
agtttctgcggttggtaaaga

SEQ ID NO: 2227 - DNA, 21 bp, Artificial Sequence
  Synthetic Polynucleotide
atttggaagacaagtgaatgc

SEQ ID NO: 2228 - DNA, 21 bp, Artificial Sequence
  Synthetic Polynucleotide
ttatagaacacccaatctggg

SEQ ID NO: 2229 - DNA, 21 bp, Artificial Sequence
  Synthetic Polynucleotide
agtgatgctctccaagtatga

SEQ ID NO: 2230 - DNA, 21 bp, Artificial Sequence
  Synthetic Polynucleotide
tgacacacttcaaccagaagc

SEQ ID NO: 2231 - DNA, 21 bp, Artificial Sequence
  Synthetic Polynucleotide
accaagctcggctgtggagag

SEQ ID NO: 2232 - DNA, 21 bp, Artificial Sequence
  Synthetic Polynucleotide
gttttccgaagcagataatgt

SEQ ID NO: 2233 - DNA, 21 bp, Artificial Sequence
  Synthetic Polynucleotide
tccaagtatgatcgtctgcag

SEQ ID NO: 2234 - DNA, 21 bp, Artificial Sequence
  Synthetic Polynucleotide
ggaagaatgcctagcaagctc

SEQ ID NO: 2235 - DNA, 21 bp, Artificial Sequence
  Synthetic Polynucleotide
acttttctgccaatgcctgcc

SEQ ID NO: 2236 - DNA, 21 bp, Artificial Sequence
  Synthetic Polynucleotide
aatatgctgtttcctatgatt

SEQ ID NO: 2237 - DNA, 21 bp, Artificial Sequence
  Synthetic Polynucleotide
aaccaagctcggctgtggaga

SEQ ID NO: 2238 - DNA, 21 bp, Artificial Sequence
  Synthetic Polynucleotide
ctaacactgtgcccaacacct

SEQ ID NO: 2239 - DNA, 21 bp, Artificial Sequence
  Synthetic Polynucleotide
ttgttgcaaaaatgttggggg

SEQ ID NO: 2240 - DNA, 21 bp, Artificial Sequence
  Synthetic Polynucleotide
ttgtttcatttccgctgatga

SEQ ID NO: 2241 - DNA, 21 bp, Artificial Sequence
  Synthetic Polynucleotide
tttgccatcaaagatgccatc

SEQ ID NO: 2242 - DNA, 21 bp, Artificial Sequence
  Synthetic Polynucleotide
gggctacagctttgagactaa

SEQ ID NO: 2243 - DNA, 21 bp, Artificial Sequence
  Synthetic Polynucleotide
acacaggtaataacgtgaagg

SEQ ID NO: 2244 - DNA, 21 bp, Artificial Sequence
  Synthetic Polynucleotide
ttgtgttgggcatatcattgg

SEQ ID NO: 2245 - DNA, 21 bp, Artificial Sequence
  Synthetic Polynucleotide
gctgcacaggctacagaccca

SEQ ID NO: 2246 - DNA, 21 bp, Artificial Sequence
  Synthetic Polynucleotide
ggaataggaagcaccaagacg

SEQ ID NO: 2247 - DNA, 21 bp, Artificial Sequence
  Synthetic Polynucleotide
aagtttctgcggttggtaaag

SEQ ID NO: 2248 - DNA, 21 bp, Artificial Sequence
  Synthetic Polynucleotide
aaggaataggaagcaccaaga

SEQ ID NO: 2249 - DNA, 21 bp, Artificial Sequence
  Synthetic Polynucleotide
acctctagaagcttaaaccga

SEQ ID NO: 2250 - DNA, 21 bp, Artificial Sequence
  Synthetic Polynucleotide
tgttggctccagtctaaaccc

SEQ ID NO: 2251 - DNA, 21 bp, Artificial Sequence
  Synthetic Polynucleotide
acatggacaactgctataaaa

SEQ ID NO: 2252 - DNA, 21 bp, Artificial Sequence
  Synthetic Polynucleotide
ggacatgctgataactggtgg

SEQ ID NO: 2253 - DNA, 21 bp, Artificial Sequence
  Synthetic Polynucleotide
ctctcagtatcatgctcggaa

SEQ ID NO: 2254 - DNA, 21 bp, Artificial Sequence
  Synthetic Polynucleotide
ctggacagcctgtataacctc

SEQ ID NO: 2255 - DNA, 21 bp, Artificial Sequence
  Synthetic Polynucleotide
aaccggattgtggttcgagtg

SEQ ID NO: 2256 - DNA, 21 bp, Artificial Sequence
  Synthetic Polynucleotide
tattccacatggacaactgct

SEQ ID NO: 2257 - DNA, 21 bp, Artificial Sequence
  Synthetic Polynucleotide
tgcaccattgctgttccaaaa

SEQ ID NO: 2258 - DNA, 21 bp, Artificial Sequence
  Synthetic Polynucleotide
ccagagacaacccttttggcc

SEQ ID NO: 2259 - DNA, 21 bp, Artificial Sequence
  Synthetic Polynucleotide
agttggactggtgcttgatgt

SEQ ID NO: 2260 - DNA, 21 bp, Artificial Sequence
  Synthetic Polynucleotide
tgagcccggaggagatactgc

SEQ ID NO: 2261 - DNA, 21 bp, Artificial Sequence
  Synthetic Polynucleotide
aattcccaccaagtttggaat

SEQ ID NO: 2262 - DNA, 21 bp, Artificial Sequence
  Synthetic Polynucleotide
gcttgtcagaccatcttgaaa

SEQ ID NO: 2263 - DNA, 21 bp, Artificial Sequence
  Synthetic Polynucleotide
atctgcagctatctaaggctt

SEQ ID NO: 2264 - DNA, 21 bp, Artificial Sequence
  Synthetic Polynucleotide
tacacactgctccggaatcag

SEQ ID NO: 2265 - DNA, 21 bp, Artificial Sequence
  Synthetic Polynucleotide
agaattgccaaaagccacggc

SEQ ID NO: 2266 - DNA, 21 bp, Artificial Sequence
  Synthetic Polynucleotide
gatactgctctccatagagat

SEQ ID NO: 2267 - DNA, 21 bp, Artificial Sequence
  Synthetic Polynucleotide
ctgccagtgtctcttaggagt

SEQ ID NO: 2268 - DNA, 21 bp, Artificial Sequence
  Synthetic Polynucleotide
ctcttaggagtgaggtacctg

SEQ ID NO: 2269 - DNA, 21 bp, Artificial Sequence
  Synthetic Polynucleotide
ttcgccagtgcaactttactg

SEQ ID NO: 2270 - DNA, 21 bp, Artificial Sequence
  Synthetic Polynucleotide
gcacacaggtaataacgtgaa

SEQ ID NO: 2271 - DNA, 21 bp, Artificial Sequence
  Synthetic Polynucleotide
tcccggagagaagatgacatt

SEQ ID NO: 2272 - DNA, 21 bp, Artificial Sequence
  Synthetic Polynucleotide
ggttttccgaagcagataatg

SEQ ID NO: 2273 - DNA, 21 bp, Artificial Sequence
  Synthetic Polynucleotide
gcacttctacctggagactca

SEQ ID NO: 2274 - DNA, 21 bp, Artificial Sequence
  Synthetic Polynucleotide
cttccctccaacacggccttc

SEQ ID NO: 2275 - DNA, 21 bp, Artificial Sequence
  Synthetic Polynucleotide
ccaagctcggctgtggagagg

SEQ ID NO: 2276 - DNA, 21 bp, Artificial Sequence
  Synthetic Polynucleotide
cagtatcatgctcggaagagt

SEQ ID NO: 2277 - DNA, 21 bp, Artificial Sequence
  Synthetic Polynucleotide
agctctcagtatcatgctcgg

SEQ ID NO: 2278 - DNA, 21 bp, Artificial Sequence
  Synthetic Polynucleotide
gtctaaaccctgccattgata

SEQ ID NO: 2279 - DNA, 21 bp, Artificial Sequence
  Synthetic Polynucleotide
tgagctgtctctccggctggt

SEQ ID NO: 2280 - DNA, 21 bp, Artificial Sequence
  Synthetic Polynucleotide
gcatgctggaccgtgatgagg

SEQ ID NO: 2281 - DNA, 21 bp, Artificial Sequence
  Synthetic Polynucleotide
aagaacctccgcacagatatt

SEQ ID NO: 2282 - DNA, 21 bp, Artificial Sequence
  Synthetic Polynucleotide
agtaatttccacctagaaatg

SEQ ID NO: 2283 - DNA, 21 bp, Artificial Sequence
  Synthetic Polynucleotide
gaacctccgcacagatattgt

SEQ ID NO: 2284 - DNA, 21 bp, Artificial Sequence
  Synthetic Polynucleotide
tataaatccaaccttctgcca

SEQ ID NO: 2285 - DNA, 21 bp, Artificial Sequence
  Synthetic Polynucleotide
cagggtttgtttgtttcattt

SEQ ID NO: 2286 - DNA, 21 bp, Artificial Sequence
  Synthetic Polynucleotide
gagaattgccaaaagccacgg

SEQ ID NO: 2287 - DNA, 21 bp, Artificial Sequence
  Synthetic Polynucleotide
gacaagcactaacactgtgcc

SEQ ID NO: 2288 - DNA, 21 bp, Artificial Sequence
  Synthetic Polynucleotide
tcccggcatttggcagcatcc

SEQ ID NO: 2289 - DNA, 21 bp, Artificial Sequence
  Synthetic Polynucleotide
ccggagagaagatgacattgc

SEQ ID NO: 2290 - DNA, 21 bp, Artificial Sequence
  Synthetic Polynucleotide
ttcccaccaagtttggaataa

SEQ ID NO: 2291 - DNA, 21 bp, Artificial Sequence
  Synthetic Polynucleotide
ctttacatttgaagccaaagt

SEQ ID NO: 2292 - DNA, 21 bp, Artificial Sequence
  Synthetic Polynucleotide
catggacaactgctataaaat

SEQ ID NO: 2293 - DNA, 21 bp, Artificial Sequence
  Synthetic Polynucleotide
aaactgcaaaccctggtctgt

SEQ ID NO: 2294 - DNA, 21 bp, Artificial Sequence
  Synthetic Polynucleotide
tctttacatttgaagccaaag

SEQ ID NO: 2295 - DNA, 21 bp, Artificial Sequence
  Synthetic Polynucleotide
ttctcagcattcaagcaggcc

SEQ ID NO: 2296 - DNA, 21 bp, Artificial Sequence
  Synthetic Polynucleotide
acaaattcaaggttaattgga

SEQ ID NO: 2297 - DNA, 21 bp, Artificial Sequence
  Synthetic Polynucleotide
gtcactaataaatgaaactgt

SEQ ID NO: 2298 - DNA, 21 bp, Artificial Sequence
  Synthetic Polynucleotide
ttctgcggttggtaaagagaa

SEQ ID NO: 2299 - DNA, 21 bp, Artificial Sequence
  Synthetic Polynucleotide
atcttctttgccatcaaagat

SEQ ID NO: 2300 - DNA, 21 bp, Artificial Sequence
  Synthetic Polynucleotide
tataacctcaagttctgatgg

SEQ ID NO: 2301 - DNA, 21 bp, Artificial Sequence
  Synthetic Polynucleotide
taagaacctccgcacagatat

SEQ ID NO: 2302 - DNA, 21 bp, Artificial Sequence
  Synthetic Polynucleotide
atcgtcatgagtatgtacaca

SEQ ID NO: 2303 - DNA, 21 bp, Artificial Sequence
  Synthetic Polynucleotide
cagtgacaactgtggaaggaa

SEQ ID NO: 2304 - DNA, 21 bp, Artificial Sequence
  Synthetic Polynucleotide
tatggacctgagctgaagatc

SEQ ID NO: 2305 - DNA, 21 bp, Artificial Sequence
  Synthetic Polynucleotide
ttcaagaatatgctgtttcct

SEQ ID NO: 2306 - DNA, 21 bp, Artificial Sequence
  Synthetic Polynucleotide
ctcaaccctcaaggagctgct

SEQ ID NO: 2307 - DNA, 21 bp, Artificial Sequence
  Synthetic Polynucleotide
aacataccacagaacatggat

SEQ ID NO: 2308 - DNA, 21 bp, Artificial Sequence
  Synthetic Polynucleotide
gtcatgagtatgtacacactg

SEQ ID NO: 2309 - DNA, 21 bp, Artificial Sequence
  Synthetic Polynucleotide
gacacacttcaaccagaagct

SEQ ID NO: 2310 - DNA, 21 bp, Artificial Sequence
  Synthetic Polynucleotide
tcagcacacaggtaataacgt

SEQ ID NO: 2311 - DNA, 21 bp, Artificial Sequence
  Synthetic Polynucleotide
catggatctattaaagtcaca

SEQ ID NO: 2312 - DNA, 21 bp, Artificial Sequence
  Synthetic Polynucleotide
gaaccaagctcggctgtggag

SEQ ID NO: 2313 - DNA, 21 bp, Artificial Sequence
  Synthetic Polynucleotide
gccattgatattggacaggtg

SEQ ID NO: 2314 - DNA, 21 bp, Artificial Sequence
  Synthetic Polynucleotide
caccctcagcttcttcttcaa

SEQ ID NO: 2315 - DNA, 21 bp, Artificial Sequence
  Synthetic Polynucleotide
aggagagaattgccaaaagcc

SEQ ID NO: 2316 - DNA, 21 bp, Artificial Sequence
  Synthetic Polynucleotide
catgctgataactggtggcag

SEQ ID NO: 2317 - DNA, 21 bp, Artificial Sequence
  Synthetic Polynucleotide
agattatgtttaatctttaca

SEQ ID NO: 2318 - DNA, 21 bp, Artificial Sequence
  Synthetic Polynucleotide
ggagagaattgccaaaagcca

SEQ ID NO: 2319 - DNA, 21 bp, Artificial Sequence
  Synthetic Polynucleotide
aagattatgtttaatctttac

SEQ ID NO: 2320 - DNA, 21 bp, Artificial Sequence
  Synthetic Polynucleotide
gccttcataccaaaatggtcc

SEQ ID NO: 2321 - DNA, 21 bp, Artificial Sequence
  Synthetic Polynucleotide
cgctggtttgctgggaagcaa

SEQ ID NO: 2322 - DNA, 21 bp, Artificial Sequence
  Synthetic Polynucleotide
tgttctagatttctttccctt

SEQ ID NO: 2323 - DNA, 21 bp, Artificial Sequence
  Synthetic Polynucleotide
ctcgagctcagcacacaggta

SEQ ID NO: 2324 - DNA, 21 bp, Artificial Sequence
  Synthetic Polynucleotide
ccattgctgttccaaaaggcg

SEQ ID NO: 2325 - DNA, 21 bp, Artificial Sequence
  Synthetic Polynucleotide
agatcgtccacttttctgcca

SEQ ID NO: 2326 - DNA, 21 bp, Artificial Sequence
  Synthetic Polynucleotide
ggtaataacgtgaaggaactc

SEQ ID NO: 2327 - DNA, 21 bp, Artificial Sequence
  Synthetic Polynucleotide
ggcatgagagttttattcaag

SEQ ID NO: 2328 - DNA, 21 bp, Artificial Sequence
  Synthetic Polynucleotide
tcaaaccaatgaacagcaaag

SEQ ID NO: 2329 - DNA, 21 bp, Artificial Sequence
  Synthetic Polynucleotide
caaaccaatgaacagcaaagc

SEQ ID NO: 2330 - DNA, 21 bp, Artificial Sequence
  Synthetic Polynucleotide
ccaaccaactcaattattgag

SEQ ID NO: 2331 - DNA, 21 bp, Artificial Sequence
  Synthetic Polynucleotide
aaaaagattatgtttaatctt

SEQ ID NO: 2332 - DNA, 21 bp, Artificial Sequence
  Synthetic Polynucleotide
gtgggatgcctgcagaggagg

SEQ ID NO: 2333 - DNA, 21 bp, Artificial Sequence
  Synthetic Polynucleotide
ctcaagaccactcagaggcag

SEQ ID NO: 2334 - DNA, 21 bp, Artificial Sequence
  Synthetic Polynucleotide
gatgcctgcagaggaggtgcg

SEQ ID NO: 2335 - DNA, 21 bp, Artificial Sequence
  Synthetic Polynucleotide
agtggcatgagagttttattc

SEQ ID NO: 2336 - DNA, 21 bp, Artificial Sequence
  Synthetic Polynucleotide
atctattaaagtcacagaatg

SEQ ID NO: 2337 - DNA, 21 bp, Artificial Sequence
  Synthetic Polynucleotide
tcctgtgatccattttactgc

SEQ ID NO: 2338 - DNA, 21 bp, Artificial Sequence
  Synthetic Polynucleotide
ggctgcttctatcttctttgc

SEQ ID NO: 2339 - DNA, 21 bp, Artificial Sequence
  Synthetic Polynucleotide
tacacagatggctctgtgctg

SEQ ID NO: 2340 - DNA, 21 bp, Artificial Sequence
  Synthetic Polynucleotide
aagttctacctgacagtcctt

(19) United States
(12) Patent Application Publication
(10) Pub. No.: US 2025/0295688 A1
(43) Pub. Date: Sep. 25, 2025

(54) COMPOSITIONS AND METHODS OF MODULATING XANTHINE DEHYDROGENASE

(71) Applicant: HORIZON THERAPEUTICS IRELAND DAC, Dublin (IE)

(72) Inventors: Duncan BROWN, Berkeley, CA (US); Andrew NYBORG, Lincolnshire, IL (US); Michael Scott BOWERS, Evanston, IL (US)

(21) Appl. No.: 18/572,698

(22) PCT Filed: Jun. 17, 2022
(86) PCT No.: PCT/IB2022/000337

§ 371 Date: Dec. 20, 2023

Related U.S. Application Data
(60) Provisional application No. 63/213,170, filed on Jun. 21, 2021.

(51) Int. Cl. A61K 31/713; C12N 15/113

(52) U.S. Cl. CPC A61K 31/713; C12N 15/1137; C12N 2310/14; C12N 2310/351

(57) ABSTRACT

Compositions, methods for making and using polynucleotide inhibitors modulating xanthine dehydrogenase expression or activity are provided.

### CROSS REFERENCE TO RELATED APPLICATIONS

This application claims priority from U.S. Provisional Patent Application Ser. No. 63/213,170, filed on Jun. 21, 2021, the contents of which are incorporated herein by reference in their entirety.

### REFERENCE TO AN ELECTRONIC SEQUENCE LISTING

The contents of the electronic sequence listing (10805-US03-PCT Sequence Listing.xml; Size: 1.97 MB; and Date of Creation: Jan. 13, 2025) are herein incorporated by reference in its entirety.

### FIELD OF THE INVENTION

The present disclosure relates to polynucleic acid molecules (e.g., siRNAs) that modulates expression of Xanthine dehydrogenase (XDH) gene, pharmaceutical compositions that include polynucleic acid molecules and methods of use thereof.

### BACKGROUND

Serum uric acid (SUA) concentration is a significant parameter for human health. Alteration of SUA homeostasis has been linked to a number of diseases such as hyperuricemia, and is the underlying cause of gout and has been correlated with cardiovascular disease, hypertension, and renal disease. Xanthine dehydrogenase (XDH) is a critical for uric acid production by catalyzing the oxidation of hypoxanthine and xanthine to uric acid. While some XDH-inhibitor drugs, such as allopurinol and febuxostat, are clinically and commercially available, currently available drugs often result in serious adverse effects such as hypersensitivity drug reactions.

### SUMMARY

There is a need for developing novel XDH inhibitors for long-term use with fewer or no adverse effects. This disclosure addresses this unmet need.

The instant disclosure provides a polynucleic acid molecule that modulates expression of Xanthine dehydrogenase (XDH) gene, wherein the polynucleic acid molecule comprises a nucleic acid sequence that is at least 80% complementary to the nucleic acid sequence of at least one of SEQ ID NOs: 1-50, 201-410. In some aspects, the polynucleic acid molecule comprises a nucleic acid sequence that is at least 80% complementary to the nucleic acid sequence of at least one of SEQ ID NOs: 1-50. In some aspects, the polynucleic acid molecule comprises a nucleic acid sequence that is at least 80%, at least 85%, at least 90%, at least 95%, at least 96%, at least 97%, at least 98%, at least 99%, or 100% complementary to at least 15, 16, 17 contiguous nucleotides of at least one of SEQ ID NOs: 1-50, 201-410. In some aspects, the polynucleic acid molecule comprises a nucleic acid sequence that is at least 80%, at least 85%, at least 90%, at least 95%, at least 96%, at least 97%, at least 98%, at least 99%, or 100% complementary to at least 15, 16, 17 contiguous nucleotides of at least one of SEQ ID NOs: 1-50. In some aspects, the polynucleic acid molecule comprises a nucleic acid sequence that has less than 4 or less than 3 non-complementary nucleotides with the nucleic acid sequence of at least one of SEQ ID NO: 1-50, 201-410. In some aspects, the polynucleic acid molecule comprises a nucleic acid sequence that has less than 4 or less than 3 non-complementary nucleotides with the nucleic acid sequence of at least one of SEQ ID NO: 1-50. In some aspects, the polynucleic acid molecule comprises a nucleic acid sequence of at least 15 contiguous nucleotides differing by no more than 3 nucleotides, no more than 2 nucleotides, or 0 or 1 nucleotide from any one of SEQ ID NO: 1-50, 201-410. In some aspects, the polynucleic acid molecule comprises a nucleic acid sequence complementary to at least 13, at least 14, at least 15, at least 16, at least 17 contiguous nucleotides differing by no more than 3 nucleotides, no more than 2 nucleotides, or 0 or 1 nucleotide from any one of SEQ ID NO: 1-50, 201-410. In some aspects, the polynucleic acid molecule is single-stranded. In some aspects, the polynucleic acid molecule is double-stranded.

In some instances of the disclosed aspects, the polynucleic acid molecule comprises a sense strand and antisense strand.

In some instances of some of the disclosed aspects, the sense strand comprises a nucleic acid sequence that is at least 80%, at least 90%, at least 95% identical to at least one of the SEQ ID NOs: 1-50, 201-410. In some instances, the sense strand comprises a nucleic acid sequence of at least 15 contiguous nucleotides differing by no more than 3 nucleotides, no more than 2 nucleotides, or 0 or 1 nucleotide from SEQ ID NOs: 1-50, 201-410.

In some instances of some of the disclosed aspects, the sense strand comprises a nucleic acid sequence that is at least 80%, at least 90%, at least 95% identical to at least one of SEQ ID NOs: 1-50. In some instances, the sense strand comprises a nucleic acid sequence of at least 15 contiguous nucleotides differing by no more than 3 nucleotides, no more than 2 nucleotides, or 0 or 1 nucleotide from SEQ ID NOs: 1-50.

In some instances of some of the disclosed aspects, the sense strand comprises a nucleic acid sequence that is 80%, at least 90%, at least 95% identical to at least one of SEQ ID NOs: 51-100, 411-620. In some instances, the sense strand comprises a nucleic acid sequence of at least 15 contiguous nucleotides differing by no more than 3 nucleotides, no more than 2 nucleotides, or 0 or 1 nucleotide from SEQ ID NOs: 51-100, 411-620.

In some instances of some of the disclosed aspects, the sense strand comprises a nucleic acid sequence that is 80%, at least 90%, at least 95% identical to at least one of SEQ ID NOs: 51-100. In some instances, the sense strand comprises a nucleic acid sequence of at least 15 contiguous nucleotides differing by no more than 3 nucleotides, no more than 2 nucleotides, or 0 or 1 nucleotide from SEQ ID NOs: 51-100.

In some instances of some of the disclosed aspects, the sense strand comprises a nucleic acid sequence that is 100% identical to at least 15, 16, or 17 contiguous nucleotides of at least one of SEQ ID NOs: 51-100, 411-620.

In some instances of some of the disclosed aspects, the sense strand comprises a nucleic acid sequence that is 100% identical to at least 15, 16, or 17 contiguous nucleotides of at least one of SEQ ID NOs: 51-100.

In some instances of some of the disclosed aspects, the antisense strand comprises a nucleic acid sequence that is at least 80%, at least 90%, at least 95% identical to one of SEQ ID NOs: 101-150, 621-830.

In some instances of some of the disclosed aspects, the antisense strand comprises a nucleic acid sequence that is at least 80%, at least 90%, at least 95% identical to one of SEQ ID NOs: 101-150.

In some instances of some of the disclosed aspects, the antisense strand comprises a nucleic acid sequence that is 100% identical to at least 15, 16, or 17 contiguous nucleotides of at least one of SEQ ID NOs: 101-150, 621-830.

In some instances of some of the disclosed aspects, the antisense strand comprises a nucleic acid sequence that is 100% identical to at least 15, 16, or 17 contiguous nucleotides of at least one of SEQ ID NOs: 101-150.

In some instances of some of the disclosed aspects, the antisense strand comprises a nucleic acid sequence that is at least 80%, at least 90%, at least 95% identical to at least one of SEQ ID NOs: 151-200, 831-1040.

In some instances of some of the disclosed aspects, the antisense strand comprises a nucleic acid sequence that is at least 80%, at least 90%, at least 95% identical to at least one of SEQ ID NOs: 151-200.

In some instances of some of the disclosed aspects, the antisense strand comprises a nucleic acid sequence that is 100% identical to at least 15, 16, or 17 contiguous nucleotides of at least one of SEQ ID NOs: 151-200, 831-1040.

In some instances of some of the disclosed aspects, the antisense strand comprises a nucleic acid sequence that is 100% identical to at least 15, 16, or 17 contiguous nucleotides of at least one of SEQ ID NOs: 151-200.

In some instances of some of the disclosed aspects, the sense strand comprises a nucleic acid sequence that is 80%, at least 90%, at least 95% identical to at least one of SEQ ID NOs: 51-100, 411-620, and the antisense strand comprises a nucleic acid sequence that is at least 80%, at least 90%, at least 95% identical to at least one of SEQ ID NOs: 101-150, 621-830.

In some instances of some of the disclosed aspects, the sense strand comprises a nucleic acid sequence that is 80%, at least 90%, at least 95% identical to at least one of SEQ ID NOs: 51-100, and the antisense strand comprises a nucleic acid sequence that is at least 80%, at least 90%, at least 95% identical to at least one of SEQ ID NOs: 101-150.

In some instances of some of the disclosed aspects, the sense strand comprises a nucleic acid sequence that is 80%, at least 90%, at least 95% identical to SEQ ID NOs: 51-100, 411-620, and the antisense strand comprises a nucleic acid sequence that is at least 80%, at least 90%, at least 95% identical to SEQ ID NOs: 151-200, 831-1040.

In some instances of some of the disclosed aspects, the sense strand comprises a nucleic acid sequence that is 80%, at least 90%, at least 95% identical to SEQ ID NOs: 51-100, and the antisense strand comprises a nucleic acid sequence that is at least 80%, at least 90%, at least 95% identical to SEQ ID NOs: 151-200.

In some instances of some of the disclosed aspects, the polynucleic acid molecule comprises a sense strand comprising at least 13, 14, or 15 contiguous nucleotides differing by no more than 1, 2, or 3 nucleotides from any one of SEQ ID NOs: 21, 71, 267, 477, 1321, 1417, 2021, and 2197. In some instances, polynucleic acid molecule comprises an antisense strand comprising at least 13, 14, or 15 contiguous nucleotides differing by no more than 1, 2, or 3 nucleotides from any one of SEQ ID NOs: 121, 171, 687, 897, 1581, 1677, 1841, and 1937.

In some aspects, the polynucleic acid molecule comprises 17-30 nucleotides in length. In some aspects, the polynucleic acid molecule comprises 19-23 nucleotides in length. In some instances of some of the disclosed aspects, each of the sense strand and antisense strand is 17-30 nucleotides in length. In some instances of some of the disclosed aspects, each of the sense strand and antisense strand is 19-23 nucleotides in length.

In some aspects, the polynucleic acid molecule comprises at least one 2′-modified nucleoside, at least one modified internucleotide linkage, or at least one inverted abasic moiety. In some instances of some of the disclosed aspects, the polynucleic acid molecule comprises from 90% to 100% modification. In some instances of some of the disclosed aspects, the sense strand or the antisense strand comprises from 80% to 100% modification.

In some instances of some of the disclosed aspects, the at least one 2′ modified nucleotide: comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide.

In some instances of one of the disclosed aspects, the at least one modified internucleotide linkage comprises a phosphorothioate linkage or a phosphorodithioate linkage.

In some aspects, the polynucleic acid molecule comprises a phosphorodiamidate morpholino oligomer (PMO), locked nucleic acid (LNA) or constrained ethyl (cEt) sugar. In some aspects, the polynucleic acid molecule is conjugated with a peptide, antibody, lipid, carbohydrates, or a polymer. In some aspects, the polymer comprises N-Acetylgalactosamine (GalNAc) or a derivative thereof. Disclosed herein is a pharmaceutical composition comprising a polynucleic acid molecule of any one of claims 1-39 and a pharmaceutically acceptable excipient. In some aspects of the pharmaceutical composition, the composition is formulated for parenteral administration.

Disclosed herein is a method of inhibiting Xanthine dehydrogenase (XDH) activity in a cell comprising: contacting a polynucleic acid molecule of any one of claims 1-39 or a pharmaceutical composition of any one of claims 40-41, thereby inhibiting XDH activity in a cell. In some aspects, the contacting a polynucleic acid molecule reduces the XDH activity in the cell by at least 30%, 40%, 50%, 60%, 70%, 80%, or 90%. In some aspects, the contacting a polynucleic acid molecule reduces XDH mRNA expression level in the cell by at least 30%, 40%, 50%, 60%, 70%, 80%, or 90%.

Disclosed herein is a method of treating a disorder associated with Xanthine dehydrogenase (XDH) activity in a subject comprising: a) providing a pharmaceutical composition comprising a polynucleic acid molecule of any one of claims 1-39; b) administering the pharmaceutical composition to the subject in a dose and schedule sufficient to modulate the XDH activity in the subject, thereby treating the disorder associated with XDH activity. In some instances of some of the disclosed aspects, the pharmaceutical composition comprises a sense strand comprising at least 13, 14, or 15 contiguous nucleotides differing by no more than 1, 2, or 3 nucleotides from any one of SEQ ID NOs: 21, 71, 267, 477, 1321, 1417, 2021, and 2197. In some instances, pharmaceutical composition comprises an antisense strand comprising at least 13, 14, or 15 contiguous nucleotides differing by no more than 1, 2, or 3 nucleotides from any one of SEQ ID NOs: 121, 171, 687, 897, 1581, 1677, 1841, and 1937.

In some aspect, the disorder is associated with the increased expression or activity of the XDH gene or protein. In some aspects, the disorder comprises hyperuricemia, gout, NAFLD, NASH, metabolic disorder, insulin resistance, type 2 diabetes, or a cardiovascular disease. Disclosed herein is a method of treating gout in a subject comprising: a) providing a pharmaceutical composition comprising a polynucleic acid molecule as described herein; b) administering the pharmaceutical composition to the subject in a dose and schedule sufficient to modulate the XDH activity in the subject, thereby treating gout. In some aspects the dose is between about 0.01 mg/kg to 50 mg/kg.

In certain aspects, the pharmaceutical composition is administered parenterally. In certain aspects, the pharmaceutical composition is administered intravenously. In certain aspects, the pharmaceutical composition is administered subcutaneously. In some aspects, the pharmaceutical composition is administered intrathecally.

In some aspects, the administration reduces serum uric acid level in the subject at least by about 20%, about 30%, about 40% about 50%, about 60%, about 70%, or about 80% compared to serum uric acid levels of an untreated subject or the subject before the treatment. In some aspects, the subject failed one or more first line standard of care therapies prior to the treatment. In some aspects, the subject failed allopurinol or febuxostat treatment prior to the treatment INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

### Overview

Described herein are compositions and methods for modulating gene expression or pathway associated with xanthine dehydrogenase (XDH) gene expression or activity. Also described herein are composition and methods for treating a disease, disorder, or symptom associated with XDH gene expression or activity (e.g., hyperuricemia, gout, etc.). The composition comprises at least one oligonucleotide or polynucleotide that, upon delivery into a cell, binds to an endogenous target nucleic acid, which leads to the degradation of the target nucleic acid, XDH mRNA. Also described herein is a method for utilizing the composition or the oligonucleotide described herein. In some aspects, the methods treat a disease, disorder, or symptom associated with XDH gene expression or activity by contacting a cell with the oligonucleotide or polynucleotide to decrease the XDH expression or activity.

### Certain Terminology

All terms are intended to be understood as they would be understood by a person skilled in the art. Unless defined otherwise, all technical and scientific terms used herein have the same meaning as commonly understood by one of ordinary skill in the art to which the disclosure pertains. It is to be understood that the foregoing general description and the following detailed description are exemplary and explanatory only and are not restrictive of any subject matter claimed.

In this disclosure, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone. As used herein, the term “and/or” includes any and all combinations of one or more of the associated listed items.

The term “about” or “approximately” as used herein when referring to a measurable value such as a parameter, an amount, a temporal duration, and the like, is meant to encompass variations of +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

An “agent” is any small molecule chemical compound, antibody, nucleic acid molecule, or polypeptide, or fragments thereof.

An “alteration”, “modulation”, or “change” of gene or protein expression or gene or protein activity is an increase or decrease of gene, mRNA, or protein expression, or its activity thereof Δn alteration can be by as little as 1%, 2%, 3%, 4%, 5%, 10%, 20%, 30%, or by 40%, 50%, 60%, or even by as much as 70%, 75%, 80%, 90%, or 100%.

A “biologic sample” is any tissue, cell, fluid, or other material derived from an organism. As used herein, the term “sample” includes a biologic sample such as any tissue, cell, fluid, or other material derived from an organism.

“Specifically binds” refers to a compound (e.g., peptide, nucleotide, oligonucleotide, oligonucleotide conjugate) that recognizes and binds a molecule (e.g., polypeptide, nucleotide, etc.), but does not substantially recognize and bind other molecules in a sample, for example, a biological sample.

As used herein, “oligonucleotides” are stretches of more than 2 nucleotides linked by phosphate bond or phosphorothioate bond; wherein more than 2 nucleotides comprises 3, 4, 5, 6, 7, 8, 9, 10 or 15 nucleotides in the stretch of nucleotides. Oligonucleotides can be used interchangeably with the term polynucleotides, wherein the oligonucleotide is for example, more than 8, more than 10, more than 15 or more than 20 nucleotides long.

“Off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

The terms “sequence” and “nucleotide sequence” refer a succession or order of nucleobases or nucleotides, described with a succession of letters using standard nomenclature. A nucleic acid molecule can comprise unmodified and/or modified nucleotides. A nucleotide sequence can comprise unmodified and/or modified nucleotides. The term “nucleotide” refers to a glycoside comprising a sugar moiety, a base moiety and a covalently linked group (linkage group), such as a phosphate or phosphorothioate internucleoside linkage group, and covers both naturally occurring nucleotides (e.g., DNA or RNA), and non-naturally occurring nucleotides comprising modified sugar and/or base moieties, which are also referred to as nucleotide analogs herein.

As used herein, the terms “determining”, “assessing”, “assaying”, “measuring”, “detecting” and their grammatical equivalents refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

### Xanthine Oxidoreductase System

Xanthine oxidoreductase (XOR) catalyzes oxidative hydroxylation of hypoxanthine to xanthine to uric acid, accompanying the production of reactive oxygen species (ROS). Its member enzyme, Xanthine dehydrogenase, belongs to the group of molybdenum-containing hydroxylases involved in the oxidative metabolism of purines. In its usual form as xanthine dehydrogenase catalyzes the reaction, XH+H2O+NAD+→X═O+NADH. The most common substrates are purines. Uric acid forms the metabolic endpoint of purine degradation. The last metabolic steps in the process (from hypoxanthine to xanthine and from xanthine to uric acid) are promoted by xanthine dehydrogenase (oxidoreductase, EC1.1.3.22). Xanthine dehydrogenase is a flavoprotein that contains both iron and Mo and uses NAD+ as electron acceptor (Mendel and Bittner, 2006; Schwarz and Mendel, 2006).

Xanthine dehydrogenase exists in two interconvertible forms, xanthine dehydrogenase and xanthine oxidase. Xanthine dehydrogenase can be converted to xanthine oxidase by reversible sulfhydryl oxidation or by irreversible proteolytic modification. In its oxidase form, the enzyme transfers the reducing equivalent generated by oxidation of substrates to molecular oxygen with the resultant production of superoxide anion and hydrogen peroxide. Hydrogen peroxide can be converted to free hydroxyl radicals. For example, during ischemia, reperfusion, or reoxygenation of an injured tissue can occur, and xanthine dehydrogenase can be converted to xanthine oxidase (Mendel and Bittner, 2006; Schartz, 2005). In this latter form, the reaction sequence is XH+H2O+O2→X═O+H2O2. Given that in such conditions ATP is depleted and there is an increase in the purine pool, such available substrate promotes production of large quantities of superoxide radicals are released, which can be a major source of tissue peroxidation. A major source of ROS in the cytosol of hepatocytes is XDH (xanthine dehydrogenase/oxidase). Under normal conditions, this enzyme predominantly exhibits XD (xanthine dehydrogenase) activity. However, oxidation of sulfhydryl groups on the protein or proteolytic cleavage results in loss of the ability to bind NAD+. When this occurs, the enzyme behaves as an oxidase and uses oxygen as an electron acceptor instead. Estimates of total enzyme in the form of XO (xanthine oxidase) range from approximately 2% to 25% in the liver.

Human XDH gene is located on chromosome 2 (NC_000002.12). The protein expression is predominantly detectable in liver, small intestine, duodenum, colon, gall bladder and appendix.

Diseases Associated with Xanthine Oxidoreductase Enzymes

Defects in xanthine dehydrogenase cause xanthinuria, may contribute to adult respiratory stress syndrome, and may potentiate influenza infection through an oxygen metabolite-dependent mechanism. XDH activity is associated with glycemic control in patients with T1DM and is associated with vascular endothelial dysfunction. XDH activity leads to generation of uric acid. Uric acid usually forms ions and salts known as urates and acid urates in serum. Clinically, overproduction or under-excretion of uric acid results in the elevated level of serum uric acid (SUA), termed hyperuricemia, which has long been established as the major etiologic factor in gout. Alteration of SUA homeostasis has been linked to a number of diseases. For example, an abnormally high SUA level, termed hyperuricemia, is the underlying cause of gout and has been correlated with cardiovascular disease, hypertension, and renal disease. More recent studies have demonstrated that hyperuricemia may directly contribute to the development or progression of these diseases. Most patients suffering from gout are treated with oral urate-reducing therapies, although there is a high there exists a high proportion of these patients who do not respond adequately to the therapy and therefore continue to experience the painful symptoms, leading up to bone and joint damage and organ failure. A further target-specific therapeutic approach is demanded for the large population worldwide who suffer from the debilitating disease.

### Posttranscriptional Inhibitor Polynucleotides of XDH

Provided herein are post-transcriptional regulators of an XDH gene as a targeted, specific approach to address the unmet need. The post-transcriptional regulators described herein are polynucleotides or oligonucleotides. In some aspects, the post-transcriptional regulators described herein comprise RNA molecules. In some aspects, the post-transcriptional regulators described herein comprise DNA molecules. In some aspects, the post-transcriptional regulators described herein are single-stranded. In some aspects, the post-transcriptional regulators described herein are double-stranded. In some aspects the post-transcriptional regulators described herein are inhibitory RNA, for use as an RNAi based therapeutic. The composition and methods described herein use the RNA-interference mechanism for fast, effective, targeted and durable inhibition of target genes, thereby affecting the expression of the proteins encoded by the genes by effectively inhibiting and knocking down the expression of the proteins; the RNAi being delivered into effective cell types via efficient modifications and improvements of the RNAi (siRNA).

In some aspects, the polynucleotide of the disclosure is an siRNA. siRNA molecules are the effector molecules of RNAi. In some aspects, the siRNA molecule comprises 19+2mer structure (that is, a duplex of two 21-nucleotide RNA molecules with 19 complementary bases and terminal 2-nucleotide 3′ overhangs). In some aspects, the siRNA molecule comprises 19, 20, 21+2-3mer structure (that is, a duplex of two 21-24-nucleotide RNA molecules with 19, 20, 21 complementary bases and terminal 2-3-nucleotide 3′ overhangs or 5′ overhangs and/or a cap molecule). One of the strands of the siRNA (the guide or antisense strand) is complementary to a target transcript, whereas the other strand is designated the passenger or sense strand. siRNAs act to guide the Argonaute 2 protein (AGO2), as part of the RNA-induced silencing complex (RISC), to complementary target transcripts. Complete complementarity between the siRNA and the target transcript results in cleavage (that is, slicer activity) of the target opposite position 10-11 of the guide strand, catalyzed by AGO2, leading to gene silencing.

In some instances, the polynucleotide disclosed herein comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides. In some aspects, the polynucleotide comprises a sense strand and an antisense strand, and the antisense strand includes two non-base pairing nucleotides as an overhang at the 3′-end while the sense strand has no overhang. Optionally, in such embodiments, the non-base pairing nucleotides have a sequence of TT, dTdT, or UU. In some aspects, the polynucleic acid molecule comprises a sense strand and an antisense strand, and the sense strand has one or more nucleotides at the 5′-end that are complementary to the antisense sequence.

In some aspects, the polynucleotide of the disclosure is a double-strand RNA (dsRNA) that triggers RNA interference.

In some aspects, the polynucleic acid molecule comprises a first polynucleotide. In some aspects, the first polynucleotide is from about 10 to about 50 nucleotides in length. In some instances, the first polynucleotide is from about 10 to about 30, from about 15 to about 30, from about 18 to about 25, form about 18 to about 24, from about 19 to about 23, or from about 20 to about 22 nucleotides in length.

In some instances, the first polynucleotide is about 50 nucleotides in length. In some instances, the first polynucleotide is about 45 nucleotides in length. In some instances, the first polynucleotide is about 40 nucleotides in length. In some instances, the first polynucleotide is about 35 nucleotides in length. In some instances, the first polynucleotide is about 30 nucleotides in length. In some instances, the first polynucleotide is about 25 nucleotides in length. In some instances, the first polynucleotide is about 20 nucleotides in length. In some instances, the first polynucleotide is about 19 nucleotides in length. In some instances, the first polynucleotide is about 18 nucleotides in length. In some instances, the first polynucleotide is about 17 nucleotides in length. In some instances, the first polynucleotide is about 16 nucleotides in length. In some instances, the first polynucleotide is about 15 nucleotides in length. In some instances, the first polynucleotide is about 14 nucleotides in length. In some instances, the first polynucleotide is about 13 nucleotides in length. In some instances, the first polynucleotide is about 12 nucleotides in length. In some instances, the first polynucleotide is about 11 nucleotides in length. In some instances, the first polynucleotide is about 10 nucleotides in length. In some instances, the first polynucleotide is between about 10 and about 50 nucleotides in length. In some instances, the first polynucleotide is between about 10 and about 45 nucleotides in length. In some instances, the first polynucleotide is between about 10 and about 40 nucleotides in length. In some instances, the first polynucleotide is between about 10 and about 35 nucleotides in length. In some instances, the first polynucleotide is between about 10 and about 30 nucleotides in length. In some instances, the first polynucleotide is between about 10 and about 25 nucleotides in length. In some instances, the first polynucleotide is between about 10 and about 20 nucleotides in length. In some instances, the first polynucleotide is between about 15 and about 25 nucleotides in length. In some instances, the first polynucleotide is between about 15 and about 30 nucleotides in length. In some instances, the first polynucleotide is between about 12 and about 30 nucleotides in length.

In some aspects, the polynucleic acid molecule comprises a second polynucleotide. In some aspects, the second polynucleotide is from about 10 to about 50 nucleotides in length. In some instances, the second polynucleotide is from about 10 to about 30, from about 15 to about 30, from about 18 to about 25, form about 18 to about 24, from about 19 to about 23, or from about 20 to about 22 nucleotides in length.

In some instances, the second polynucleotide is about 50 nucleotides in length. In some instances, the second polynucleotide is about 45 nucleotides in length. In some instances, the second polynucleotide is about 40 nucleotides in length. In some instances, the second polynucleotide is about 35 nucleotides in length. In some instances, the second polynucleotide is about 30 nucleotides in length. In some instances, the second polynucleotide is about 25 nucleotides in length. In some instances, the second polynucleotide is about 20 nucleotides in length. In some instances, the second polynucleotide is about 19 nucleotides in length. In some instances, the second polynucleotide is about 18 nucleotides in length. In some instances, the second polynucleotide is about 17 nucleotides in length. In some instances, the second polynucleotide is about 16 nucleotides in length. In some instances, the second polynucleotide is about 15 nucleotides in length. In some instances, the second polynucleotide is about 14 nucleotides in length. In some instances, the second polynucleotide is about 13 nucleotides in length. In some instances, the second polynucleotide is about 12 nucleotides in length. In some instances, the second polynucleotide is about 11 nucleotides in length. In some instances, the second polynucleotide is about 10 nucleotides in length. In some instances, the second polynucleotide is between about 10 and about 50 nucleotides in length. In some instances, the second polynucleotide is between about 10 and about 45 nucleotides in length. In some instances, the second polynucleotide is between about 10 and about 40 nucleotides in length. In some instances, the second polynucleotide is between about 10 and about 35 nucleotides in length. In some instances, the second polynucleotide is between about 10 and about 30 nucleotides in length. In some instances, the second polynucleotide is between about 10 and about 25 nucleotides in length. In some instances, the second polynucleotide is between about 10 and about 20 nucleotides in length. In some instances, the second polynucleotide is between about 15 and about 25 nucleotides in length. In some instances, the second polynucleotide is between about 15 and about 30 nucleotides in length. In some instances, the second polynucleotide is between about 12 and about 30 nucleotides in length.

In some aspects, a polynucleotide of the disclosure comprises a region is complementary to a portion of XDH mRNA. In some aspects, the XDH mRNA is Homo sapiens xanthine dehydrogenase (XDH), mRNA (e.g., NCBI gene accession reference sequence number NM_000379.4. In some aspects, the human XDH mRNA RefSeq ID is NM_000379.3, or NM_000379.2.

In some aspects, the polynucleotide described herein comprises a nucleic acid sequence that is complementary to at least 10, at least 11, at least 12, at least 13, at least 14, at least 15, at least 16, at least 17, at least 18, at least 19, or at least 20 contiguous nucleotide residues selected from any one of the sequences of SEQ ID NOs: 1-100, 201-620, 1041-1300, 1301-1560, or 2081-2340.

In some aspects, the polynucleotide described herein comprises a nucleic acid sequence that is complementary to a sequence having at least about 50%, about 55%, about 60%, about 65%, about 70%, about 75%, about 80%, about 85%, about 90%, about 95%, about 96%, about 97%, about 98%, about 99%, or 100% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 60% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 70% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 80% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 90% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 91% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 92% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 93% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 94% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 95% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 96% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 97% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 98% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is complementary to a sequence having at least 99% sequence identity to a target sequence as set forth in SEQ ID NOs: 1-100, 201-620, 1041-1300, 1301-1560, or 2081-2340. In some aspects, the polynucleotide described herein comprises a nucleic acid sequence that is fully complementary to a target sequence as set forth in SEQ ID NOs: 1-100, 201-620, 1041-1300, 1301-1560, or 2081-2340.

In some aspects, the polynucleotide of the disclosure comprises an antisense oligonucleotide (ASO). ASO is an inhibitory polynucleotide that is small (˜18-30 nucleotides), synthetic, single-stranded nucleic acid polymers of diverse chemistries, which can be employed to modulate gene expression via various mechanisms. ASOs can be subdivided into two major categories: RNase H competent and steric block. The endogenous RNase H enzyme RNASEH1 recognizes RNA-DNA heteroduplex substrates that are formed when DNA-based oligonucleotides bind to their cognate mRNA transcripts and catalyzes the degradation of RNA. Cleavage at the site of ASO binding results in destruction of the target RNA, thereby silencing target gene expression. This approach has been widely used as a means of downregulating disease-causing or disease-modifying genes.

In some aspects, the ASO can target one or more regions of the human XDH gene transcript. In some aspects, the antisense polynucleotides can target a region starting at nucleotide residue 240. The nucleotide residues are numbered in reference to the NCBI gene accession reference sequence number NM_000379.4. In some aspects, the ASO can target a region starting at nucleotide residue 258. In some aspects, the ASO can target a region starting at nucleotide residue 274. In some aspects, the ASO can target a region starting at nucleotide residue 402. In some aspects, the ASO can target a region starting at nucleotide residue 859. In some aspects, the v can target a region starting at nucleotide residue 860. In some aspects, the ASO can target a region starting at nucleotide residue 1355. In some aspects, the ASO can target a region starting at nucleotide residue 1380. In some aspects, the ASO can target a region starting at nucleotide residue 1830. In some aspects, the ASO can target a region starting at nucleotide residue 1840. In some aspects, the ASO can target a region starting at nucleotide residue 1913. In some aspects, the ASO can target a region starting at nucleotide residue 1923. In some aspects, the ASO can target a region starting at nucleotide residue 2066. In some aspects, the ASO can target a region starting at nucleotide residue 2077. In some aspects, the ASO can target a region starting at nucleotide residue 2431. In some aspects, the ASO can target a region starting at nucleotide residue 2434. In some aspects, the ASO can target a region starting at nucleotide residue 2437. In some aspects, the ASO can target a region starting at nucleotide residue 2557. In some aspects, the ASO can target a region starting at nucleotide residue 2569. In some aspects, the ASO can target a region starting at nucleotide residue 2611. In some aspects, the v can target a region starting at nucleotide residue 2698. In some aspects, the ASO can target a region starting at nucleotide residue 2789. In some aspects, the ASO can target a region starting at nucleotide residue 2993. In some aspects, the ASO can target a region starting at nucleotide residue 2996. In some aspects, the ASO can target a region starting at nucleotide residue 3004. In some aspects, the ASO can target a region starting at nucleotide residue 3084. In some aspects, the ASO can target a region starting at nucleotide residue 3600. In some aspects, the ASO can target a region starting at nucleotide residue 3760. In some aspects, the ASO can target a region starting at nucleotide residue 3930. In some aspects, the ASO can target a region starting at nucleotide residue 4057. In some aspects, the ASO can target a region starting at nucleotide residue 4144. In some aspects, the ASO can target a region starting at nucleotide residue 4151. In some aspects, the ASO can target a region starting at nucleotide residue 4153. In some aspects, the ASO can target a region starting at nucleotide residue 4359. In some aspects, the ASO can target a region starting at nucleotide residue 4360. In some aspects, the ASO can target a region starting at nucleotide residue 4404. In some aspects, the ASO can target a region starting at nucleotide residue 4405. In some aspects, the ASO can target a region starting at nucleotide residue 4441. In some aspects, the ASO can target a region starting at nucleotide residue 4443. In some aspects, the ASO can target a region starting at nucleotide residue 4507. In some aspects, the ASO can target a region starting at nucleotide residue 4517. In some aspects, the ASO can target a region starting at nucleotide residue 4628. In some aspects, the ASO can target a region starting at nucleotide residue 4662. In some aspects, the ASO can target a region starting at nucleotide residue 4666. In some aspects, the ASO can target a region starting at nucleotide residue 4802. In some aspects, the ASO can target a region starting at nucleotide residue 5413. In some aspects, the ASO can target a region starting at nucleotide residue 5420. In some aspects, the ASO can target a region starting at nucleotide residue 5474. In some aspects, the ASO can target a region starting at nucleotide residue 5475. In some aspects, the v can target a region starting at nucleotide residue 5671.

In some aspects, the ASO comprises a nucleic acid sequence complementary to any one of the sequences selected from SEQ ID NOs: 1-100, SEQ ID NOs: 201-620, or a sequence that is at least 80%, at least 90%, at least 95% or at least 98% identical to any one of the sequences selected from SEQ ID NOs: 1-100, SEQ ID NOs: 201-620.

In some aspects, the ASO comprises a nucleic acid sequence that is complementary to at least 10, at least 11, at least 12, at least 13, at least 14, at least 15, at least 16 at least 17, at least 18, at least 19, or at least 20 contiguous nucleotide residues selected from any one of the sequences of SEQ ID NOs: 1-100, 201-620.

In some aspects, the ASO comprises a nucleic acid sequence complementary to a sequence having 1, 2, or 3 nucleotide mismatch (or non-complementary nucleotide) with any one of the sequences selected from SEQ ID NOs: 1-100, SEQ ID NOs: 201-620. In some aspects, the ASO comprises a nucleic acid sequence that has less than 4 or less than 3 non-complementary nucleotides with any one of the sequences selected from SEQ ID NOs: 1-100, SEQ ID NOs: 201-620. In some aspects, the ASO comprises a nucleic acid sequence that In some aspects, the ASO comprises a nucleic acid sequence that is complementary to at least 12, at least 13, at least 14, at least 15, at least 16, at least 17, at least 18, at least 19 consecutive nucleotides of one of the sequences selected from SEQ ID NOs: 1-100, SEQ ID NOs: 201-620, differing by no more than 0, 1, 2, 3, 4 nucleotides.

In some aspects, the post-transcriptional regulators described herein comprise a morpholino nucleotide. In some aspects, the polynucleotide comprising the morpholino nucleotide comprises a region complementary to any one of the sequences selected from SEQ ID NOs: 1-100, SEQ ID NOs: 201-620, or a sequence that is at least 80%, at least 90%, at least 95% or at least 98% identical to any one of the sequences selected from SEQ ID NOs: 1-100, SEQ ID NOs: 201-620, SEQ ID NOs 1041-1300, 1301-1560, 2081-2340.

In some aspects, the polynucleotide comprising the morpholino nucleotide comprises a nucleic acid sequence that is complementary to at least 10, at least 11, at least 12, at least 13, at least 14, at least 15, at least 16 at least 17, at least 18, at least 19, at least 20 contiguous nucleotide residues selected from any one of the sequences of SEQ ID NOs: 1-100, 201-620, 1041-1300, 1301-1560, 2081-2340.

In some aspects, the polynucleotide comprising the morpholino nucleotide can comprise a region complementary to a sequence having 1, 2, or 3 nucleotide mismatch with any one of the sequences selected from SEQ ID NOs: 1-100, SEQ ID NOs: 201-620, SEQ ID NOs: 1041-1300, SEQ ID NOs: 1301-1560, SEQ ID NOs: 2081-2340

In an aspect, the disclosure provides double stranded ribonucleic acids (dsRNA) for inhibiting expression of a XDH gene, wherein the dsRNA agent can comprise at least one oligonucleotide selected from the Table 1. In some aspects the dsRNA agent is an siRNA. In some aspects, the siRNA strand that is complimentary to the mRNA strand is termed guide RNA and the other strand in the siRNA duplex (double strand), is termed the passenger strand. In some aspects, the siRNA guide strand can be referred to as the antisense strand.

The nucleotide residues are numbered in reference to the NCBI gene accession reference sequence number NM_000379.4. In some aspects, the siRNA can target a region starting at nucleotide 240. In some aspects, the siRNA can target a region starting at nucleotide 258. In some aspects, the siRNA can target a region starting at nucleotide 274. In some aspects, the siRNA can target a region starting at nucleotide 402. In some aspects, the siRNA can target a region starting at nucleotide 859. In some aspects, the siRNA can target a region starting at nucleotide 860. In some aspects, the siRNA can target a region starting at nucleotide 1355. In some aspects, the siRNA can target a region starting at nucleotide 1380. In some aspects, the siRNA can target a region starting at nucleotide 1830. In some aspects, the siRNA can target a region starting at nucleotide 1840. In some aspects, the siRNA can target a region starting at nucleotide 1913. In some aspects, the siRNA can target a region starting at nucleotide 1923. In some aspects, the siRNA can target a region starting at nucleotide 2066. In some aspects, the siRNA can target a region starting at nucleotide 2077. In some aspects, the siRNA can target a region starting at nucleotide 2431. In some aspects, the siRNA can target a region starting at nucleotide 2434. In some aspects, the siRNA can target a region starting at nucleotide 2437. In some aspects, the siRNA can target a region starting at nucleotide 2557. In some aspects, the siRNA can target a region starting at nucleotide 2569. In some aspects, the siRNA can target a region starting at nucleotide 2611. In some aspects, the siRNA can target a region starting at nucleotide 2698. In some aspects, the siRNA can target a region starting at nucleotide 2789. In some aspects, the siRNA can target a region starting at nucleotide 2993. In some aspects, the siRNA can target a region starting at nucleotide 2996. In some aspects, the siRNA can target a region starting at nucleotide 3004. In some aspects, the siRNA can target a region starting at nucleotide 3084. In some aspects, the siRNA can target a region starting at nucleotide 3600. In some aspects, the siRNA can target a region starting at nucleotide 3760. In some aspects, the siRNA can target a region starting at nucleotide 3930. In some aspects, the siRNA can target a region starting at nucleotide 4057. In some aspects, the siRNA can target a region starting at nucleotide 4144. In some aspects, the siRNA can target a region starting at nucleotide 4151. In some aspects, the siRNA can target a region starting at nucleotide 4153. In some aspects, the siRNA can target a region starting at nucleotide 4359. In some aspects, the siRNA can target a region starting at nucleotide 4360. In some aspects, the siRNA can target a region starting at nucleotide 4404. In some aspects, the siRNA can target a region starting at nucleotide 4405. In some aspects, the siRNA can target a region starting at nucleotide 4441. In some aspects, the siRNA can target a region starting at nucleotide 4443. In some aspects, the siRNA can target a region starting at nucleotide 4507. In some aspects, the siRNA can target a region starting at nucleotide 4517. In some aspects, the siRNA can target a region starting at nucleotide 4628. In some aspects, the siRNA can target a region starting at nucleotide 4662. In some aspects, the siRNA can target a region starting at nucleotide 4666. In some aspects, the siRNA can target a region starting at nucleotide 4802. In some aspects, the siRNA can target a region starting at nucleotide 5413. In some aspects, the siRNA can target a region starting at nucleotide 5420. In some aspects, the siRNA can target a region starting at nucleotide 5474. In some aspects, the siRNA can target a region starting at nucleotide 5475. In some aspects, the siRNA can target a region starting at nucleotide 5671.

In some aspects, the siRNA comprises a 19-mer, 20-mer, or 21-mer duplex, wherein each strand comprises at least 19, 20, 21 nucleotide residues. In some aspects, a 19-mer duplex can comprise a stretch of 19 nucleotides in each strand. In some aspects, a 19-mer duplex comprises 19 nucleotides spanning the double-stranded region of the siRNA. In some aspects, 19-mer duplex can comprise a stretch of 19 nucleotides in at least one strand, and a stretch of at least 19, or 20 or 21 nucleotides in the complementary strand within the double stranded siRNA. In some aspects, the antisense (guide) RNA strand comprises 19 nucleotide that is at least 60% complementary to the nucleic acid sequence of SEQ ID NOs: 1-50. In some aspects, the antisense strand comprises 19 nucleotide that is at least 65% complementary to the nucleic acid sequence of SEQ ID NOs: 1-50. In some aspects, the antisense strand comprises 19 nucleotide that is at least 70% complementary to the nucleic acid sequence of SEQ ID NOs: 1-50. In some aspects, the antisense strand comprises 19 nucleotide that is at least 75% complementary to the nucleic acid sequence of SEQ ID NOs: 1-50. In some aspects, the antisense strand comprises 19 nucleotide that is at least 80% complementary to the nucleic acid sequence of SEQ ID NOs: 1-50. In some aspects, the antisense strand comprises 19 nucleotide that is at least 85% complementary to the nucleic acid sequence of SEQ ID NOs: 1-50. In some aspects, the antisense strand comprises 19 nucleotide that is at least 90% complementary to the nucleic acid sequence of SEQ ID NOs: 1-50. In some aspects, the antisense strand comprises 19 nucleotide that is at least 95% complementary to the nucleic acid sequence of SEQ ID NOs: 1-50. In some aspects, the antisense strand comprises 19 nucleotide that is at least 96% complementary to the nucleic acid sequence of SEQ ID NOs: 1-50. In some aspects, the antisense strand comprises 19 nucleotide that is at least 97% complementary to the nucleic acid sequence of SEQ ID NOs: 1-50. In some aspects, the antisense strand comprises 19 nucleotide that is at least 98% complementary to the nucleic acid sequence of SEQ ID NOs: 1-50. In some aspects, the antisense strand comprises 19 nucleotide that is at least 99% complementary to the nucleic acid sequence of SEQ ID NOs: 1-50. In some aspects, the antisense strand comprises 19 nucleotide that is 100% complementary to the nucleic acid sequence of SEQ ID NOs: 1-50.

In some aspects, the antisense strand comprises a nucleic acid sequence that is 100% complementary to at least 13 contiguous nucleotides of the SEQ ID NOs: 1-50. In some aspects, the antisense strand comprises a nucleic acid sequence that is 100% complementary to at least 14 contiguous nucleotides of the SEQ ID NOs: 1-50. In some aspects, the antisense strand comprises a nucleic acid sequence that is 100% complementary to at least 15 contiguous nucleotides of the SEQ ID NOs: 1-50. In some aspects, the antisense strand comprises a nucleic acid sequence that is 100% complementary to at least 16 contiguous nucleotides of the SEQ ID NOs: 1-50. In some aspects, the antisense strand comprises a nucleic acid sequence that is 100% complementary to at least 17 contiguous nucleotides of the SEQ ID NOs: 1-50. In some aspects, the antisense strand comprises a nucleic acid sequence that is 100% complementary to at least 18 contiguous nucleotides of the SEQ ID NOs: 1-50. In some aspects, the antisense strand comprises a nucleic acid sequence that is 100% complementary to at least 19 contiguous nucleotides of the SEQ ID NOs: 1-50. In some aspects, the antisense strand comprises a nucleic acid sequence that is 100% complementary to at least 20 contiguous nucleotides of the SEQ ID NOs: 1-50.

In some aspects, the antisense strand comprises a nucleic acid sequence that has 1, 2, 3, 4 non-complementary nucleotides with respect to the nucleic acid sequence of SEQ ID NO: 1-50. In some aspects, the antisense strand comprises a nucleic acid sequence that has less than 4 non-complementary nucleotides with respect to the nucleic acid sequence of SEQ ID NO: 1-50. In some aspects, the antisense strand comprises a nucleic acid sequence that has less than 3 non-complementary nucleotides with respect to the nucleic acid sequence of SEQ ID NO: 1-50. In some aspects, the antisense strand comprises a nucleic acid sequence that has less than 2 non-complementary nucleotides with respect to the nucleic acid sequence of SEQ ID NO: 1-50.

In some aspects, the siRNA molecule comprises a 19-mer duplex, wherein each sense and antisense strand comprises at least 19 nucleotide residues. In some aspects, a 19-mer duplex can comprise a stretch of 19 nucleotides in each strand. In some aspects, 19-mer duplex can comprise a stretch of 19 nucleotides in at least one strand, and a stretch of at least 19, or 20 or 21 nucleotides in the complementary strand within the double stranded siRNA. In some aspects, the guide RNA strand comprises 19 nucleotide that is at least 60% complementary to the nucleic acid sequence of SEQ ID NOs: 201-410. In some aspects, the antisense strand comprises 19 nucleotide that is at least 65% complementary to the nucleic acid sequence of SEQ ID NOs: 201-410. In some aspects, the antisense strand comprises 19 nucleotide that is at least 70% complementary to the nucleic acid sequence of SEQ ID NOs: 201-410. In some aspects, the antisense strand comprises 19 nucleotide that is at least 75% complementary to the nucleic acid sequence of SEQ ID NOs: 201-410. In some aspects, the antisense strand comprises 19 nucleotide that is at least 80% complementary to the nucleic acid sequence of SEQ ID NOs: 201-410. In some aspects, the antisense strand comprises 19 nucleotide that is at least 85% complementary to the nucleic acid sequence of SEQ ID NOs: 201-410. In some aspects, the antisense strand comprises 19 nucleotide that is at least 90% complementary to the nucleic acid sequence of SEQ ID NOs: 201-410. In some aspects, the antisense strand comprises 19 nucleotide that is at least 95% complementary to the nucleic acid sequence of SEQ ID NOs: 201-410. In some aspects, the antisense strand comprises 19 nucleotide that is at least 96% complementary to the nucleic acid sequence of SEQ ID NOs: 201-410. In some aspects, the antisense strand comprises 19 nucleotide that is at least 97% complementary to the nucleic acid sequence of SEQ ID NOs: 201-410. In some aspects, the antisense strand comprises 19 nucleotide that is at least 98% complementary to the nucleic acid sequence of SEQ ID NOs: 201-410. In some aspects, the antisense strand comprises 19 nucleotide that is at least 99% complementary to the nucleic acid sequence of SEQ ID NOs: 201-410. In some aspects, the antisense strand comprises 19 nucleotide that is 100% complementary to the nucleic acid sequence of SEQ ID NOs: 201-410.

In some aspects, the antisense strand comprises a nucleic acid sequence that is 100% complementary to at least 13 contiguous nucleotides of the SEQ ID NOs: 201-410. In some aspects, the antisense strand comprises a nucleic acid sequence that is 100% complementary to at least 14 contiguous nucleotides of the SEQ ID NOs: 201-410. In some aspects, the antisense strand comprises a nucleic acid sequence that is 100% complementary to at least 15 contiguous nucleotides of the SEQ ID NOs: 201-410. In some aspects, the antisense strand comprises a nucleic acid sequence that is 100% complementary to at least 16 contiguous nucleotides of the SEQ ID NOs: 201-410. In some aspects, the antisense strand comprises a nucleic acid sequence that is 100% complementary to at least 17 contiguous nucleotides of the SEQ ID NOs: 201-410. In some aspects, the antisense strand comprises a nucleic acid sequence that is 100% complementary to at least 18 contiguous nucleotides of the SEQ ID NOs: 201-410. In some aspects, the antisense strand comprises a nucleic acid sequence that is 100% complementary to at least 19 contiguous nucleotides of the SEQ ID NOs: 201-410. In some aspects, the antisense strand comprises a nucleic acid sequence that is 100% complementary to at least 20 contiguous nucleotides of the SEQ ID NOs: 201-410.

In some aspects, the antisense strand comprises a nucleic acid sequence that has 1, 2, 3, 4 non-complementary nucleotides with respect to the nucleic acid sequence of SEQ ID NOs: 201-410. In some aspects, the antisense strand comprises a nucleic acid sequence that has less than 4 non-complementary nucleotides with respect to the nucleic acid sequence of SEQ ID NOs: 201-410. In some aspects, the antisense strand comprises a nucleic acid sequence that has less than 3 non-complementary nucleotides with respect to the nucleic acid sequence of SEQ ID NOs: 201-410. In some aspects, the antisense strand comprises a nucleic acid sequence that has less than 2 non-complementary nucleotides with respect to the nucleic acid sequence of SEQ ID NOs: 201-410.

In some aspects, the siRNA comprises a guide strand (antisense strand) having a sequence of any one of the sequences selected from SEQ ID NOs: 101-150, 621-830. In some aspects, the siRNA comprises a guide strand (antisense strand) having at least 60%, 65%, 70%, 75%, 80%, 85%, 90%, 91%, 92%, 93%, 94%, 95%, 96%, 97%, 98%, 99% sequence identity with any one of the sequences selected from SEQ ID NOs: 101-150, 621-830.

In some aspects, the siRNA comprises a guide strand (antisense strand) having a sequence that is 100% identical to at least 13, 14, 15, 16, 17, 18, 19 or 20 contiguous nucleotide residues of any one of the sequences selected from SEQ ID NOs: 101-150, 621-830. In some aspects, the siRNA comprises a guide strand (antisense strand) having a sequence with 100% identity to any one of the sequences selected from SEQ ID NOs: 101-150, 621-830.

In some aspects, the guide strand (antisense strand) comprises a nucleic acid sequence that has 1, 2, 3, 4 nucleotide mismatches with respect to the nucleic acid sequence of SEQ ID NOs: 101-150, 621-830. In some aspects, the guide strand (antisense strand) comprises a nucleic acid sequence that has less than 4 nucleotide mismatches with respect to the nucleic acid sequence of SEQ ID NOs: 101-150, 621-830. In some aspects, the guide strand (antisense strand) comprises a nucleic acid sequence that has less than 3 nucleotide mismatches with respect to the nucleic acid sequence of SEQ ID NOs: 101-150, 621-830. In some aspects, the guide strand (antisense strand) comprises a nucleic acid sequence that has less than 2 nucleotide mismatches with respect to the nucleic acid sequence of SEQ ID NOs: 101-150, 621-830.

In some aspects, the siRNA comprises a 21-23-mer duplex, wherein each strand comprises at least 21 nucleotide residues. In some aspects, each strand comprises 23 nucleotides (a 23-mer duplex). In some aspects, 21-mer duplex can comprise a stretch of 21 nucleotides in at least one strand, and a stretch of at least 21, or 22, or 23 nucleotides in the complementary strand of the double stranded siRNA. In some aspects, a 21-mer duplex comprises 21 nucleotides spanning the double-stranded region of the siRNA. In some aspects, the antisense strand comprises 21-23 nucleotides that is at least 60% complementary to the nucleic acid sequence of SEQ ID NOs: 51-100. In some aspects, the antisense strand comprises 21-23 nucleotides that is at least 65% complementary to the nucleic acid sequence of SEQ ID NOs: 51-100. In some aspects, the antisense strand comprises 21-23 nucleotides that is at least 70% complementary to the nucleic acid sequence of SEQ ID NOs: 51-100. In some aspects, the antisense strand comprises 21-23 nucleotides that is at least 75% complementary to the nucleic acid sequence of SEQ ID NOs: 51-100. In some aspects, the antisense strand comprises 21-23 nucleotides that is at least 80% complementary to the nucleic acid sequence of SEQ ID NOs: 51-100. In some aspects, the antisense strand comprises 21-23 nucleotides that is at least 85% complementary to the nucleic acid sequence of SEQ ID NOs: 51-100. In some aspects, the antisense strand comprises 21-23 nucleotides that is at least 90% complementary to the nucleic acid sequence of SEQ ID NOs: 51-100. In some aspects, the antisense strand comprises 21-23 nucleotides that is at least 95% complementary to the nucleic acid sequence of SEQ ID NOs: 51-100. In some aspects, the antisense strand comprises 21-23 nucleotides that is at least 96% complementary to the nucleic acid sequence of SEQ ID NOs: 51-100. In some aspects, the antisense strand comprises 21-23 nucleotides that is at least 97% complementary to the nucleic acid sequence of SEQ ID NOs: 51-100. In some aspects, the antisense strand comprises 21-23 nucleotides that is at least 98% complementary to the nucleic acid sequence of SEQ ID NOs: 51-100. In some aspects, the antisense strand comprises 21-23 nucleotides that is at least 99% complementary to the nucleic acid sequence of SEQ ID NOs: 51-100. In some aspects, the antisense strand comprises 21-23 nucleotides that is 100% complementary to the nucleic acid sequence of SEQ ID NOs: 51-100.

In some aspects, the antisense strand comprises a nucleic acid sequence that is 100% complementary to at least 13 contiguous nucleotides of the SEQ ID NOs: 51-100. In some aspects, the antisense strand comprises a nucleic acid sequence that is 100% complementary to at least 14 contiguous nucleotides of the SEQ ID NOs: 51-100. In some aspects, the antisense strand comprises a nucleic acid sequence that is 100% complementary to at least 15 contiguous nucleotides of the SEQ ID NOs: 51-100. In some aspects, the antisense strand comprises a nucleic acid sequence that is 100% complementary to at least 16 contiguous nucleotides of the SEQ ID NOs: 51-100. In some aspects, the antisense strand comprises a nucleic acid sequence that is 100% complementary to at least 17 contiguous nucleotides of the SEQ ID NOs: 51-100. In some aspects, the antisense strand comprises a nucleic acid sequence that is 100% complementary to at least 18 contiguous nucleotides of the SEQ ID NOs: 51-100. In some aspects, the antisense strand comprises a nucleic acid sequence that is 100% complementary to at least 19 contiguous nucleotides of the SEQ ID NOs: 51-100. In some aspects, the antisense strand comprises a nucleic acid sequence that is 100% complementary to at least 20 contiguous nucleotides of the SEQ ID NOs: 51-100.

In some aspects, the antisense strand comprises a nucleic acid sequence that has 1, 2, 3, 4 non-complementary nucleotides with respect to the nucleic acid sequence of SEQ ID NOs: 51-100. In some aspects, the antisense strand comprises a nucleic acid sequence that has less than 4 non-complementary nucleotides with respect to the nucleic acid sequence of SEQ ID NOs: 51-100. In some aspects, the antisense strand comprises a nucleic acid sequence that has less than 3 non-complementary nucleotides with respect to the nucleic acid sequence of SEQ ID NOs: 51-100. In some aspects, the antisense strand comprises a nucleic acid sequence that has less than 2 non-complementary nucleotides with respect to the nucleic acid sequence of SEQ ID NOs: 51-100. In some aspects, the antisense strand comprises at least 15, 16, 17, 18, 19 consecutive nucleotides complementary to a nucleic acid sequence of SEQ ID NOs: 1-50 or 101-150, differing by no more than 0, 1, 2, 3, 4 nucleotides.

In some aspects, the siRNA comprises an antisense strand having a sequence of any one of the sequences selected from SEQ ID NOs: 101-150, 621-830. In some aspects, the siRNA comprises a antisense strand having at least 60%, 65%, 70%, 75%, 80%, 85%, 90%, 91%, 92%, 93%, 94%, 95%, 96%, 97%, 98%, 99% sequence of any one of the sequences selected from SEQ ID NOs: 101-150, 621-830.

In some aspects, the siRNA comprises an antisense strand having a sequence of any one of the sequences selected from SEQ ID NOs: 151-200, 831-1040. In some aspects, the siRNA comprises a antisense strand having at least 60%, 65%, 70%, 75%, 80%, 85%, 90%, 91%, 92%, 93%, 94%, 95%, 96%, 97%, 98%, 99% sequence of any one of the sequences selected from SEQ ID NOs: 151-200, 831-1040.

In some aspects, the siRNA comprises an antisense strand having a sequence that is 100% identical to at least 13, 14, 15, 16, 17, 18, 19 or 20 contiguous nucleotide residues of any one of the sequences selected from SEQ ID NOs: 101-150, 621-830, 151-200, 831-1040. In some aspects, the siRNA comprises a antisense strand having a sequence with 100% identity to any one of the sequences selected from SEQ ID NOs: 101-150, 621-830, 151-200, 831-1040.

In some aspects, the antisense strand comprises a nucleic acid sequence that has 1, 2, 3, 4 nucleotide mismatches with respect to the nucleic acid sequence of SEQ ID NOs: 101-150, 621-830, 151-200, 831-1040. In some aspects, the antisense strand comprises a nucleic acid sequence that has less than 4 nucleotide mismatches with respect to the nucleic acid sequence of SEQ ID NOs: 101-150, 621-830, 151-200, 831-1040. In some aspects, the antisense strand comprises a nucleic acid sequence that has less than 3 nucleotide mismatches with respect to the nucleic acid sequence of SEQ ID NOs: 101-150, 621-830, 151-200, 831-1040. In some aspects, the antisense strand comprises a nucleic acid sequence that has less than 2 nucleotide mismatches with respect to the nucleic acid sequence of SEQ ID NOs: 101-150, 621-830, 151-200, 831-1040. In some aspects, the antisense strand comprises at least 15, 16, 17, 18, 19 consecutive nucleotides of a nucleic acid sequence of SEQ ID NOs: 101-150 or 151-200, differing by no more than 0, 1, 2, 3, 4 nucleotides. In some aspects, the antisense strand comprises at least 15, 16, 17, 18, 19 consecutive nucleotides of a nucleic acid sequence of SEQ ID NOs: 101-150, 621-830, 151-200, 831-1040, differing by no more than 0, 1, 2, 3, 4 nucleotides.

In some aspects, the siRNA comprises an antisense strand having a sequence of any one of the sequences selected from SEQ ID NOs: 1561-1820, SEQ ID NOs: 1821-2080. In some aspects, the siRNA comprises a passenger strand having at least 60%, 65%, 70%, 75%, 80%, 85%, 90%, 91%, 92%, 93%, 94%, 95%, 96%, 97%, 98%, 99% sequence identity of any one of the sequences selected from SEQ ID NOs: 1561-1820, SEQ ID NOs: 1821-2080.

In some aspects, the siRNA comprises an antisense strand having a sequence that is 100% identical to at least 13, 14, 15, 16, 17, 18, 19 or 20 contiguous nucleotide residues of any one of the sequences selected from SEQ ID NOs: 1561-1820, SEQ ID NOs: 1821-2080. In some aspects, the siRNA comprises a antisense strand having a sequence with 100% identity to any one of the sequences selected from SEQ ID NOs: 1561-1820, SEQ ID NOs: 1821-2080.

In some aspects, the antisense strand comprises a nucleic acid sequence that has 1, 2, 3, 4 nucleotide mismatches with respect to the nucleic acid sequence of SEQ ID NOs: 1561-1820, SEQ ID NOs: 1821-2080. In some aspects, the antisense strand comprises a nucleic acid sequence that has less than 4 nucleotide mismatches with respect to the nucleic acid sequence of SEQ ID NOs: 1561-1820, SEQ ID NOs: 1821-2080. In some aspects, the antisense strand comprises a nucleic acid sequence that has less than 3 nucleotide mismatches with respect to the nucleic acid sequence of SEQ ID NOs: 1561-1820, SEQ ID NOs: 1821-2080. In some aspects, the antisense strand comprises a nucleic acid sequence that has less than 2 nucleotide mismatches with respect to the nucleic acid sequence of SEQ ID NOs: 1561-1820, SEQ ID NOs: 1821-2080. In some aspects, the antisense strand comprises at least 15, 16, 17, 18, 19 consecutive nucleotides of a nucleic acid sequence of SEQ ID NOs: 1561-1820, SEQ ID NOs: 1821-2080, differing by no more than 0, 1, 2, 3, 4 nucleotides. In some aspects, methods include administering to a subject in need thereof a therapeutically effective amount of an oligonucleotide (polynucleic acid molecule) that includes a sense strand comprising at least 13, 14, or 15 contiguous nucleotides differing by no more than 1, 2, or 3 nucleotides from any one of SEQ ID NOs: 21, 71, 267, 477, 1321, 1417, 2021, and 2197. In some aspects, methods include administering to a subject in need thereof a therapeutically effective amount of an oligonucleotide (polynucleic acid molecule) that includes a sense strand comprising a nucleic acid sequence having at least 80%, at least 85%, at least 90%, at least 95%, or 100% identical to any one of SEQ ID NOs: 21, 71, 267, 477, 1321, 1417, 2021, and 2197. In some aspects, methods include administering to a subject in need thereof a therapeutically effective amount of an oligonucleotide (polynucleic acid molecule) that includes an antisense strand comprising a nucleic acid sequence having at least 80%, at least 85%, at least 90%, at least 95%, or 100% identical to any one of SEQ ID NOs: 121, 171, 687, 897, 1581, 1677, 1841, and 1937. In some aspects, methods include administering to a subject in need thereof a therapeutically effective amount of an oligonucleotide (polynucleic acid molecule) that includes an antisense strand comprising at least 13, 14, or 15 contiguous nucleotides differing by no more than 1, 2, or 3 nucleotides from to any one of SEQ ID NOs: 121, 171, 687, 897, 1581, 1677, 1841, and 1937.

Tables 1-3 disclose the sequences discussed in the above section. Each sequence is presented in a 5′-3′ read direction. Table 1: An antisense strand base can pair (at least partially) with a sense strand shown at the adjacent right hand column in the same row forming a siRNA duplex. Table 2: A passenger RNA strand base can pair (at least partially) with a guide strand shown at the adjacent right hand column in the same row forming a siRNA duplex. Table 3: A guide strand in the left hand column base can pair (at least partially) with a passenger strand shown at the adjacent right hand column in the same row forming a siRNA duplex.

TABLE 1

| | Sense strand_19 mer | | |
|---|---|---|---|
| Antisense (Guide) | (Passenger strand) | Antisense (Guide) | Sense (Target) strand 21- |
| strand_19 mer | or Target 19-mer | strand 21_23 mer | 23 mer (Passenger strand) |
| AGACGATCATACTT | TCTCCAAGTATGATC | AGACGATCATACTTG | GCTCTCCAAGTATGAT |
| GGAGA (SEQ ID NO: | GTCT (SEQ ID NO: 1) | GAGAGCAT (SEQ ID | CGTCT (SEQ ID NO: 51) |
| 101) | | NO: 151) | |
| TGGACGATCTTGTTC | TGCAGAACAAGATC | TGGACGATCTTGTTCT | TCTGCAGAACAAGATC |
| TGCA (SEQ ID NO: | GTCCA (SEQ ID NO: | GCAGACG (SEQ ID NO: | GTCCA (SEQ ID NO: 52) |
| 102) | 2) | 152) | |
| GGCATTGGCAGAAA | CCACTTTTCTGCCAA | GGCATTGGCAGAAAA | GTCCACTTTTCTGCCA |
| AGTGG (SEQ ID NO: | TGCC (SEQ ID NO: 3) | GTGGACGA (SEQ ID | ATGCC (SEQ ID NO: 53) |
| 103) | | NO: 153) | |
| AACCCGCACTGGGA | ACGGCTCCCAGTGC | AACCCGCACTGGGAG | CCACGGCTCCCAGTGC |
| GCCGT (SEQ ID NO: | GGGTT (SEQ ID NO: | CCGTGGCT (SEQ ID | GGGTT (SEQ ID NO: 54) |
| 104) | 4) | NO: 154) | |
| CTCAATGCCAATCTC | CACGGAGATTGGCA | CTCAATGCCAATCTCC | AACACGGAGATTGGCA |
| CGTG (SEQ ID NO: | TTGAG (SEQ ID NO: | GTGTTCC (SEQ ID NO: | TTGAG (SEQ ID NO: 55) |
| 105) | 5) | 155) | |
| TCTCAATGCCAATCT | ACGGAGATTGGCAT | TCTCAATGCCAATCTC | ACACGGAGATTGGCAT |
| CCGT (SEQ ID NO: | TGAGA (SEQ ID NO: | CGTGTTC (SEQ ID NO: | TGAGA (SEQ ID NO: 56) |
| 106) | 6) | 156) | |
| TGGCAATGTCATCTT | AGAGAAGATGACAT | TGGCAATGTCATCTTC | GGAGAGAAGATGACA |
| CTCT (SEQ ID NO: | TGCCA (SEQ ID NO: | TCTCCGG (SEQ ID NO: | TTGCCA (SEQ ID NO: |
| 107) | 7) | 157) | 57) |
| AAAACTCTCATGCC | CCAGTGGCATGAGA | AAAACTCTCATGCCA | AACCAGTGGCATGAGA |
| ACTGG (SEQ ID NO: | GTTTT (SEQ ID NO: 8) | CTGGTTAC (SEQ ID | GTTTT (SEQ ID NO: 58) |
| 108) | | NO: 158) | |
| GCCTCACCAGAGGC | TGCAGGCCTCTGGT | GCCTCACCAGAGGCC | CATGCAGGCCTCTGGT |
| CTGCA (SEQ ID NO: | GAGGC (SEQ ID NO: | TGCATGTC (SEQ ID | GAGGC (SEQ ID NO: 59) |
| 109) | 9) | NO: 159) | |
| ACAGTACACGGCCT | TGGTGAGGCCGTGT | ACAGTACACGGCCTC | TCTGGTGAGGCCGTGT |
| CACCA (SEQ ID NO: | ACTGT (SEQ ID NO: | ACCAGAGG (SEQ ID | ACTGT (SEQ ID NO: 60) |
| 110) | 10) | NO: 160) | |
| TGATCTTGGCGTGG | CGGGCCCACGCCAA | TGATCTTGGCGTGGG | CCCGGGCCCACGCCAA |
| GCCCG (SEQ ID NO: | GATCA (SEQ ID NO: | CCCGGGTG (SEQ ID | GATCA (SEQ ID NO: 61) |
| 111) | 11) | NO: 161) | |
| TCTATGGACTTGATC | CCAAGATCAAGTCC | TCTATGGACTTGATCT | CGCCAAGATCAAGTCC |
| TTGG (SEQ ID NO: | ATAGA (SEQ ID NO: | TGGCGTG (SEQ ID NO: | ATAGA (SEQ ID NO: 62) |
| 112) | 12) | 162) | |
| CACCAATGATATGC | GTTGGGCATATCATT | CACCAATGATATGCC | GTGTTGGGCATATCAT |
| CCAAC (SEQ ID NO: | GGTG (SEQ ID NO: 13) | CAACACAA (SEQ ID | TGGTG (SEQ ID NO: 63) |
| 113) | | NO: 163) | |
| AGCAACCACAGCAC | CATTGGTGCTGTGGT | AGCAACCACAGCACC | ATCATTGGTGCTGTGG |
| CAATG (SEQ ID NO: | TGCT (SEQ ID NO: 14) | AATGATAT (SEQ ID | TTGCT (SEQ ID NO: 64) |
| 114) | | NO: 164) | |
| TCGAACCACAATCC | AAACCGGATTGTGG | TCGAACCACAATCCG | GCAAACCGGATTGTGG |
| GGTTT (SEQ ID NO: | TTCGA (SEQ ID NO: | GTTTGCTG (SEQ ID | TTCGA (SEQ ID NO: 65) |
| 115) | 15) | NO: 165) | |
| CACTCGAACCACAA | CCGGATTGTGGTTCG | CACTCGAACCACAAT | AACCGGATTGTGGTTC |
| TCCGG (SEQ ID NO: | AGTG (SEQ ID NO: 16) | CCGGTTTG (SEQ ID | GAGTG (SEQ ID NO: 66) |
| 116) | | NO: 166) | |
| CTTCACTCGAACCA | GATTGTGGTTCGAGT | CTTCACTCGAACCAC | CGGATTGTGGTTCGAG |
| CAATC (SEQ ID NO: | GAAG (SEQ ID NO: | AATCCGGT (SEQ ID | TGAAG (SEQ ID NO: 67) |
| 117) | 17) | NO: 167) | |
| GTCCTCATCACGGTC | GCTGGACCGTGATG | GTCCTCATCACGGTCC | ATGCTGGACCGTGATG |
| CAGC (SEQ ID NO: | AGGAC (SEQ ID NO: | AGCATGC (SEQ ID NO: | AGGAC (SEQ ID NO: 68) |
| 118) | 18) | 168) | |
| AGTTATCAGCATGT | TGAGGACATGCTGA | AGTTATCAGCATGTCC | GATGAGGACATGCTGA |
| CCTCA (SEQ ID NO: | TAACT (SEQ ID NO: | TCATCAC (SEQ ID NO: | TAACT (SEQ ID NO: 69) |
| 119) | 19) | 169) | |
| GAAGCCAACCTTGT | CAGATACAAGGTTG | GAAGCCAACCTTGTA | GCCAGATACAAGGTTG |
| ATCTG (SEQ ID NO: | GCTTC (SEQ ID NO: | TCTGGCCA (SEQ ID | GCTTC (70) |
| 120) | 20) | NO: 170) | |
| TTCCATAATACTCTG | CTCTCAGAGTATTAT | TTCCATAATACTCTGA | CTCTCTCAGAGTATTA |
| AGAG (SEQ ID NO: | GGAA (SEQ ID NO: | GAGAGAT (SEQ ID NO: | TGGAA (SEQ ID NO: 71) |
| 121) | 21) | 171) | |
| CCGTGTTGGAGGGA | AACCTTCCCTCCAAC | CCGTGTTGGAGGGAA | CCAACCTTCCCTCCAA |
| AGGTT (SEQ ID NO: | ACGG (SEQ ID NO: | GGTTGGTT (SEQ ID | CACGG (SEQ ID NO: 72) |
| 122) | 22) | NO: 172) | |
| GATACTGAGAGCTT | CTAGCAAGCTCTCA | GATACTGAGAGCTTG | GCCTAGCAAGCTCTCA |
| GCTAG (SEQ ID NO: | GTATC (SEQ ID NO: | CTAGGCAT (SEQ ID | GTATC (SEQ ID NO: 73) |
| 123) | 23) | NO: 173) | |
| CATGATACTGAGAG | GCAAGCTCTCAGTA | CATGATACTGAGAGC | TAGCAAGCTCTCAGTA |
| CTTGC (SEQ ID NO: | TCATG (SEQ ID NO: | TTGCTAGG (SEQ ID | TCATG (SEQ ID NO: 74) |
| 124) | 24) | NO: 174) | |
| CTTCCGAGCATGAT | TCAGTATCATGCTCG | CTTCCGAGCATGATA | TCTCAGTATCATGCTC |
| ACTGA (SEQ ID NO: | GAAG (SEQ ID NO: | CTGAGAGC (SEQ ID | GGAAG (SEQ ID NO: 75) |
| 125) | 25) | NO: 175) | |
| CTTATTCCAAACTTG | CCACCAAGTTTGGA | CTTATTCCAAACTTGG | TCCCACCAAGTTTGGA |
| GTGG (SEQ ID NO: | ATAAG (SEQ ID NO: | TGGGAAT (SEQ ID NO: | ATAAG (SEQ ID NO: 76) |
| 126) | 26) | 176) | |
| ATGACAATATCTGT | TCCGCACAGATATT | ATGACAATATCTGTG | CCTCCGCACAGATATT |
| GCGGA (SEQ ID NO: | GTCAT (SEQ ID NO: | CGGAGGTT (SEQ ID | GTCAT (SEQ ID NO: 77) |
| 127) | 27) | NO: 177) | |
| GCCAAATGCCGGGA | CAAGATCCCGGCAT | GCCAAATGCCGGGAT | TACAAGATCCCGGCAT |
| TCTTG (SEQ ID NO: | TTGGC (SEQ ID NO: | CTTGTAGG (SEQ ID | TTGGC (SEQ ID NO: |
| 128) | 28) | NO: 178) | 78) |
| ACGTTATTACCTGTG | AGCACACAGGTAAT | ACGTTATTACCTGTGT | TCAGCACACAGGTAAT |
| TGCT (SEQ ID NO: | AACGT (SEQ ID NO: | GCTGAGC (SEQ ID NO: | AACGT(SEQ ID NO: 79) |
| 129) | 29) | 179) | |
| GACCCTCACAGACC | ACCCTGGTCTGTGA | GACCCTCACAGACCA | AAACCCTGGTCTGTGA |
| AGGGT (SEQ ID NO: | GGGTC (SEQ ID NO: | GGGTTTGC (SEQ ID | GGGTC (SEQ ID NO: 80) |
| 130) | 30) | NO: 180) | |
| ATAGATCCATGTTCT | CCACAGAACATGGA | ATAGATCCATGTTCTG | TACCACAGAACATGGA |
| GTGG (SEQ ID NO: | TCTAT (SEQ ID NO: | TGGTATG (SEQ ID NO: | TCTAT(SEQ ID NO: 81) |
| 131) | 31) | 181) | |
| GACTTTAATAGATC | ACATGGATCTATTA | GACTTTAATAGATCC | GAACATGGATCTATTA |
| CATGT (SEQ ID NO: | AAGTC (SEQ ID NO: | ATGTTCTG (SEQ ID | AAGTC (SEQ ID NO: 82) |
| 132) | 32) | NO: 182) | |
| GTGACTTTAATAGA | ATGGATCTATTAAA | GTGACTTTAATAGATC | ACATGGATCTATTAAA |
| TCCAT (SEQ ID NO: | GTCAC (SEQ ID NO: | CATGTTC (SEQ ID NO: | GTCAC (SEQ ID NO: 83) |
| 133) | 33) | 183) | |
| GAATAGCACAAACC | GGAAGGGTTTGTGC | GAATAGCACAAACCC | CGGGAAGGGTTTGTGC |
| CTTCC (SEQ ID NO: | TATTC (SEQ ID NO: | TTCCCGAC (SEQ ID | TATTC (SEQ ID NO: 84) |
| 134) | 34) | NO: 184) | |
| GGAATAGCACAAAC | GAAGGGTTTGTGCT | GGAATAGCACAAACC | GGGAAGGGTTTGTGCT |
| CCTTC (SEQ ID NO: | ATTCC (SEQ ID NO: | CTTCCCGA (SEQ ID | ATTCC (SEQ ID NO: 85) |
| 135) | 35) | NO: 185) | |
| GACACCATCAGAAC | CTCAAGTTCTGATGG | GACACCATCAGAACT | ACCTCAAGTTCTGATG |
| TTGAG (SEQ ID NO: | TGTC (SEQ ID NO: 36) | TGAGGTTA (SEQ ID | GTGTC (SEQ ID NO: 86) |
| 136) | | NO: 186) | |
| AGACACCATCAGAA | TCAAGTTCTGATGGT | AGACACCATCAGAAC | CCTCAAGTTCTGATGG |
| CTTGA (SEQ ID NO: | GTCT (SEQ ID NO: 37) | TTGAGGTT (SEQ ID | TGTCT (SEQ ID NO: 87) |
| 137) | | NO: 187) | |
| GCTTCTAGAGGTTTG | CCCACAAACCTCTA | GCTTCTAGAGGTTTGT | TTCCCACAAACCTCTA |
| TGGG (SEQ ID NO: | GAAGC (SEQ ID NO: | GGGAATC (SEQ ID NO: | GAAGC (SEQ ID NO: 88) |
| 138) | 38) | 188) | |
| AAGCTTCTAGAGGT | CACAAACCTCTAGA | AAGCTTCTAGAGGTTT | CCCACAAACCTCTAGA |
| TTGTG (SEQ ID NO: | AGCTT (SEQ ID NO: | GTGGGAA (SEQ ID NO: | AGCTT ((SEQ ID NO: 89) |
| 139) | 39) | 189) | |
| CTGTTCATTGGTTTG | CCTTCAAACCAATG | CTGTTCATTGGTTTGA | GGCCTTCAAACCAATG |
| AAGG (SEQ ID NO: | AACAG (SEQ ID NO: | AGGCCAG (SEQ ID NO: | AACAG (SEQ ID NO: 90) |
| 140) | 40) | 190) | |
| TTATGCTTTGCTGTT | AATGAACAGCAAAG | TTATGCTTTGCTGTTC | CCAATGAACAGCAAAG |
| CATT (SEQ ID NO: | CATAA (SEQ ID NO: | ATTGGTT (SEQ ID NO: | CATAA (SEQ ID NO: 91) |
| 141) | 41) | 191) | |
| AATTAACCTTGAATT | AACAAATTCAAGGT | AATTAACCTTGAATTT | GAAACAAATTCAAGGT |
| TGTT (SEQ ID NO: | TAATT (SEQ ID NO: | GTTTCAT (SEQ ID NO: | TAATT (SEQ ID NO: 92) |
| 142) | 42) | 192) | |
| ATCTTGCTTTATGCA | AAGCTGCATAAAGC | ATCTTGCTTTATGCAG | TGAAGCTGCATAAAGC |
| GCTT (SEQ ID NO: | AAGAT (SEQ ID NO: | CTTCACA (SEQ ID NO: | AAGAT (SEQ ID NO: 93) |
| 143) | 43) | 193) | |
| AGTAATCTTGCTTTA | TGCATAAAGCAAGA | AGTAATCTTGCTTTAT | GCTGCATAAAGCAAGA |
| TGCA (SEQ ID NO: | TTACT (SEQ ID NO: | GCAGCTT (SEQ ID NO: | TTACT (SEQ ID NO: 94) |
| 144) | 44) | 194) | |
| AAGATTAAACATAA | AAAGATTATGTTTA | AAGATTAAACATAAT | AAAAAGATTATGTTTA |
| TCTTT (SEQ ID NO: | ATCTT (SEQ ID NO: | CTTTTTTG (SEQ ID | ATCTT (SEQ ID NO: 95) |
| 145) | 45) | NO: 195) | |
| AGTAAGAAAACCAA | AAGGCTTGGTTTTCT | AGTAAGAAAACCAAG | CTAAGGCTTGGTTTTCT |
| GCCTT (SEQ ID NO: | TACT (SEQ ID NO: 46) | CCTTAGAT (SEQ ID | TACT (SEQ ID NO: 96) |
| 146) | | NO: 196) | |
| ATATGACAGTAAGA | GGTTTTCTTACTGTC | ATATGACAGTAAGAA | TTGGTTTTCTTACTGTC |
| AAACC (SEQ ID NO: | ATAT (SEQ ID NO: 47) | AACCAAGC (SEQ ID | ATAT (SEQ ID NO: 97) |
| 147) | | NO: 197) | |
| ACCAACCGCAGAAA | TCAAGTTTCTGCGGT | ACCAACCGCAGAAAC | CCTCAAGTTTCTGCGG |
| CTTGA (SEQ ID NO: | TGGT (SEQ ID NO: 48) | TTGAGGTG (SEQ ID | TTGGT (SEQ ID NO: 98) |
| 148) | | NO: 198) | |
| TACCAACCGCAGAA | CAAGTTTCTGCGGTT | TACCAACCGCAGAAA | CTCAAGTTTCTGCGGT |
| ACTTG (SEQ ID NO: | GGTA (SEQ ID NO: 49) | CTTGAGGT (SEQ ID | TGGTA (SEQ ID NO: 99) |
| 149) | | NO: 199) | |
| ACATCAAGCACCAG | TTGGACTGGTGCTTG | ACATCAAGCACCAGT | AGTTGGACTGGTGCTT |
| TCCAA (SEQ ID NO: | ATGT (SEQ ID NO: 50) | CCAACTAT (SEQ ID | GATGT (SEQ ID NO: 100) |
| 150) | | NO: 200) | |
| CATTCACTTGTCTTC | TTTGGAAGACAAGT | CATTCACTTGTCTTCC | GATTTGGAAGACAAGT |
| CAAA (SEQ ID NO: | GAATG (SEQ ID NO: | AAATCCC (SEQ ID NO: | GAATG (SEQ ID NO: |
| 621) | 201) | 831) | 411) |
| CATACTTGGAGAGC | GTGATGCTCTCCAA | CATACTTGGAGAGCA | CAGTGATGCTCTCCAA |
| ATCAC (SEQ ID NO: | GTATG (SEQ ID NO: | TCACTGTG (SEQ ID | GTATG (SEQ ID NO: 412) |
| 622) | 202) | NO: 832) | |
| ACTCGAACCACAAT | ACCGGATTGTGGTTC | ACTCGAACCACAATC | AAACCGGATTGTGGTT |
| CCGGT (SEQ ID NO: | GAGT (SEQ ID NO: | CGGTTTGC (SEQ ID | CGAGT (SEQ ID NO: |
| 623) | 203) | NO: 833) | 413) |
| TCTTTACCAACCGCA | TTTCTGCGGTTGGTA | TCTTTACCAACCGCAG | AGTTTCTGCGGTTGGT |
| GAAA (SEQ ID NO: | AAGA (SEQ ID NO: | AAACTTG (SEQ ID NO: | AAAGA (SEQ ID NO: |
| 624) | 204) | 834) | 414) |
| ATGGCAAAGAAGAT | CTTCTATCTTCTTTG | ATGGCAAAGAAGATA | TGCTTCTATCTTCTTTG |
| AGAAG (SEQ ID NO: | CCAT (SEQ ID NO: | GAAGCAGC (SEQ ID | CCAT (SEQ ID NO: 415) |
| 625) | 205) | NO: 835) | |
| CATGTTCTGTGGTAT | GAACATACCACAGA | CATGTTCTGTGGTATG | AGGAACATACCACAGA |
| GTTC (SEQ ID NO: | ACATG(SEQ ID NO: | TTCCTCC (SEQ ID NO: | ACATG (SEQ ID NO: |
| 626) | 206) | 836) | 416) |
| CTTCAGCTCAGGTCC | TTATGGACCTGAGCT | CTTCAGCTCAGGTCCA | TTTTATGGACCTGAGC |
| ATAA (SEQ ID NO: | GAAG (SEQ ID NO: | TAAAAGG (SEQ ID NO: | TGAAG (SEQ ID NO: |
| 627) | 207) | 837) | 417) |
| CTTGATCTTGGCGTG | GGCCCACGCCAAGA | CTTGATCTTGGCGTGG | CGGGCCCACGCCAAGA |
| GGCC (SEQ ID NO: | TCAAG (SEQ ID NO: | GCCCGGG (SEQ ID NO: | TCAAG (SEQ ID NO: |
| 628) | 208) | 838) | 418) |
| GGGAATAGCACAAA | AAGGGTTTGTGCTAT | GGGAATAGCACAAAC | GGAAGGGTTTGTGCTA |
| CCCTT (SEQ ID NO: | TCCC (SEQ ID NO: | CCTTCCCG (SEQ ID | TTCCC (SEQ ID NO: 419) |
| 629) | 209) | NO: 839) | |
| TGCAGTAAAATGGA | TGTGATCCATTTTAC | TGCAGTAAAATGGAT | CCTGTGATCCATTTTAC |
| TCACA (SEQ ID NO: | TGCA (SEQ ID NO: | CACAGGAA (SEQ ID | TGCA (SEQ ID NO: 420) |
| 630) | 210) | NO: 840) | |
| GCTCAATAATTGAG | ACCAACTCAATTATT | GCTCAATAATTGAGTT | CAACCAACTCAATTAT |
| TTGGT (SEQ ID NO: | GAGC (SEQ ID NO: | GGTTGGA (SEQ ID NO: | TGAGC (SEQ ID NO: |
| 631) | 211) | 841) | 421) |
| AGCCTTAGATAGCT | TCTGCAGCTATCTAA | AGCCTTAGATAGCTG | GATCTGCAGCTATCTA |
| GCAGA (SEQ ID NO: | GGCT (SEQ ID NO: | CAGATCCT (SEQ ID | AGGCT (SEQ ID NO: |
| 632) | 212) | NO: 842) | 422) |
| ACAACATTATCTGCT | CCGAAGCAGATAAT | ACAACATTATCTGCTT | TTCCGAAGCAGATAAT |
| TCGG (SEQ ID NO: | GTTGT (SEQ ID NO: | CGGAAAA (SEQ ID NO: | GTTGT (SEQ ID NO: 423) |
| 633) | 213) | 843) | |
| TTCCGGAGCAGTGT | TGTACACACTGCTCC | TTCCGGAGCAGTGTG | TATGTACACACTGCTC |
| GTACA (SEQ ID NO: | GGAA (SEQ ID NO: | TACATACT (SEQ ID | CGGAA (SEQ ID NO: |
| 634) | 214) | NO: 844) | 424) |
| GCCAAAAGGGTTGT | CAGAGACAACCCTT | GCCAAAAGGGTTGTC | TCCAGAGACAACCCTT |
| CTCTG (SEQ ID NO: | TTGGC (SEQ ID NO: | TCTGGATC (SEQ ID | TTGGC (SEQ ID NO: 425) |
| 635) | 215) | NO: 845) | |
| GGATGCTGCCAAAT | CCGGCATTTGGCAG | GGATGCTGCCAAATG | TCCCGGCATTTGGCAG |
| GCCGG (SEQ ID NO: | CATCC (SEQ ID NO: | CCGGGATC (SEQ ID | CATCC (SEQ ID NO: 426) |
| 636) | 216) | NO: 846) | |
| TATGCCCAACACAA | GTTACTTGTGTTGGG | TATGCCCAACACAAG | AGGTTACTTGTGTTGG |
| GTAAC (SEQ ID NO: | CATA (SEQ ID NO: | TAACCTTA (SEQ ID | GCATA (SEQ ID NO: |
| 637) | 217) | NO: 847) | 427) |
| CTTGCTAGGCATTCT | GGGAAGAATGCCTA | CTTGCTAGGCATTCTT | CTGGGAAGAATGCCTA |
| TCCC (SEQ ID NO: | GCAAG (SEQ ID NO: | CCCAGCA (SEQ ID NO: | GCAAG (SEQ ID NO: |
| 638) | 218) | 848) | 428) |
| TGCATTCACTTGTCT | TGGAAGACAAGTGA | TGCATTCACTTGTCTT | TTTGGAAGACAAGTGA |
| TCCA (SEQ ID NO: | ATGCA (SEQ ID NO: | CCAAATC (SEQ ID NO: | ATGCA (SEQ ID NO: |
| 639) | 219) | 849) | 429) |
| AGAGTAATCTTGCTT | CATAAAGCAAGATT | AGAGTAATCTTGCTTT | TGCATAAAGCAAGATT |
| TATG (SEQ ID NO: | ACTCT (SEQ ID NO: | ATGCAGC (SEQ ID NO: | ACTCT (SEQ ID NO: 430) |
| 640) | 220) | 850) | |
| AGCTTATTCCAAACT | ACCAAGTTTGGAAT | AGCTTATTCCAAACTT | CCACCAAGTTTGGAAT |
| TGGT (SEQ ID NO: | AAGCT (SEQ ID NO: | GGTGGGA (SEQ ID NO: | AAGCT (SEQ ID NO: |
| 641) | 221) | 851) | 431) |
| ACAATTCTCCTTGTT | GTTCAACAAGGAGA | ACAATTCTCCTTGTTG | AAGTTCAACAAGGAGA |
| GAAC (SEQ ID NO: | ATTGT (SEQ ID NO: | AACTTGT (SEQ ID NO: | ATTGT (SEQ ID NO: 432) |
| 642) | 222) | 852) | |
| CTGGATCTGCATTTT | GAGAAAAATGCAGA | CTGGATCTGCATTTTT | TGGAGAAAAATGCAG |
| TCTC (SEQ ID NO: | TCCAG (SEQ ID NO: | CTCCACC (SEQ ID NO: | ATCCAG (SEQ ID NO: |
| 643) | 223) | 853) | 433) |
| ATGATACTGAGAGC | AGCAAGCTCTCAGT | ATGATACTGAGAGCT | CTAGCAAGCTCTCAGT |
| TTGCT (SEQ ID NO: | ATCAT (SEQ ID NO: | TGCTAGGC (SEQ ID | ATCAT (SEQ ID NO: 434) |
| 644) | 224) | NO: 854) | |
| TACATACTCATGAC | GCATCGTCATGAGT | TACATACTCATGACG | TGGCATCGTCATGAGT |
| GATGC (SEQ ID NO: | ATGTA (SEQ ID NO: | ATGCCAGG (SEQ ID | ATGTA (SEQ ID NO: 435) |
| 645) | 225) | NO: 855) | |
| GCAACATGGTGCAA | GCTCCTTGCACCATG | GCAACATGGTGCAAG | CTGCTCCTTGCACCAT |
| GGAGC (SEQ ID NO: | TTGC (SEQ ID NO: | GAGCAGAT (SEQ ID | GTTGC (SEQ ID NO: 436) |
| 646) | 226) | NO: 856) | |
| CTCAATAATTGAGTT | AACCAACTCAATTA | CTCAATAATTGAGTTG | CCAACCAACTCAATTA |
| GGTT (SEQ ID NO: | TTGAG (SEQ ID NO: | GTTGGAT (SEQ ID NO: | TTGAG (SEQ ID NO: 437) |
| 647) | 227) | 857) | |
| ATGATATGCCCAAC | CTTGTGTTGGGCATA | ATGATATGCCCAACA | TACTTGTGTTGGGCAT |
| ACAAG (SEQ ID NO: | TCAT (SEQ ID NO: | CAAGTAAC (SEQ ID | ATCAT (SEQ ID NO: 438) |
| 648) | 228) | NO: 858) | |
| CAACACAAGTAACC | GATAAGGTTACTTGT | CAACACAAGTAACCT | AGGATAAGGTTACTTG |
| TTATC (SEQ ID NO: | GTTG (SEQ ID NO: | TATCCTTC (SEQ ID | TGTTG (SEQ ID NO: 439) |
| 649) | 229) | NO: 859) | |
| GAGCATCACTGTGC | GGCTTGCACAGTGA | GAGCATCACTGTGCA | GGGGCTTGCACAGTGA |
| AAGCC (SEQ ID NO: | TGCTC (SEQ ID NO: | AGCCCCGC (SEQ ID | TGCTC (SEQ ID NO: 440) |
| 650) | 230) | NO: 860) | |
| TTAACCTTGAATTTG | GAAACAAATTCAAG | TTAACCTTGAATTTGT | ATGAAACAAATTCAAG |
| TTTC (SEQ ID NO: | GTTAA (SEQ ID NO: | TTCATTC (SEQ ID NO: | GTTAA (SEQ ID NO: 441) |
| 651) | 231) | 861) | |
| GACAGTCCAAGATC | TTTGTGATCTTGGAC | GACAGTCCAAGATCA | TCTTTGTGATCTTGGAC |
| ACAAA (SEQ ID NO: | TGTC (SEQ ID NO: | CAAAGATA (SEQ ID | TGTC (SEQ ID NO: 442) |
| 652) | 232) | NO: 862) | |
| TGGATCTGCATTTTT | GGAGAAAAATGCAG | TGGATCTGCATTTTTC | GTGGAGAAAAATGCA |
| CTCC (SEQ ID NO: | ATCCA (SEQ ID NO: | TCCACCA (SEQ ID NO: | GATCCA (SEQ ID NO: |
| 653) | 233) | 863) | 443) |
| CCCTGACACAACAT | AGATAATGTTGTGTC | CCCTGACACAACATT | GCAGATAATGTTGTGT |
| TATCT (SEQ ID NO: | AGGG (SEQ ID NO: | ATCTGCTT (SEQ ID | CAGGG (SEQ ID NO: |
| 654) | 234) | NO: 864) | 444) |
| GCTTATTCCAAACTT | CACCAAGTTTGGAA | GCTTATTCCAAACTTG | CCCACCAAGTTTGGAA |
| GGTG (SEQ ID NO: | TAAGC (SEQ ID NO: | GTGGGAA (SEQ ID NO: | TAAGC (SEQ ID NO: |
| 655) | 235) | 865) | 445) |
| AGATTCAAGGTTAT | AAAGCATAACCTTG | AGATTCAAGGTTATG | GCAAAGCATAACCTTG |
| GCTTT (SEQ ID NO: | AATCT (SEQ ID NO: | CTTTGCTG (SEQ ID | AATCT (SEQ ID NO: 446) |
| 656) | 236) | NO: 866) | |
| CTTCACGTTATTACC | CACAGGTAATAACG | CTTCACGTTATTACCT | CACACAGGTAATAACG |
| TGTG (SEQ ID NO: | TGAAG (SEQ ID NO: | GTGTGCT (SEQ ID NO: | TGAAG (SEQ ID NO: |
| 657) | 237) | 867) | 447) |
| TCAATTGTGATAAT | CAGCCATTATCACA | TCAATTGTGATAATG | ACCAGCCATTATCACA |
| GGCTG (SEQ ID NO: | ATTGA (SEQ ID NO: | GCTGGTAG (SEQ ID | ATTGA (SEQ ID NO: 448) |
| 658) | 238) | NO: 868) | |
| TTGAGTTGGTTGGAT | AAAAATCCAACCAA | TTGAGTTGGTTGGATT | ACAAAAATCCAACCAA |
| TTTT (SEQ ID NO: | CTCAA (SEQ ID NO: | TTTGTAT (SEQ ID NO: | CTCAA (SEQ ID NO: 449) |
| 659) | 239) | 869) | |
| TCGTCTTGGTGCTTC | ATAGGAAGCACCAA | TCGTCTTGGTGCTTCC | GAATAGGAAGCACCA |
| CTAT (SEQ ID NO: | GACGA (SEQ ID NO: | TATTCCT (SEQ ID NO: | AGACGA (SEQ ID NO: |
| 660) | 240) | 870) | 450) |
| TGTCCATTGAGGTC | GCGCTGACCTCAAT | TGTCCATTGAGGTCA | CAGCGCTGACCTCAAT |
| AGCGC (SEQ ID NO: | GGACA (SEQ ID NO: | GCGCTGAC (SEQ ID | GGACA (SEQ ID NO: |
| 661) | 241) | NO: 871) | 451) |
| ATGTCATCTTCTCTC | CCCGGAGAGAAGAT | ATGTCATCTTCTCTCC | CTCCCGGAGAGAAGAT |
| CGGG (SEQ ID NO: | GACAT (SEQ ID NO: | GGGAGGC (SEQ ID NO: | GACAT (SEQ ID NO: |
| 662) | 242) | 872) | 452) |
| CACCTGTCCAATATC | CATTGATATTGGAC | CACCTGTCCAATATCA | GCCATTGATATTGGAC |
| AATG (SEQ ID NO: | AGGTG (SEQ ID NO: | ATGGCAG (SEQ ID NO: | AGGTG (SEQ ID NO: |
| 663) | 243) | 873) | 453) |
| TCACGTTATTACCTG | CACACAGGTAATAA | TCACGTTATTACCTGT | AGCACACAGGTAATAA |
| TGTG (SEQ ID NO: | CGTGA (SEQ ID NO: | GTGCTGA (SEQ ID NO: | CGTGA (SEQ ID NO: |
| 664) | 244) | 874) | 454) |
| TGCATATTCACCATT | CCTAAATGGTGAAT | TGCATATTCACCATTT | TGCCTAAATGGTGAAT |
| TAGG (SEQ ID NO: | ATGCA (SEQ ID NO: | AGGCATA (SEQ ID NO: | ATGCA (SEQ ID NO: |
| 665) | 245) | 875) | 455) |
| TTCTATAAAACCCA | GCCACTGGGTTTTAT | TTCTATAAAACCCAGT | CTGCCACTGGGTTTTA |
| GTGGC (SEQ ID NO: | AGAA (SEQ ID NO: | GGCAGAC (SEQ ID NO: | TAGAA (SEQ ID NO: |
| 666) | 246) | 876) | 456) |
| CTGATTCCGGAGCA | CACACTGCTCCGGA | CTGATTCCGGAGCAG | TACACACTGCTCCGGA |
| GTGTG (SEQ ID NO: | ATCAG (SEQ ID NO: | TGTGTACA (SEQ ID | ATCAG (SEQ ID NO: |
| 667) | 247) | NO: 877) | 457) |
| GATATGCCCAACAC | TACTTGTGTTGGGCA | GATATGCCCAACACA | GTTACTTGTGTTGGGC |
| AAGTA (SEQ ID NO: | TATC (SEQ ID NO: | AGTAACCT (SEQ ID | ATATC (SEQ ID NO: 458) |
| 668) | 248) | NO: 878) | |
| TCGTCACAGTACAC | AGGCCGTGTACTGT | TCGTCACAGTACACG | TGAGGCCGTGTACTGT |
| GGCCT (SEQ ID NO: | GACGA(SEQ ID NO: | GCCTCACC (SEQ ID | GACGA (SEQ ID NO: |
| 669) | 249) | NO: 879) | 459) |
| TGATACTGAGAGCT | TAGCAAGCTCTCAG | TGATACTGAGAGCTT | CCTAGCAAGCTCTCAG |
| TGCTA (SEQ ID NO: | TATCA (SEQ ID NO: | GCTAGGCA (SEQ ID | TATCA (SEQ ID NO: 460) |
| 670) | 250) | NO: 880) | |
| TGAACTTCATCTCAA | GGCATTGAGATGAA | TGAACTTCATCTCAAT | TTGGCATTGAGATGAA |
| TGCC (SEQ ID NO: | GTTCA (SEQ ID NO: | GCCAATC (SEQ ID NO: | GTTCA (SEQ ID NO: 461) |
| 671) | 251) | 881) | |
| GTAGCCCAGATTGG | AACACCCAATCTGG | GTAGCCCAGATTGGG | AGAACACCCAATCTGG |
| GTGTT (SEQ ID NO: | GCTAC (SEQ ID NO: | TGTTCTAT (SEQ ID | GCTAC (SEQ ID NO: 462) |
| 672) | 252) | NO: 882) | |
| TCAGCGGAAATGAA | TTTGTTTCATTTCCG | TCAGCGGAAATGAAA | TGTTTGTTTCATTTCCG |
| ACAAA (SEQ ID NO: | CTGA (SEQ ID NO: | CAAACAAA (SEQ ID | CTGA (SEQ ID NO: 463) |
| 673) | 253) | NO: 883) | |
| CCCATAGCTGAAGT | CCACTACTTCAGCTA | CCCATAGCTGAAGTA | TTCCACTACTTCAGCT |
| AGTGG (SEQ ID NO: | TGGG (SEQ ID NO: | GTGGAAGG (SEQ ID | ATGGG (SEQ ID NO: |
| 674) | 254) | NO: 884) | 464) |
| TAGATCCATGTTCTG | ACCACAGAACATGG | TAGATCCATGTTCTGT | ATACCACAGAACATGG |
| TGGT (SEQ ID NO: | ATCTA (SEQ ID NO: | GGTATGT (SEQ ID NO: | ATCTA (SEQ ID NO: 465) |
| 675) | 255) | 885) | |
| ATATCTGTGCGGAG | AGAACCTCCGCACA | ATATCTGTGCGGAGG | TAAGAACCTCCGCACA |
| GTTCT (SEQ ID NO: | GATAT (SEQ ID NO: | TTCTTATG (SEQ ID | GATAT (SEQ ID NO: 466) |
| 676) | 256) | NO: 886) | |
| CAAATGCCGGGATC | TACAAGATCCCGGC | CAAATGCCGGGATCT | CCTACAAGATCCCGGC |
| TTGTA (SEQ ID NO: | ATTTG (SEQ ID NO: | TGTAGGTG (SEQ ID | ATTTG (SEQ ID NO: 467) |
| 677) | 257) | NO: 887) | |
| CCGGAGCAGTGTGT | TATGTACACACTGCT | CCGGAGCAGTGTGTA | AGTATGTACACACTGC |
| ACATA (SEQ ID NO: | CCGG (SEQ ID NO: | CATACTCA (SEQ ID | TCCGG (SEQ ID NO: 468) |
| 678) | 258) | NO: 888) | |
| CTGCAACATGGTGC | TCCTTGCACCATGTT | CTGCAACATGGTGCA | GCTCCTTGCACCATGT |
| AAGGA (SEQ ID NO: | GCAG (SEQ ID NO: | AGGAGCAG (SEQ ID | TGCAG (SEQ ID NO: |
| 679) | 259) | NO: 889) | 469) |
| TTCTAGGTGGAAAT | AAGTAATTTCCACCT | TTCTAGGTGGAAATT | CAAAGTAATTTCCACC |
| TACTT (SEQ ID NO: | AGAA (SEQ ID NO: | ACTTTGGC (SEQ ID | TAGAA (SEQ ID NO: |
| 680) | 260) | NO: 890) | 470) |
| CCCGCACTGGGAGC | CCACGGCTCCCAGT | CCCGCACTGGGAGCC | AGCCACGGCTCCCAGT |
| CGTGG (SEQ ID NO: | GCGGG (SEQ ID NO: | GTGGCTTT (SEQ ID | GCGGG (SEQ ID NO: |
| 681) | 261) | NO: 891) | 471) |
| AAACCCAGTGGCAG | CTTGTCTGCCACTGG | AAACCCAGTGGCAGA | AGCTTGTCTGCCACTG |
| ACAAG (SEQ ID NO: | GTTT (SEQ ID NO: | CAAGCTCA (SEQ ID | GGTTT (SEQ ID NO: 472) |
| 682) | 262) | NO: 892) | |
| TTATTAGTGACATCA | TGCTTGATGTCACTA | TTATTAGTGACATCAA | GGTGCTTGATGTCACT |
| AGCA (SEQ ID NO: | ATAA (SEQ ID NO: | GCACCAG (SEQ ID NO: | AATAA (SEQ ID NO: |
| 683) | 263) | 893) | 473) |
| CACGGATGGCATCT | ATCAAAGATGCCAT | CACGGATGGCATCTTT | CCATCAAAGATGCCAT |
| TTGAT (SEQ ID NO: | CCGTG (SEQ ID NO: | GATGGCA (SEQ ID NO: | CCGTG (SEQ ID NO: 474) |
| 684) | 264) | 894) | |
| CAGACGATCATACT | CTCCAAGTATGATC | CAGACGATCATACTT | CTCTCCAAGTATGATC |
| TGGAG (SEQ ID NO: | GTCTG (SEQ ID NO: | GGAGAGCA (SEQ ID | GTCTG (SEQ ID NO: 475) |
| 685) | 265) | NO: 895) | |
| GTGGTATGTTCCTCC | AGCAGGAGGAACAT | GTGGTATGTTCCTCCT | GGAGCAGGAGGAACA |
| TGCT (SEQ ID NO: | ACCAC (SEQ ID NO: | GCTCCAT (SEQ ID NO: | TACCAC (SEQ ID NO: |
| 686) | 266) | 896) | 476) |
| CCATAATACTCTGA | CTCTCTCAGAGTATT | CCATAATACTCTGAG | ATCTCTCTCAGAGTAT |
| GAGAG (SEQ ID NO: | ATGG (SEQ ID NO: | AGAGATCC (SEQ ID | TATGG (SEQ ID NO: 477) |
| 687) | 267) | NO: 897) | |
| GTCAACCTCACTCTT | TCGGAAGAGTGAGG | GTCAACCTCACTCTTC | GCTCGGAAGAGTGAGG |
| CCGA (SEQ ID NO: | TTGAC (SEQ ID NO: | CGAGCAT (SEQ ID NO: | TTGAC (SEQ ID NO: 478) |
| 688) | 268) | 898) | |
| GTCCATTGAGGTCA | AGCGCTGACCTCAA | GTCCATTGAGGTCAG | TCAGCGCTGACCTCAA |
| GCGCT (SEQ ID NO: | TGGAC (SEQ ID NO: | CGCTGACA (SEQ ID | TGGAC (SEQ ID NO: |
| 689) | 269) | NO: 899) | 479) |
| GCAGTTGTCCATGT | ATTCCACATGGACA | GCAGTTGTCCATGTG | TTATTCCACATGGACA |
| GGAAT (SEQ ID NO: | ACTGC (SEQ ID NO: | GAATAAAG (SEQ ID | ACTGC (SEQ ID NO: 480) |
| 690) | 270) | NO: 900) | |
| TGCATAGATGGCCT | CAAGAAGGCCATCT | TGCATAGATGGCCTTC | AACAAGAAGGCCATCT |
| TCTTG (SEQ ID NO: | ATGCA (SEQ ID NO: | TTGTTGG (SEQ ID NO: | ATGCA (SEQ ID NO: |
| 691) | 271) | 901) | 481) |
| CAATTGTGATAATG | CCAGCCATTATCAC | CAATTGTGATAATGG | TACCAGCCATTATCAC |
| GCTGG (SEQ ID NO: | AATTG (SEQ ID NO: | CTGGTAGT (SEQ ID | AATTG (SEQ ID NO: 482) |
| 692) | 272) | NO: 902) | |
| CAGACAAGCTCACT | GACACAGTGAGCTT | CAGACAAGCTCACTG | TGGACACAGTGAGCTT |
| GTGTC (SEQ ID NO: | GTCTG (SEQ ID NO: | TGTCCATG (SEQ ID | GTCTG (SEQ ID NO: 483) |
| 693) | 273) | NO: 903) | |
| CAATATCTGTGCGG | AACCTCCGCACAGA | CAATATCTGTGCGGA | AGAACCTCCGCACAGA |
| AGGTT (SEQ ID NO: | TATTG (SEQ ID NO: | GGTTCTTA (SEQ ID | TATTG (SEQ ID NO: 484) |
| 694) | 274) | NO: 904) | |
| ACCCAGTGGCAGAC | AGCTTGTCTGCCACT | ACCCAGTGGCAGACA | TGAGCTTGTCTGCCAC |
| AAGCT (SEQ ID NO: | GGGT (SEQ ID NO: | AGCTCACT (SEQ ID | TGGGT (SEQ ID NO: 485) |
| 695) | 275) | NO: 905) | |
| TTGGGCAGGAAGCT | TGCTAAGCTTCCTGC | TTGGGCAGGAAGCTT | GTTGCTAAGCTTCCTG |
| TAGCA (SEQ ID NO: | CCAA (SEQ ID NO: | AGCAACAG (SEQ ID | CCCAA (SEQ ID NO: |
| 696) | 276) | NO: 906) | 486) |
| GGTTGGTTTTGCACA | CGGCTGTGCAAAAC | GGTTGGTTTTGCACAG | GGCGGCTGTGCAAAAC |
| GCCG (SEQ ID NO: | CAACC (SEQ ID NO: | CCGCCCA (SEQ ID NO: | CAACC (SEQ ID NO: |
| 697) | 277) | 907) | 487) |
| GTACATACTCATGA | CATCGTCATGAGTAT | GTACATACTCATGAC | GGCATCGTCATGAGTA |
| CGATG (SEQ ID NO: | GTAC (SEQ ID NO: | GATGCCAG (SEQ ID | TGTAC (SEQ ID NO: 488) |
| 698) | 278) | NO: 908) | |
| TTTGGGCAGGAAGC | GCTAAGCTTCCTGCC | TTTGGGCAGGAAGCT | TTGCTAAGCTTCCTGC |
| TTAGC (SEQ ID NO: | CAAA (SEQ ID NO: | TAGCAACA (SEQ ID | CCAAA (SEQ ID NO: |
| 699) | 279) | NO: 909) | 489) |
| CATGCCACTGGTTA | CAAGGTAACCAGTG | CATGCCACTGGTTACC | GCCAAGGTAACCAGTG |
| CCTTG (SEQ ID NO: | GCATG (SEQ ID NO: | TTGGCAA (SEQ ID NO: | GCATG (SEQ ID NO: |
| 700) | 280) | 910) | 490) |
| GTGTCAGCAACCAC | GTGCTGTGGTTGCTG | GTGTCAGCAACCACA | TGGTGCTGTGGTTGCT |
| AGCAC (SEQ ID NO: | ACAC (SEQ ID NO: | GCACCAAT (SEQ ID | GACAC (SEQ ID NO: |
| 701) | 281) | NO: 911) | 491) |
| TCTCCACCACCTTTC | GGCAGAAAGGTGGT | TCTCCACCACCTTTCT | ATGGCAGAAAGGTGGT |
| TGCC (SEQ ID NO: | GGAGA (SEQ ID NO: | GCCATTC (SEQ ID NO: | GGAGA (SEQ ID NO: |
| 702) | 282) | 912) | 492) |
| AGCCTCGTCTTGGTG | GAAGCACCAAGACG | AGCCTCGTCTTGGTGC | AGGAAGCACCAAGAC |
| CTTC (SEQ ID NO: | AGGCT (SEQ ID NO: | TTCCTAT (SEQ ID NO: | GAGGCT (SEQ ID NO: |
| 703) | 283) | 913) | 493) |
| GGGTTCCCTGAGTT | AGACTAACTCAGGG | GGGTTCCCTGAGTTA | TGAGACTAACTCAGGG |
| AGTCT (SEQ ID NO: | AACCC (SEQ ID NO: | GTCTCAAA (SEQ ID | AACCC (SEQ ID NO: |
| 704) | 284) | NO: 914) | 494) |
| CCCTCACAGACCAG | AAACCCTGGTCTGT | CCCTCACAGACCAGG | GCAAACCCTGGTCTGT |
| GGTTT (SEQ ID NO: | GAGGG (SEQ ID NO: | GTTTGCAG (SEQ ID | GAGGG (SEQ ID NO: |
| 705) | 285) | NO: 915) | 495) |
| TCATGGTGTTCTGTG | TCTACACAGAACAC | TCATGGTGTTCTGTGT | TGTCTACACAGAACAC |
| TAGA (SEQ ID NO: | CATGA (SEQ ID NO: | AGACACA (SEQ ID NO: | CATGA (SEQ ID NO: |
| 706) | 286) | 916) | 496) |
| TCATAGGTGATTTTC | GGGTGAAAATCACC | TCATAGGTGATTTTCA | AGGGGTGAAAATCACC |
| ACCC (SEQ ID NO: | TATGA (SEQ ID NO: | CCCCTTG (SEQ ID NO: | TATGA (SEQ ID NO: 497) |
| 707) | 287) | 917) | |
| GTCCAATATCAATG | CCTGCCATTGATATT | GTCCAATATCAATGG | ACCCTGCCATTGATAT |
| GCAGG (SEQ ID NO: | GGAC (SEQ ID NO: | CAGGGTTT (SEQ ID | TGGAC (SEQ ID NO: |
| 708) | 288) | NO: 918) | 498) |
| GGTTTAGACTGGAG | GTTGGCTCCAGTCTA | GGTTTAGACTGGAGC | ATGTTGGCTCCAGTCT |
| CCAAC (SEQ ID NO: | AACC (SEQ ID NO: | CAACATCC (SEQ ID | AAACC (SEQ ID NO: |
| 709) | 289) | NO: 919) | 499) |
| CACCAGTTATCAGC | GACATGCTGATAAC | CACCAGTTATCAGCA | AGGACATGCTGATAAC |
| ATGTC (SEQ ID NO: | TGGTG (SEQ ID NO: | TGTCCTCA (SEQ ID | TGGTG (SEQ ID NO: 500) |
| 710) | 290) | NO: 920) | |
| TTCAGCTCAGGTCC | TTTATGGACCTGAGC | TTCAGCTCAGGTCCAT | CTTTTATGGACCTGAG |
| ATAAA (SEQ ID NO: | TGAA (SEQ ID NO: | AAAAGGA (SEQ ID NO: | CTGAA (SEQ ID NO: |
| 711) | 291) | 921) | 501) |
| CGAGCATGATACTG | GCTCTCAGTATCATG | CGAGCATGATACTGA | AAGCTCTCAGTATCAT |
| AGAGC (SEQ ID NO: | CTCG (SEQ ID NO: | GAGCTTGC (SEQ ID | GCTCG (SEQ ID NO: 502) |
| 712) | 292) | NO: 922) | |
| GTAACTGGAGTTTTC | ACCTGAAAACTCCA | GTAACTGGAGTTTTCA | TGACCTGAAAACTCCA |
| AGGT (SEQ ID NO: | GTTAC (SEQ ID NO: | GGTCATA (SEQ ID NO: | GTTAC (SEQ ID NO: 503) |
| 713) | 293) | 923) | |
| TCAAGCACCAGTCC | TAGTTGGACTGGTG | TCAAGCACCAGTCCA | TATAGTTGGACTGGTG |
| AACTA (SEQ ID NO: | CTTGA (SEQ ID NO: | ACTATATC (SEQ ID | CTTGA (SEQ ID NO: 504) |
| 714) | 294) | NO: 924) | |
| ATCTTGGCGTGGGC | CCCGGGCCCACGCC | ATCTTGGCGTGGGCC | CACCCGGGCCCACGCC |
| CCGGG (SEQ ID NO: | AAGAT (SEQ ID NO: | CGGGTGCT (SEQ ID | AAGAT (SEQ ID NO: |
| 715) | 295) | NO: 925) | 505) |
| TTTACCAACCGCAG | AGTTTCTGCGGTTGG | TTTACCAACCGCAGA | CAAGTTTCTGCGGTTG |
| AAACT (SEQ ID NO: | TAAA (SEQ ID NO: | AACTTGAG (SEQ ID | GTAAA (SEQ ID NO: |
| 716) | 296) | NO: 926) | 506) |
| ATTCACTTGTCTTCC | ATTTGGAAGACAAG | ATTCACTTGTCTTCCA | GGATTTGGAAGACAAG |
| AAAT (SEQ ID NO: | TGAAT (SEQ ID NO: | AATCCCA (SEQ ID NO: | TGAAT (SEQ ID NO: 507) |
| 717) | 297) | 927) | |
| CAGATTGGGTGTTCT | TTATAGAACACCCA | CAGATTGGGTGTTCTA | TTTTATAGAACACCCA |
| ATAA (SEQ ID NO: | ATCTG (SEQ ID NO: | TAAAACC (SEQ ID NO: | ATCTG (SEQ ID NO: 508) |
| 718) | 298) | 928) | |
| ATACTTGGAGAGCA | AGTGATGCTCTCCA | ATACTTGGAGAGCAT | ACAGTGATGCTCTCCA |
| TCACT (SEQ ID NO: | AGTAT (SEQ ID NO: | CACTGTGC (SEQ ID | AGTAT (SEQ ID NO: 509) |
| 719) | 299) | NO: 929) | |
| TTCTGGTTGAAGTGT | TGACACACTTCAAC | TTCTGGTTGAAGTGTG | CCTGACACACTTCAAC |
| GTCA (SEQ ID NO: | CAGAA (SEQ ID NO: | TCAGGTC (SEQ ID NO: | CAGAA (SEQ ID NO: |
| 720) | 300) | 930) | 510) |
| CTCCACAGCCGAGC | ACCAAGCTCGGCTG | CTCCACAGCCGAGCT | GAACCAAGCTCGGCTG |
| TTGGT (SEQ ID NO: | TGGAG (SEQ ID NO: | TGGTTCCA (SEQ ID | TGGAG (SEQ ID NO: |
| 721) | 301) | NO: 931) | 511) |
| ATTATCTGCTTCGGA | GTTTTCCGAAGCAG | ATTATCTGCTTCGGAA | GGGTTTTCCGAAGCAG |
| AAAC (SEQ ID NO: | ATAAT (SEQ ID NO: | AACCCCT (SEQ ID NO: | ATAAT (SEQ ID NO: 512) |
| 722) | 302) | 932) | |
| GCAGACGATCATAC | TCCAAGTATGATCGT | GCAGACGATCATACT | TCTCCAAGTATGATCG |
| TTGGA (SEQ ID NO: | CTGC (SEQ ID NO: | TGGAGAGC (SEQ ID | TCTGC (SEQ ID NO: 513) |
| 723) | 303) | NO: 933) | |
| GCTTGCTAGGCATTC | GGAAGAATGCCTAG | GCTTGCTAGGCATTCT | TGGGAAGAATGCCTAG |
| TTCC (SEQ ID NO: | CAAGC (SEQ ID NO: | TCCCAGC (SEQ ID NO: | CAAGC (SEQ ID NO: |
| 724) | 304) | 934) | 514) |
| CAGGCATTGGCAGA | ACTTTTCTGCCAATG | CAGGCATTGGCAGAA | CCACTTTTCTGCCAAT |
| AAAGT (SEQ ID NO: | CCTG (SEQ ID NO: | AAGTGGAC (SEQ ID | GCCTG (SEQ ID NO: 515) |
| 725) | 305) | NO: 935) | |
| TCATAGGAAACAGC | AATATGCTGTTTCCT | TCATAGGAAACAGCA | AGAATATGCTGTTTCC |
| ATATT (SEQ ID NO: | ATGA (SEQ ID NO: | TATTCTTG (SEQ ID | TATGA (SEQ ID NO: 516) |
| 726) | 306) | NO: 936) | |
| TCCACAGCCGAGCT | AACCAAGCTCGGCT | TCCACAGCCGAGCTT | GGAACCAAGCTCGGCT |
| TGGTT (SEQ ID NO: | GTGGA (SEQ ID NO: | GGTTCCAC (SEQ ID | GTGGA (SEQ ID NO: |
| 727) | 307) | NO: 937) | 517) |
| GTGTTGGGCACAGT | CTAACACTGTGCCC | GTGTTGGGCACAGTG | CACTAACACTGTGCCC |
| GTTAG (SEQ ID NO: | AACAC (SEQ ID NO: | TTAGTGCT (SEQ ID | AACAC (SEQ ID NO: |
| 728) | 308) | NO: 938) | 518) |
| CCCAACATTTTTGCA | TTGTTGCAAAAATGT | CCCAACATTTTTGCAA | CTTTGTTGCAAAAATG |
| ACAA (SEQ ID NO: | TGGG (SEQ ID NO: | CAAAGCT (SEQ ID NO: | TTGGG (SEQ ID NO: 519) |
| 729) | 309) | 939) | |
| ATCAGCGGAAATGA | TTGTTTCATTTCCGC | ATCAGCGGAAATGAA | GTTTGTTTCATTTCCGC |
| AACAA (SEQ ID NO: | TGAT (SEQ ID NO: | ACAAACAA (SEQ ID | TGAT (SEQ ID NO: 520) |
| 730) | 310) | NO: 940) | |
| TGGCATCTTTGATGG | TTTGCCATCAAAGAT | TGGCATCTTTGATGGC | TCTTTGCCATCAAAGA |
| CAAA (SEQ ID NO: | GCCA (SEQ ID NO: | AAAGAAG (SEQ ID NO: | TGCCA (SEQ ID NO: 521) |
| 731) | 311) | 941) | |
| AGTCTCAAAGCTGT | GGGCTACAGCTTTG | AGTCTCAAAGCTGTA | CTGGGCTACAGCTTTG |
| AGCCC (SEQ ID NO: | AGACT (SEQ ID NO: | GCCCAGAT (SEQ ID | AGACT (SEQ ID NO: |
| 732) | 312) | NO: 942) | 522) |
| TTCACGTTATTACCT | ACACAGGTAATAAC | TTCACGTTATTACCTG | GCACACAGGTAATAAC |
| GTGT (SEQ ID NO: | GTGAA (SEQ ID NO: | TGTGCTG (SEQ ID NO: | GTGAA (SEQ ID NO: |
| 733) | 313) | 943) | 523) |
| AATGATATGCCCAA | TTGTGTTGGGCATAT | AATGATATGCCCAAC | ACTTGTGTTGGGCATA |
| CACAA (SEQ ID NO: | CATT (SEQ ID NO: | ACAAGTAA (SEQ ID | TCATT (SEQ ID NO: 524) |
| 734) | 314) | NO: 944) | |
| GGTCTGTAGCCTGT | GCTGCACAGGCTAC | GGTCTGTAGCCTGTGC | CCGCTGCACAGGCTAC |
| GCAGC (SEQ ID NO: | AGACC (SEQ ID NO: | AGCGGCA (SEQ ID NO: | AGACC (SEQ ID NO: |
| 735) | 315) | 945) | 525) |
| TCTTGGTGCTTCCTA | GGAATAGGAAGCAC | TCTTGGTGCTTCCTAT | AAGGAATAGGAAGCA |
| TTCC (SEQ ID NO: | CAAGA (SEQ ID NO: | TCCTTCC (SEQ ID NO: | CCAAGA (SEQ ID NO: |
| 736) | 316) | 946) | 526) |
| TTACCAACCGCAGA | AAGTTTCTGCGGTTG | TTACCAACCGCAGAA | TCAAGTTTCTGCGGTT |
| AACTT (SEQ ID NO: | GTAA (SEQ ID NO: | ACTTGAGG (SEQ ID | GGTAA (SEQ ID NO: |
| 737) | 317) | NO: 947) | 527) |
| TTGGTGCTTCCTATT | AAGGAATAGGAAGC | TTGGTGCTTCCTATTC | GGAAGGAATAGGAAG |
| CCTT (SEQ ID NO: | ACCAA (SEQ ID NO: | CTTCCAC (SEQ ID NO: | CACCAA (SEQ ID NO: |
| 738) | 318) | 948) | 528) |
| GGTTTAAGCTTCTAG | ACCTCTAGAAGCTT | GGTTTAAGCTTCTAGA | AAACCTCTAGAAGCTT |
| AGGT (SEQ ID NO: | AAACC (SEQ ID NO: | GGTTTGT (SEQ ID NO: | AAACC (SEQ ID NO: |
| 739) | 319) | 949) | 529) |
| GTTTAGACTGGAGC | TGTTGGCTCCAGTCT | GTTTAGACTGGAGCC | GATGTTGGCTCCAGTC |
| CAACA (SEQ ID NO: | AAAC (SEQ ID NO: | AACATCCA (SEQ ID | TAAAC (SEQ ID NO: |
| 740) | 320) | NO: 950) | 530) |
| TTATAGCAGTTGTCC | ACATGGACAACTGC | TTATAGCAGTTGTCCA | CCACATGGACAACTGC |
| ATGT (SEQ ID NO: | TATAA (SEQ ID NO: | TGTGGAA (SEQ ID NO: | TATAA (SEQ ID NO: 531) |
| 741) | 321) | 951) | |
| ACCAGTTATCAGCA | GGACATGCTGATAA | ACCAGTTATCAGCAT | GAGGACATGCTGATAA |
| TGTCC (SEQ ID NO: | CTGGT (SEQ ID NO: | GTCCTCAT (SEQ ID | CTGGT (SEQ ID NO: 532) |
| 742) | 322) | NO: 952) | |
| CCGAGCATGATACT | CTCTCAGTATCATGC | CCGAGCATGATACTG | AGCTCTCAGTATCATG |
| GAGAG (SEQ ID NO: | TCGG (SEQ ID NO: | AGAGCTTG (SEQ ID | CTCGG (SEQ ID NO: 533) |
| 743) | 323) | NO: 953) | |
| GGTTATACAGGCTG | CTGGACAGCCTGTA | GGTTATACAGGCTGT | TACTGGACAGCCTGTA |
| TCCAG (SEQ ID NO: | TAACC (SEQ ID NO: | CCAGTAAG (SEQ ID | TAACC (SEQ ID NO: 534) |
| 744) | 324) | NO: 954) | |
| CTCGAACCACAATC | AACCGGATTGTGGT | CTCGAACCACAATCC | CAAACCGGATTGTGGT |
| CGGTT (SEQ ID NO: | TCGAG (SEQ ID NO: | GGTTTGCT (SEQ ID | TCGAG (SEQ ID NO: |
| 745) | 325) | NO: 955) | 535) |
| CAGTTGTCCATGTG | TATTCCACATGGAC | CAGTTGTCCATGTGG | TTTATTCCACATGGAC |
| GAATA (SEQ ID NO: | AACTG (SEQ ID NO: | AATAAAGC (SEQ ID | AACTG (SEQ ID NO: |
| 746) | 326) | NO: 956) | 536) |
| TTGGAACAGCAATG | TGCACCATTGCTGTT | TTGGAACAGCAATGG | ACTGCACCATTGCTGT |
| GTGCA (SEQ ID NO: | CCAA (SEQ ID NO: | TGCAGTGA (SEQ ID | TCCAA (SEQ ID NO: 537) |
| 747) | 327) | NO: 957) | |
| CCAAAAGGGTTGTC | CCAGAGACAACCCT | CCAAAAGGGTTGTCT | ATCCAGAGACAACCCT |
| TCTGG (SEQ ID NO: | TTTGG (SEQ ID NO: | CTGGATCT (SEQ ID | TTTGG (SEQ ID NO: 538) |
| 748) | 328) | NO: 958) | |
| ATCAAGCACCAGTC | AGTTGGACTGGTGC | ATCAAGCACCAGTCC | ATAGTTGGACTGGTGC |
| CAACT (SEQ ID NO: | TTGAT (SEQ ID NO: | AACTATAT (SEQ ID | TTGAT (SEQ ID NO: 539) |
| 749) | 329) | NO: 959) | |
| AGTATCTCCTCCGG | TGAGCCCGGAGGAG | AGTATCTCCTCCGGGC | GCTGAGCCCGGAGGAG |
| GCTCA (SEQ ID NO: | ATACT (SEQ ID NO: | TCAGCAG (SEQ ID NO: | ATACT (SEQ ID NO: 540) |
| 750) | 330) | 960) | |
| TCCAAACTTGGTGG | AATTCCCACCAAGTT | TCCAAACTTGGTGGG | ATAATTCCCACCAAGT |
| GAATT (SEQ ID NO: | TGGA (SEQ ID NO: | AATTATGC (SEQ ID | TTGGA (SEQ ID NO: 541) |
| 751) | 331) | NO: 961) | |
| TCAAGATGGTCTGA | GCTTGTCAGACCATC | TCAAGATGGTCTGAC | CGGCTTGTCAGACCAT |
| CAAGC (SEQ ID NO: | TTGA (SEQ ID NO: | AAGCCGCA (SEQ ID | CTTGA (SEQ ID NO: 542) |
| 752) | 332) | NO: 962) | |
| GCCTTAGATAGCTG | ATCTGCAGCTATCTA | GCCTTAGATAGCTGC | GGATCTGCAGCTATCT |
| CAGAT (SEQ ID NO: | AGGC (SEQ ID NO: | AGATCCTT (SEQ ID | AAGGC (SEQ ID NO: |
| 753) | 333) | NO: 963) | 543) |
| GATTCCGGAGCAGT | TACACACTGCTCCG | GATTCCGGAGCAGTG | TGTACACACTGCTCCG |
| GTGTA (SEQ ID NO: | GAATC (SEQ ID NO: | TGTACATA (SEQ ID | GAATC (SEQ ID NO: |
| 754) | 334) | NO: 964) | 544) |
| CGTGGCTTTTGGCA | AGAATTGCCAAAAG | CGTGGCTTTTGGCAAT | AGAGAATTGCCAAAAG |
| ATTCT (SEQ ID NO: | CCACG (SEQ ID NO: | TCTCTCC (SEQ ID NO: | CCACG (SEQ ID NO: |
| 755) | 335) | 965) | 545) |
| CTCTATGGAGAGCA | GATACTGCTCTCCAT | CTCTATGGAGAGCAG | GAGATACTGCTCTCCA |
| GTATC (SEQ ID NO: | AGAG (SEQ ID NO: | TATCTCCT (SEQ ID | TAGAG (SEQ ID NO: |
| 756) | 336) | NO: 966) | 546) |
| TCCTAAGAGACACT | CTGCCAGTGTCTCTT | TCCTAAGAGACACTG | ACCTGCCAGTGTCTCT |
| GGCAG (SEQ ID NO: | AGGA (SEQ ID NO: | GCAGGTAG (SEQ ID | TAGGA (SEQ ID NO: |
| 757) | 337) | NO: 967) | 547) |
| GGTACCTCACTCCTA | CTCTTAGGAGTGAG | GGTACCTCACTCCTAA | GTCTCTTAGGAGTGAG |
| AGAG (SEQ ID NO: | GTACC (SEQ ID NO: | GAGACAC (SEQ ID NO: | GTACC (SEQ ID NO: 548) |
| 758) | 338) | 968) | |
| GTAAAGTTGCACTG | TTCGCCAGTGCAACT | GTAAAGTTGCACTGG | CTTTCGCCAGTGCAAC |
| GCGAA (SEQ ID NO: | TTAC (SEQ ID NO: | CGAAAGTG (SEQ ID | TTTAC (SEQ ID NO: 549) |
| 759) | 339) | NO: 969) | |
| CACGTTATTACCTGT | GCACACAGGTAATA | CACGTTATTACCTGTG | CAGCACACAGGTAATA |
| GTGC (SEQ ID NO: | ACGTG (SEQ ID NO: | TGCTGAG (SEQ ID NO: | ACGTG (SEQ ID NO: |
| 760) | 340) | 970) | 550) |
| TGTCATCTTCTCTCC | TCCCGGAGAGAAGA | TGTCATCTTCTCTCCG | CCTCCCGGAGAGAAGA |
| GGGA (SEQ ID NO: | TGACA (SEQ ID NO: | GGAGGCC (SEQ ID NO: | TGACA (SEQ ID NO: |
| 761) | 341) | 971) | 551) |
| TTATCTGCTTCGGAA | GGTTTTCCGAAGCA | TTATCTGCTTCGGAAA | GGGGTTTTCCGAAGCA |
| AACC (SEQ ID NO: | GATAA (SEQ ID NO: | ACCCCTT (SEQ ID NO: | GATAA (SEQ ID NO: |
| 762) | 342) | 972) | 552) |
| AGTCTCCAGGTAGA | GCACTTCTACCTGGA | AGTCTCCAGGTAGAA | GAGCACTTCTACCTGG |
| AGTGC (SEQ ID NO: | GACT (SEQ ID NO: | GTGCTCTT (SEQ ID | AGACT (SEQ ID NO: |
| 763) | 343) | NO: 973) | 553) |
| AGGCCGTGTTGGAG | CTTCCCTCCAACACG | AGGCCGTGTTGGAGG | ACCTTCCCTCCAACAC |
| GGAAG (SEQ ID NO: | GCCT (SEQ ID NO: | GAAGGTTG (SEQ ID | GGCCT (SEQ ID NO: 554) |
| 764) | 344) | NO: 974) | |
| TCTCCACAGCCGAG | CCAAGCTCGGCTGT | TCTCCACAGCCGAGC | AACCAAGCTCGGCTGT |
| CTTGG (SEQ ID NO: | GGAGA (SEQ ID NO: | TTGGTTCC (SEQ ID | GGAGA (SEQ ID NO: |
| 765) | 345) | NO: 975) | 555) |
| TCTTCCGAGCATGAT | CAGTATCATGCTCG | TCTTCCGAGCATGATA | CTCAGTATCATGCTCG |
| ACTG (SEQ ID NO: | GAAGA (SEQ ID NO: | CTGAGAG (SEQ ID NO: | GAAGA (SEQ ID NO: |
| 766) | 346) | 976) | 556) |
| GAGCATGATACTGA | AGCTCTCAGTATCAT | GAGCATGATACTGAG | CAAGCTCTCAGTATCA |
| GAGCT (SEQ ID NO: | GCTC (SEQ ID NO: | AGCTTGCT (SEQ ID | TGCTC (SEQ ID NO: 557) |
| 767) | 347) | NO: 977) | |
| TCAATGGCAGGGTT | GTCTAAACCCTGCC | TCAATGGCAGGGTTT | CAGTCTAAACCCTGCC |
| TAGAC (SEQ ID NO: | ATTGA (SEQ ID NO: | AGACTGGA (SEQ ID | ATTGA (SEQ ID NO: 558) |
| 768) | 348) | NO: 978) | |
| CAGCCGGAGAGACA | TGAGCTGTCTCTCCG | CAGCCGGAGAGACAG | AATGAGCTGTCTCTCC |
| GCTCA (SEQ ID NO: | GCTG (SEQ ID NO: | CTCATTCT (SEQ ID | GGCTG (SEQ ID NO: |
| 769) | 349) | NO: 979) | 559) |
| TCATCACGGTCCAG | GCATGCTGGACCGT | TCATCACGGTCCAGC | ATGCATGCTGGACCGT |
| CATGC (SEQ ID NO: | GATGA (SEQ ID NO: | ATGCATCG (SEQ ID | GATGA (SEQ ID NO: |
| 770) | 350) | NO: 980) | 560) |
| TATCTGTGCGGAGG | AAGAACCTCCGCAC | TATCTGTGCGGAGGTT | ATAAGAACCTCCGCAC |
| TTCTT (SEQ ID NO: | AGATA(SEQ ID NO: | CTTATGA (SEQ ID NO: | AGATA (SEQ ID NO: |
| 771) | 351) | 981) | 561) |
| TTTCTAGGTGGAAA | AGTAATTTCCACCTA | TTTCTAGGTGGAAATT | AAAGTAATTTCCACCT |
| TTACT (SEQ ID NO: | GAAA (SEQ ID NO: | ACTTTGG (SEQ ID NO: | AGAAA (SEQ ID NO: |
| 772) | 352) | 982) | 562) |
| AATATCTGTGCGGA | GAACCTCCGCACAG | AATATCTGTGCGGAG | AAGAACCTCCGCACAG |
| GGTTC (SEQ ID NO: | ATATT (SEQ ID NO: | GTTCTTAT (SEQ ID | ATATT (SEQ ID NO: 563) |
| 773) | 353) | NO: 983) | |
| GCAGAAGGTTGGAT | TATAAATCCAACCTT | GCAGAAGGTTGGATT | TGTATAAATCCAACCT |
| TTATA (SEQ ID NO: | CTGC (SEQ ID NO: | TATACAGT (SEQ ID | TCTGC (SEQ ID NO: 564) |
| 774) | 354) | NO: 984) | |
| ATGAAACAAACAAA | CAGGGTTTGTTTGTT | ATGAAACAAACAAAC | TCCAGGGTTTGTTTGTT |
| CCCTG (SEQ ID NO: | TCAT (SEQ ID NO: | CCTGGAAC (SEQ ID | TCAT (SEQ ID NO: 565) |
| 775) | 355) | NO: 985) | |
| GTGGCTTTTGGCAAT | GAGAATTGCCAAAA | GTGGCTTTTGGCAATT | GAGAGAATTGCCAAAA |
| TCTC (SEQ ID NO: | GCCAC (SEQ ID NO: | CTCTCCT (SEQ ID NO: | GCCAC (SEQ ID NO: |
| 776) | 356) | 986) | 566) |
| CACAGTGTTAGTGC | GACAAGCACTAACA | CACAGTGTTAGTGCTT | GAGACAAGCACTAACA |
| TTGTC (SEQ ID NO: | CTGTG (SEQ ID NO: | GTCTCGC (SEQ ID NO: | CTGTG (SEQ ID NO: 567) |
| 777) | 357) | 987) | |
| ATGCTGCCAAATGC | TCCCGGCATTTGGCA | ATGCTGCCAAATGCC | GATCCCGGCATTTGGC |
| CGGGA (SEQ ID NO: | GCAT (SEQ ID NO: | GGGATCTT (SEQ ID | AGCAT (SEQ ID NO: |
| 778) | 358) | NO: 988) | 568) |
| AATGTCATCTTCTCT | CCGGAGAGAAGATG | AATGTCATCTTCTCTC | TCCCGGAGAGAAGATG |
| CCGG (SEQ ID NO: | ACATT (SEQ ID NO: | CGGGAGG (SEQ ID NO: | ACATT (SEQ ID NO: 569) |
| 779) | 359) | 989) | |
| ATTCCAAACTTGGT | TTCCCACCAAGTTTG | ATTCCAAACTTGGTG | AATTCCCACCAAGTTT |
| GGGAA (SEQ ID NO: | GAAT (SEQ ID NO: | GGAATTAT (SEQ ID | GGAAT (SEQ ID NO: |
| 780) | 360) | NO: 990) | 570) |
| TTTGGCTTCAAATGT | CTTTACATTTGAAGC | TTTGGCTTCAAATGTA | ATCTTTACATTTGAAG |
| AAAG (SEQ ID NO: | CAAA (SEQ ID NO: | AAGATTA (SEQ ID NO: | CCAAA (SEQ ID NO: |
| 781) | 361) | 991) | 571) |
| TTTATAGCAGTTGTC | CATGGACAACTGCT | TTTATAGCAGTTGTCC | CACATGGACAACTGCT |
| CATG (SEQ ID NO: | ATAAA (SEQ ID NO: | ATGTGGA (SEQ ID NO: | ATAAA (SEQ ID NO: |
| 782) | 362) | 992) | 572) |
| AGACCAGGGTTTGC | AAACTGCAAACCCT | AGACCAGGGTTTGCA | GAAAACTGCAAACCCT |
| AGTTT (SEQ ID NO: | GGTCT (SEQ ID NO: | GTTTTCTG (SEQ ID | GGTCT (SEQ ID NO: 573) |
| 783) | 363) | NO: 993) | |
| TTGGCTTCAAATGTA | TCTTTACATTTGAAG | TTGGCTTCAAATGTAA | AATCTTTACATTTGAA |
| AAGA (SEQ ID NO: | CCAA (SEQ ID NO: | AGATTAA (SEQ ID NO: | GCCAA (SEQ ID NO: |
| 784) | 364) | 994) | 574) |
| CCTGCTTGAATGCTG | TTCTCAGCATTCAAG | CCTGCTTGAATGCTGA | ATTTCTCAGCATTCAA |
| AGAA (SEQ ID NO: | CAGG (SEQ ID NO: | GAAATAC (SEQ ID NO: | GCAGG (SEQ ID NO: |
| 785) | 365) | 995) | 575) |
| CAATTAACCTTGAA | ACAAATTCAAGGTT | CAATTAACCTTGAATT | AAACAAATTCAAGGTT |
| TTTGT (SEQ ID NO: | AATTG (SEQ ID NO: | TGTTTCA (SEQ ID NO: | AATTG (SEQ ID NO: 576) |
| 786) | 366) | 996) | |
| AGTTTCATTTATTAG | GTCACTAATAAATG | AGTTTCATTTATTAGT | ATGTCACTAATAAATG |
| TGAC (SEQ ID NO: | AAACT (SEQ ID NO: | GACATCA (SEQ ID NO: | AAACT (SEQ ID NO: |
| 787) | 367) | 997) | 577) |
| CTCTTTACCAACCGC | TTCTGCGGTTGGTAA | CTCTTTACCAACCGCA | GTTTCTGCGGTTGGTA |
| AGAA (SEQ ID NO: | AGAG (SEQ ID NO: | GAAACTT (SEQ ID NO: | AAGAG (SEQ ID NO: |
| 788) | 368) | 998) | 578) |
| CTTTGATGGCAAAG | ATCTTCTTTGCCATC | CTTTGATGGCAAAGA | CTATCTTCTTTGCCATC |
| AAGAT (SEQ ID NO: | AAAG (SEQ ID NO: | AGATAGAA (SEQ ID | AAAG (SEQ ID NO: 579) |
| 789) | 369) | NO: 999) | |
| ATCAGAACTTGAGG | TATAACCTCAAGTTC | ATCAGAACTTGAGGT | TGTATAACCTCAAGTT |
| TTATA (SEQ ID NO: | TGAT (SEQ ID NO: | TATACAGG (SEQ ID | CTGAT (SEQ ID NO: 580) |
| 790) | 370) | NO: 1000) | |
| ATCTGTGCGGAGGT | TAAGAACCTCCGCA | ATCTGTGCGGAGGTT | CATAAGAACCTCCGCA |
| TCTTA (SEQ ID NO: | CAGAT (SEQ ID NO: | CTTATGAT (SEQ ID | CAGAT (SEQ ID NO: |
| 791) | 371) | NO: 1001) | 581) |
| TGTACATACTCATG | ATCGTCATGAGTAT | TGTACATACTCATGAC | GCATCGTCATGAGTAT |
| ACGAT (SEQ ID NO: | GTACA (SEQ ID NO: | GATGCCA (SEQ ID NO: | GTACA (SEQ ID NO: |
| 792) | 372) | 1002) | 582) |
| CCTTCCACAGTTGTC | CAGTGACAACTGTG | CCTTCCACAGTTGTCA | TGCAGTGACAACTGTG |
| ACTG (SEQ ID NO: | GAAGG (SEQ ID NO: | CTGCAAC (SEQ ID NO: | GAAGG (SEQ ID NO: |
| 793) | 373) | 1003) | 583) |
| TCTTCAGCTCAGGTC | TATGGACCTGAGCT | TCTTCAGCTCAGGTCC | TTTATGGACCTGAGCT |
| CATA (SEQ ID NO: | GAAGA (SEQ ID NO: | ATAAAAG (SEQ ID NO: | GAAGA (SEQ ID NO: |
| 794) | 374) | 1004) | 584) |
| GAAACAGCATATTC | TTCAAGAATATGCT | GAAACAGCATATTCT | AGTTCAAGAATATGCT |
| TTGAA (SEQ ID NO: | GTTTC (SEQ ID NO: | TGAACTTC (SEQ ID | GTTTC (SEQ ID NO: 585) |
| 795) | 375) | NO: 1005) | |
| CAGCTCCTTGAGGG | CTCAACCCTCAAGG | CAGCTCCTTGAGGGTT | GCCTCAACCCTCAAGG |
| TTGAG (SEQ ID NO: | AGCTG (SEQ ID NO: | GAGGCCT (SEQ ID NO: | AGCTG (SEQ ID NO: |
| 796) | 376) | 1006) | 586) |
| CCATGTTCTGTGGTA | AACATACCACAGAA | CCATGTTCTGTGGTAT | GGAACATACCACAGAA |
| TGTT (SEQ ID NO: | CATGG (SEQ ID NO: | GTTCCTC (SEQ ID NO: | CATGG (SEQ ID NO: |
| 797) | 377) | 1007) | 587) |
| GTGTGTACATACTC | GTCATGAGTATGTA | GTGTGTACATACTCAT | TCGTCATGAGTATGTA |
| ATGAC (SEQ ID NO: | CACAC (SEQ ID NO: | GACGATG (SEQ ID NO: | CACAC (SEQ ID NO: |
| 798) | 378) | 1008) | 588) |
| CTTCTGGTTGAAGTG | GACACACTTCAACC | CTTCTGGTTGAAGTGT | CTGACACACTTCAACC |
| TGTC (SEQ ID NO: | AGAAG (SEQ ID NO: | GTCAGGT (SEQ ID NO: | AGAAG (SEQ ID NO: |
| 799) | 379) | 1009) | 589) |
| GTTATTACCTGTGTG | TCAGCACACAGGTA | GTTATTACCTGTGTGC | GCTCAGCACACAGGTA |
| CTGA (SEQ ID NO: | ATAAC (SEQ ID NO: | TGAGCTC (SEQ ID NO: | ATAAC (SEQ ID NO: |
| 800) | 380) | 1010) | 590) |
| TGACTTTAATAGATC | CATGGATCTATTAA | TGACTTTAATAGATCC | AACATGGATCTATTAA |
| CATG (SEQ ID NO: | AGTCA (SEQ ID NO: | ATGTTCT (SEQ ID NO: | AGTCA (SEQ ID NO: |
| 801) | 381) | 1011) | 591) |
| CCACAGCCGAGCTT | GAACCAAGCTCGGC | CCACAGCCGAGCTTG | TGGAACCAAGCTCGGC |
| GGTTC (SEQ ID NO: | TGTGG (SEQ ID NO: | GTTCCACT (SEQ ID | TGTGG (SEQ ID NO: 592) |
| 802) | 382) | NO: 1012) | |
| CCTGTCCAATATCA | GCCATTGATATTGG | CCTGTCCAATATCAAT | CTGCCATTGATATTGG |
| ATGGC (SEQ ID NO: | ACAGG (SEQ ID NO: | GGCAGGG (SEQ ID NO: | ACAGG (SEQ ID NO: |
| 803) | 383) | 1013) | 593) |
| GAAGAAGAAGCTGA | CACCCTCAGCTTCTT | GAAGAAGAAGCTGAG | CTCACCCTCAGCTTCTT |
| GGGTG (SEQ ID NO: | CTTC (SEQ ID NO: | GGTGAGGG (SEQ ID | CTTC (SEQ ID NO: 594) |
| 804) | 384) | NO: 1014) | |
| CTTTTGGCAATTCTC | AGGAGAGAATTGCC | CTTTTGGCAATTCTCT | GCAGGAGAGAATTGCC |
| TCCT (SEQ ID NO: | AAAAG (SEQ ID NO: | CCTGCAC (SEQ ID NO: | AAAAG (SEQ ID NO: |
| 805) | 385) | 1015) | 595) |
| GCCACCAGTTATCA | CATGCTGATAACTG | GCCACCAGTTATCAG | GACATGCTGATAACTG |
| GCATG (SEQ ID NO: | GTGGC (SEQ ID NO: | CATGTCCT (SEQ ID | GTGGC (SEQ ID NO: |
| 806) | 386) | NO: 1016) | 596) |
| TAAAGATTAAACAT | AGATTATGTTTAATC | TAAAGATTAAACATA | AAAGATTATGTTTAAT |
| AATCT (SEQ ID NO: | TTTA (SEQ ID NO: | ATCTTTTT (SEQ ID NO: | CTTTA (SEQ ID NO: 597) |
| 807) | 387) | 1017) | |
| GCTTTTGGCAATTCT | GGAGAGAATTGCCA | GCTTTTGGCAATTCTC | CAGGAGAGAATTGCCA |
| CTCC (SEQ ID NO: | AAAGC(SEQ ID NO: | TCCTGCA (SEQ ID NO: | AAAGC (SEQ ID NO: |
| 808) | 388) | 1018) | 598) |
| AAAGATTAAACATA | AAGATTATGTTTAAT | AAAGATTAAACATAA | AAAAGATTATGTTTAA |
| ATCTT (SEQ ID NO: | CTTT (SEQ ID NO: | TCTTTTTT (SEQ ID NO: | TCTTT (SEQ ID NO: 599) |
| 809) | 389) | 1019) | |
| ACCATTTTGGTATGA | GCCTTCATACCAAA | ACCATTTTGGTATGAA | AGGCCTTCATACCAAA |
| AGGC (SEQ ID NO: | ATGGT (SEQ ID NO: | GGCCTTG (SEQ ID NO: | ATGGT (SEQ ID NO: 600) |
| 810) | 390) | 1020) | |
| GCTTCCCAGCAAAC | CGCTGGTTTGCTGGG | GCTTCCCAGCAAACC | TGCGCTGGTTTGCTGG |
| CAGCG (SEQ ID NO: | AAGC (SEQ ID NO: | AGCGCAGC (SEQ ID | GAAGC (SEQ ID NO: |
| 811) | 391) | NO: 1021) | 601) |
| GGGAAAGAAATCTA | TGTTCTAGATTTCTT | GGGAAAGAAATCTAG | AATGTTCTAGATTTCTT |
| GAACA (SEQ ID NO: | TCCC (SEQ ID NO: | AACATTGT (SEQ ID | TCCC (SEQ ID NO: 602) |
| 812) | 392) | NO: 1022) | |
| CCTGTGTGCTGAGCT | CTCGAGCTCAGCAC | CCTGTGTGCTGAGCTC | AGCTCGAGCTCAGCAC |
| CGAG (SEQ ID NO: | ACAGG (SEQ ID NO: | GAGCTGC (SEQ ID NO: | ACAGG (SEQ ID NO: |
| 813) | 393) | 1023) | 603) |
| CCTTTTGGAACAGC | CCATTGCTGTTCCAA | CCTTTTGGAACAGCA | CACCATTGCTGTTCCA |
| AATGG (SEQ ID NO: | AAGG (SEQ ID NO: | ATGGTGCA (SEQ ID | AAAGG (SEQ ID NO: |
| 814) | 394) | NO: 1024) | 604) |
| GCAGAAAAGTGGAC | AGATCGTCCACTTTT | GCAGAAAAGTGGACG | CAAGATCGTCCACTTT |
| GATCT (SEQ ID NO: | CTGC (SEQ ID NO: | ATCTTGTT (SEQ ID | TCTGC (SEQ ID NO: 605) |
| 815) | 395) | NO: 1025) | |
| GTTCCTTCACGTTAT | GGTAATAACGTGAA | GTTCCTTCACGTTATT | CAGGTAATAACGTGAA |
| TACC (SEQ ID NO: | GGAAC (SEQ ID NO: | ACCTGTG (SEQ ID NO: | GGAAC (SEQ ID NO: |
| 816) | 396) | 1026) | 606) |
| TGAATAAAACTCTC | GGCATGAGAGTTTT | TGAATAAAACTCTCA | GTGGCATGAGAGTTTT |
| ATGCC (SEQ ID NO: | ATTCA (SEQ ID NO: | TGCCACTG (SEQ ID | ATTCA (SEQ ID NO: 607) |
| 817) | 397) | NO: 1027) | |
| TTGCTGTTCATTGGT | TCAAACCAATGAAC | TTGCTGTTCATTGGTT | CTTCAAACCAATGAAC |
| TTGA (SEQ ID NO: | AGCAA (SEQ ID NO: | TGAAGGC (SEQ ID NO: | AGCAA (SEQ ID NO: |
| 818) | 398) | 1028) | 608) |
| TTTGCTGTTCATTGG | CAAACCAATGAACA | TTTGCTGTTCATTGGT | TTCAAACCAATGAACA |
| TTTG (SEQ ID NO: | GCAAA (SEQ ID NO: | TTGAAGG (SEQ ID NO: | GCAAA (SEQ ID NO: |
| 819) | 399) | 1029) | 609) |
| CAATAATTGAGTTG | CCAACCAACTCAAT | CAATAATTGAGTTGG | ATCCAACCAACTCAAT |
| GTTGG (SEQ ID NO: | TATTG (SEQ ID NO: | TTGGATTT (SEQ ID | TATTG (SEQ ID NO: 610) |
| 820) | 400) | NO: 1030) | |
| GATTAAACATAATC | AAAAAGATTATGTT | GATTAAACATAATCTT | CAAAAAAGATTATGTT |
| TTTTT (SEQ ID NO: | TAATC (SEQ ID NO: | TTTTGTA (SEQ ID NO: | TAATC (SEQ ID NO: 611) |
| 821) | 401) | 1031) | |
| TCCTCTGCAGGCATC | GTGGGATGCCTGCA | TCCTCTGCAGGCATCC | CTGTGGGATGCCTGCA |
| CCAC (SEQ ID NO: | GAGGA (SEQ ID NO: | CACAGGT (SEQ ID NO: | GAGGA (SEQ ID NO: |
| 822) | 402) | 1032) | 612) |
| GCCTCTGAGTGGTCT | CTCAAGACCACTCA | GCCTCTGAGTGGTCTT | CCCTCAAGACCACTCA |
| TGAG (SEQ ID NO: | GAGGC (SEQ ID NO: | GAGGGCT (SEQ ID NO: | GAGGC (SEQ ID NO: |
| 823) | 403) | 1033) | 613) |
| CACCTCCTCTGCAG | GATGCCTGCAGAGG | CACCTCCTCTGCAGGC | GGGATGCCTGCAGAGG |
| GCATC (SEQ ID NO: | AGGTG (SEQ ID NO: | ATCCCAC (SEQ ID NO: | AGGTG (SEQ ID NO: |
| 824) | 404) | 1034) | 614) |
| ATAAAACTCTCATG | AGTGGCATGAGAGT | ATAAAACTCTCATGC | CCAGTGGCATGAGAGT |
| CCACT (SEQ ID NO: | TTTAT (SEQ ID NO: | CACTGGTT (SEQ ID | TTTAT (SEQ ID NO: 615) |
| 825) | 405) | NO: 1035) | |
| TTCTGTGACTTTAAT | ATCTATTAAAGTCAC | TTCTGTGACTTTAATA | GGATCTATTAAAGTCA |
| AGAT (SEQ ID NO: | AGAA (SEQ ID NO: | GATCCAT (SEQ ID NO: | CAGAA (SEQ ID NO: |
| 826) | 406) | 1036) | 616) |
| AGTAAAATGGATCA | TCCTGTGATCCATTT | AGTAAAATGGATCAC | CTTCCTGTGATCCATTT |
| CAGGA (SEQ ID NO: | TACT (SEQ ID NO: | AGGAAGGG (SEQ ID | TACT (SEQ ID NO: 617) |
| 827) | 407) | NO: 1037) | |
| AAAGAAGATAGAAG | GGCTGCTTCTATCTT | AAAGAAGATAGAAGC | CTGGCTGCTTCTATCTT |
| CAGCC (SEQ ID NO: | CTTT (SEQ ID NO: | AGCCAGGA (SEQ ID | CTTT (SEQ ID NO: 618) |
| 828) | 408) | NO: 1038) | |
| GCACAGAGCCATCT | TACACAGATGGCTC | GCACAGAGCCATCTG | TGTACACAGATGGCTC |
| GTGTA (SEQ ID NO: | TGTGC (SEQ ID NO: | TGTACACA (SEQ ID | TGTGC (SEQ ID NO: 619) |
| 829) | 409) | NO: 1039) | |
| GGACTGTCAGGTAG | AAGTTCTACCTGAC | GGACTGTCAGGTAGA | TCAAGTTCTACCTGAC |
| AACTT (SEQ ID NO: | AGTCC (SEQ ID NO: | ACTTGAAG (SEQ ID | AGTCC (SEQ ID NO: 620) |
| 830) | 410) | NO: 1040) | |

TABLE 2

| SEQ | | SEQ | Sense strand | SEQ | Antisense strand |
|---|---|---|---|---|---|
| ID NO | Target_23mer | ID NO | (Passenger)_21mer | ID NO | (Guide)_23mer |
| 1041 | ATGCTCTCCAAGTAT | 1301 | GCTCTCCAAGTATG | 1561 | AGACGATCATACTT |
| | GATCGTCT | | ATCGTCT | | GGAGAGCAT |
| 1042 | CGTCTGCAGAACAAG | 1302 | TCTGCAGAACAAG | 1562 | TGGACGATCTTGTTC |
| | ATCGTCCA | | ATCGTCCA | | TGCAGACG |
| 1043 | TCGTCCACTTTTCTGC | 1303 | GTCCACTTTTCTGC | 1563 | GGCATTGGCAGAAA |
| | CAATGCC | | CAATGCC | | AGTGGACGA |
| 1044 | AGCCACGGCTCCCAG | 1304 | CCACGGCTCCCAGT | 1564 | AACCCGCACTGGGA |
| | TGCGGGTT | | GCGGGTT | | GCCGTGGCT |
| 1045 | GGAACACGGAGATT | 1305 | AACACGGAGATTG | 1565 | CTCAATGCCAATCT |
| | GGCATTGAG | | GCATTGAG | | CCGTGTTCC |
| 1046 | GAACACGGAGATTG | 1306 | ACACGGAGATTGG | 1566 | TCTCAATGCCAATCT |
| | GCATTGAGA | | CATTGAGA | | CCGTGTTC |
| 1047 | CCGGAGAGAAGATG | 1307 | GGAGAGAAGATGA | 1567 | TGGCAATGTCATCTT |
| | ACATTGCCA | | CATTGCCA | | CTCTCCGG |
| 1048 | GTAACCAGTGGCATG | 1308 | AACCAGTGGCATG | 1568 | AAAACTCTCATGCC |
| | AGAGTTTT | | AGAGTTTT | | ACTGGTTAC |
| 1049 | GACATGCAGGCCTCT | 1309 | CATGCAGGCCTCTG | 1569 | GCCTCACCAGAGGC |
| | GGTGAGGC | | GTGAGGC | | CTGCATGTC |
| 1050 | CCTCTGGTGAGGCCG | 1310 | TCTGGTGAGGCCGT | 1570 | ACAGTACACGGCCT |
| | TGTACTGT | | GTACTGT | | CACCAGAGG |
| 1051 | CACCCGGGCCCACGC | 1311 | CCCGGGCCCACGCC | 1571 | TGATCTTGGCGTGG |
| | CAAGATCA | | AAGATCA | | GCCCGGGTG |
| 1052 | CACGCCAAGATCAAG | 1312 | CGCCAAGATCAAG | 1572 | TCTATGGACTTGATC |
| | TCCATAGA | | TCCATAGA | | TTGGCGTG |
| 1053 | TTGTGTTGGGCATAT | 1313 | GTGTTGGGCATATC | 1573 | CACCAATGATATGC |
| | CATTGGTG | | ATTGGTG | | CCAACACAA |
| 1054 | ATATCATTGGTGCTG | 1314 | ATCATTGGTGCTGT | 1574 | AGCAACCACAGCAC |
| | TGGTTGCT | | GGTTGCT | | CAATGATAT |
| 1055 | CAGCAAACCGGATTG | 1315 | GCAAACCGGATTGT | 1575 | TCGAACCACAATCC |
| | TGGTTCGA | | GGTTCGA | | GGTTTGCTG |
| 1056 | CAAACCGGATTGTGG | 1316 | AACCGGATTGTGGT | 1576 | CACTCGAACCACAA |
| | TTCGAGTG | | TCGAGTG | | TCCGGTTTG |
| 1057 | ACCGGATTGTGGTTC | 1317 | CGGATTGTGGTTCG | 1577 | CTTCACTCGAACCA |
| | GAGTGAAG | | AGTGAAG | | CAATCCGGT |
| 1058 | GCATGCTGGACCGTG | 1318 | ATGCTGGACCGTGA | 1578 | GTCCTCATCACGGT |
| | ATGAGGAC | | TGAGGAC | | CCAGCATGC |
| 1059 | GTGATGAGGACATGC | 1319 | GATGAGGACATGC | 1579 | AGTTATCAGCATGT |
| | TGATAACT | | TGATAACT | | CCTCATCAC |
| 1060 | TGGCCAGATACAAGG | 1320 | GCCAGATACAAGG | 1580 | GAAGCCAACCTTGT |
| | TTGGCTTC | | TTGGCTTC | | ATCTGGCCA |
| 1061 | ATCTCTCTCAGAGTA | 1321 | CTCTCTCAGAGTAT | 1581 | TTCCATAATACTCTG |
| | TTATGGAA | | TATGGAA | | AGAGAGAT |
| 1062 | AACCAACCTTCCCTC | 1322 | CCAACCTTCCCTCC | 1582 | CCGTGTTGGAGGGA |
| | CAACACGG | | AACACGG | | AGGTTGGTT |
| 1063 | ATGCCTAGCAAGCTC | 1323 | GCCTAGCAAGCTCT | 1583 | GATACTGAGAGCTT |
| | TCAGTATC | | CAGTATC | | GCTAGGCAT |
| 1064 | CCTAGCAAGCTCTCA | 1324 | TAGCAAGCTCTCAG | 1584 | CATGATACTGAGAG |
| | GTATCATG | | TATCATG | | CTTGCTAGG |
| 1065 | GCTCTCAGTATCATG | 1325 | TCTCAGTATCATGC | 1585 | CTTCCGAGCATGAT |
| | CTCGGAAG | | TCGGAAG | | ACTGAGAGC |
| 1066 | ATTCCCACCAAGTTT | 1326 | TCCCACCAAGTTTG | 1586 | CTTATTCCAAACTTG |
| | GGAATAAG | | GAATAAG | | GTGGGAAT |
| 1067 | AACCTCCGCACAGAT | 1327 | CCTCCGCACAGATA | 1587 | ATGACAATATCTGT |
| | ATTGTCAT | | TTGTCAT | | GCGGAGGTT |
| 1068 | CCTACAAGATCCCGG | 1328 | TACAAGATCCCGGC | 1588 | GCCAAATGCCGGGA |
| | CATTTGGC | | ATTTGGC | | TCTTGTAGG |
| 1069 | GCTCAGCACACAGGT | 1329 | TCAGCACACAGGT | 1589 | ACGTTATTACCTGTG |
| | AATAACGT | | AATAACGT | | TGCTGAGC |
| 1070 | GCAAACCCTGGTCTG | 1330 | AAACCCTGGTCTGT | 1590 | GACCCTCACAGACC |
| | TGAGGGTC | | GAGGGTC | | AGGGTTTGC |
| 1071 | CATACCACAGAACAT | 1331 | TACCACAGAACAT | 1591 | ATAGATCCATGTTCT |
| | GGATCTAT | | GGATCTAT | | GTGGTATG |
| 1072 | CAGAACATGGATCTA | 1332 | GAACATGGATCTAT | 1592 | GACTTTAATAGATC |
| | TTAAAGTC | | TAAAGTC | | CATGTTCTG |
| 1073 | GAACATGGATCTATT | 1333 | ACATGGATCTATTA | 1593 | GTGACTTTAATAGA |
| | AAAGTCAC | | AAGTCAC | | TCCATGTTC |
| 1074 | GTCGGGAAGGGTTTG | 1334 | CGGGAAGGGTTTGT | 1594 | GAATAGCACAAACC |
| | TGCTATTC | | GCTATTC | | CTTCCCGAC |
| 1075 | TCGGGAAGGGTTTGT | 1335 | GGGAAGGGTTTGT | 1595 | GGAATAGCACAAAC |
| | GCTATTCC | | GCTATTCC | | CCTTCCCGA |
| 1076 | TAACCTCAAGTTCTG | 1336 | ACCTCAAGTTCTGA | 1596 | GACACCATCAGAAC |
| | ATGGTGTC | | TGGTGTC | | TTGAGGTTA |
| 1077 | AACCTCAAGTTCTGA | 1337 | CCTCAAGTTCTGAT | 1597 | AGACACCATCAGAA |
| | TGGTGTCT | | GGTGTCT | | CTTGAGGTT |
| 1078 | GATTCCCACAAACCT | 1338 | TTCCCACAAACCTC | 1598 | GCTTCTAGAGGTTT |
| | CTAGAAGC | | TAGAAGC | | GTGGGAATC |
| 1079 | TTCCCACAAACCTCT | 1339 | CCCACAAACCTCTA | 1599 | AAGCTTCTAGAGGT |
| | AGAAGCTT | | GAAGCTT | | TTGTGGGAA |
| 1080 | CTGGCCTTCAAACCA | 1340 | GGCCTTCAAACCAA | 1600 | CTGTTCATTGGTTTG |
| | ATGAACAG | | TGAACAG | | AAGGCCAG |
| 1081 | AACCAATGAACAGC | 1341 | CCAATGAACAGCA | 1601 | TTATGCTTTGCTGTT |
| | AAAGCATAA | | AAGCATAA | | CATTGGTT |
| 1082 | ATGAAACAAATTCAA | 1342 | GAAACAAATTCAA | 1602 | AATTAACCTTGAAT |
| | GGTTAATT | | GGTTAATT | | TTGTTTCAT |
| 1083 | TGTGAAGCTGCATAA | 1343 | TGAAGCTGCATAA | 1603 | ATCTTGCTTTATGCA |
| | AGCAAGAT | | AGCAAGAT | | GCTTCACA |
| 1084 | AAGCTGCATAAAGCA | 1344 | GCTGCATAAAGCA | 1604 | AGTAATCTTGCTTTA |
| | AGATTACT | | AGATTACT | | TGCAGCTT |
| 1085 | CAAAAAAGATTATGT | 1345 | AAAAAGATTATGTT | 1605 | AAGATTAAACATAA |
| | TTAATCTT | | TAATCTT | | TCTTTTTTG |
| 1086 | ATCTAAGGCTTGGTT | 1346 | CTAAGGCTTGGTTT | 1606 | AGTAAGAAAACCAA |
| | TTCTTACT | | TCTTACT | | GCCTTAGAT |
| 1087 | GCTTGGTTTTCTTACT | 1347 | TTGGTTTTCTTACT | 1607 | ATATGACAGTAAGA |
| | GTCATAT | | GTCATAT | | AAACCAAGC |
| 1088 | CACCTCAAGTTTCTG | 1348 | CCTCAAGTTTCTGC | 1608 | ACCAACCGCAGAAA |
| | CGGTTGGT | | GGTTGGT | | CTTGAGGTG |
| 1089 | ACCTCAAGTTTCTGC | 1349 | CTCAAGTTTCTGCG | 1609 | TACCAACCGCAGAA |
| | GGTTGGTA | | GTTGGTA | | ACTTGAGGT |
| 1090 | ATAGTTGGACTGGTG | 1350 | AGTTGGACTGGTGC | 1610 | ACATCAAGCACCAG |
| | CTTGATGT | | TTGATGT | | TCCAACTAT |
| 1091 | GGGATTTGGAAGACA | 1351 | GATTTGGAAGACA | 1611 | CATTCACTTGTCTTC |
| | AGTGAATG | | AGTGAATG | | CAAATCCC |
| 1092 | CACAGTGATGCTCTC | 1352 | CAGTGATGCTCTCC | 1612 | CATACTTGGAGAGC |
| | CAAGTATG | | AAGTATG | | ATCACTGTG |
| 1093 | GCAAACCGGATTGTG | 1353 | AAACCGGATTGTG | 1613 | ACTCGAACCACAAT |
| | GTTCGAGT | | GTTCGAGT | | CCGGTTTGC |
| 1094 | CAAGTTTCTGCGGTT | 1354 | AGTTTCTGCGGTTG | 1614 | TCTTTACCAACCGC |
| | GGTAAAGA | | GTAAAGA | | AGAAACTTG |
| 1095 | GCTGCTTCTATCTTCT | 1355 | TGCTTCTATCTTCTT | 1615 | ATGGCAAAGAAGAT |
| | TTGCCAT | | TGCCAT | | AGAAGCAGC |
| 1096 | GGAGGAACATACCA | 1356 | AGGAACATACCAC | 1616 | CATGTTCTGTGGTAT |
| | CAGAACATG | | AGAACATG | | GTTCCTCC |
| 1097 | CCTTTTATGGACCTG | 1357 | TTTTATGGACCTGA | 1617 | CTTCAGCTCAGGTC |
| | AGCTGAAG | | GCTGAAG | | CATAAAAGG |
| 1098 | CCCGGGCCCACGCCA | 1358 | CGGGCCCACGCCA | 1618 | CTTGATCTTGGCGTG |
| | AGATCAAG | | AGATCAAG | | GGCCCGGG |
| 1099 | CGGGAAGGGTTTGTG | 1359 | GGAAGGGTTTGTGC | 1619 | GGGAATAGCACAAA |
| | CTATTCCC | | TATTCCC | | CCCTTCCCG |
| 1100 | TTCCTGTGATCCATTT | 1360 | CCTGTGATCCATTT | 1620 | TGCAGTAAAATGGA |
| | TACTGCA | | TACTGCA | | TCACAGGAA |
| 1101 | TCCAACCAACTCAAT | 1361 | CAACCAACTCAATT | 1621 | GCTCAATAATTGAG |
| | TATTGAGC | | ATTGAGC | | TTGGTTGGA |
| 1102 | AGGATCTGCAGCTAT | 1362 | GATCTGCAGCTATC | 1622 | AGCCTTAGATAGCT |
| | CTAAGGCT | | TAAGGCT | | GCAGATCCT |
| 1103 | TTTTCCGAAGCAGAT | 1363 | TTCCGAAGCAGATA | 1623 | ACAACATTATCTGC |
| | AATGTTGT | | ATGTTGT | | TTCGGAAAA |
| 1104 | AGTATGTACACACTG | 1364 | TATGTACACACTGC | 1624 | TTCCGGAGCAGTGT |
| | CTCCGGAA | | TCCGGAA | | GTACATACT |
| 1105 | GATCCAGAGACAACC | 1365 | TCCAGAGACAACC | 1625 | GCCAAAAGGGTTGT |
| | CTTTTGGC | | CTTTTGGC | | CTCTGGATC |
| 1106 | GATCCCGGCATTTGG | 1366 | TCCCGGCATTTGGC | 1626 | GGATGCTGCCAAAT |
| | CAGCATCC | | AGCATCC | | GCCGGGATC |
| 1107 | TAAGGTTACTTGTGT | 1367 | AGGTTACTTGTGTT | 1627 | TATGCCCAACACAA |
| | TGGGCATA | | GGGCATA | | GTAACCTTA |
| 1108 | TGCTGGGAAGAATGC | 1368 | CTGGGAAGAATGC | 1628 | CTTGCTAGGCATTCT |
| | CTAGCAAG | | CTAGCAAG | | TCCCAGCA |
| 1109 | GATTTGGAAGACAAG | 1369 | TTTGGAAGACAAGT | 1629 | TGCATTCACTTGTCT |
| | TGAATGCA | | GAATGCA | | TCCAAATC |
| 1110 | GCTGCATAAAGCAAG | 1370 | TGCATAAAGCAAG | 1630 | AGAGTAATCTTGCT |
| | ATTACTCT | | ATTACTCT | | TTATGCAGC |
| 1111 | TCCCACCAAGTTTGG | 1371 | CCACCAAGTTTGGA | 1631 | AGCTTATTCCAAAC |
| | AATAAGCT | | ATAAGCT | | TTGGTGGGA |
| 1112 | ACAAGTTCAACAAGG | 1372 | AAGTTCAACAAGG | 1632 | ACAATTCTCCTTGTT |
| | AGAATTGT | | AGAATTGT | | GAACTTGT |
| 1113 | GGTGGAGAAAAATG | 1373 | TGGAGAAAAATGC | 1633 | CTGGATCTGCATTTT |
| | CAGATCCAG | | AGATCCAG | | TCTCCACC |
| 1114 | GCCTAGCAAGCTCTC | 1374 | CTAGCAAGCTCTCA | 1634 | ATGATACTGAGAGC |
| | AGTATCAT | | GTATCAT | | TTGCTAGGC |
| 1115 | CCTGGCATCGTCATG | 1375 | TGGCATCGTCATGA | 1635 | TACATACTCATGAC |
| | AGTATGTA | | GTATGTA | | GATGCCAGG |
| 1116 | ATCTGCTCCTTGCAC | 1376 | CTGCTCCTTGCACC | 1636 | GCAACATGGTGCAA |
| | CATGTTGC | | ATGTTGC | | GGAGCAGAT |
| 1117 | ATCCAACCAACTCAA | 1377 | CCAACCAACTCAAT | 1637 | CTCAATAATTGAGT |
| | TTATTGAG | | TATTGAG | | TGGTTGGAT |
| 1118 | GTTACTTGTGTTGGG | 1378 | TACTTGTGTTGGGC | 1638 | ATGATATGCCCAAC |
| | CATATCAT | | ATATCAT | | ACAAGTAAC |
| 1119 | GAAGGATAAGGTTAC | 1379 | AGGATAAGGTTACT | 1639 | CAACACAAGTAACC |
| | TTGTGTTG | | TGTGTTG | | TTATCCTTC |
| 1120 | GCGGGGCTTGCACAG | 1380 | GGGGCTTGCACAGT | 1640 | GAGCATCACTGTGC |
| | TGATGCTC | | GATGCTC | | AAGCCCCGC |
| 1121 | GAATGAAACAAATTC | 1381 | ATGAAACAAATTC | 1641 | TTAACCTTGAATTTG |
| | AAGGTTAA | | AAGGTTAA | | TTTCATTC |
| 1122 | TATCTTTGTGATCTTG | 1382 | TCTTTGTGATCTTG | 1642 | GACAGTCCAAGATC |
| | GACTGTC | | GACTGTC | | ACAAAGATA |
| 1123 | TGGTGGAGAAAAAT | 1383 | GTGGAGAAAAATG | 1643 | TGGATCTGCATTTTT |
| | GCAGATCCA | | CAGATCCA | | CTCCACCA |
| 1124 | AAGCAGATAATGTTG | 1384 | GCAGATAATGTTGT | 1644 | CCCTGACACAACAT |
| | TGTCAGGG | | GTCAGGG | | TATCTGCTT |
| 1125 | TTCCCACCAAGTTTG | 1385 | CCCACCAAGTTTGG | 1645 | GCTTATTCCAAACTT |
| | GAATAAGC | | AATAAGC | | GGTGGGAA |
| 1126 | CAGCAAAGCATAACC | 1386 | GCAAAGCATAACC | 1646 | AGATTCAAGGTTAT |
| | TTGAATCT | | TTGAATCT | | GCTTTGCTG |
| 1127 | AGCACACAGGTAATA | 1387 | CACACAGGTAATA | 1647 | CTTCACGTTATTACC |
| | ACGTGAAG | | ACGTGAAG | | TGTGTGCT |
| 1128 | CTACCAGCCATTATC | 1388 | ACCAGCCATTATCA | 1648 | TCAATTGTGATAAT |
| | ACAATTGA | | CAATTGA | | GGCTGGTAG |
| 1129 | ATACAAAAATCCAAC | 1389 | ACAAAAATCCAAC | 1649 | TTGAGTTGGTTGGA |
| | CAACTCAA | | CAACTCAA | | TTTTTGTAT |
| 1130 | AGGAATAGGAAGCA | 1390 | GAATAGGAAGCAC | 1650 | TCGTCTTGGTGCTTC |
| | CCAAGACGA | | CAAGACGA | | CTATTCCT |
| 1131 | GTCAGCGCTGACCTC | 1391 | CAGCGCTGACCTCA | 1651 | TGTCCATTGAGGTC |
| | AATGGACA | | ATGGACA | | AGCGCTGAC |
| 1132 | GCCTCCCGGAGAGAA | 1392 | CTCCCGGAGAGAA | 1652 | ATGTCATCTTCTCTC |
| | GATGACAT | | GATGACAT | | CGGGAGGC |
| 1133 | CTGCCATTGATATTG | 1393 | GCCATTGATATTGG | 1653 | CACCTGTCCAATAT |
| | GACAGGTG | | ACAGGTG | | CAATGGCAG |
| 1134 | TCAGCACACAGGTAA | 1394 | AGCACACAGGTAA | 1654 | TCACGTTATTACCTG |
| | TAACGTGA | | TAACGTGA | | TGTGCTGA |
| 1135 | TATGCCTAAATGGTG | 1395 | TGCCTAAATGGTGA | 1655 | TGCATATTCACCATT |
| | AATATGCA | | ATATGCA | | TAGGCATA |
| 1136 | GTCTGCCACTGGGTT | 1396 | CTGCCACTGGGTTT | 1656 | TTCTATAAAACCCA |
| | TTATAGAA | | TATAGAA | | GTGGCAGAC |
| 1137 | TGTACACACTGCTCC | 1397 | TACACACTGCTCCG | 1657 | CTGATTCCGGAGCA |
| | GGAATCAG | | GAATCAG | | GTGTGTACA |
| 1138 | AGGTTACTTGTGTTG | 1398 | GTTACTTGTGTTGG | 1658 | GATATGCCCAACAC |
| | GGCATATC | | GCATATC | | AAGTAACCT |
| 1139 | GGTGAGGCCGTGTAC | 1399 | TGAGGCCGTGTACT | 1659 | TCGTCACAGTACAC |
| | TGTGACGA | | GTGACGA | | GGCCTCACC |
| 1140 | TGCCTAGCAAGCTCT | 1400 | CCTAGCAAGCTCTC | 1660 | TGATACTGAGAGCT |
| | CAGTATCA | | AGTATCA | | TGCTAGGCA |
| 1141 | GATTGGCATTGAGAT | 1401 | TTGGCATTGAGATG | 1661 | TGAACTTCATCTCA |
| | GAAGTTCA | | AAGTTCA | | ATGCCAATC |
| 1142 | ATAGAACACCCAATC | 1402 | AGAACACCCAATCT | 1662 | GTAGCCCAGATTGG |
| | TGGGCTAC | | GGGCTAC | | GTGTTCTAT |
| 1143 | TTTGTTTGTTTCATTT | 1403 | TGTTTGTTTCATTTC | 1663 | TCAGCGGAAATGAA |
| | CCGCTGA | | CGCTGA | | ACAAACAAA |
| 1144 | CCTTCCACTACTTCA | 1404 | TTCCACTACTTCAG | 1664 | CCCATAGCTGAAGT |
| | GCTATGGG | | CTATGGG | | AGTGGAAGG |
| 1145 | ACATACCACAGAACA | 1405 | ATACCACAGAACA | 1665 | TAGATCCATGTTCTG |
| | TGGATCTA | | TGGATCTA | | TGGTATGT |
| 1146 | CATAAGAACCTCCGC | 1406 | TAAGAACCTCCGCA | 1666 | ATATCTGTGCGGAG |
| | ACAGATAT | | CAGATAT | | GTTCTTATG |
| 1147 | CACCTACAAGATCCC | 1407 | CCTACAAGATCCCG | 1667 | CAAATGCCGGGATC |
| | GGCATTTG | | GCATTTG | | TTGTAGGTG |
| 1148 | TGAGTATGTACACAC | 1408 | AGTATGTACACACT | 1668 | CCGGAGCAGTGTGT |
| | TGCTCCGG | | GCTCCGG | | ACATACTCA |
| 1149 | CTGCTCCTTGCACCA | 1409 | GCTCCTTGCACCAT | 1669 | CTGCAACATGGTGC |
| | TGTTGCAG | | GTTGCAG | | AAGGAGCAG |
| 1150 | GCCAAAGTAATTTCC | 1410 | CAAAGTAATTTCCA | 1670 | TTCTAGGTGGAAAT |
| | ACCTAGAA | | CCTAGAA | | TACTTTGGC |
| 1151 | AAAGCCACGGCTCCC | 1411 | AGCCACGGCTCCCA | 1671 | CCCGCACTGGGAGC |
| | AGTGCGGG | | GTGCGGG | | CGTGGCTTT |
| 1152 | TGAGCTTGTCTGCCA | 1412 | AGCTTGTCTGCCAC | 1672 | AAACCCAGTGGCAG |
| | CTGGGTTT | | TGGGTTT | | ACAAGCTCA |
| 1153 | CTGGTGCTTGATGTC | 1413 | GGTGCTTGATGTCA | 1673 | TTATTAGTGACATC |
| | ACTAATAA | | CTAATAA | | AAGCACCAG |
| 1154 | TGCCATCAAAGATGC | 1414 | CCATCAAAGATGCC | 1674 | CACGGATGGCATCT |
| | CATCCGTG | | ATCCGTG | | TTGATGGCA |
| 1155 | TGCTCTCCAAGTATG | 1415 | CTCTCCAAGTATGA | 1675 | CAGACGATCATACT |
| | ATCGTCTG | | TCGTCTG | | TGGAGAGCA |
| 1156 | ATGGAGCAGGAGGA | 1416 | GGAGCAGGAGGAA | 1676 | GTGGTATGTTCCTCC |
| | ACATACCAC | | CATACCAC | | TGCTCCAT |
| 1157 | GGATCTCTCTCAGAG | 1417 | ATCTCTCTCAGAGT | 1677 | CCATAATACTCTGA |
| | TATTATGG | | ATTATGG | | GAGAGATCC |
| 1158 | ATGCTCGGAAGAGTG | 1418 | GCTCGGAAGAGTG | 1678 | GTCAACCTCACTCTT |
| | AGGTTGAC | | AGGTTGAC | | CCGAGCAT |
| 1159 | TGTCAGCGCTGACCT | 1419 | TCAGCGCTGACCTC | 1679 | GTCCATTGAGGTCA |
| | CAATGGAC | | AATGGAC | | GCGCTGACA |
| 1160 | CTTTATTCCACATGG | 1420 | TTATTCCACATGGA | 1680 | GCAGTTGTCCATGT |
| | ACAACTGC | | CAACTGC | | GGAATAAAG |
| 1161 | CCAACAAGAAGGCC | 1421 | AACAAGAAGGCCA | 1681 | TGCATAGATGGCCT |
| | ATCTATGCA | | TCTATGCA | | TCTTGTTGG |
| 1162 | ACTACCAGCCATTAT | 1422 | TACCAGCCATTATC | 1682 | CAATTGTGATAATG |
| | CACAATTG | | ACAATTG | | GCTGGTAGT |
| 1163 | CATGGACACAGTGAG | 1423 | TGGACACAGTGAG | 1683 | CAGACAAGCTCACT |
| | CTTGTCTG | | CTTGTCTG | | GTGTCCATG |
| 1164 | TAAGAACCTCCGCAC | 1424 | AGAACCTCCGCAC | 1684 | CAATATCTGTGCGG |
| | AGATATTG | | AGATATTG | | AGGTTCTTA |
| 1165 | AGTGAGCTTGTCTGC | 1425 | TGAGCTTGTCTGCC | 1685 | ACCCAGTGGCAGAC |
| | CACTGGGT | | ACTGGGT | | AAGCTCACT |
| 1166 | CTGTTGCTAAGCTTC | 1426 | GTTGCTAAGCTTCC | 1686 | TTGGGCAGGAAGCT |
| | CTGCCCAA | | TGCCCAA | | TAGCAACAG |
| 1167 | TGGGCGGCTGTGCAA | 1427 | GGCGGCTGTGCAA | 1687 | GGTTGGTTTTGCAC |
| | AACCAACC | | AACCAACC | | AGCCGCCCA |
| 1168 | CTGGCATCGTCATGA | 1428 | GGCATCGTCATGAG | 1688 | GTACATACTCATGA |
| | GTATGTAC | | TATGTAC | | CGATGCCAG |
| 1169 | TGTTGCTAAGCTTCC | 1429 | TTGCTAAGCTTCCT | 1689 | TTTGGGCAGGAAGC |
| | TGCCCAAA | | GCCCAAA | | TTAGCAACA |
| 1170 | TTGCCAAGGTAACCA | 1430 | GCCAAGGTAACCA | 1690 | CATGCCACTGGTTA |
| | GTGGCATG | | GTGGCATG | | CCTTGGCAA |
| 1171 | ATTGGTGCTGTGGTT | 1431 | TGGTGCTGTGGTTG | 1691 | GTGTCAGCAACCAC |
| | GCTGACAC | | CTGACAC | | AGCACCAAT |
| 1172 | GAATGGCAGAAAGG | 1432 | ATGGCAGAAAGGT | 1692 | TCTCCACCACCTTTC |
| | TGGTGGAGA | | GGTGGAGA | | TGCCATTC |
| 1173 | ATAGGAAGCACCAA | 1433 | AGGAAGCACCAAG | 1693 | AGCCTCGTCTTGGT |
| | GACGAGGCT | | ACGAGGCT | | GCTTCCTAT |
| 1174 | TTTGAGACTAACTCA | 1434 | TGAGACTAACTCAG | 1694 | GGGTTCCCTGAGTT |
| | GGGAACCC | | GGAACCC | | AGTCTCAAA |
| 1175 | CTGCAAACCCTGGTC | 1435 | GCAAACCCTGGTCT | 1695 | CCCTCACAGACCAG |
| | TGTGAGGG | | GTGAGGG | | GGTTTGCAG |
| 1176 | TGTGTCTACACAGAA | 1436 | TGTCTACACAGAAC | 1696 | TCATGGTGTTCTGTG |
| | CACCATGA | | ACCATGA | | TAGACACA |
| 1177 | CAAGGGGTGAAAAT | 1437 | AGGGGTGAAAATC | 1697 | TCATAGGTGATTTTC |
| | CACCTATGA | | ACCTATGA | | ACCCCTTG |
| 1178 | AAACCCTGCCATTGA | 1438 | ACCCTGCCATTGAT | 1698 | GTCCAATATCAATG |
| | TATTGGAC | | ATTGGAC | | GCAGGGTTT |
| 1179 | GGATGTTGGCTCCAG | 1439 | ATGTTGGCTCCAGT | 1699 | GGTTTAGACTGGAG |
| | TCTAAACC | | CTAAACC | | CCAACATCC |
| 1180 | TGAGGACATGCTGAT | 1440 | AGGACATGCTGAT | 1700 | CACCAGTTATCAGC |
| | AACTGGTG | | AACTGGTG | | ATGTCCTCA |
| 1181 | TCCTTTTATGGACCT | 1441 | CTTTTATGGACCTG | 1701 | TTCAGCTCAGGTCC |
| | GAGCTGAA | | AGCTGAA | | ATAAAAGGA |
| 1182 | GCAAGCTCTCAGTAT | 1442 | AAGCTCTCAGTATC | 1702 | CGAGCATGATACTG |
| | CATGCTCG | | ATGCTCG | | AGAGCTTGC |
| 1183 | TATGACCTGAAAACT | 1443 | TGACCTGAAAACTC | 1703 | GTAACTGGAGTTTT |
| | CCAGTTAC | | CAGTTAC | | CAGGTCATA |
| 1184 | GATATAGTTGGACTG | 1444 | TATAGTTGGACTGG | 1704 | TCAAGCACCAGTCC |
| | GTGCTTGA | | TGCTTGA | | AACTATATC |
| 1185 | AGCACCCGGGCCCAC | 1445 | CACCCGGGCCCAC | 1705 | ATCTTGGCGTGGGC |
| | GCCAAGAT | | GCCAAGAT | | CCGGGTGCT |
| 1186 | CTCAAGTTTCTGCGG | 1446 | CAAGTTTCTGCGGT | 1706 | TTTACCAACCGCAG |
| | TTGGTAAA | | TGGTAAA | | AAACTTGAG |
| 1187 | TGGGATTTGGAAGAC | 1447 | GGATTTGGAAGAC | 1707 | ATTCACTTGTCTTCC |
| | AAGTGAAT | | AAGTGAAT | | AAATCCCA |
| 1188 | GGTTTTATAGAACAC | 1448 | TTTTATAGAACACC | 1708 | CAGATTGGGTGTTC |
| | CCAATCTG | | CAATCTG | | TATAAAACC |
| 1189 | GCACAGTGATGCTCT | 1449 | ACAGTGATGCTCTC | 1709 | ATACTTGGAGAGCA |
| | CCAAGTAT | | CAAGTAT | | TCACTGTGC |
| 1190 | GACCTGACACACTTC | 1450 | CCTGACACACTTCA | 1710 | TTCTGGTTGAAGTGT |
| | AACCAGAA | | ACCAGAA | | GTCAGGTC |
| 1191 | TGGAACCAAGCTCGG | 1451 | GAACCAAGCTCGG | 1711 | CTCCACAGCCGAGC |
| | CTGTGGAG | | CTGTGGAG | | TTGGTTCCA |
| 1192 | AGGGGTTTTCCGAAG | 1452 | GGGTTTTCCGAAGC | 1712 | ATTATCTGCTTCGGA |
| | CAGATAAT | | AGATAAT | | AAACCCCT |
| 1193 | GCTCTCCAAGTATGA | 1453 | TCTCCAAGTATGAT | 1713 | GCAGACGATCATAC |
| | TCGTCTGC | | CGTCTGC | | TTGGAGAGC |
| 1194 | GCTGGGAAGAATGCC | 1454 | TGGGAAGAATGCC | 1714 | GCTTGCTAGGCATT |
| | TAGCAAGC | | TAGCAAGC | | CTTCCCAGC |
| 1195 | GTCCACTTTTCTGCC | 1455 | CCACTTTTCTGCCA | 1715 | CAGGCATTGGCAGA |
| | AATGCCTG | | ATGCCTG | | AAAGTGGAC |
| 1196 | CAAGAATATGCTGTT | 1456 | AGAATATGCTGTTT | 1716 | TCATAGGAAACAGC |
| | TCCTATGA | | CCTATGA | | ATATTCTTG |
| 1197 | GTGGAACCAAGCTCG | 1457 | GGAACCAAGCTCG | 1717 | TCCACAGCCGAGCT |
| | GCTGTGGA | | GCTGTGGA | | TGGTTCCAC |
| 1198 | AGCACTAACACTGTG | 1458 | CACTAACACTGTGC | 1718 | GTGTTGGGCACAGT |
| | CCCAACAC | | CCAACAC | | GTTAGTGCT |
| 1199 | AGCTTTGTTGCAAAA | 1459 | CTTTGTTGCAAAAA | 1719 | CCCAACATTTTTGCA |
| | ATGTTGGG | | TGTTGGG | | ACAAAGCT |
| 1200 | TTGTTTGTTTCATTTC | 1460 | GTTTGTTTCATTTC | 1720 | ATCAGCGGAAATGA |
| | CGCTGAT | | CGCTGAT | | AACAAACAA |
| 1201 | CTTCTTTGCCATCAA | 1461 | TCTTTGCCATCAAA | 1721 | TGGCATCTTTGATG |
| | AGATGCCA | | GATGCCA | | GCAAAGAAG |
| 1202 | ATCTGGGCTACAGCT | 1462 | CTGGGCTACAGCTT | 1722 | AGTCTCAAAGCTGT |
| | TTGAGACT | | TGAGACT | | AGCCCAGAT |
| 1203 | CAGCACACAGGTAAT | 1463 | GCACACAGGTAAT | 1723 | TTCACGTTATTACCT |
| | AACGTGAA | | AACGTGAA | | GTGTGCTG |
| 1204 | TTACTTGTGTTGGGC | 1464 | ACTTGTGTTGGGCA | 1724 | AATGATATGCCCAA |
| | ATATCATT | | TATCATT | | CACAAGTAA |
| 1205 | TGCCGCTGCACAGGC | 1465 | CCGCTGCACAGGCT | 1725 | GGTCTGTAGCCTGT |
| | TACAGACC | | ACAGACC | | GCAGCGGCA |
| 1206 | GGAAGGAATAGGAA | 1466 | AAGGAATAGGAAG | 1726 | TCTTGGTGCTTCCTA |
| | GCACCAAGA | | CACCAAGA | | TTCCTTCC |
| 1207 | CCTCAAGTTTCTGCG | 1467 | TCAAGTTTCTGCGG | 1727 | TTACCAACCGCAGA |
| | GTTGGTAA | | TTGGTAA | | AACTTGAGG |
| 1208 | GTGGAAGGAATAGG | 1468 | GGAAGGAATAGGA | 1728 | TTGGTGCTTCCTATT |
| | AAGCACCAA | | AGCACCAA | | CCTTCCAC |
| 1209 | ACAAACCTCTAGAAG | 1469 | AAACCTCTAGAAG | 1729 | GGTTTAAGCTTCTA |
| | CTTAAACC | | CTTAAACC | | GAGGTTTGT |
| 1210 | TGGATGTTGGCTCCA | 1470 | GATGTTGGCTCCAG | 1730 | GTTTAGACTGGAGC |
| | GTCTAAAC | | TCTAAAC | | CAACATCCA |
| 1211 | TTCCACATGGACAAC | 1471 | CCACATGGACAACT | 1731 | TTATAGCAGTTGTCC |
| | TGCTATAA | | GCTATAA | | ATGTGGAA |
| 1212 | ATGAGGACATGCTGA | 1472 | GAGGACATGCTGA | 1732 | ACCAGTTATCAGCA |
| | TAACTGGT | | TAACTGGT | | TGTCCTCAT |
| 1213 | CAAGCTCTCAGTATC | 1473 | AGCTCTCAGTATCA | 1733 | CCGAGCATGATACT |
| | ATGCTCGG | | TGCTCGG | | GAGAGCTTG |
| 1214 | CTTACTGGACAGCCT | 1474 | TACTGGACAGCCTG | 1734 | GGTTATACAGGCTG |
| | GTATAACC | | TATAACC | | TCCAGTAAG |
| 1215 | AGCAAACCGGATTGT | 1475 | CAAACCGGATTGTG | 1735 | CTCGAACCACAATC |
| | GGTTCGAG | | GTTCGAG | | CGGTTTGCT |
| 1216 | GCTTTATTCCACATG | 1476 | TTTATTCCACATGG | 1736 | CAGTTGTCCATGTG |
| | GACAACTG | | ACAACTG | | GAATAAAGC |
| 1217 | TCACTGCACCATTGC | 1477 | ACTGCACCATTGCT | 1737 | TTGGAACAGCAATG |
| | TGTTCCAA | | GTTCCAA | | GTGCAGTGA |
| 1218 | AGATCCAGAGACAA | 1478 | ATCCAGAGACAAC | 1738 | CCAAAAGGGTTGTC |
| | CCCTTTTGG | | CCTTTTGG | | TCTGGATCT |
| 1219 | ATATAGTTGGACTGG | 1479 | ATAGTTGGACTGGT | 1739 | ATCAAGCACCAGTC |
| | TGCTTGAT | | GCTTGAT | | CAACTATAT |
| 1220 | CTGCTGAGCCCGGAG | 1480 | GCTGAGCCCGGAG | 1740 | AGTATCTCCTCCGG |
| | GAGATACT | | GAGATACT | | GCTCAGCAG |
| 1221 | GCATAATTCCCACCA | 1481 | ATAATTCCCACCAA | 1741 | TCCAAACTTGGTGG |
| | AGTTTGGA | | GTTTGGA | | GAATTATGC |
| 1222 | TGCGGCTTGTCAGAC | 1482 | CGGCTTGTCAGACC | 1742 | TCAAGATGGTCTGA |
| | CATCTTGA | | ATCTTGA | | CAAGCCGCA |
| 1223 | AAGGATCTGCAGCTA | 1483 | GGATCTGCAGCTAT | 1743 | GCCTTAGATAGCTG |
| | TCTAAGGC | | CTAAGGC | | CAGATCCTT |
| 1224 | TATGTACACACTGCT | 1484 | TGTACACACTGCTC | 1744 | GATTCCGGAGCAGT |
| | CCGGAATC | | CGGAATC | | GTGTACATA |
| 1225 | GGAGAGAATTGCCA | 1485 | AGAGAATTGCCAA | 1745 | CGTGGCTTTTGGCA |
| | AAAGCCACG | | AAGCCACG | | ATTCTCTCC |
| 1226 | AGGAGATACTGCTCT | 1486 | GAGATACTGCTCTC | 1746 | CTCTATGGAGAGCA |
| | CCATAGAG | | CATAGAG | | GTATCTCCT |
| 1227 | CTACCTGCCAGTGTC | 1487 | ACCTGCCAGTGTCT | 1747 | TCCTAAGAGACACT |
| | TCTTAGGA | | CTTAGGA | | GGCAGGTAG |
| 1228 | GTGTCTCTTAGGAGT | 1488 | GTCTCTTAGGAGTG | 1748 | GGTACCTCACTCCT |
| | GAGGTACC | | AGGTACC | | AAGAGACAC |
| 1229 | CACTTTCGCCAGTGC | 1489 | CTTTCGCCAGTGCA | 1749 | GTAAAGTTGCACTG |
| | AACTTTAC | | ACTTTAC | | GCGAAAGTG |
| 1230 | CTCAGCACACAGGTA | 1490 | CAGCACACAGGTA | 1750 | CACGTTATTACCTGT |
| | ATAACGTG | | ATAACGTG | | GTGCTGAG |
| 1231 | GGCCTCCCGGAGAGA | 1491 | CCTCCCGGAGAGA | 1751 | TGTCATCTTCTCTCC |
| | AGATGACA | | AGATGACA | | GGGAGGCC |
| 1232 | AAGGGGTTTTCCGAA | 1492 | GGGGTTTTCCGAAG | 1752 | TTATCTGCTTCGGAA |
| | GCAGATAA | | CAGATAA | | AACCCCTT |
| 1233 | AAGAGCACTTCTACC | 1493 | GAGCACTTCTACCT | 1753 | AGTCTCCAGGTAGA |
| | TGGAGACT | | GGAGACT | | AGTGCTCTT |
| 1234 | CAACCTTCCCTCCAA | 1494 | ACCTTCCCTCCAAC | 1754 | AGGCCGTGTTGGAG |
| | CACGGCCT | | ACGGCCT | | GGAAGGTTG |
| 1235 | GGAACCAAGCTCGGC | 1495 | AACCAAGCTCGGCT | 1755 | TCTCCACAGCCGAG |
| | TGTGGAGA | | GTGGAGA | | CTTGGTTCC |
| 1236 | CTCTCAGTATCATGC | 1496 | CTCAGTATCATGCT | 1756 | TCTTCCGAGCATGA |
| | TCGGAAGA | | CGGAAGA | | TACTGAGAG |
| 1237 | AGCAAGCTCTCAGTA | 1497 | CAAGCTCTCAGTAT | 1757 | GAGCATGATACTGA |
| | TCATGCTC | | CATGCTC | | GAGCTTGCT |
| 1238 | TCCAGTCTAAACCCT | 1498 | CAGTCTAAACCCTG | 1758 | TCAATGGCAGGGTT |
| | GCCATTGA | | CCATTGA | | TAGACTGGA |
| 1239 | AGAATGAGCTGTCTC | 1499 | AATGAGCTGTCTCT | 1759 | CAGCCGGAGAGACA |
| | TCCGGCTG | | CCGGCTG | | GCTCATTCT |
| 1240 | CGATGCATGCTGGAC | 1500 | ATGCATGCTGGACC | 1760 | TCATCACGGTCCAG |
| | CGTGATGA | | GTGATGA | | CATGCATCG |
| 1241 | TCATAAGAACCTCCG | 1501 | ATAAGAACCTCCGC | 1761 | TATCTGTGCGGAGG |
| | CACAGATA | | ACAGATA | | TTCTTATGA |
| 1242 | CCAAAGTAATTTCCA | 1502 | AAAGTAATTTCCAC | 1762 | TTTCTAGGTGGAAA |
| | CCTAGAAA | | CTAGAAA | | TTACTTTGG |
| 1243 | ATAAGAACCTCCGCA | 1503 | AAGAACCTCCGCA | 1763 | AATATCTGTGCGGA |
| | CAGATATT | | CAGATATT | | GGTTCTTAT |
| 1244 | ACTGTATAAATCCAA | 1504 | TGTATAAATCCAAC | 1764 | GCAGAAGGTTGGAT |
| | CCTTCTGC | | CTTCTGC | | TTATACAGT |
| 1245 | GTTCCAGGGTTTGTT | 1505 | TCCAGGGTTTGTTT | 1765 | ATGAAACAAACAAA |
| | TGTTTCAT | | GTTTCAT | | CCCTGGAAC |
| 1246 | AGGAGAGAATTGCC | 1506 | GAGAGAATTGCCA | 1766 | GTGGCTTTTGGCAA |
| | AAAAGCCAC | | AAAGCCAC | | TTCTCTCCT |
| 1247 | GCGAGACAAGCACT | 1507 | GAGACAAGCACTA | 1767 | CACAGTGTTAGTGC |
| | AACACTGTG | | ACACTGTG | | TTGTCTCGC |
| 1248 | AAGATCCCGGCATTT | 1508 | GATCCCGGCATTTG | 1768 | ATGCTGCCAAATGC |
| | GGCAGCAT | | GCAGCAT | | CGGGATCTT |
| 1249 | CCTCCCGGAGAGAAG | 1509 | TCCCGGAGAGAAG | 1769 | AATGTCATCTTCTCT |
| | ATGACATT | | ATGACATT | | CCGGGAGG |
| 1250 | ATAATTCCCACCAAG | 1510 | AATTCCCACCAAGT | 1770 | ATTCCAAACTTGGT |
| | TTTGGAAT | | TTGGAAT | | GGGAATTAT |
| 1251 | TAATCTTTACATTTG | 1511 | ATCTTTACATTTGA | 1771 | TTTGGCTTCAAATGT |
| | AAGCCAAA | | AGCCAAA | | AAAGATTA |
| 1252 | TCCACATGGACAACT | 1512 | CACATGGACAACT | 1772 | TTTATAGCAGTTGTC |
| | GCTATAAA | | GCTATAAA | | CATGTGGA |
| 1253 | CAGAAAACTGCAAA | 1513 | GAAAACTGCAAAC | 1773 | AGACCAGGGTTTGC |
| | CCCTGGTCT | | CCTGGTCT | | AGTTTTCTG |
| 1254 | TTAATCTTTACATTTG | 1514 | AATCTTTACATTTG | 1774 | TTGGCTTCAAATGT |
| | AAGCCAA | | AAGCCAA | | AAAGATTAA |
| 1255 | GTATTTCTCAGCATT | 1515 | ATTTCTCAGCATTC | 1775 | CCTGCTTGAATGCT |
| | CAAGCAGG | | AAGCAGG | | GAGAAATAC |
| 1256 | TGAAACAAATTCAAG | 1516 | AAACAAATTCAAG | 1776 | CAATTAACCTTGAA |
| | GTTAATTG | | GTTAATTG | | TTTGTTTCA |
| 1257 | TGATGTCACTAATAA | 1517 | ATGTCACTAATAAA | 1777 | AGTTTCATTTATTAG |
| | ATGAAACT | | TGAAACT | | TGACATCA |
| 1258 | AAGTTTCTGCGGTTG | 1518 | GTTTCTGCGGTTGG | 1778 | CTCTTTACCAACCGC |
| | GTAAAGAG | | TAAAGAG | | AGAAACTT |
| 1259 | TTCTATCTTCTTTGCC | 1519 | CTATCTTCTTTGCC | 1779 | CTTTGATGGCAAAG |
| | ATCAAAG | | ATCAAAG | | AAGATAGAA |
| 1260 | CCTGTATAACCTCAA | 1520 | TGTATAACCTCAAG | 1780 | ATCAGAACTTGAGG |
| | GTTCTGAT | | TTCTGAT | | TTATACAGG |
| 1261 | ATCATAAGAACCTCC | 1521 | CATAAGAACCTCCG | 1781 | ATCTGTGCGGAGGT |
| | GCACAGAT | | CACAGAT | | TCTTATGAT |
| 1262 | TGGCATCGTCATGAG | 1522 | GCATCGTCATGAGT | 1782 | TGTACATACTCATG |
| | TATGTACA | | ATGTACA | | ACGATGCCA |
| 1263 | GTTGCAGTGACAACT | 1523 | TGCAGTGACAACTG | 1783 | CCTTCCACAGTTGTC |
| | GTGGAAGG | | TGGAAGG | | ACTGCAAC |
| 1264 | CTTTTATGGACCTGA | 1524 | TTTATGGACCTGAG | 1784 | TCTTCAGCTCAGGTC |
| | GCTGAAGA | | CTGAAGA | | CATAAAAG |
| 1265 | GAAGTTCAAGAATAT | 1525 | AGTTCAAGAATATG | 1785 | GAAACAGCATATTC |
| | GCTGTTTC | | CTGTTTC | | TTGAACTTC |
| 1266 | AGGCCTCAACCCTCA | 1526 | GCCTCAACCCTCAA | 1786 | CAGCTCCTTGAGGG |
| | AGGAGCTG | | GGAGCTG | | TTGAGGCCT |
| 1267 | GAGGAACATACCAC | 1527 | GGAACATACCACA | 1787 | CCATGTTCTGTGGTA |
| | AGAACATGG | | GAACATGG | | TGTTCCTC |
| 1268 | CATCGTCATGAGTAT | 1528 | TCGTCATGAGTATG | 1788 | GTGTGTACATACTC |
| | GTACACAC | | TACACAC | | ATGACGATG |
| 1269 | ACCTGACACACTTCA | 1529 | CTGACACACTTCAA | 1789 | CTTCTGGTTGAAGT |
| | ACCAGAAG | | CCAGAAG | | GTGTCAGGT |
| 1270 | GAGCTCAGCACACAG | 1530 | GCTCAGCACACAG | 1790 | GTTATTACCTGTGTG |
| | GTAATAAC | | GTAATAAC | | CTGAGCTC |
| 1271 | AGAACATGGATCTAT | 1531 | AACATGGATCTATT | 1791 | TGACTTTAATAGAT |
| | TAAAGTCA | | AAAGTCA | | CCATGTTCT |
| 1272 | AGTGGAACCAAGCTC | 1532 | TGGAACCAAGCTC | 1792 | CCACAGCCGAGCTT |
| | GGCTGTGG | | GGCTGTGG | | GGTTCCACT |
| 1273 | CCCTGCCATTGATAT | 1533 | CTGCCATTGATATT | 1793 | CCTGTCCAATATCA |
| | TGGACAGG | | GGACAGG | | ATGGCAGGG |
| 1274 | CCCTCACCCTCAGCT | 1534 | CTCACCCTCAGCTT | 1794 | GAAGAAGAAGCTGA |
| | TCTTCTTC | | CTTCTTC | | GGGTGAGGG |
| 1275 | GTGCAGGAGAGAATT | 1535 | GCAGGAGAGAATT | 1795 | CTTTTGGCAATTCTC |
| | GCCAAAAG | | GCCAAAAG | | TCCTGCAC |
| 1276 | AGGACATGCTGATAA | 1536 | GACATGCTGATAAC | 1796 | GCCACCAGTTATCA |
| | CTGGTGGC | | TGGTGGC | | GCATGTCCT |
| 1277 | AAAAAGATTATGTTT | 1537 | AAAGATTATGTTTA | 1797 | TAAAGATTAAACAT |
| | AATCTTTA | | ATCTTTA | | AATCTTTTT |
| 1278 | TGCAGGAGAGAATTG | 1538 | CAGGAGAGAATTG | 1798 | GCTTTTGGCAATTCT |
| | CCAAAAGC | | CCAAAAGC | | CTCCTGCA |
| 1279 | AAAAAAGATTATGTT | 1539 | AAAAGATTATGTTT | 1799 | AAAGATTAAACATA |
| | TAATCTTT | | AATCTTT | | ATCTTTTTT |
| 1280 | CAAGGCCTTCATACC | 1540 | AGGCCTTCATACCA | 1800 | ACCATTTTGGTATG |
| | AAAATGGT | | AAATGGT | | AAGGCCTTG |
| 1281 | GCTGCGCTGGTTTGC | 1541 | TGCGCTGGTTTGCT | 1801 | GCTTCCCAGCAAAC |
| | TGGGAAGC | | GGGAAGC | | CAGCGCAGC |
| 1282 | ACAATGTTCTAGATT | 1542 | AATGTTCTAGATTT | 1802 | GGGAAAGAAATCTA |
| | TCTTTCCC | | CTTTCCC | | GAACATTGT |
| 1283 | GCAGCTCGAGCTCAG | 1543 | AGCTCGAGCTCAGC | 1803 | CCTGTGTGCTGAGC |
| | CACACAGG | | ACACAGG | | TCGAGCTGC |
| 1284 | TGCACCATTGCTGTT | 1544 | CACCATTGCTGTTC | 1804 | CCTTTTGGAACAGC |
| | CCAAAAGG | | CAAAAGG | | AATGGTGCA |
| 1285 | AACAAGATCGTCCAC | 1545 | CAAGATCGTCCACT | 1805 | GCAGAAAAGTGGAC |
| | TTTTCTGC | | TTTCTGC | | GATCTTGTT |
| 1286 | CACAGGTAATAACGT | 1546 | CAGGTAATAACGT | 1806 | GTTCCTTCACGTTAT |
| | GAAGGAAC | | GAAGGAAC | | TACCTGTG |
| 1287 | CAGTGGCATGAGAGT | 1547 | GTGGCATGAGAGTT | 1807 | TGAATAAAACTCTC |
| | TTTATTCA | | TTATTCA | | ATGCCACTG |
| 1288 | GCCTTCAAACCAATG | 1548 | CTTCAAACCAATGA | 1808 | TTGCTGTTCATTGGT |
| | AACAGCAA | | ACAGCAA | | TTGAAGGC |
| 1289 | CCTTCAAACCAATGA | 1549 | TTCAAACCAATGAA | 1809 | TTTGCTGTTCATTGG |
| | ACAGCAAA | | CAGCAAA | | TTTGAAGG |
| 1290 | AAATCCAACCAACTC | 1550 | ATCCAACCAACTCA | 1810 | CAATAATTGAGTTG |
| | AATTATTG | | ATTATTG | | GTTGGATTT |
| 1291 | TACAAAAAAGATTAT | 1551 | CAAAAAAGATTAT | 1811 | GATTAAACATAATC |
| | GTTTAATC | | GTTTAATC | | TTTTTTGTA |
| 1292 | ACCTGTGGGATGCCT | 1552 | CTGTGGGATGCCTG | 1812 | TCCTCTGCAGGCAT |
| | GCAGAGGA | | CAGAGGA | | CCCACAGGT |
| 1293 | AGCCCTCAAGACCAC | 1553 | CCCTCAAGACCACT | 1813 | GCCTCTGAGTGGTC |
| | TCAGAGGC | | CAGAGGC | | TTGAGGGCT |
| 1294 | GTGGGATGCCTGCAG | 1554 | GGGATGCCTGCAG | 1814 | CACCTCCTCTGCAG |
| | AGGAGGTG | | AGGAGGTG | | GCATCCCAC |
| 1295 | AACCAGTGGCATGAG | 1555 | CCAGTGGCATGAG | 1815 | ATAAAACTCTCATG |
| | AGTTTTAT | | AGTTTTAT | | CCACTGGTT |
| 1296 | ATGGATCTATTAAAG | 1556 | GGATCTATTAAAGT | 1816 | TTCTGTGACTTTAAT |
| | TCACAGAA | | CACAGAA | | AGATCCAT |
| 1297 | CCCTTCCTGTGATCC | 1557 | CTTCCTGTGATCCA | 1817 | AGTAAAATGGATCA |
| | ATTTTACT | | TTTTACT | | CAGGAAGGG |
| 1298 | TCCTGGCTGCTTCTA | 1558 | CTGGCTGCTTCTAT | 1818 | AAAGAAGATAGAAG |
| | TCTTCTTT | | CTTCTTT | | CAGCCAGGA |
| 1299 | TGTGTACACAGATGG | 1559 | TGTACACAGATGGC | 1819 | GCACAGAGCCATCT |
| | CTCTGTGC | | TCTGTGC | | GTGTACACA |
| 1300 | CTTCAAGTTCTACCT | 1560 | TCAAGTTCTACCTG | 1820 | GGACTGTCAGGTAG |
| | GACAGTCC | | ACAGTCC | | AACTTGAAG |

TABLE 3

| SEQ ID NO | D21_21_Guide (Antisense) | SEQ ID NO | D21_21_Passenger (Sense) |
|---|---|---|---|
| 1821 | AGACGATCATACTTGGAGAGC | 2081 | TCTCCAAGTATGATCGTCTGC |
| 1822 | TGGACGATCTTGTTCTGCAGA | 2082 | TGCAGAACAAGATCGTCCACT |
| 1823 | GGCATTGGCAGAAAAGTGGAC | 2083 | CCACTTTTCTGCCAATGCCTG |
| 1824 | AACCCGCACTGGGAGCCGTGG | 2084 | ACGGCTCCCAGTGCGGGTTCT |
| 1825 | CTCAATGCCAATCTCCGTGTT | 2085 | CACGGAGATTGGCATTGAGAT |
| 1826 | TCTCAATGCCAATCTCCGTGT | 2086 | ACGGAGATTGGCATTGAGATG |
| 1827 | TGGCAATGTCATCTTCTCTCC | 2087 | AGAGAAGATGACATTGCCAAG |
| 1828 | AAAACTCTCATGCCACTGGTT | 2088 | CCAGTGGCATGAGAGTTTTAT |
| 1829 | GCCTCACCAGAGGCCTGCATG | 2089 | TGCAGGCCTCTGGTGAGGCCG |
| 1830 | ACAGTACACGGCCTCACCAGA | 2090 | TGGTGAGGCCGTGTACTGTGA |
| 1831 | TGATCTTGGCGTGGGCCCGGG | 2091 | CGGGCCCACGCCAAGATCAAG |
| 1832 | TCTATGGACTTGATCTTGGCG | 2092 | CCAAGATCAAGTCCATAGATA |
| 1833 | CACCAATGATATGCCCAACAC | 2093 | GTTGGGCATATCATTGGTGCT |
| 1834 | AGCAACCACAGCACCAATGAT | 2094 | CATTGGTGCTGTGGTTGCTGA |
| 1835 | TCGAACCACAATCCGGTTTGC | 2095 | AAACCGGATTGTGGTTCGAGT |
| 1836 | CACTCGAACCACAATCCGGTT | 2096 | CCGGATTGTGGTTCGAGTGAA |
| 1837 | CTTCACTCGAACCACAATCCG | 2097 | GATTGTGGTTCGAGTGAAGAG |
| 1838 | GTCCTCATCACGGTCCAGCAT | 2098 | GCTGGACCGTGATGAGGACAT |
| 1839 | AGTTATCAGCATGTCCTCATC | 2099 | TGAGGACATGCTGATAACTGG |
| 1840 | GAAGCCAACCTTGTATCTGGC | 2100 | CAGATACAAGGTTGGCTTCAT |
| 1841 | TTCCATAATACTCTGAGAGAG | 2101 | CTCTCAGAGTATTATGGAACG |
| 1842 | CCGTGTTGGAGGGAAGGTTGG | 2102 | AACCTTCCCTCCAACACGGCC |
| 1843 | GATACTGAGAGCTTGCTAGGC | 2103 | CTAGCAAGCTCTCAGTATCAT |
| 1844 | CATGATACTGAGAGCTTGCTA | 2104 | GCAAGCTCTCAGTATCATGCT |
| 1845 | CTTCCGAGCATGATACTGAGA | 2105 | TCAGTATCATGCTCGGAAGAG |
| 1846 | CTTATTCCAAACTTGGTGGGA | 2106 | CCACCAAGTTTGGAATAAGCT |
| 1847 | ATGACAATATCTGTGCGGAGG | 2107 | TCCGCACAGATATTGTCATGG |
| 1848 | GCCAAATGCCGGGATCTTGTA | 2108 | CAAGATCCCGGCATTTGGCAG |
| 1849 | ACGTTATTACCTGTGTGCTGA | 2109 | AGCACACAGGTAATAACGTGA |
| 1850 | GACCCTCACAGACCAGGGTTT | 2110 | ACCCTGGTCTGTGAGGGTCTA |
| 1851 | ATAGATCCATGTTCTGTGGTA | 2111 | CCACAGAACATGGATCTATTA |
| 1852 | GACTTTAATAGATCCATGTTC | 2112 | ACATGGATCTATTAAAGTCAC |
| 1853 | GTGACTTTAATAGATCCATGT | 2113 | ATGGATCTATTAAAGTCACAG |
| 1854 | GAATAGCACAAACCCTTCCCG | 2114 | GGAAGGGTTTGTGCTATTCCC |
| 1855 | GGAATAGCACAAACCCTTCCC | 2115 | GAAGGGTTTGTGCTATTCCCC |
| 1856 | GACACCATCAGAACTTGAGGT | 2116 | CTCAAGTTCTGATGGTGTCTG |
| 1857 | AGACACCATCAGAACTTGAGG | 2117 | TCAAGTTCTGATGGTGTCTGT |
| 1858 | GCTTCTAGAGGTTTGTGGGAA | 2118 | CCCACAAACCTCTAGAAGCTT |
| 1859 | AAGCTTCTAGAGGTTTGTGGG | 2119 | CACAAACCTCTAGAAGCTTAA |
| 1860 | CTGTTCATTGGTTTGAAGGCC | 2120 | CCTTCAAACCAATGAACAGCA |
| 1861 | TTATGCTTTGCTGTTCATTGG | 2121 | AATGAACAGCAAAGCATAACC |
| 1862 | AATTAACCTTGAATTTGTTTC | 2122 | AACAAATTCAAGGTTAATTGG |
| 1863 | ATCTTGCTTTATGCAGCTTCA | 2123 | AAGCTGCATAAAGCAAGATTA |
| 1864 | AGTAATCTTGCTTTATGCAGC | 2124 | TGCATAAAGCAAGATTACTCT |
| 1865 | AAGATTAAACATAATCTTTTT | 2125 | AAAGATTATGTTTAATCTTTA |
| 1866 | AGTAAGAAAACCAAGCCTTAG | 2126 | AAGGCTTGGTTTTCTTACTGT |
| 1867 | ATATGACAGTAAGAAAACCAA | 2127 | GGTTTTCTTACTGTCATATGA |
| 1868 | ACCAACCGCAGAAACTTGAGG | 2128 | TCAAGTTTCTGCGGTTGGTAA |
| 1869 | TACCAACCGCAGAAACTTGAG | 2129 | CAAGTTTCTGCGGTTGGTAAA |
| 1870 | ACATCAAGCACCAGTCCAACT | 2130 | TTGGACTGGTGCTTGATGTCA |
| 1871 | CATTCACTTGTCTTCCAAATC | 2131 | TTTGGAAGACAAGTGAATGCA |
| 1872 | CATACTTGGAGAGCATCACTG | 2132 | GTGATGCTCTCCAAGTATGAT |
| 1873 | ACTCGAACCACAATCCGGTTT | 2133 | ACCGGATTGTGGTTCGAGTGA |
| 1874 | TCTTTACCAACCGCAGAAACT | 2134 | TTTCTGCGGTTGGTAAAGAGA |
| 1875 | ATGGCAAAGAAGATAGAAGCA | 2135 | CTTCTATCTTCTTTGCCATCA |
| 1876 | CATGTTCTGTGGTATGTTCCT | 2136 | GAACATACCACAGAACATGGA |
| 1877 | CTTCAGCTCAGGTCCATAAAA | 2137 | TTATGGACCTGAGCTGAAGAT |
| 1878 | CTTGATCTTGGCGTGGGCCCG | 2138 | GGCCCACGCCAAGATCAAGTC |
| 1879 | GGGAATAGCACAAACCCTTCC | 2139 | AAGGGTTTGTGCTATTCCCCA |
| 1880 | TGCAGTAAAATGGATCACAGG | 2140 | TGTGATCCATTTTACTGCAAA |
| 1881 | GCTCAATAATTGAGTTGGTTG | 2141 | ACCAACTCAATTATTGAGCAC |
| 1882 | AGCCTTAGATAGCTGCAGATC | 2142 | TCTGCAGCTATCTAAGGCTTG |
| 1883 | ACAACATTATCTGCTTCGGAA | 2143 | CCGAAGCAGATAATGTTGTGT |
| 1884 | TTCCGGAGCAGTGTGTACATA | 2144 | TGTACACACTGCTCCGGAATC |
| 1885 | GCCAAAAGGGTTGTCTCTGGA | 2145 | CAGAGACAACCCTTTTGGCCT |
| 1886 | GGATGCTGCCAAATGCCGGGA | 2146 | CCGGCATTTGGCAGCATCCCC |
| 1887 | TATGCCCAACACAAGTAACCT | 2147 | GTTACTTGTGTTGGGCATATC |
| 1888 | CTTGCTAGGCATTCTTCCCAG | 2148 | GGGAAGAATGCCTAGCAAGCT |
| 1889 | TGCATTCACTTGTCTTCCAAA | 2149 | TGGAAGACAAGTGAATGCAAT |
| 1890 | AGAGTAATCTTGCTTTATGCA | 2150 | CATAAAGCAAGATTACTCTAT |
| 1891 | AGCTTATTCCAAACTTGGTGG | 2151 | ACCAAGTTTGGAATAAGCTTT |
| 1892 | ACAATTCTCCTTGTTGAACTT | 2152 | GTTCAACAAGGAGAATTGTTG |
| 1893 | CTGGATCTGCATTTTTCTCCA | 2153 | GAGAAAAATGCAGATCCAGAG |
| 1894 | ATGATACTGAGAGCTTGCTAG | 2154 | AGCAAGCTCTCAGTATCATGC |
| 1895 | TACATACTCATGACGATGCCA | 2155 | GCATCGTCATGAGTATGTACA |
| 1896 | GCAACATGGTGCAAGGAGCAG | 2156 | GCTCCTTGCACCATGTTGCAG |
| 1897 | CTCAATAATTGAGTTGGTTGG | 2157 | AACCAACTCAATTATTGAGCA |
| 1898 | ATGATATGCCCAACACAAGTA | 2158 | CTTGTGTTGGGCATATCATTG |
| 1899 | CAACACAAGTAACCTTATCCT | 2159 | GATAAGGTTACTTGTGTTGGG |
| 1900 | GAGCATCACTGTGCAAGCCCC | 2160 | GGCTTGCACAGTGATGCTCTC |
| 1901 | TTAACCTTGAATTTGTTTCAT | 2161 | GAAACAAATTCAAGGTTAATT |
| 1902 | GACAGTCCAAGATCACAAAGA | 2162 | TTTGTGATCTTGGACTGTCAA |
| 1903 | TGGATCTGCATTTTTCTCCAC | 2163 | GGAGAAAAATGCAGATCCAGA |
| 1904 | CCCTGACACAACATTATCTGC | 2164 | AGATAATGTTGTGTCAGGGGA |
| 1905 | GCTTATTCCAAACTTGGTGGG | 2165 | CACCAAGTTTGGAATAAGCTT |
| 1906 | AGATTCAAGGTTATGCTTTGC | 2166 | AAAGCATAACCTTGAATCTAT |
| 1907 | CTTCACGTTATTACCTGTGTG | 2167 | CACAGGTAATAACGTGAAGGA |
| 1908 | TCAATTGTGATAATGGCTGGT | 2168 | CAGCCATTATCACAATTGAGG |
| 1909 | TTGAGTTGGTTGGATTTTTGT | 2169 | AAAAATCCAACCAACTCAATT |
| 1910 | TCGTCTTGGTGCTTCCTATTC | 2170 | ATAGGAAGCACCAAGACGAGG |
| 1911 | TGTCCATTGAGGTCAGCGCTG | 2171 | GCGCTGACCTCAATGGACAGG |
| 1912 | ATGTCATCTTCTCTCCGGGAG | 2172 | CCCGGAGAGAAGATGACATTG |
| 1913 | CACCTGTCCAATATCAATGGC | 2173 | CATTGATATTGGACAGGTGGA |
| 1914 | TCACGTTATTACCTGTGTGCT | 2174 | CACACAGGTAATAACGTGAAG |
| 1915 | TGCATATTCACCATTTAGGCA | 2175 | CCTAAATGGTGAATATGCAAT |
| 1916 | TTCTATAAAACCCAGTGGCAG | 2176 | GCCACTGGGTTTTATAGAACA |
| 1917 | CTGATTCCGGAGCAGTGTGTA | 2177 | CACACTGCTCCGGAATCAGCC |
| 1918 | GATATGCCCAACACAAGTAAC | 2178 | TACTTGTGTTGGGCATATCAT |
| 1919 | TCGTCACAGTACACGGCCTCA | 2179 | AGGCCGTGTACTGTGACGACA |
| 1920 | TGATACTGAGAGCTTGCTAGG | 2180 | TAGCAAGCTCTCAGTATCATG |
| 1921 | TGAACTTCATCTCAATGCCAA | 2181 | GGCATTGAGATGAAGTTCAAG |
| 1922 | GTAGCCCAGATTGGGTGTTCT | 2182 | AACACCCAATCTGGGCTACAG |
| 1923 | TCAGCGGAAATGAAACAAACA | 2183 | TTTGTTTCATTTCCGCTGATG |
| 1924 | CCCATAGCTGAAGTAGTGGAA | 2184 | CCACTACTTCAGCTATGGGGT |
| 1925 | TAGATCCATGTTCTGTGGTAT | 2185 | ACCACAGAACATGGATCTATT |
| 1926 | ATATCTGTGCGGAGGTTCTTA | 2186 | AGAACCTCCGCACAGATATTG |
| 1927 | CAAATGCCGGGATCTTGTAGG | 2187 | TACAAGATCCCGGCATTTGGC |
| 1928 | CCGGAGCAGTGTGTACATACT | 2188 | TATGTACACACTGCTCCGGAA |
| 1929 | CTGCAACATGGTGCAAGGAGC | 2189 | TCCTTGCACCATGTTGCAGTG |
| 1930 | TTCTAGGTGGAAATTACTTTG | 2190 | AAGTAATTTCCACCTAGAAAT |
| 1931 | CCCGCACTGGGAGCCGTGGCT | 2191 | CCACGGCTCCCAGTGCGGGTT |
| 1932 | AAACCCAGTGGCAGACAAGCT | 2192 | CTTGTCTGCCACTGGGTTTTA |
| 1933 | TTATTAGTGACATCAAGCACC | 2193 | TGCTTGATGTCACTAATAAAT |
| 1934 | CACGGATGGCATCTTTGATGG | 2194 | ATCAAAGATGCCATCCGTGCA |
| 1935 | CAGACGATCATACTTGGAGAG | 2195 | CTCCAAGTATGATCGTCTGCA |
| 1936 | GTGGTATGTTCCTCCTGCTCC | 2196 | AGCAGGAGGAACATACCACAG |
| 1937 | CCATAATACTCTGAGAGAGAT | 2197 | CTCTCTCAGAGTATTATGGAA |
| 1938 | GTCAACCTCACTCTTCCGAGC | 2198 | TCGGAAGAGTGAGGTTGACAA |
| 1939 | GTCCATTGAGGTCAGCGCTGA | 2199 | AGCGCTGACCTCAATGGACAG |
| 1940 | GCAGTTGTCCATGTGGAATAA | 2200 | ATTCCACATGGACAACTGCTA |
| 1941 | TGCATAGATGGCCTTCTTGTT | 2201 | CAAGAAGGCCATCTATGCATC |
| 1942 | CAATTGTGATAATGGCTGGTA | 2202 | CCAGCCATTATCACAATTGAG |
| 1943 | CAGACAAGCTCACTGTGTCCA | 2203 | GACACAGTGAGCTTGTCTGCC |
| 1944 | CAATATCTGTGCGGAGGTTCT | 2204 | AACCTCCGCACAGATATTGTC |
| 1945 | ACCCAGTGGCAGACAAGCTCA | 2205 | AGCTTGTCTGCCACTGGGTTT |
| 1946 | TTGGGCAGGAAGCTTAGCAAC | 2206 | TGCTAAGCTTCCTGCCCAAAA |
| 1947 | GGTTGGTTTTGCACAGCCGCC | 2207 | CGGCTGTGCAAAACCAACCTT |
| 1948 | GTACATACTCATGACGATGCC | 2208 | CATCGTCATGAGTATGTACAC |
| 1949 | TTTGGGCAGGAAGCTTAGCAA | 2209 | GCTAAGCTTCCTGCCCAAAAG |
| 1950 | CATGCCACTGGTTACCTTGGC | 2210 | CAAGGTAACCAGTGGCATGAG |
| 1951 | GTGTCAGCAACCACAGCACCA | 2211 | GTGCTGTGGTTGCTGACACCC |
| 1952 | TCTCCACCACCTTTCTGCCAT | 2212 | GGCAGAAAGGTGGTGGAGAAA |
| 1953 | AGCCTCGTCTTGGTGCTTCCT | 2213 | GAAGCACCAAGACGAGGCTGC |
| 1954 | GGGTTCCCTGAGTTAGTCTCA | 2214 | AGACTAACTCAGGGAACCCCT |
| 1955 | CCCTCACAGACCAGGGTTTGC | 2215 | AAACCCTGGTCTGTGAGGGTC |
| 1956 | TCATGGTGTTCTGTGTAGACA | 2216 | TCTACACAGAACACCATGAAG |
| 1957 | TCATAGGTGATTTTCACCCCT | 2217 | GGGTGAAAATCACCTATGAAG |
| 1958 | GTCCAATATCAATGGCAGGGT | 2218 | CCTGCCATTGATATTGGACAG |
| 1959 | GGTTTAGACTGGAGCCAACAT | 2219 | GTTGGCTCCAGTCTAAACCCT |
| 1960 | CACCAGTTATCAGCATGTCCT | 2220 | GACATGCTGATAACTGGTGGC |
| 1961 | TTCAGCTCAGGTCCATAAAAG | 2221 | TTTATGGACCTGAGCTGAAGA |
| 1962 | CGAGCATGATACTGAGAGCTT | 2222 | GCTCTCAGTATCATGCTCGGA |
| 1963 | GTAACTGGAGTTTTCAGGTCA | 2223 | ACCTGAAAACTCCAGTTACAT |
| 1964 | TCAAGCACCAGTCCAACTATA | 2224 | TAGTTGGACTGGTGCTTGATG |
| 1965 | ATCTTGGCGTGGGCCCGGGTG | 2225 | CCCGGGCCCACGCCAAGATCA |
| 1966 | TTTACCAACCGCAGAAACTTG | 2226 | AGTTTCTGCGGTTGGTAAAGA |
| 1967 | ATTCACTTGTCTTCCAAATCC | 2227 | ATTTGGAAGACAAGTGAATGC |
| 1968 | CAGATTGGGTGTTCTATAAAA | 2228 | TTATAGAACACCCAATCTGGG |
| 1969 | ATACTTGGAGAGCATCACTGT | 2229 | AGTGATGCTCTCCAAGTATGA |
| 1970 | TTCTGGTTGAAGTGTGTCAGG | 2230 | TGACACACTTCAACCAGAAGC |
| 1971 | CTCCACAGCCGAGCTTGGTTC | 2231 | ACCAAGCTCGGCTGTGGAGAG |
| 1972 | ATTATCTGCTTCGGAAAACCC | 2232 | GTTTTCCGAAGCAGATAATGT |
| 1973 | GCAGACGATCATACTTGGAGA | 2233 | TCCAAGTATGATCGTCTGCAG |
| 1974 | GCTTGCTAGGCATTCTTCCCA | 2234 | GGAAGAATGCCTAGCAAGCTC |
| 1975 | CAGGCATTGGCAGAAAAGTGG | 2235 | ACTTTTCTGCCAATGCCTGCC |
| 1976 | TCATAGGAAACAGCATATTCT | 2236 | AATATGCTGTTTCCTATGATT |
| 1977 | TCCACAGCCGAGCTTGGTTCC | 2237 | AACCAAGCTCGGCTGTGGAGA |
| 1978 | GTGTTGGGCACAGTGTTAGTG | 2238 | CTAACACTGTGCCCAACACCT |
| 1979 | CCCAACATTTTTGCAACAAAG | 2239 | TTGTTGCAAAAATGTTGGGGG |
| 1980 | ATCAGCGGAAATGAAACAAAC | 2240 | TTGTTTCATTTCCGCTGATGA |
| 1981 | TGGCATCTTTGATGGCAAAGA | 2241 | TTTGCCATCAAAGATGCCATC |
| 1982 | AGTCTCAAAGCTGTAGCCCAG | 2242 | GGGCTACAGCTTTGAGACTAA |
| 1983 | TTCACGTTATTACCTGTGTGC | 2243 | ACACAGGTAATAACGTGAAGG |
| 1984 | AATGATATGCCCAACACAAGT | 2244 | TTGTGTTGGGCATATCATTGG |
| 1985 | GGTCTGTAGCCTGTGCAGCGG | 2245 | GCTGCACAGGCTACAGACCCA |
| 1986 | TCTTGGTGCTTCCTATTCCTT | 2246 | GGAATAGGAAGCACCAAGACG |
| 1987 | TTACCAACCGCAGAAACTTGA | 2247 | AAGTTTCTGCGGTTGGTAAAG |
| 1988 | TTGGTGCTTCCTATTCCTTCC | 2248 | AAGGAATAGGAAGCACCAAGA |
| 1989 | GGTTTAAGCTTCTAGAGGTTT | 2249 | ACCTCTAGAAGCTTAAACCGA |
| 1990 | GTTTAGACTGGAGCCAACATC | 2250 | TGTTGGCTCCAGTCTAAACCC |
| 1991 | TTATAGCAGTTGTCCATGTGG | 2251 | ACATGGACAACTGCTATAAAA |
| 1992 | ACCAGTTATCAGCATGTCCTC | 2252 | GGACATGCTGATAACTGGTGG |
| 1993 | CCGAGCATGATACTGAGAGCT | 2253 | CTCTCAGTATCATGCTCGGAA |
| 1994 | GGTTATACAGGCTGTCCAGTA | 2254 | CTGGACAGCCTGTATAACCTC |
| 1995 | CTCGAACCACAATCCGGTTTG | 2255 | AACCGGATTGTGGTTCGAGTG |
| 1996 | CAGTTGTCCATGTGGAATAAA | 2256 | TATTCCACATGGACAACTGCT |
| 1997 | TTGGAACAGCAATGGTGCAGT | 2257 | TGCACCATTGCTGTTCCAAAA |
| 1998 | CCAAAAGGGTTGTCTCTGGAT | 2258 | CCAGAGACAACCCTTTTGGCC |
| 1999 | ATCAAGCACCAGTCCAACTAT | 2259 | AGTTGGACTGGTGCTTGATGT |
| 2000 | AGTATCTCCTCCGGGCTCAGC | 2260 | TGAGCCCGGAGGAGATACTGC |
| 2001 | TCCAAACTTGGTGGGAATTAT | 2261 | AATTCCCACCAAGTTTGGAAT |
| 2002 | TCAAGATGGTCTGACAAGCCG | 2262 | GCTTGTCAGACCATCTTGAAA |
| 2003 | GCCTTAGATAGCTGCAGATCC | 2263 | ATCTGCAGCTATCTAAGGCTT |
| 2004 | GATTCCGGAGCAGTGTGTACA | 2264 | TACACACTGCTCCGGAATCAG |
| 2005 | CGTGGCTTTTGGCAATTCTCT | 2265 | AGAATTGCCAAAAGCCACGGC |
| 2006 | CTCTATGGAGAGCAGTATCTC | 2266 | GATACTGCTCTCCATAGAGAT |
| 2007 | TCCTAAGAGACACTGGCAGGT | 2267 | CTGCCAGTGTCTCTTAGGAGT |
| 2008 | GGTACCTCACTCCTAAGAGAC | 2268 | CTCTTAGGAGTGAGGTACCTG |
| 2009 | GTAAAGTTGCACTGGCGAAAG | 2269 | TTCGCCAGTGCAACTTTACTG |
| 2010 | CACGTTATTACCTGTGTGCTG | 2270 | GCACACAGGTAATAACGTGAA |
| 2011 | TGTCATCTTCTCTCCGGGAGG | 2271 | TCCCGGAGAGAAGATGACATT |
| 2012 | TTATCTGCTTCGGAAAACCCC | 2272 | GGTTTTCCGAAGCAGATAATG |
| 2013 | AGTCTCCAGGTAGAAGTGCTC | 2273 | GCACTTCTACCTGGAGACTCA |
| 2014 | AGGCCGTGTTGGAGGGAAGGT | 2274 | CTTCCCTCCAACACGGCCTTC |
| 2015 | TCTCCACAGCCGAGCTTGGTT | 2275 | CCAAGCTCGGCTGTGGAGAGG |
| 2016 | TCTTCCGAGCATGATACTGAG | 2276 | CAGTATCATGCTCGGAAGAGT |
| 2017 | GAGCATGATACTGAGAGCTTG | 2277 | AGCTCTCAGTATCATGCTCGG |
| 2018 | TCAATGGCAGGGTTTAGACTG | 2278 | GTCTAAACCCTGCCATTGATA |
| 2019 | CAGCCGGAGAGACAGCTCATT | 2279 | TGAGCTGTCTCTCCGGCTGGT |
| 2020 | TCATCACGGTCCAGCATGCAT | 2280 | GCATGCTGGACCGTGATGAGG |
| 2021 | TATCTGTGCGGAGGTTCTTAT | 2281 | AAGAACCTCCGCACAGATATT |
| 2022 | TTTCTAGGTGGAAATTACTTT | 2282 | AGTAATTTCCACCTAGAAATG |
| 2023 | AATATCTGTGCGGAGGTTCTT | 2283 | GAACCTCCGCACAGATATTGT |
| 2024 | GCAGAAGGTTGGATTTATACA | 2284 | TATAAATCCAACCTTCTGCCA |
| 2025 | ATGAAACAAACAAACCCTGGA | 2285 | CAGGGTTTGTTTGTTTCATTT |
| 2026 | GTGGCTTTTGGCAATTCTCTC | 2286 | GAGAATTGCCAAAAGCCACGG |
| 2027 | CACAGTGTTAGTGCTTGTCTC | 2287 | GACAAGCACTAACACTGTGCC |
| 2028 | ATGCTGCCAAATGCCGGGATC | 2288 | TCCCGGCATTTGGCAGCATCC |
| 2029 | AATGTCATCTTCTCTCCGGGA | 2289 | CCGGAGAGAAGATGACATTGC |
| 2030 | ATTCCAAACTTGGTGGGAATT | 2290 | TTCCCACCAAGTTTGGAATAA |
| 2031 | TTTGGCTTCAAATGTAAAGAT | 2291 | CTTTACATTTGAAGCCAAAGT |
| 2032 | TTTATAGCAGTTGTCCATGTG | 2292 | CATGGACAACTGCTATAAAAT |
| 2033 | AGACCAGGGTTTGCAGTTTTC | 2293 | AAACTGCAAACCCTGGTCTGT |
| 2034 | TTGGCTTCAAATGTAAAGATT | 2294 | TCTTTACATTTGAAGCCAAAG |
| 2035 | CCTGCTTGAATGCTGAGAAAT | 2295 | TTCTCAGCATTCAAGCAGGCC |
| 2036 | CAATTAACCTTGAATTTGTTT | 2296 | ACAAATTCAAGGTTAATTGGA |
| 2037 | AGTTTCATTTATTAGTGACAT | 2297 | GTCACTAATAAATGAAACTGT |
| 2038 | CTCTTTACCAACCGCAGAAAC | 2298 | TTCTGCGGTTGGTAAAGAGAA |
| 2039 | CTTTGATGGCAAAGAAGATAG | 2299 | ATCTTCTTTGCCATCAAAGAT |
| 2040 | ATCAGAACTTGAGGTTATACA | 2300 | TATAACCTCAAGTTCTGATGG |
| 2041 | ATCTGTGCGGAGGTTCTTATG | 2301 | TAAGAACCTCCGCACAGATAT |
| 2042 | TGTACATACTCATGACGATGC | 2302 | ATCGTCATGAGTATGTACACA |
| 2043 | CCTTCCACAGTTGTCACTGCA | 2303 | CAGTGACAACTGTGGAAGGAA |
| 2044 | TCTTCAGCTCAGGTCCATAAA | 2304 | TATGGACCTGAGCTGAAGATC |
| 2045 | GAAACAGCATATTCTTGAACT | 2305 | TTCAAGAATATGCTGTTTCCT |
| 2046 | CAGCTCCTTGAGGGTTGAGGC | 2306 | CTCAACCCTCAAGGAGCTGCT |
| 2047 | CCATGTTCTGTGGTATGTTCC | 2307 | AACATACCACAGAACATGGAT |
| 2048 | GTGTGTACATACTCATGACGA | 2308 | GTCATGAGTATGTACACACTG |
| 2049 | CTTCTGGTTGAAGTGTGTCAG | 2309 | GACACACTTCAACCAGAAGCT |
| 2050 | GTTATTACCTGTGTGCTGAGC | 2310 | TCAGCACACAGGTAATAACGT |
| 2051 | TGACTTTAATAGATCCATGTT | 2311 | CATGGATCTATTAAAGTCACA |
| 2052 | CCACAGCCGAGCTTGGTTCCA | 2312 | GAACCAAGCTCGGCTGTGGAG |
| 2053 | CCTGTCCAATATCAATGGCAG | 2313 | GCCATTGATATTGGACAGGTG |
| 2054 | GAAGAAGAAGCTGAGGGTGAG | 2314 | CACCCTCAGCTTCTTCTTCAA |
| 2055 | CTTTTGGCAATTCTCTCCTGC | 2315 | AGGAGAGAATTGCCAAAAGCC |
| 2056 | GCCACCAGTTATCAGCATGTC | 2316 | CATGCTGATAACTGGTGGCAG |
| 2057 | TAAAGATTAAACATAATCTTT | 2317 | AGATTATGTTTAATCTTTACA |
| 2058 | GCTTTTGGCAATTCTCTCCTG | 2318 | GGAGAGAATTGCCAAAAGCCA |
| 2059 | AAAGATTAAACATAATCTTTT | 2319 | AAGATTATGTTTAATCTTTAC |
| 2060 | ACCATTTTGGTATGAAGGCCT | 2320 | GCCTTCATACCAAAATGGTCC |
| 2061 | GCTTCCCAGCAAACCAGCGCA | 2321 | CGCTGGTTTGCTGGGAAGCAA |
| 2062 | GGGAAAGAAATCTAGAACATT | 2322 | TGTTCTAGATTTCTTTCCCTT |
| 2063 | CCTGTGTGCTGAGCTCGAGCT | 2323 | CTCGAGCTCAGCACACAGGTA |
| 2064 | CCTTTTGGAACAGCAATGGTG | 2324 | CCATTGCTGTTCCAAAAGGCG |
| 2065 | GCAGAAAAGTGGACGATCTTG | 2325 | AGATCGTCCACTTTTCTGCCA |
| 2066 | GTTCCTTCACGTTATTACCTG | 2326 | GGTAATAACGTGAAGGAACTC |
| 2067 | TGAATAAAACTCTCATGCCAC | 2327 | GGCATGAGAGTTTTATTCAAG |
| 2068 | TTGCTGTTCATTGGTTTGAAG | 2328 | TCAAACCAATGAACAGCAAAG |
| 2069 | TTTGCTGTTCATTGGTTTGAA | 2329 | CAAACCAATGAACAGCAAAGC |
| 2070 | CAATAATTGAGTTGGTTGGAT | 2330 | CCAACCAACTCAATTATTGAG |
| 2071 | GATTAAACATAATCTTTTTTG | 2331 | AAAAAGATTATGTTTAATCTT |
| 2072 | TCCTCTGCAGGCATCCCACAG | 2332 | GTGGGATGCCTGCAGAGGAGG |
| 2073 | GCCTCTGAGTGGTCTTGAGGG | 2333 | CTCAAGACCACTCAGAGGCAG |
| 2074 | CACCTCCTCTGCAGGCATCCC | 2334 | GATGCCTGCAGAGGAGGTGCG |
| 2075 | ATAAAACTCTCATGCCACTGG | 2335 | AGTGGCATGAGAGTTTTATTC |
| 2076 | TTCTGTGACTTTAATAGATCC | 2336 | ATCTATTAAAGTCACAGAATG |
| 2077 | AGTAAAATGGATCACAGGAAG | 2337 | TCCTGTGATCCATTTTACTGC |
| 2078 | AAAGAAGATAGAAGCAGCCAG | 2338 | GGCTGCTTCTATCTTCTTTGC |
| 2079 | GCACAGAGCCATCTGTGTACA | 2339 | TACACAGATGGCTCTGTGCTG |
| 2080 | GGACTGTCAGGTAGAACTTGA | 2340 | AAGTTCTACCTGACAGTCCTT |

### Chemical Modifications

Described herein, in some aspects, is an oligonucleotide or a polynucleotide comprising at least one chemical modification. In some aspects, the oligonucleotide is single-stranded. In some aspects, the oligonucleotide is an antisense oligonucleotide. In some aspects, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more chemical modifications. In some aspects, the oligonucleotide does not have an intramolecular structure feature. In some aspects, the oligonucleotide comprises at least one gap segment comprising at least one, two, three, four, five, six, seven, eight, nine, ten, 11, 12, 13, 14, 15, or more chemically modified nucleotides. In some aspects, the oligonucleotide comprises at least one wing segment comprising at least one, two, three, four, five, six, seven, eight, nine, ten, or more chemically modified nucleotides. In some aspects, the oligonucleotide comprises a 5′-end wing segment comprising at least one, two, three, four, five, six, seven, eight, nine, ten, or more chemically modified nucleotides. In some aspects, the oligonucleotide comprises a 3′-end wing segment comprising at least one, two, three, four, five, six, seven, eight, nine, ten, or more chemically modified nucleotides. In some aspects, the at least one wing segment is covalently fused to the 5′-end of the gap segment. In some aspects, the at least one wing segment is covalently fused to the 3′-end of the gap segment.

In some aspects, a polynucleic acid molecule comprises natural or synthetic or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof. In some instances, a polynucleotide or polynucleic acid molecule is a stretch of 10, 20, 30, 40, 50, 60, 70, 80, 90, 100 or more nucleotides or any number in between, linked to each other by natural phosphate bond. In some aspects a polynucleotide or polynucleic acid molecule can comprise a phosphorothioate bond.

In some aspects, the oligonucleotide comprises at least one, two, three, four, five, six, seven, eight, nine, ten, 11, 12, 13, 14, 15, 16, 17, 18, 19, 20 or more chemically modified nucleotides at the 5′ end of the oligonucleotide. In some aspects, the oligonucleotide comprises at least one, two, three, four, five, six, seven, eight, nine, ten, 11, 12, 13, 14, 15, 16, 17, 18, 19, 20 or more chemically modified nucleotides at the 3′ end of the oligonucleotide. In some aspects, the oligonucleotide comprises at least one, two, three, four, five, six, seven, eight, nine, ten, 11, 12, 13, 14, 15, 16, 17, 18, 19, 20 or more chemically modified nucleotides at both the 5′ and the 3′ end of the oligonucleotide. In some aspects, the oligonucleotide comprises at least one chemical modification in the gap segment of the oligonucleotide. In some aspects, the oligonucleotide comprises at least one chemical modification in the nucleotide bases adjacent the gap segment. In some aspects, at least 10%, 20%, 30%, 40%, 50%, 60%, 70%, 80%, 90% of the bases or internucleotide linkage of the oligonucleotide comprises modifications. In some aspects, the oligonucleotide comprises 100% modified nucleotide bases.

In some aspects, chemical modification can occur at 3′-OH group, 5′-OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′-OH or 5′-OH group, the backbone, the sugar component, or the nucleotide base, or addition of non-naturally occurring linker molecules. In some aspects, chemically modified backbone comprises a backbone other than a phosphodiester backbone. In some aspects, a modified sugar comprises a sugar other than deoxyribose (in modified DNA) or other than ribose (modified RNA). In some aspects, a modified base comprises a base other than adenine, guanine, cytosine, thymine or uracil. In some aspects, the oligonucleotide comprises at least one chemically modified base. In some instances, the comprises at least one, two, three, four, five, six, seven, eight, nine, 10, 15, 20, or more modified bases. In some cases, chemical modifications to the base moiety include natural and synthetic modifications of adenine, guanine, cytosine, thymine, or uracil, and purine or pyrimidine bases.

In some aspects, the at least one chemical modification of the oligonucleotide comprises a modification of any one of or any combination of: 2′ modified nucleotide comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non-limiting examples of chemical modification to the oligonucleotide can include: modification of one or both of non-linking or linking phosphate oxygens in the phosphodiester backbone linkage (e.g., sulfur (S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap —OH) or 3′ end of the nucleic acid sequence (3′ tail or modification of 3′ end —OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some aspects, the chemical modification of the oligonucleotide comprises at least one substitution of one or both of non-linking phosphate oxygen atoms in a phosphodiester backbone linkage of the oligonucleotide. In some aspects, the at least one chemical modification of the oligonucleotide comprises a substitution of one or more of linking phosphate oxygen atoms in a phosphodiester backbone linkage of the oligonucleotide. A non-limiting example of a chemical modification of a phosphate oxygen atom is a sulfur atom. In some aspects, the chemical modifications of the oligonucleotide comprise at least one chemical modification to a sugar of a nucleotide of the oligonucleotide. In some aspects, the chemical modifications of the oligonucleotide comprise at least one chemical modification to the sugar of the nucleotide, where the chemical modification comprises at least one locked nucleic acid (LNA). In some aspects, the chemical modifications of the oligonucleotide comprise at least one chemical modification to the sugar of the nucleotide of the oligonucleotide comprising at least one unlocked nucleic acid (UNA). In some aspects, the chemical modifications of the oligonucleotide comprise at least one chemical modification to the sugar of the nucleotide of the oligonucleotide comprising at least one ethylene nucleic acid (ENA). In some aspects, the chemical modifications of the oligonucleotide comprise at least one chemical modification to the sugar comprising a modification of a constituent of the sugar, where the sugar is a ribose sugar. In some aspects, the chemical modifications of the oligonucleotide comprise at least one chemical modification to the constituent of the ribose sugar of the nucleotide of the oligonucleotide comprising a 2′-O-methyl group. In some aspects, the chemical modifications of the oligonucleotide comprise at least one chemical modification comprising replacement of a phosphate moiety of the oligonucleotide with a dephospho linker. In some aspects, the chemical modifications of the oligonucleotide comprise at least one chemical modification of a phosphate backbone of the oligonucleotide. In some aspects, the oligonucleotide comprises a phosphorothioate group. In some aspects, the chemical modifications of the oligonucleotide comprise at least one chemical modification comprising a modification to a base of a nucleotide of the oligonucleotide. In some aspects, the chemical modifications of the oligonucleotide comprise at least one chemical modification comprising an unnatural base of a nucleotide. In some aspects, the chemical modifications of the oligonucleotide comprise at least one chemical modification comprising a morpholino group (e.g., a phosphorodiamidate morpholino oligomer, PMO), a cyclobutyl group, pyrrolidine group, or peptide nucleic acid (PNA) nucleoside surrogate. In some aspects, the chemical modifications of the oligonucleotide comprise at least one chemical modification comprising at least one stereopure nucleic acid. In some aspects, the at least one chemical modification can be positioned proximal to a 5′ end of the oligonucleotide. In some aspects, the at least one chemical modification can be positioned proximal to a 3′ end of the oligonucleotide. In some aspects, the at least one chemical modification can be positioned proximal to both 5′ and 3′ ends of the oligonucleotide.

In some aspects, an oligonucleotide comprises a backbone comprising a plurality of sugar and phosphate moieties covalently linked together. In some cases, a backbone of an oligonucleotide comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some aspects, a backbone of an oligonucleotide can lack a 5′ reducing hydroxyl, a 3′ reducing hydroxyl, or both, capable of being exposed to a solvent. In some aspects, a backbone of an oligonucleotide can lack a 5′ reducing hydroxyl, a 3′ reducing hydroxyl, or both, capable of being exposed to nucleases. In some aspects, a backbone of an oligonucleotide can lack a 5′ reducing hydroxyl, a 3′ reducing hydroxyl, or both, capable of being exposed to hydrolytic enzymes. In some instances, a backbone of an oligonucleotide can be represented as a polynucleotide sequence in a circular 2-dimensional format with one nucleotide after the other. In some instances, a backbone of an oligonucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some aspects, the oligonucleotide described herein comprises at least one chemical modification. A chemical modification can be a substitution, insertion, deletion, chemical modification, physical modification, stabilization, purification, or any combination thereof. In some cases, a modification is a chemical modification. Suitable chemical modifications comprise any one of: 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphorothioate DNA, phosphorothioate RNA, UNA, LNA, cEt, pseudouridine-5′-triphosphate, 5-methylcytidine-5′-triphosphate, 2′-O-methyl 3phosphorothioate or any combinations thereof.

In some aspects the chemical modifications and analogs used for siRNA and ASO designs can be phosphonate modifications. In one aspect, the phosphonate modification is a phosphorothioate (PS Rp isomer). In one aspect, the phosphonate modification is a phosphorothioate (PS Sp isomer). In one aspect, the phosphonate modification is a phosphorothioate (PS2). In one aspect, the phosphonate modification is a methylphosphonate (MP). In one aspect, the phosphonate modification is a methoxypropyl phosphonate (MOP). In one aspect, the phosphonate modification is a 5′-(E)-vinyl phosphonate (5′-(E)-VP). In one aspect, the phosphonate modification is a 5′methyl phosphonate (5′-MP). In one aspect, the phosphonate modification is an (S)-5′-C-methyl with phosphate. In one aspect, the phosphonate modification is 5′-phosphorothioate (5′-PS). In one aspect, the phosphonate modification can be peptide nucleic acid (PNA).

In some aspects, the chemical modifications and analogs used for siRNA and ASO designs comprise a ribose modification. In one aspect, the ribose modification is a 2′-O-Methyl (2′-OME) modification. In one aspect, the ribose modification is 2′-O-Methoxyethyl (2′-O-MOE) modification. In one aspect, the ribose modification is 2′-deoxy-2′-fluoro (2′-F). In one aspect, the ribose modification is 2′-arabino fluoro (2′-Ara-F). In one aspect, the ribose modification is 2′-O-benzyl. In one aspect, the ribose modification is 2′-O-methyl-4-pyridine (2′-O—CH2Py(4)). In one aspect the ribose modification is a locked nucleic acid (LNA). In one aspect, the ribose modification is S-cEt-BNA. In one aspect, the ribose modification is tricyclo-DNA. In one aspect, the ribose modification is PMO. In one aspect, the ribose modification is unlocked nucleic acid (UNA). In one aspect, the ribose modification is Glycol Nucleic Acid (GNA).

In one aspect, the ribose modification can be a base modification. In one aspect the base modification is pseudouridine (Ψ). In one aspect, the ribose modification is 2′-thiouridine (s2U). In one aspect, the ribose modification is N6′-methyladenosine (m6C). In one aspect, the ribose modification is 5′-methylcytidine (m5C). In one aspect, the ribose modification is 5′-fluoro-2′-dioxyuridine. In one aspect, the ribose modification is N-ethyl-piperidine (7′-EAA triazole modified adenine. In one aspect, the ribose modification is N-ethylpiperidine 6′ triazole modified adenine. In one aspect, the ribose modification is 6-phenylpyrrolocytosine, In one aspect, the ribose modification is 2′-4′-difluorotoluyl ribonucleoside (rF). In one aspect, the ribose modification is 5′-nitroindole.

In some cases, a modification can be permanent. In other cases, a modification can be transient. In some cases, multiple modifications are made to the oligonucleotide. the oligonucleotide modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some instances, chemical modifications to enhance guide stability, synthesis, localization, intracellular retention, or lengthen half-lives may not be genetically encodable. An oligonucleotide can be circular, substantially circular, or otherwise linked in a contiguous fashion (e.g. can be arranged as a loop) and can also retain a substantially similar secondary structure as a substantially similar oligonucleotide that may not be circular or may not be a loop.

### Modification of Phosphate Backbone

In some aspects, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some aspects, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some aspects, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the oligonucleotide comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, the chiral phosphate product is present in a diastereomeric excess of 95%. In some aspects, the chiral phosphate product is present in a diastereomeric excess of 96%. In some aspects, the chiral phosphate product is present in a diastereomeric excess of 97%. In some aspects, the chiral phosphate product is present in a diastereomeric excess of 98%. In some aspects, the chiral phosphate product is present in a diastereomeric excess of 99%. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N*-dimethylhydrazine (—CH2-N(CH3)-N(CH3)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some aspects, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some aspects, the chemical modification described herein comprises modification of a phosphate backbone. In some aspects, the oligonucleotide described herein comprises at least one chemically modified phosphate backbone. Exemplary chemically modification of the phosphate group or backbone can include replacing one or more of the oxygens with a different substituent. Furthermore, the modified nucleotide present in the oligonucleotide can include the replacement of an unmodified phosphate moiety with a modified phosphate as described herein. In some aspects, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified oligonucleotide can be stereopure (e.g. S or R confirmation). In some cases, the chemically modified oligonucleotide comprises stereopure phosphate modification. For example, the chemically modified oligonucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some aspects, at least one phosphate group of the oligonucleotide can be chemically modified. In some aspects, the phosphate group can be replaced by non-phosphorus containing connectors. In some aspects, the phosphate moiety can be replaced by dephospho linker. In some aspects, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some aspects, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g., 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some aspects, the chemical modification described herein comprises modification by replacement of a phosphate group. In some aspects, the oligonucleotide described herein comprises at least one chemically modification comprising a phosphate group substitution or replacement. Exemplary phosphate group replacement can include non-phosphorus containing connectors. In some aspects, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some aspects, the chemical modification described herein comprises modifying ribophosphate backbone of the oligonucleotide. In some aspects, the oligonucleotide described herein comprises at least one chemically modified ribophosphate backbone. Exemplary chemically modified ribophosphate backbone can include scaffolds that can mimic nucleic acids can also be constructed wherein the phosphate linker and ribose sugar are replaced by nuclease resistant nucleoside or nucleotide surrogates. In some aspects, the nucleobases can be tethered by a surrogate backbone. Examples can include morpholino such as a phosphorodiamidate morpholino oligomer (PMO), cyclobutyl, pyrrolidine and peptide nucleic acid (PNA) nucleoside surrogates.

### Modification of Sugar

In some aspects, the chemical modification described herein comprises modification of sugar. In some aspects, the oligonucleotide described herein comprises at least one chemically modified sugar. Exemplary chemically modified sugar can include 2′ hydroxyl group (OH) modified or replaced with a number of different “oxy” or “deoxy” substituents. In some aspects, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH2CH2O)nCH2CH2OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some aspects, the “oxy”-2′ hydroxyl group modification can include (LNA, in which the 2′ hydroxyl can be connected, e.g., by a Ci-6 alkylene or Cj-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some aspects, the “oxy”-2′ hydroxyl group modification can include the methoxyethyl group (MOE), (OCH2CH2OCH3, e.g., a PEG derivative). In some cases, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the α or γ position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the oligonucleotide described herein includes the sugar group ribose, which is a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some aspects, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some aspects, the modifications to the sugar of the oligonucleotide comprises modifying the oligonucleotide to include locked nucleic acid (LNA), unlocked nucleic acid (UNA), ethylene nucleic acid (ENA), constrained ethyl (cEt) sugar, or bridged nucleic acid (BNA).

### Modification of a Constituent of the Ribose Sugar

In some aspects, the oligonucleotide described herein comprises at least one chemical modification of a constituent of the ribose sugar. In some aspects, the chemical modification of the constituent of the ribose sugar can include 2′-O-methyl, 2′-O-methoxyethyl (2′-O-MOE), 2′-fluoro, 2′-aminoethyl, 2′-deoxy-2′-fuloarabinoucleic acid, 2′-deoxy, 2′-deoxy-2′-fluoro, 2′-O-methyl, 3′-phosphorothioate, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) 3′-phosphonoacetate (PACE), or 3′-phosphonothioacetate (thioPACE). In some aspects, the chemical modification of the constituent of the ribose sugar comprises unnatural nucleic acid. In some instances, the unnatural nucleic acids include modifications at the 5′-position and the 2′-position of the sugar ring, such as 5′-CH2-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH3 and a 5′-(S)—CH3. Unnatural nucleic acids can include 2′-substituted 5′-CH2 (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH3)3) (and analogs thereof); a methyleneamino group (CH2NH2) (and analogs thereof) or a cyano group (CN) (and analogs thereof).

In some aspects, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R1)(R2) (R=H, C1-C12 alkyl or a protecting group); and combinations thereof.

In some instances, the oligonucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH2)nO]m CH3, —O(CH2)nOCH3, —O(CH2)nNH2, —O(CH2)nCH3, —O(CH2)nONH2, and —O(CH2)nON[(CH2)n CH3)]2, where n and m are from 1 to about 10. Other chemical modifications at the 2′ position include but are not limited to: C1 to C10 lower alkyl, substituted lower alkyl, alkaryl, aralkyl, O-alkaryl, O-aralkyl, SH, SCH3, OCN, Cl, Br, CN, CF3, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH2 and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH3, and 2′-O(CH2)2OCH3 substituent groups. The substituent at the 2′ position can also be selected from allyl, amino, azido, thio, O-allyl, O—(C1-C10 alkyl), OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH2)-O-2′ (LNA); 4′-(CH2)-S-2′; 4′-(CH2)2-O-2′ (ENA); 4′-CH(CH3)-O-2′ and 4′-CH(CH2OCH3)-O-2′, and analogs thereof; 4′-C(CH3)(CH3)-O-2′ and analogs thereof.

### Modifications on the Base of Nucleotide

In some aspects, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the oligonucleotide described herein. The nucleobase of the nucleotide can be independently selected from a purine, a pyrimidine, a purine or pyrimidine analog. In some aspects, the nucleobase can be naturally occurring or synthetic derivatives of a base.

In some aspects, the chemical modification described herein comprises modifying an uracil. In some aspects, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, l-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, l-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some aspects, the chemical modification described herein comprises modifying a cytosine. In some aspects, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some aspects, the chemical modification described herein comprises modifying a adenine. In some aspects, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some aspects, the chemical modification described herein comprises modifying a guanine. In some aspects, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, 06-Methyl-2′-deoxyguanosine, 2′-F-ara-guanosine, and 2′-F-guanosine.

In some cases, the chemical modification of the oligonucleotide can include introducing or substituting a nucleic acid analog or an unnatural nucleic acid into the oligonucleotide. In some aspects, nucleic acid analog can be any one of the chemically modified nucleic acid described herein. all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some aspects, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some aspects, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some aspects, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some aspects, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH3) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification comprises chemically modifying the 5′ or 3′ end such as 5′ cap or 3′ tail of the oligonucleotide. In some aspects, the oligonucleotide comprises a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some aspects, deaza nucleotides, e.g., 7-deaza-adenosine, can be incorporated into the gRNA. In some aspects, O- and N-alkylated nucleotides, e.g., N6-methyladenosine, can be incorporated into the gRNA. In some aspects, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH2; alkylamino, dialkylamino, heterocyclyl, arylamino, diarylamino, heteroarylamino, diheteroarylamino, or amino acid); or cyano (—CN). In some aspects, the phosphate backbone can be modified as described herein, e.g., with a phosphorothioate group. In some aspects, the nucleotides in the overhang region of the gRNA can each independently be a modified or unmodified nucleotide including, but not limited to 2′-sugar modified, such as, 2′-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

In some aspects, the polynucleotides as described herein (e.g., antisense strand and/or sense strand siRNA molecules, ASOs, etc.) comprises modifications in the pattern described in Hu et al., Signal Transduction and Targeted Therapy (2020) 5:101, which is incorporated in its entirety herein.

In some aspects, the oligonucleotide comprising at least one chemical modification, upon binding to the target RNA, is more specific in recruiting the endogenous nuclease for decreasing expression the target RNA compared to an oligonucleotide sharing identical nucleic acid sequence, but without any chemical modification, with the oligonucleotide comprising at least one chemical modification. In some aspects, the oligonucleotide comprising at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more specific in recruiting the endogenous nuclease for decreasing expression the target RNA compared to an oligonucleotide sharing identical nucleic acid sequence, but without any chemical modification, with the oligonucleotide comprising at least one chemical modification.

In some aspects, the oligonucleotide comprising at least one chemical modification comprises an increased resistance towards degradation by hydrolysis compared to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis compared to an oligonucleotide sharing identical nucleic acid sequence, but without any chemical modification, with the oligonucleotide comprising at least one chemical modification.

In some aspects, the oligonucleotide comprising at least one chemical modification comprises an increased resistance towards degradation by nuclease digestion compared to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion compared to an oligonucleotide sharing identical nucleic acid sequence, but without any chemical modification, with the oligonucleotide comprising at least one chemical modification.

In some aspects, the oligonucleotide comprising at least one chemical modification induces less immunogenicity compared an oligonucleotide sharing identical nucleic acid sequence, but without any chemical modification, with the oligonucleotide comprising at least one chemical modification. In some aspects, the oligonucleotide comprising the at least chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce immunogenicity compared to immunogenicity induced by an oligonucleotide sharing identical nucleic acid sequence, but without any chemical modification, with the oligonucleotide comprising at least one chemical modification.

In some aspects, the oligonucleotide comprising at least one chemical modification induces less innate immune response relative to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce innate immune response compared to innate immune response induced by an oligonucleotide sharing identical nucleic acid sequence, but without any chemical modification, with the oligonucleotide comprising at least one chemical modification.

In some aspects, the oligonucleotide comprising at least one chemical modification, when contacted with the target RNA, is less likely to induce off-target modulating of the target RNA compared to the off-target modulating of the target RNA induced by an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce off-target modulating compared to off-target modulating induced by an oligonucleotide sharing identical nucleic acid sequence, but without any chemical modification, with the oligonucleotide comprising at least one chemical modification.

### Conjugation and Method of Delivery

Described herein, in some aspects, are methods of delivering the oligonucleotide (e.g., ASO, siRNA, dsRNA, etc.) described herein to a cell. In some aspects, the method comprises delivering directly or indirectly an oligonucleotide to the cell. In some aspects, the method comprises contacting the cell with the composition or the oligonucleotide described herein. In some aspects, the method comprises expressing the composition or the oligonucleotide described herein in the cell. In some aspects, the oligonucleotide or vector encoding the oligonucleotide can be delivered into the cell via any of the transfection methods described herein. In some aspects, the oligonucleotide can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the oligonucleotide or vector encoding the oligonucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of oligonucleotide or vector encoding the oligonucleotide into a host cell is calcium phosphate transfection.

Chemical means for introducing the oligonucleotide or vector encoding the oligonucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of oligonucleotide or vector encoding the oligonucleotide with targeted nanoparticles or other suitable sub-micron sized delivery system.

In the case where a non-viral delivery system is utilized, an exemplary delivery vehicle is a liposome. The use of lipid formulations is contemplated for the introduction of the oligonucleotide or vector encoding the oligonucleotide into a cell (in vitro, ex vivo, or in vivo). In another aspect, the oligonucleotide or vector encoding the oligonucleotide can be associated with a lipid. The oligonucleotide or vector encoding the oligonucleotide associated with a lipid, in some aspects, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some aspects, they are present in a bilayer structure, as micelles, or with a “collapsed” structure. Alternately, they are simply be interspersed in a solution, possibly forming aggregates that are not uniform in size or shape. Lipids are fatty substances which are, in some aspects, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some aspects, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some aspects, the delivery method comprises conjugating or encapsulating the compositions or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the oligonucleotide or vector encoding the oligonucleotide described herein can be packaged and delivered to the cell via extracellular vesicles. The extracellular vesicles can be any membrane-bound particles. In some aspects, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the oligonucleotide or vector encoding the oligonucleotide described herein can be administered to the subject in need thereof via the use of the transgenic cells generated by introduction of the oligonucleotide or vector encoding the oligonucleotide first into allogeneic or autologous cells. In some cases, the cell can be isolated. In some aspects, the cell can be isolated from the subject.

In some aspects, the oligonucleotide described herein is conjugated. In some aspects, the oligonucleotide is conjugated to with a peptide, antibody, lipid, carbohydrate, or polymer. In some aspects, the oligonucleotide is conjugated to with a peptide, antibody, lipid, carbohydrate, or polymer at the 5′ end of the oligonucleotide. In some aspects, the oligonucleotide is conjugated to with a peptide, antibody, lipid, carbohydrate, or polymer at the 3′ end of the oligonucleotide. In some aspects, the oligonucleotide is conjugated to with a peptide, antibody, lipid, carbohydrate, or polymer at any nucleic acid residue of the oligonucleotide. In some aspects, the peptide, antibody, lipid, carbohydrate, or polymer conjugated to the oligonucleotide confers therapeutic effect. For example, the peptide, antibody, lipid, carbohydrate, or polymer conjugated to the oligonucleotide can be cytotoxic drug or drug for treating gout or XDH-related disorders, diseases, or symptoms. In some aspects, the peptide, antibody, lipid, carbohydrate, or polymer conjugated to the oligonucleotide increases the efficiency of the oligonucleotide binding to the endogenous nucleic acid. In some aspects, the peptide, antibody, lipid, carbohydrate, or polymer conjugated to the oligonucleotide confers targeting specificity of the oligonucleotide to specific types of cells (e.g., liver cells, hepatocytes, etc.). In some aspects, the peptide, antibody, lipid, carbohydrate, or polymer conjugated to the oligonucleotide confers stability of the oligonucleotide in vitro, ex vivo, or in vivo. For example, the oligonucleotide can be conjugated with polyethylene glycol (PEG) or endosomolytic agent to decrease immunogenicity or degradation. In some aspects, the peptide, antibody, lipid, carbohydrate, or polymer conjugated to the oligonucleotide to facilitate the oligonucleotide for entering cell. In some aspects, the peptide, antibody, lipid, carbohydrate, or polymer conjugated to the oligonucleotide to facilitate and release to the oligonucleotide in the cell. In some aspects, the peptide, antibody, lipid, carbohydrate, or polymer conjugated to the oligonucleotide comprises at least one targeting moiety for targeting the cell. Non-limiting examples of the targeting moiety comprises a signaling peptide, a chemokine, a chemokine receptor, an adhesion molecule, an antigen, or an antibody.

The linker for conjugating the oligonucleotide to the peptide, antibody, lipid, or polymer can be any linker that connects biomolecules. In some aspects, a linker described herein is a cleavable linker or a non-cleavable linker. In some instances, the linker is a cleavable linker. In other instances, the linker is a non-cleavable linker. In some cases, the linker is a non-polymeric linker. A non-polymeric linker refers to a linker that does not contain a repeating unit of monomers generated by a polymerization process. In some aspects, the linker comprises a peptide moiety. In some instances, the peptide moiety comprises at least 2, 3, 4, 5, or 6 more amino acid residues. In some aspects, the linker comprises a benzoic acid group, or its derivatives thereof. In some aspects, the linker can comprise nucleic acid linker such as DNA linker. In such case, the peptide, antibody, lipid, or polymer can be conjugated on one end of the nucleic acid linker or intercalated into the nucleotide pairing of the nucleic acid linker. In some aspects, the linker can be a peptide linker. The peptide linker can be flexible (e.g., poly-glycine linker) or rigid (e.g., EAAAK repeat linker). In some aspects, the peptide linker can be cleaved (e.g., a disulfide bond). In some aspects, the linker comprises polymers such PEG, polylactic acid (PLA), or polyacrylic acid (PAA).

In some aspects, the polynucleic acid or polynucleotide of the disclosure is conjugated to a targeting moiety, for example, a sugar that helps in the uptake of the polynucleotide by a specific cell that is targeted by the targeting moiety. In some aspects, the targeting moiety helps to bind to a cell surface molecule, e.g. a membrane protein, a receptor, a glycosylated membrane protein. In some aspects, the polynucleotide is modified by a GalNAc conjugation. In some aspects, the cell targeting moiety is GalNAc. GalNAc is a carbohydrate moiety that binds to the highly liver-expressed asialoglycoprotein receptor 1 (ASGR1, ASPGR) with high affinity (Kd=2.5 nM) and facilitates the uptake of the ASOs and siRNAs into hepatocytes by endocytosis. In some aspects, the siRNA is designed to be directly conjugated a triantennary GalNAc sugar. GalNAc-siRNA conjugates can lead to the siRNA delivery problem for hepatocytes and have shown the RNAi field the path forward for targeting other tissue types. In some aspects, the GalNAc modification comprises a GalNAc moiety comprising a branch point group with linker replacement moiety, one or more tethers, and one or more targeting moieties, wherein n is an integer between 1 and 4 (e.g., 1, 2, 3, or 4), and wherein the linker replacement moiety includes one or more substituted or unsubstituted cycloalkyl (e.g., cyclohexyl, cyclopropyl, cyclobutyl, cyclopentyl, cycloheptyl, cycloocty, etc.), substituted or unsubstituted cycloalkenyl (e.g., cyclohexenyl, cyclobutenyl, cyclopentenyl, cycloheptenyl, cyclooctenyl, cyclohexadienyl, cyclopentadienyl, cycloheptadienyl, cyclooctadienyl, etc.), substituted or unsubstituted aryl (e.g., phenyl, naphthyl, binapthyl, anthracenyl, etc.), substituted or unsubstituted heteroaryl (e.g., pyridyl, pyrimidinyl, pyrrole, imidazole, furan, benzofuran, indole, etc.), or substituted or unsubstituted heterocyclyl (e.g., tetrahydrofuran, tetrahydropyran, piperidine, pyrrolidine, etc.), or any covalently linked combination thereof, is located within the branch point group.

### Methods of Treatment

Disclosed herein, in some aspects, are methods of modulating XDH gene expression in a cell using the oligonucleotide, composition, or pharmaceutical composition described herein to the subject. Also disclosed herein, in some aspects, are methods of modulating XDH gene expression for treating a disease or condition associated with activity or expression of XDH using the oligonucleotide, composition, or pharmaceutical composition described herein to the subject. In the methods disclosed herein, any XDH siRNA known in the art may be used in place of an oligonucleotide, composition, or pharmaceutical composition described herein.

Also disclosed herein, in some aspects, are methods of treating a subject in need thereof by administrating a therapeutic effective amount of the oligonucleotide, composition, or pharmaceutical composition described herein to the subject. Provided herein is a method of treating a disorder associated with Xanthine dehydrogenase (XDH) activity in a subject comprising: providing a pharmaceutical composition comprising a polynucleic acid molecule described herein, and administering the pharmaceutical composition to the subject in a dose and schedule sufficient to modulate the XDH activity in the subject, thereby treating the disorder associated with XDH activity. In some aspects, the method treats the subject by modulating gene expression or the signaling pathway associated with XDH activity or expression in the subject. In some aspects, the method comprises decreasing gene expression by contacting an endogenous nucleic acid (e.g., endogenous mRNA) with the oligonucleotide described herein. In some aspects, the method comprises decreasing the expression of XDH. In some aspects, the administration reduces serum uric acid level in the subject at least by about 20%, about 30%, about 40% about 50%, about 60%, about 70%, or about 80% compared to serum uric acid levels of an untreated subject or the subject before the treatment. In some aspects, methods include administering to a subject in need thereof a therapeutically effective amount of an oligonucleotide that includes an antisense strand that is at least partially complementary to the portion of SEQ ID NOs: 1-50, 51-100, or an antisense strand comprising at least 12, 13, 14, 15, 16, 17, 18 consecutive nucleotides of any one of SEQ ID NOs: 101-150, 151-200, differing by no more than 1, 2, 3, 4 nucleotides. In some aspects, methods include administering to a subject in need thereof a therapeutically effective amount of an oligonucleotide (polynucleic acid molecule) that includes a sense strand comprising at least 13, 14, or 15 contiguous nucleotides differing by no more than 1, 2, or 3 nucleotides from any one of SEQ ID NOs: 21, 71, 267, 477, 1321, 1417, 2021, and 2197. In some aspects, methods include administering to a subject in need thereof a therapeutically effective amount of an oligonucleotide (polynucleic acid molecule) that includes a sense strand comprising a nucleic acid sequence having at least 80%, at least 85%, at least 90%, at least 95%, or 100% identical to any one of SEQ ID NOs: 21, 71, 267, 477, 1321, 1417, 2021, and 2197. In some aspects, methods include administering to a subject in need thereof a therapeutically effective amount of an oligonucleotide (polynucleic acid molecule) that includes an antisense strand comprising a nucleic acid sequence having at least 80%, at least 85%, at least 90%, at least 95%, or 100% identical to any one of SEQ ID NOs: 121, 171, 687, 897, 1581, 1677, 1841, and 1937. In some aspects, methods include administering to a subject in need thereof a therapeutically effective amount of an oligonucleotide (polynucleic acid molecule) that includes an antisense strand comprising at least 13, 14, or 15 contiguous nucleotides differing by no more than 1, 2, or 3 nucleotides from to any one of SEQ ID NOs: 121, 171, 687, 897, 1581, 1677, 1841, and 1937. In some aspects, the therapeutically effective amount of an oligonucleotide is administered to a subject that has failed allopurinol, febuxostat, pegloticase, Lesinurad, or any combination thereof.

In some instances, the subject has serum uric acid (sUA) level between about 4 mg/dl and about 7 mg/dl. In some instances, the subject has serum uric acid (sUA) level of at or over 6 mg/dl, 7 mg/dl, or 8 mg/dl. In some instances, the subject has serum uric acid (sUA) level of at or over 7 mg/dl, or 8 mg/dl when the subject does not receive urate-lowering therapy (e.g., diet modification or administration of pegloticase, Lesinurad, allopurinol, etc.) or after the urate-lowering therapy is washed out (e.g., at least 1 week, at least 10 days, etc.). In some instances, the subject fails to respond to the treatment of urate-lowering therapy (e.g., allopurinol at a stable dose) for at least 1 month, at least 6 weeks or at least 2 months.

In some aspects, the oligonucleotide, composition, or pharmaceutical composition described herein is administered to a subject in need thereof as a first line therapy. In some aspects, the oligonucleotide, composition, or pharmaceutical composition described herein is administered to a subject in need thereof as a second line therapy. In certain embodiments, the oligonucleotide, composition, or pharmaceutical composition described herein is administered as a second line therapy to patients who have failed one or more first line standard of care therapies. In certain embodiments, the oligonucleotide, composition, or pharmaceutical composition described herein is administered as a maintenance therapy following the administration of one or more prior therapies. In certain embodiments, the oligonucleotide, composition, or pharmaceutical composition described herein is administered as a maintenance therapy following the administration of one or more standard of care therapies. In some aspects, the oligonucleotide, composition, or pharmaceutical composition described herein is administered in combination with one or more additional therapies. In some embodiments, the one or more additional therapies is a standard of care therapy. In some aspects, the one or more additional therapies is an oral therapy.

Provided herein are methods for treating gout using the oligonucleotide, composition, or pharmaceutical composition described herein. In some aspects, the gout is uncontrolled gout. In embodiments, the oligonucleotide, composition, or pharmaceutical composition described herein is administered as a second line therapy to patients who have failed allopurinol and/or febuxostat. In some aspects, the oligonucleotide, composition, or pharmaceutical composition described herein is administered prior to KRYSTEXXA. In some aspects, the oligonucleotide, composition, or pharmaceutical composition described herein is administered as a maintenance therapy following the administration of KRYSTEXXA. In the methods disclosed herein, any XDH siRNA known in the art may be used in place of an oligonucleotide, composition, or pharmaceutical composition described herein.

Suitable dose and dosage administrated to a subject is determined by factors including, but no limited to, the particular the oligonucleotide, composition, or pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some aspects, the administration of the oligonucleotide, composition, or pharmaceutical composition described herein to the subject in a dose that is sufficient to inhibit the XDH mRNA or protein expression by at least 50%, at least 60%, at least 70%, at least 80%, at least 90%.

The in vivo drug kinetics, metabolic status, and/or potential toxicity/adverse effect by treatment with the pharmaceutical composition described herein can be measured or indicated by one or more methods or assays. In some aspects, the pharmaceutical composition, the siRNA in the pharmaceutical composition, or its one or more metabolites can be measured by measuring area under the plasma concentration-time curve AUC), maximum Plasma Concentration (Cmax), time to Maximum Plasma Concentration (tmax), Fractional Excreted in Urine (fe), percent Change from Baseline in sUA, or any combination thereof.

The effect of the treatment with the pharmaceutical composition described herein can be measured by one or more methods or assays. In some aspects, the effect or outcome of the treatment can be measured by percentage change from baseline in sUA level, plasma concentrations of the pharmaceutical composition, the siRNA in the pharmaceutical composition, or its one or more metabolites, frequency of treatment-associated gout flares, percent change from baseline in 24 hour urine uric acid levels, percent change from base line in serum xanthine, percent change from baseline in 24-hr urine xanthine, percent change from base line in serum hypoxanthine, and/or percent change from baseline in 24-hr urine hypoxanthine.

In some aspects, once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some aspects, the daily dosage amount of the composition described herein lies within a range of circulating concentrations that include the ED₅₀ with minimal toxicity. In certain embodiments, the daily dosage range and/or the unit dosage amount varies within this range depending upon the dosage form employed and the route of administration utilized.

In some aspects, the disease or condition described herein is an XDH-related disease. In some aspects, the disorder is associated with the increased expression or activity of the XDH gene or protein. In some aspects, the disorder is hyperuricemia, gout, NAFLD, NASH, metabolic disorder, insulin resistance, type 2 diabetes, or a cardiovascular disease. In some aspects, the dose is between about 0.01 mg/kg to 50 mg/kg. In some aspects, the pharmaceutical composition is administered parenterally. In some aspects, the pharmaceutical composition is administered subcutaneously. In some aspects, the pharmaceutical composition is administered intravenously. In some aspects, the pharmaceutical composition is administered intrathecally.

In some aspects, the disease or condition is a disease of the brain. In some aspects, the pharmaceutical composition is administered systemically, which successfully crosses the blood-brain barrier.

### Pharmaceutical Compositions

Described herein, in some aspects, is a pharmaceutical composition comprising at least one oligonucleotide or the composition described herein. Pharmaceutical composition, as used herein, refers to a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated, e.g., an inflammatory disease, fibrostenotic disease, and/or fibrotic disease. In some aspects, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the oligonucleotide, the compositions, the cells contacted with the oligonucleotide or contacted with the composition comprising the oligonucleotide, or a combination thereof.

The pharmaceutical formulations described herein are administered to a subject by appropriate administration routes, including but not limited to, intravenous, intraarterial, parenteral, intramuscular, subcutaneous, or intraperitoneal administration routes. The pharmaceutical formulations described herein include, but are not limited to, aqueous liquid dispersions, self-emulsifying dispersions, solid solutions, liposomal dispersions, immediate release formulations, controlled release formulations, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some aspects, pharmaceutical compositions exist in unsolvated form or in solvated forms with pharmaceutically acceptable solvents such as water, ethanol, and the like. The solvated forms of the pharmaceutical compositions are also considered to be disclosed herein.

In some aspects, pharmaceutical compositions described herein can be prepared as prodrugs. A “prodrug” refers to an agent that is converted into the parent drug in vivo. Prodrugs are often useful because, in some situations, they can be easier to administer than the parent drug. They may, for instance, be bioavailable by oral administration whereas the parent is not. The prodrug may also have improved solubility in pharmaceutical compositions over the parent drug. An example, without limitation, of a prodrug would be a pharmaceutical composition described herein, which is administered as an ester (the “prodrug”) to facilitate transmittal across a cell membrane where water solubility is detrimental to mobility but which then is metabolically hydrolyzed to the carboxylic acid, the active enzyme, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the pharmaceutical composition. In certain embodiments, a prodrug is enzymatically metabolized by one or more steps or processes to the biologically, pharmaceutically or therapeutically active form of the pharmaceutical composition.

Prodrug forms of the pharmaceutical compositions, wherein the prodrug is metabolized in vivo to produce an agent as set forth herein are included within the scope of the claims. Prodrug forms of the herein described pharmaceutical compositions, wherein the prodrug is metabolized in vivo to produce an agent as set forth herein are included within the scope of the claims. In some cases, some of the pharmaceutical compositions described herein can be a prodrug for another derivative or active compound. In some aspects described herein, hydrazones are metabolized in vivo to produce a pharmaceutical composition.

### Kits

Described herein, in some aspects, are kits for using the oligonucleotide, the compositions, or the pharmaceutical compositions described herein. In some aspects, the kits disclosed herein may be used to treat a disease or condition in a subject. In some aspects, the kit comprises an assemblage of materials or components apart from the oligonucleotide, the composition, or the pharmaceutical composition. In some aspects, the kit comprises the components for assaying and selecting for suitable oligonucleotide for treating a disease or a condition. In some aspects, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein (e.g., gout) in a subject. In some aspects, the kit is configured particularly for the purpose of treating mammalian subjects. In some aspects, the kit is configured particularly for the purpose of treating human subjects.

Instructions for use may be included in the kit. In some aspects, the kit comprises instructions for administering the composition to a subject in need thereof. In some aspects, the kit comprises instructions for further engineering the oligonucleotide. In some aspects, the kit comprises instructions thawing or otherwise restoring biological activity of the oligonucleotide, which may have been cryopreserved or lyophilized during storage or transportation. In some aspects, the kit comprises instructions for measuring efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the oligonucleotide, the composition, or the pharmaceutical composition may be in dissolved, dehydrated, or lyophilized form. The components are typically contained in suitable packaging material(s).

### EXAMPLES

These examples are provided for illustrative purposes only and not to limit the scope of the claims provided herein.

### Example 1. Bioinformatic siRNA Library Design Against Human Full Length XDH Transcript

In this section, an exemplary design for identification of potential siRNA sequences for human XDH gene transcript is described. Sequences of all siRNAs that can binds to human XDH mRNA transcript, or a pre-determined region of the human XDH mRNA transcript were collected to generate a starting set of human XDH siRNAs. From the starting set of XDH siRNAs, the first set of 260 siRNA sequences and target sequences were selected. Then, from the first set of 260 siRNA sequences, 50 siRNA sequences and target sequences were selected that were predicted to be effective and/or potent to downregulate the XDH mRNA expression or to induce post-transcriptional degradation of the XDH mRNA expression with low off-target effect.

### Example 2. Screening for the Validation of Candidate siRNAs

### Ex Vivo Efficacy Measures

Cell culture: Human liver sample is perfused with warmed HEPES buffer containing EGTA, collagenase, antibiotics, and antivirals. Following dissociation, cells from the human liver sample are plated onto collagen for confluency at 12×10⁴ viable cells/cm² in FBS-containing media. Afterwards, cells are maintained in defined, long-term grown media.

Efficacy: GalNAc conjugated polynucleotide molecules (e.g., siRNA molecules) (or lipid-mediated transfection reagent complexed with polynucleotide molecules (e.g., siRNA molecules) in antibiotic free media) (in 0.1-30 nM final concentration) are applied to cells and target gene expression are measured 24-72 h later via RT-qPCR and immunoblotting. Dose-response curves are generated for target mRNA and protein expression change as well as impact on both intracellular and intracellular uric acid levels are quantified by light or mass spectrometry. Off target effects are assessed by RNAseq and immunogenicity assessed in freshly isolated human PBMCs via a cytokine protein panel. All measures are compared to a non-targeting control sequence of equivalent GC content.

### In Vivo Efficacy Measures

Animal maintenance condition. Experiments are conducted (±potassium oxonate) with ad libitum food and water access under 12 h light/dark cycle, climate control, and environmental enrichment.

Pharmacokinetics. C57BL/6 mice (6-8 wks old) are administered GalNAc conjugated polynucleotide molecules (e.g., siRNA molecules) (1-5 mg/kg, 1-8 μg/g in isotonic buffer, sc). Cynomolgus monkeys (2-5 kg) are administered GalNAc conjugated polynucleotide molecules (e.g., siRNA molecules) (1-5 mg/kg, 1 mL/Kg in isotonic buffer, sc). Plasma and liver biopsies are harvested at day 7 and 21 post-dose and polynucleotide molecules (e.g., siRNA molecules) are quantified via RT-qPCR using sequence specific primers on antisense cDNA and compared to non-targeting polynucleotide molecules (e.g., siRNA molecules) control of equivalent GC content. Efficacy. Mouse and non-human primate receive GalNAc conjugates (sc) at the dose where polynucleotide molecule (e.g., siRNA molecule) concentration target gene protein levels, and liver T½ are deemed optimal. Liver and plasma are collected from mice on days 7, 21, 60, 90, and 120 and subjected to RT-qPCR and immunoblotting for target expression measures and to spectroscopy for uric acid measures. Liver and plasma samples are collected from primates every 10 days for 100 days post-dose for target gene and protein expression as well as uric acid levels, as above. For both species, aspartate aminotransferase (AST) and alanine aminotransferase (ALT) levels serve a proxy measure for overt toxicity.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1. A polynucleic acid molecule that modulates expression of Xanthine dehydrogenase (XDH) gene, wherein the polynucleic acid molecule comprises a nucleic acid sequence that is at least 90% complementary to the nucleic acid sequence of at least one of SEQ ID NOs: 2-4, 9-11, 14, 18, 22, 25, 28, 30, 31, 34-37, 42, 45, 48-50.

2. The polynucleic acid molecule of claim 1, wherein the polynucleic acid molecule comprises a nucleic acid sequence that is at least 90% complementary to the nucleic acid sequence of at least one of SEQ ID NOs: 2-4, 9-11, 14, 18, 22, 25, 28, 30, 31, 34-37, 42, 45, 48-50.

3. The polynucleic acid molecule of claim 1, wherein the polynucleic acid molecule comprises a nucleic acid sequence that is at least 95%, at least 96%, at least 97%, at least 98%, at least 99%, or 100% complementary to at least 15, 16, 17 contiguous nucleotides of at least one of SEQ ID NOs: 2-4, 9-11, 14, 18, 22, 25, 28, 30, 31, 34-37, 42, 45, 48-50.

4. (canceled)

5. The polynucleic acid molecule of claim 1, wherein the polynucleic acid molecule comprises a nucleic acid sequence that has less than 4 or less than 3 noncomplementary nucleotides with the nucleic acid sequence of at least one of SEQ ID NO: 2-4, 9-11, 14, 18, 22, 25, 28, 30, 31, 34-37, 42, 45, 48-50.

6. (canceled)

7. The polynucleic acid molecule of claim 1, wherein the polynucleic acid molecule is single-stranded.

8. The polynucleic acid molecule of claim 1, wherein the polynucleic acid molecule is double-stranded.

9. The polynucleic acid molecule of claim 8, wherein the polynucleic acid molecule comprises a sense strand and antisense strand.

10. The polynucleic acid molecule of claim 9, wherein the sense strand comprises a nucleic acid sequence that is at least 90%, at least 95% or 100% identical to at least one of the SEQ ID NOs: 2-4, 9-11, 14, 18, 22, 25, 28, 30, 31, 34-37, 42, 45, 48-50.

11. (canceled)

12. (canceled)

13. (canceled)

14. (canceled)

15. (canceled)

16. The polynucleic acid molecule of claim 10, wherein the antisense strand comprises a nucleic acid sequence that is at least 90%, at least 95% identical to one of SEQ ID NOs: 102-104, 109-111, 114, 118, 122, 125, 128, 130, 131, 134-137, 142, 145, 148-150.

17. The polynucleic acid molecule of claim 16, wherein the antisense strand comprises a nucleic acid sequence that is at least 90%, at least 95% identical to one of SEQ ID NOs: 102-104, 109-111, 114, 118, 122, 125, 128, 130, 131, 134-137, 142, 145, 148-150.

18. The polynucleic acid molecule of claim 16, wherein the antisense strand comprises a nucleic acid sequence that is 100% identical to at least 15, 16, or 17 contiguous nucleotides of at least one of SEQ ID NOs: 102-104, 109-111, 114, 118, 122, 125, 128, 130, 131, 134-137, 142, 145, 148-150.

19. (canceled)

20. (canceled)

21. (canceled)

22. (canceled)

23. (canceled)

24. The polynucleic acid molecule of claim 9, wherein the sense strand comprises a nucleic acid sequence that is at least 90%, at least 95% identical to at least one of SEQ ID NOs: 2-4, 9-11, 14, 18, 22, 25, 28, 30, 31, 34-37, 42, 45, and the anti sense strand comprises a nucleic acid sequence that is at least 90%, at least 95% identical to at least one of SEQ ID NOs: 102-104, 109-111, 114, 118, 122, 125, 128, 130, 131, 134-137, 142, 145, 148-150.

25. (canceled)

26. (canceled)

27. (canceled)

28. (canceled)

29. (canceled)

30. The polynucleic acid molecule of claim 1, wherein the polynucleic acid molecule comprises 17-30 nucleotides in length.

31. The polynucleic acid molecule of claim 1, wherein the polynucleic acid molecule comprises 19-23 nucleotides in length.

32. The polynucleic acid molecule of claim 9, wherein each of the sense strand and antisense strand is 17-30 nucleotides in length.

33. The polynucleic acid molecule of claim 9, wherein each of the sense strand and antisense strand is 19-23 nucleotides in length.

34. The polynucleic acid molecule of claim 1, wherein the polynucleic acid molecule comprises at least one 2′-modified nucleoside, at least one modified
internucleotide linkage, or at least one inverted abasic moiety.

35. The polynucleic acid molecule of claim 34, wherein the polynucleic acid molecule comprises from 90% to 100% modification.

36. (canceled)

37. The polynucleic acid molecule of claim 34, wherein the at least one 2′ modified nucleotide: comprises 2′-O-methyl, 2′-O-methoxyethyl (2′-O-MOE), 2′-Oaminopropyl, 2′-deoxy, 2′-deoxy-2′-fluoro, 2′-O-aminopropyl (2′-O-AP), 2′ Odimethylaminoethyl (2′-O-DMAOE), 2′-O-dimethylaminopropyl (2′-O-DMAP), 2′-Odimethylaminoethyloxyethyl (2′-O-DMAEOE), or 2′-O—N-methylacetamido (2′-O-NMA) modified nucleotide.

38. The polynucleic acid molecule of claim 34, wherein the at least one modified internucleotide linkage comprises a phosphorothioate linkage or a phosphorodithioate linkage.

39. (canceled)

40. The polynucleic acid molecule of claim 1, wherein the polynucleic acid molecule is conjugated with a peptide, antibody, lipid, carbohydrates, or a polymer.

41. (canceled)

42. A pharmaceutical composition comprising a polynucleic acid molecule of claim 1 and a pharmaceutically acceptable excipient.

43. The pharmaceutical composition of claim 42, wherein the composition is formulated for parenteral administration.

44. A method of inhibiting Xanthine dehydrogenase (XDH) activity in a cell comprising: contacting a polynucleic acid molecule of claim 1, thereby inhibiting XDH activity in a cell.

45. The method of claim 44, wherein the contacting a polynucleic acid molecule reduces the XDH activity in the cell by at least 30%, 40%, 50%, 60%, 70%, 80%, or 90%.

46. The method of claim 44, wherein the contacting a polynucleic acid molecule reduces XDH mRNA expression level in the cell by at least 30%, 40%, 50%, 60%, 70%, 80%, or 90%.

47. A method of treating a disorder associated with Xanthine dehydrogenase (XDH) activity in a subject comprising:
a) providing a pharmaceutical composition comprising a polynucleic acid molecule of claim 1;
b) administering the pharmaceutical composition to the subject in a dose and schedule sufficient to modulate the XDH activity in the subject, thereby treating the disorder associated with XDH activity.

48. The method of claim 47, wherein the disorder is associated with the increased expression or activity of the XDH gene or protein.

49. The method of claim 47, wherein the disorder comprises hyperuricemia, gout, NAFLD, NASH, metabolic disorder, insulin resistance, type 2 diabetes, or a cardiovascular disease.

50. A method of treating gout in a subject comprising:
a) providing a pharmaceutical composition comprising a polynucleic acid molecule of claim 1;
b) administering the pharmaceutical composition to the subject in a dose and schedule sufficient to modulate the XDH activity in the subject, thereby treating gout.

51. (canceled)

52. (canceled)

53. (canceled)

54. (canceled)

55. The method of claim 47, wherein the administration reduces serum uric acid level in the subject at least by about 20%, about 30%, about 40% about 50%, about 60%, about 70%, or about 80% compared to serum uric acid levels of an untreated subject or the subject before the treatment.

56. (canceled)

57. (canceled)

58. The method of claim 47, wherein the subject failed one or more first line standard of care therapies.

59. The method of claim 58, wherein the subject failed allopurinol or febuxostat treatment.
